SEQUENCE LISTING (327 sequences)

SEQ ID NO: 1 - DNA, 2116 bp, Homo sapiens
  source 1..2116: mol_type = genomic DNA
gaagaagctgccgttgttctgggtactacagcagaagggtatgcggaagcgagcaccccagtctgagatggctcctgccggtgtgagcctgagggccaccatcctctgcctcctggcctgggctggcctggctgcaggtgaccgggtgtacatacaccccttccacctcgtcatccacaatgagagtacctgtgagcagctggcaaaggccaatgccgggaagcccaaagaccccaccttcatacctgctccaattcaggccaagacatcccctgtggatgaaaaggccctacaggaccagctggtgctagtcgctgcaaaacttgacaccgaagacaagttgagggccgcaatggtcgggatgctggccaacttcttgggcttccgtatatatggcatgcacagtgagctatggggcgtggtccatggggccaccgtcctctccccaacggctgtctttggcaccctggcctctctctatctgggagccttggaccacacagctgacaggctacaggcaatcctgggtgttccttggaaggacaagaactgcacctcccggctggatgcgcacaaggtcctgtctgccctgcaggctgtacagggcctgctagtggcccagggcagggctgatagccaggcccagctgctgctgtccacggtggtgggcgtgttcacagccccaggcctgcacctgaagcagccgtttgtgcagggcctggctctctatacccctgtggtcctcccacgctctctggacttcacagaactggatgttgctgctgagaagattgacaggttcatgcaggctgtgacaggatggaagactggctgctccctgatgggagccagtgtggacagcaccctggctttcaacacctacgtccacttccaagggaagatgaagggcttctccctgctggccgagccccaggagttctgggtggacaacagcacctcagtgtctgttcccatgctctctggcatgggcaccttccagcactggagtgacatccaggacaacttctcggtgactcaagtgcccttcactgagagcgcctgcctgctgctgatccagcctcactatgcctctgacctggacaaggtggagggtctcactttccagcaaaactccctcaactggatgaagaaactatctccccggaccatccacctgaccatgccccaactggtgctgcaaggatcttatgacctgcaggacctgctcgcccaggctgagctgcccgccattctgcacaccgagctgaacctgcaaaaattgagcaatgaccgcatcagggtgggggaggtgctgaacagcattttttttgagcttgaagcggatgagagagagcccacagagtctacccaacagcttaacaagcctgaggtcttggaggtgaccctgaaccgcccattcctgtttgctgtgtatgatcaaagcgccactgccctgcacttcctgggccgcgtggccaacccgctgagcacagcatgaggccagggccccagaacacagtgcctggcaaggcctctgcccctggcctttgaggcaaaggccagcagcagataacaaccccggacaaatcagcgatgtgtcacccccagtctcccaccttttcttctaatgagtcgactttgagctggaaagcagccgtttctccttggtctaagtgtgctgcatggagtgagcagtagaagcctgcagcggcacaaatgcacctcccagtttgctgggtttattttagagaatgggggtggggaggcaagaaccagtgtttagcgcgggactactgttccaaaaagaattccaaccgaccagcttgtttgtgaaacaaaaaagtgttcccttttcaagttgagaacaaaaattgggttttaaaattaaagtatacatttttgcattgccttcggtttgtatttagtgtcttgaatgtaagaacatgacctccgtgtagtgtctgtaataccttagttttttccacagatgcttgtgatttttgaacaatacgtgaaagatgcaagcacctgaatttctgtttgaatgcggaaccatagctggttatttctcccttgtgttagtaataaacgtcttgccacaataagcctccaaaaa

SEQ ID NO: 2 - DNA, 43061 bp, Homo sapiens
  source 1..43061: mol_type = genomic DNA
agaaagtagaccctcaccaggcatggaatctgccagtgccttgatcttggacttccagcttccagaactgtgagcaacacatttctgttgtttataagccaccctgtctgtgctgttttgttatagaagcctgaatgaactaagacaaattcccagttttccctggggtgatcttgggcgagttatccatgctgtctgtccctgcttccaagctgtgaatcaggataatcggtgctgcctgctagaggcatggcgggtttgtggaagcaaagccacttgcaaagtctggcacatggaggggatcaggagctgctgattcctccctttcccctatcctctctgcaccccctgccactcccacagatgctgctgaatttcatggtctccttctgagcctgacttgcctcaacttgcaaggcatctcttttgtctctttgtttgcacaatgaaggactttggaactggggtcctctggctgggaccagagtgcagttctggccagtccaaatgattcttggctagagatgatggagctgcccaggagtgtcccattgcccactgccctggcagacggctctgtttcgaagtgattaaggagggcaacctagggagacaggtgtggcaatgtgagggccatccatttctggtgacacctcggcagggaaatgattaccagcccagatctctgggagcatccccttcaaatgccccagcaactgagaaaggaccttgaagtaaatttcagcactgaccagaactgtaaagggtttgctcagcaccagacccactcccagcttcagggatttacgttctcattctcctcccgatacaaacacctctccaggctggggtcttgtccttccacctgggacttatctatgtattggaagaacctgcctttttctttagagtcttagtatagttgctaggtaattggagaattgcatcatcattgtgagtaaaacaaacatgcctcagagtaggaaacacaccaaaaaggtttaacaatcctccagaaccacagctggaattcctcctgccccctaacactctgggactctgagaatgtctgcgggagggagtgcattctcaggatttacagcctcatcattcttcctgcccaatgtctgcccacagagccctgggtccatctttcctgcagagctcatggcaccggacttctccatgactttgtcttagcgtctaatcctgctcccagctcagcccagtgcagggcaggagcacgatgcacatttgctgagtaaacattgaaccaatgcctcaagaaagcagatggcattcttgggtgtcttcatttagttttccaaaccttcacttctcaggatgaacaatagttacagaaactgcagccatgcaatctaaggtctaccaggccttgcacacttcctcacccacactgggaaatgccctgagtggctcagagggaaagggctctgagcttgggcagagaatcatgtgcactgtttttaataagctctgccagcttgtcgctagcctagagatgcaatcagctttgagccaagcgggggccatgcatcatgttaagtgtattttcttctctttagcagagcaatctgctgaagtgatttctacatgaaatgaacctgacctggcatctggggccttcctggcctctcttttctgtgctgttctttctctgtctcctccacactcaggcctgtggcgaactccctttgtttctcttcttcttgttcatcttgaagggcagcaggaggtgaacatagtgcctgggtggctctgggatgccatggggcaaaaccccaaggacaaatgctcagaagcttcacatgggtgcagggagatgacctctaggaccccacccggtccaggccttacagcagggcctgggttttgagggatatgccaaacagcctggatcctgcagagggatctcagccctatgatgggctgagagtgagcccacccggctgcgacatttgtagccccttggcttgcagtgtggaggatggggtgggctggcgaggagggtggagagtggagaggcaggggacctgcacaggtgtccaggtgagaggggctgaggctaactccagatgtaaccagagtgaaggcaggaagggacagattcttctagaagtactgtcagggcgttctgctggggtgcaagggtggaggtgtaaaggtggcgtcctgccttcagacttgagcaaatgggtgatggtggtgacagaaggaggagaggcagacttggaagaaaagttgaacactttgggtatgttaagagtaagacgactgcgtgcaatggctcacacctgtaatcccagcactttgggaggctgagacagatgggtcacctgaagtcaggagttcaagaccagcctggccaacatggtaaaaccttgactctactaaaaatacaaaaaatgagtcaggcgtggtgggggtgcctgtaatcccagctacttgggaggctgaggcaggagaattgcttgaacccgggagtcagaggctgcagtgagccgagatcgagccacggcactccagcctgggcgaggagcgagactccatctcaaaaagaaaaagtaaaacacccccctacttgctgaagggagtgtaaattggtgtctcctttttggagaactatttatgaatacctctcagagctaccaatgcacccactctgatccagctgtttcccttctagaaatttatctttatcctgatgatagagccgcaccccgaaaagtgaaaggtataccagcttgctcatttcatggtggcattgtttacattaaaaaaggttggaaagcacttagtgccctctgttgggggaatggtaaagaaaattatggtccagctaatcatgaagtggaacagtgtacagccataaaacggaacaaggaaactctgcatacactgatgtgggaaggtccctgggatacagtgttcaactgaaaagtcaaagtggccgggtgcagtggctcacgcctgtaatcccagcactttgggaggctaaggcaggcggatcatgaggtcaggagttcaagaccagccttgccaacatagtgaaaccctgtctctattagaaatacagaaaattagccaggcatagtggtgggcacctgtaatcccagctacttgggaggcagaggtaggagaattgcttgaacccgggaggtggaggttgcagtgagccgagatggtgctactgcactccagcctgggtgacagtgcgagactctgtctcaaaaagaaaaagaaaaaagaaaattcaaagtgcagaatggcatgtagagtatgccagcatttgtggagaaagaaggcaaaaaatatttatttatatctgcataaaaatttctcggaaactacacaaagatctactcataatagtggttctgttttctgggggtaggtggagtagatgggggtagaggaatgtggggagacttttctatatgccttttatatttttttagctttaaccctatagatttacctggttgaattttttaaaattttattttagagtcggagtcttgctctgtcactcaggctggagtgcagtgacatgatcatagctcactgaaggcttgaactcctgggctcaagagatcctcctacctcagcctccccagtggctgggactacaggtgtacaccacaacactagccttaactgttgtcgacttttaaaagcctagtttgaaacttaaagcccacaatgatgttttggcctaggtgggctctgccttccctcccccgtgtatctctcctcgcaagatgtctgtacatttgcaaaggagcttatctgtcctgagggccacccccatttcctagaagatgagaactagatcttcacccagaccttgcttccctatccatcccctcccaaggtcattggtaagcctgacatcaggtgagggtctccctttcttcatccagggacttgtccatccctggaacactcctagtttcacccctgtcctagtgggcactcattcatgaaccataacaaatcttttcttcagtgggaatgagccgtgctcaggcatcacaccatagggtaagagccatctcctgaaaggcaacagagcacccagcccaaatctcccccacactcgctgctccacagggtgatgccagtctttggcctcagctgtctgactcaagagatcccagcttgtaggcgcataggctcgtggacgacctggtggttcccctggtttcccagggctgcacaagctgcctatagcttctctcagctctggccttgctttctggcttgattgtggaatatagtttcctccaaggcctaatctccctaagtaagctctagatcactgcccctatggcctaaggtgacaccgttgaaattgtacttccgtccctgagaagtagtttcactccttaaacattcattgttgaagaatgataaatatacctgtctgccttctgctaactctctgggatttctttccctggagtttgacctcagcccaagacgatttcctttctccttctcatctaggagctggaggttgatgctatgcacagatcttaggatgacttgaatcacaggtgcagcaggataggaatttactctccgcagtttagggcccaaaaaaatcctaatttttgagacagagtctcctctgttgccaggctagagtgcagtggtgcgatcttggctcaccgtaacccacctcccgggttcaagctattctcctgcctcagcctcccaagtagctaggactacaggcgtgtgccaccacatccagctaatttttgtatttttagtagagatggggtttcaccatgttggccaggatggtcttgatctcttgacctcatgatccactcacctcagcctcccaaagtgctgggattacaggcgtgagccaccgcatctggccaaaaatcctaattaaaaaaaatattatctcttcttgccctctggagtccccagttaccacctcgcaccaaaacttgtatttagattttgttgtgtgacactcttattcattttaaaatggtctctcttactctatctctcttcttctgtctctgtaaactagcagttacttcaaagttcaaatatcctgtaaggtttgtcagaggtgtttgagccagaatgtctccaacttgaataggggttgggtaaaataaggctgagacctcctggctgctttcccaggaggttaggcgttcttagtcacaggatgacataggaggtcggcaggtcacaaagatacaggtcacaaagaacccactgataaataaaacaagatgcagtaaagaagctgccaaaacccactaaaaccaagatggcgatgaaagtgacctctgttcgtcctcactgctcattatacactaattataatacattagcatactaaaagacactcccacctgcatcatgacagtttacaaatgccatggcaatgtcaggaagtcatcctctatagtctaaaaaggagaggaagccttgtttcagggaagcctctgcccctttcctggtaaactcatgaataatccaccctttgtttagcatatgaccaagaaataaccctaagtatactcagttgagcagcccatactgctgctctacctacggagtggccattctttctttacgtctttataaacttgctttcgatctatagactcgtcctgaattctttcttgcgtgaggtccaagaaccctctcctggggtctggatcaggacccctttccagtaacaggtccacttttccccaaataagtgatcttcctcctccttcctccttctgatagtggcatctaggtcagggtgaatcttaccaaacctgccactggagcagggacctctggctctctgggatcttcttggttgaggtgtctctgggaaccagcttctttccttacggtatttgctgtggaggggtcacagctagcataacttgtgtttcattttcctatcgggggcaatgcaccgtaattaccccggaagctcagcctggctccaacatttcccgttaatgatgagccctccacctggtttctctgtgcttgccgaacctcagaaactcatttgctcttctcagtggtccccagctttgggctctcagctctgcagactctgccacaccatctcatgccctgctcagggcaacaccctggtggacaaggtgactcttaattcccagccaccccccgccccatagtggaaattgtgattggacatttattattattattttattttagagacactgctggagtgcagtggcatgatcatagctcactgtggcttccaactcctgggctccagccatccttctgcctcagcctcctgagcagctgggactatagtcatacccagctaattaaaaaaaaaaaaaaacctgtagaggagggagtctcagtatgttgcccacactggtcttgaactcctagcttcaaatgattctcctgccttggcctcccaaagtgctggggttacagatgtgagccactgcacctggccttattatgtttaattggagggaacattgataaagtctgttacatagtagcaagagtatccctaacatgccttaccactgagaaataagattctttgctgctggaggtgtgtgaagtttttctatagcagaaggagatgtaatttttttaaaagtcctttgactacagagatttttatattgtctgtgtacaatattactgggtttattaaaataatttaatttaaaatatctcatttgatttatcacctacatctacagttgtgtatttagttatgggacatacatctaattgaattcccatctttttgtggtaaaggtttattttcagcatctctttatgcatctcatcctttcccactctccattttctctttttaatgcaccattttctgattcttataaatgacgtatcctccccctttggcaaggtagcattagccagcaagcatacctaatatttgtgtggatggaaggctcaggaagagaactatttgtctctttcgagcttatggctttcacaaagtgtcatagcaatttttatgactcaactttcaaaagagatttattagcttgggtctctaggaaaaaataatttataaggccactgacaatcatagctgtgaagacataaagtaacatgataagcacttatttttatagcactttttacatatgagactaccatggtatgaagttcaattatgatgactgatttataaacatcatttcttgtttaacgtgggtaatttttaaaagtatgaactggtgagaatgtcaaatggtgcagccaatttggaaaagtgtgacaggtcctcaaacaactagacatagagttactgtaggagccagcagtttcactcctgggtatctgtcaagagaactgaaaacagatgtccacacaaaacttattcatgaatgcttacaacagccttattcataacagccagaaggtgcaagcaactaattgtccatcagctgatgaatggataaataatgtgtggtatatccacgtagtggaacattattcagccataaaaaggaatgaagtactaatgctacaccttggatgaaccttgaaaacattacgctaactgaaagaagcaagtcacaaaggacaacagactatggaaatctatggagacaggaagtcattagtggttgcttagggctggggatggggtgaagtgggaggggtgggggtgatggctaaaggctttctttttgagatgatgaaatcttctaaaattgactgtggtggtggttgcacaactctaaatatgctaaaaatcattgaattgtacactttaaataggtgaattgtatgtacagtatctgaattacatctcaaagctatcttaaaaagataaataagctctgtgcctcattttgcctcctacagtgtttcgtaaagttgtagtactaaaagggagatttgatagcaataatgataataataataaacaggctgggcgcagtggctcaagcctgtaatctcagcactttgggaggctgaggcaggtggatcacctcaggtcaggagttcaagaccagcctggccaacatggtaaaaccccatctctactaaaaatacaaaaattagccaggtgtggtggtgggcacctgtattctcagctactcaggaagctggggccagggaatcgcttgaacctgggaggcagaggttgcagtgagctgagatcacatcatggcactccagtcttggtgacaaaagtgaaactccatctcaaaataataataataataataaacaatttctaagtattattcttagtagtcacctacgcatggcactttgtgcagatgactcctgcatgtgtcagggtttttcttgcgggcaatgcagtctgatcagtataaactaggcagagaggtatttgatgaagggtttggggaactcagaaaatggactgggaagctaagaaccaggcatggaaatgagcaagaactaggggagattggggagcaggagcagaaatggtcttgcaccagggtcagaaatgcctgtggttgaaagtcagctgtcttcagcgttcaaacctctgtgctccagattccagctccagggtggttggcttagtgggtacctgcctctcagcccaaggagaacagagctttcgattcacagagcttccgattcacagaatttccagcctccactcaagaacaacagggatccaggtgctgaaaagggatagatgcctcccacagactccagggaagcattcagttgtgaccacctctacccttttaagggatcgaagcccagcctagcaggaaactgagctttggggctttgaaattttgccccaaagtgctgctagaagaattggattagataggataccaccatgtcactgactacttagggggaaaagaaagctgaggtagtgaaaacatttaatgaaatcctgaacacccagagtaacctatttcttccttgctccgttcccatttatatctgagaactgtgttcatgccccagatgttgcaagaatactgcggaaacaaaagtgttcgaaataattcttttttttcgagatggagtctcgctctgtcacccaggctggagtgcagtggagtgatctcaactcactgcaagctctgcctcctgggttcacgccattctcctgcctcagcctcctgagtagctgggactacaggtgcccgccaccatgcccggctttttgtatttttagtagagatggggtttcaccgtgttagccaggatggtctcaatctcccgacctcatgatctgcccacctcagcctcccaaagtgctgggattacaggtgtgagccaccgcgcccagctgaaataattcttatgctcaggatcaaaatgataaggggtcaattgtattaaacacacacacacacacacacacacaaactttgccttaaacataaatgtgtttcagaagcttggaagcagaaaataattttaggagctgagtattattaatcagcctatgtgttttcaactgcaccaagaaatatagcaaagatatgcaaactctttggatttaagacatcacctttgtgttttgacactctttcccaaaccccgcctaaatgacaggtgggactgttttcttttaaacagtaaaggcagggtcctgagaagacacttgaatggggcaattggaggagtgtttaatcgatgaactatttacaaagatgtgggcaggaagaccatgatagactgagcagttggggggctggaagcagcagggtacccctagaatctacagctctgagggggtgaaggagaagcagtttcaggaaggagggcgggatgggagctgatgggggaggatgcagtcacctgaggccaacctaaggggaaggagaagctgtggagtggaagccagggaaggaagggagccctgtgacaccatctgtgtaatcagcctcccaggaggtggagcagggtgacaagtgcactccgagggaggggccaccaggtgagtcagcccagggcccagctgctaagaccaggggcaggcctcctccaagtggggttgggcaggggggcaaaggagaacaaaaaagccactttacctacttctgtattgagtttttaaaaaagactgtattaatttacattgtaattttaaaataaaaatgtaaaaatattaaacatgactcggaaatcattgagtttgaggatctcatgagaagttgatgacccttcttgaggtaactaacgacactttggaaggtgaaaccaggttgctcgcccactctaagaaggagcgagaaggaagaaggagaaacagcactgtgcccccttctcccgctgcagtcggggcgagatttccatgctctgggaggtagtggaaggaagcgttcagtgaggcccttaaagacaatggccctgccagccagttgtgatgttgacaatgttctcctgcaggagggcgggaggcctataaagtaccaagcgcagtcctccatgagcaacggggaggcgatgagcgaccctctcctccagggatagacacagaccctctcctccagggataaacacagccagttatggccgggcgcgatggctcacatctgtaatcccagcactttgggaggccaaggtgggcagatcacgaggtcaggagatcgagaccatcctggccaacatggtgaaaccctgtctctactaaacatacaaaaaaaaaagttagccaggcgtggtggcaggcgcctgtagtcccagctacttgggaggctgaggcaggagagtggcatgaacccgggaggtggagcttgcagtgagccgagattgcaccactgcactccagcctgggcgacagagcgagactccatctcaaaaaaaagaaaaacaaaaaaaccagccagttatatttaccagctattaattaaatttttcactggaagcagtcttgccttgtacatagggatgaaacacacacacacacacacacatacacacaccatatatatatttataaatgcatatataaaaattatggtaaaatacatgtaacataaaatttacaaccttaaccatttctaagtgctcagttcattggcatcaagtatatttgcattgttgtggactcatcacaccatccatctccacaattctttttatcttccccaactgaaattctgtgcccattaagcaaaatctccccattctcccctcccccagcccctggaaaccaccattctattttccgtctctatgaatctgactattctaagaacctcgtaaaagtggaatcatatagcatttgtcttgttgtgactggtttatgttacttagcataatgtcttcaaggtccatccatgatgtagcatatgtcagaatgtcctttctattttgcttgaataatattccatcgtatggatatgtcatattttgtctttctatttatctgttggtggacacttgagttgcattcaccttttgtaaatataacacatacacaatgacacacaaactccagtatgtgccagggacccgcacttgttaaagggtcatctataaaagagaaagagaaaatacatgtgccaacctaagtgagaagatgagaagcttcaagaagcttgagttgggcttctgtgcatcacaaaggtggagcctggactgtcatctgggcccggcctcttgggtagtggtccctgaatgactggcagaacaagaacatgaaatggtctccacgcagaagacagccacgtggaagtggctgactggtagaccagctgagatgaagactactccagtcttcacagcggtgctcaaatggccccatcaaatcaaatgtaatgaccaggcagagactagggtgaaaacagttgtggaaatccatttgtatgtctcctgtaatatatgattggattagctcatcccaactcaatgaacttcattgccaagttctccagaggaaaagtctgtccttctagtgccttctgttttcacacaataagcaaggatgacattttggcgtgacttgttaaattgtagcaaatgagacaactgtcagggtgtagacaaaggcaaacaaatcacttagtgatttgtcgagtgaaaatcgacctcaactaaaggcgctcactggagtctccttgtggcatttctcatgggcacttgctttcataagcatcgccctttgatgttattttgctaggagaatgtggaaaagtgtatttggaaacactgagtttcagagctgatagtaatgctgtatcatttcaatcagaaaggaattttgcaaaaaggaactgacttttttgtttgtttgtttttgagacggagtttagatcttgttgcccacgctggagtgcaatggcgcaatctcggctcactgcaacctctgcctcccaggtttaagtgattctcctgcctcagcctcctgagtagctgggattacaggcatgcgccaccatgcctggctaattttttgtatctttagtagagatgaggtttctccatgttggttgggctgatcttgaacgcccgacctcagatgatccacctgccttggcctcccaaagtgctgggattacaggtgtgagccaccgagaccagcagggactgacttttaacagatcagttatttaagcttgtcctctgtgcctgggatggttttaggtacctgtctgcattcactgtgttaatacagtatagtcctcaaataccgttggtccttgaacaacacagatctgaactatgtggggccacttcttggattttcttccacctctgccacccttgagacagcaagaccaattccttctcttcctcctaagcctactcaacctgaagatgataagaatgaaggccttgatgatgatccacttccatttaatggagagtaaatatgttttctctttgttacgattttcttagtaacattttattttctgtagcttactttgtgcacagtatataatatacacagtatttaatacctacgacataaaaatatgtgttaatcaacattagattatcagtaaagtttctgatcaacagtgggctattaggagttgagtttggggagagtcaaaagttattcttgcattttcaactgcgaaggaagtcagtgcctctaaccccagtgctgttcaaggatcaactgtagttcaccaagaaagctggggttgatcccattccagccctaccctgctcttggtgggcacagagagttgtgagacctacctctggccagacactgttctatgtgtggaaatattgcaatgaagaagatcaaccctccaaatgctcactgtggccaggctcggtggctcatgcgtatcattccaactctttgggaggccatggtggaatgattgcttgaggcccagaatttgagaccagcctgagcaatatagcaaaactctgtctctacaaacaaacaaataaacaaacaaacaacaaaaaataatgctttctgtacataaaccagcacataagtaatataatttcatgggatgatagtcactatagagaagataggggtggtgcagtggaataaggtgagatagggttggtgggcagggctcttctctgggggtcatcagacgggtgaaatttgagctgagatatgagtgatgaggagccgctgcacaaagatgctggatagagggaacagcaagatttgtgccttaggaagctttgagtttctgtgggttttgttgttgttgttgttgttattattttttttgagatggagtctcactctgtcacccagtttagagtgcagtggcatgatcttggttcgttgcaacctccgcctcccaggctcaagagattctccttcctcagcctcctgagtaactgggattacaggtgtgtgccaccatgcctggctaattttttgtatttttagtagagacggggtttcaccatgttggccagactggtcttgagctcctaacctcaagtgatccacccaccttggcctcccaaagtgttgggattacagacgtgagccatcgtgtctggccaagtttctgtgttaatggacacagaagaagtcagcacagctggaaggcagaggacaaggaggtgcatggtccaaggctgtagagagaagggcagcagccaggtcgtgtaagtgactgcacacggcaaggattcaggagtttcctctcattgcaatgggcagccttggggagttttcctctgggaggaaatctcatggatttatactgaagaaataactggctatgtgctgtgtgataacagatggaagggggctggaggggaagcagagaatcgggttgggaggcctgtgggaccgctagaagccacccctgcactaggtaaacagtgtggacctgaaagggacaggctgaagcacgaagtgtgatttacaaatattactcgagccagaataaagacagctgcctggaagcctcagacacaagtgaccttggatatgaactccatttggctttcgtcacaaggtttttaaaggaacaaaaagggaggagtcagggagtggcctgatacaaagttctgtcaggagttctcattggtttccagaaataacattgattagtgataggctatacattattaagctatagggtgtggtttacagtgtccagtgaggcattattaggttaattcataactactcttggcaatagcaagcagtttcagtggatgaatacatagctcaaaaggggcatgtaggatgcgattgatgtctaattttaatgcctatctgggcctaataatttgaaaaggctcacatccctcagataaaagttattttctttcttcaacagctgtgggcactgttggagaagtataatggaaaacaaaatctcccaacccagaaatcctctctacaaaggtagcaaagaaagaaaacactctcattattgagaaaaacattaaaccagaaagtgttacacaccacaagcaatctgctaagagacagccaagacagaaaggaagctcacctgcttatggagccaagcagacacagctcgttccacacacgttttcaagacggacagtaactgttaccaactgagtcagcttttcccctaaagatggtatcgtgaagccaatacatgaaacccaaagcgagcctcaagcagtgcggactttattcaatggccaaagaatggagaagcaggagcatggccgcaagtcaacttcttgtctagtgagaggtgagggggttaaaatatagggtatcctggctgggtaccctgcctcacgcctgaaatcccagcagtttgggaagccgaggtggacggatcacttgagatcaggagtttgaggctagcctggccaacatggcaaaaacccatctctactaaaaatataaaaaacaaaacaaaacaaaaaaatagccgggcatggtagctcacacctgtaatcccagcactgtgggaggctgaggcaggtggatcacctgaggtcaggagtttgagaacagcctggccaacaaggtgaaaccctgtctctactaaaaatacaaaaattagccgggcggatcacaaggtcaggagcctcctgagtagctgggactataggcgtgcaccaccacgcctggctaatcttttgtatttttagtagagatgggttttcaccgtgttagccaggatggtttcgatctcctgacctcgtgatccacctgcctcggcctcccaaagtgctgggattacaggcgtgagccaccgtgcccaggccgccttcctgtttcttaaagataagatagagaagacaggttagagaagagtagaaattcagctatgtcatgtaggcattacactgggtgacataagtagtaagagaacttgacaacactacttgtcacacacagctcattctaactttacctggcaactgaggtgaccatctgtattagctaatgggctttatctagagaaaaacaagcttcttacatctttgtgacaggaggcagttttgcagcttggaggcatgtgcccactgaaggcaggctgtcaccctcccacagacactgggagctggggccactgtctctcccttgatgtttgcctttcaaggagatggctcccgggtccttgagaaacacatgcctgggtaatcaagctgacaaaagtcctctctgatcttcacaaaaatgttatatacatttgaaagagaggagaaagtgcttataattacaagttttctaaggtaaatgctctgagaataagagagggagggaaacctcttcccccattttcaaccgggagaattaagcctcttgtttttaagttgcatttgtccttacagtggctactaggtgttgggaacactgcccgggagctaggaagagcaattagggctggaggctgtttggcgccaacaggctgttgctgagtgcggatgaaagtccagagagagagtggaaggtgggggtcaaagggcatggtggtgccctgcatggcagatggtggagaaactgcaccaggcaggtgcctcaggctttctgggtcaggggagagggtcaggtggcctttgaagttctgtttgctgttagcctgactgagttaactgcatgggtacctgactgtctttgctcagtctcctagtggacaaccacatttccggaaggcttttcacgagtgatatagcaactaatgctctcccagggctcactgcctggcaggctctgctccaaatgctttcatatgttaactaattcaatcctcactatgaggattctctactcccatgagacagggattgcagggagtataaggttgtgttggatgtgtttcctggcattagtaaaagaggatgtgaggcaagagcaaaactaatgcatgtgctgaaagagctatggtgcaaagagtacacacaaaccttttctccccttctctgtatgctgctgtggacactgttacaaagcatgaaaatgaggccgtagatgcaaaagcttaaaagggtgacccatacctcaaaagttctcagtaagtggcagcattttattatcgtggctatcattttgccagttatatcttaacaggtataaatattgaaacctccttagcgaaaattataacggtgagcaaattatgacagcggaggagatctgacctaaccatctccttcttgccttttaacctctaaactgtcctgggtcattttggggcatgggtcaagctaactttaggagaaatttagtttatagtttaaatgatgatagaccttcccaaaactaagtcatctttgtaaaactaatgaaaggccaccaggtcaaaagaatgagaggggcctgaattctgctgagatgtagacatagttaagcaatcaccagccgttattctggaggtcatgaaatttgcaactttctcaattactcctataattaacatcactactctagaccctaaaattggccttttcagattccttttcagacttttgtatttctgaaaccaggtgactttacctggacccacgacccctacctgtggtccctgacctagaagcaggctcagcatatgaggaccattttttccacacccctatgattgcatctccaaccaatcagcagcatctgttctctagccacctgcctaccaaactatcttttaaaaacccctgccttcatatttttgaggaggctgatctgagtaataataaaactctggtgtcctgttcaggtggctctatgtgtataaaactctttctctattgcaactcccctgtcttgataaatcagctctatctgggcagtggggaagatgagctaattgggtggttacaatattacctcttcaatgtgactgaggttcttgaaggcaggaaattgtcatataatttatgtctcccatgtcagtaccttatatagggtaggtatcaataattactcataaaagatgagaattattggttccctagaagtcattttcccattctaggcatggtgccttttaggggacataactctgactgaactttatatgtcctccttagttagttacttagggtcttgatttcttctggaggctgtatttgtcatttaaattttgctagatattatgcatattacttaagttttcacacttaatggcataaatctgtccctagtattccctctcaattttctaatctcctctgtaactgcagtctatttatatgtcctcagtaaatatttgttgagtctgatagtttttggttatgatcatggactagtgcagtgagggcagttggatattttcctcctgggaggacatctgggattcacaatctggatgtttggtatcatgagacaatctccctgccctggcttggggggccacccatgggttaaggcataagtaacaattcctgacaagagtggggtcattaatgtctggagcaggaagctgaggggagttgtgtttttctcttgcctcaacattgtaggaggagagagaatcctttctaggcatcagggtcatccctgagcactcctggtgtgattttagggttctccaaggcccactgtggttctaatttcctgccaccatccctgaacatcccaccattcatatcctcagtgtagaaaacagagatggtaactttgactccagaaggtcaccattcacatgaccacttaggagaaaaggagaagggagaacttccagtattttatccaaattctttattcgtcactcagctagcccagacgtcttcttaggaagcattggaagatatgagagagtggtgtggggtgggaagcaaatggttggggagacatagcatctggcttttttgggaagccctgctgggtttagacaaatctgcagacaggaggaagaatgagatgaatggcacttaagtgttattcctctccagatcatgattctcccagtttgaattaagactcttctctgagttttggaggagagatgcttggaaaggatgattgaagagattcattcacccattagactccctactattgattaaggatgtaatgaagtgatataagtcatccaataagctatgcttactatgtttttttttaaacatagtaaatgcaagccaaatagagccctctcccaccttagccagtagagtgacatcttaggatcttaggtgaagtcttttttttttggttcttttagtttttaaatttggaggattaggatgacaaaaatgacacctatgggtgaaggggtatggaaggactggaatgacattcagacttttttttcagtaggtttgctggtttaaaaactgtatctgaggacaaaatgcttggattcaaatcttggctccctaactaactgagatctgtcttctctgttctttgtcatcttcatcaataaaatgggggaaaataacactgtctatgtcttagggagaataagatggaacattgcatataaagtgtctaacacaagtcctggcacatagtcatcaataacttgtttattattgtgcttgttaaattccattcacctggaagatccagggatccattcaaagttggctctggctgccgtgatgccaagtgaagtcattccaattttgcagtctaaatagcacacactgccttgtccctaagagaaataccagccccagacattgtgaattgatcatttccactgaaccatttataaggcagagttgatccttatcattaaggtgagcaaagcagttgttgtgggcacttctcttcatgatgatgttcagtcatttaggtctctgtggaatggctggcaggaggccctaatggtgacagaaatcccagtgactactgtcctactggctctggttgggagcctttcctagcagtcctaccatgactggcctcttggatccactctgaattcttctacttctctttgcagtagaaaggcattaactgatatttctctttccaaatttctttgcaatgttttctgtagaagaactgaacattaaataaatattatttctagaactcagttttcgcaaaagatagcatttcagtcccacaagagcaatatttatatcattgcaactatggggcttcggtgggaaaaggatgacaatatttttccatttggatagctgtttcttatctaaacttattaaatggtgagcagaactcttcattgtagacttttcccaaccctgagtgccaaagcaaacacacaaagttcggaccactgacatgttgctgcagtagtataagggcaaataagtctgaatggagataacagacttggaaacggtactttgtgtctctactttcatggtccttccctctactgacatggaaatagcaccctaggcctgcccggggccatccctgaactagggatgcatcccccgtcaaaacaaactgaggacattggataattcacattttacataacagtattttactcagatttatgtggctgctgatgaattttcatcaaaaaagtattttctggaataaaatccattaaaccaatttaatcccttaatcaatttttatgtagtgtagttaaatcatttttatttttctaaaagttaatgaaacattttctatttttatttctgaaaagtaattttcatcaggatagattcttttccatttaaagcttataataaagagctaattaatcaattgatttttgtccccatcagattttcctgagtatagcttctgctactgtgagttttcttttatggccagatccaaagtttccttttgttttatttttcccctgctgtagtcaagtccaatgtctcaggctcaaattctgcacatctaaattttgccagcaatattctgtttgtttgtttgtttgtgacaatgtcttactctgttacccggactgaagtatagtggcatgatcatggctcactgcagctttgacctcctgggctcaggtgatcttcccaccccagcctcctgaggaagctgggactacaggcacgtgccaccaaacctagataattctttgtattttttgtatagatggggttttgccatgttgctgaagctggtctcaaactcctgggctcaagtgtttcacccacctcagcctcccaaagtgctggtattacaggtgtgagccactgtgcctagccagcaatattctttttaatcagttttcgattggtgaaaaagtgactatttctacctataatattcattatggattggataaaacatattgtctaattattttggtgtcattatattcagaattcccagtatagtcatcccttcatacaggaatggaataataaaattatacattaaataagtgttgtaaatacagtagatgtttctaaagctaatttttaaatgaaaggacaatatataagtctatagctaactcctctgtcatgcacaatctgagttaacagaatagattaggggttagcaagctttttctataaagggccatgtaataaatatttcaggctttgcagttcatatgttctctgttgcaacaactcaactgtgctgttgtagcacaaaagcatcctggactattcataaactaatgagcatggctgtgttgcaataaaacttgtttacacaaacagatggtggtccagcccagatttggcccatgggctgtagtttgccaatccttggaacagataaatgttcatgctattatttgtttagtaagagtattctttagttgccaggttgaccatgaactaccataaatagagagctcctggaatgtacattggaaaagtttttctttttgtccagtttttcaggatcacagctgttatacaaagctctgtgtaccaacaatgcatatatcaccaggctcatatacaattttcttagaggagctcaataccctgtggacttaaattaagaaccataaaactgcttgagagtggacaaagagaagataaccatttttcctatcttactgaaaatagaagcagaacaatcactcggactcacatcataggactaattatggatggagcatattagatgataaaataaaaactaacaggaaaggaaagcatgacactagctttggtattcagtcatggcttagagttactagcctcatctgggtatagcagacaagctctggccttaggaaaatattatactctacactttgatgactcccctaaaaacaaaacaactaaaacaattgtaaaaagcaaagcctattacaaagagggagccaaaattcaacatgaaatatactgtgcttaatagaattgaattcttgaataatgttcttctggcctccaagtgagaataaaggttgtgctatttaagaatgtttttcagctgcacataactgcctgtattagtccattttcacactgctctaaataaatacctgagacttggtaatttataagggaaagaggtttaattggctcacagttctgcatggctggggaggcctcaggaaacttacaatgatggtggaaggggaaggaggcaccatctttgtaaggctggagaagagagaaaagtgtgtgtgaaggaggaactgtcaagcacttataaagccatcagatctcatgcaaactcactctcatgagaatagcataggggaaatcgtccccatgatccagtcacctccctctcttgacacatggggattacaggtgcctcccttgacaggtggggattacaattcgagatgaaatttgggtggggacacagagctaaaccgtatcattctccaccggcccttcccaaatctcatgccttttcagatttcaaaaccaatcatgccttcccaacagtcccccagaggcttaattcattccagcattaacccaaaagtccaagtccaaagtctcatctgagacaaggcaagacccttctgcctaggagcctgtaaaatcaaaagcaagttagttacttccaagatacagtgggggtacaggcagttggataaattggccaaaacagaagggctacaggccccatgcaagtccaaaattcagtggagcagtcattcaatcttaaagctccaaaatgattgcctttgactccatgtctcacatccaggtcatgctgatgcaagcagcaaggtcccatggccttgggcagctccacccctgtggttttgcagggcacagcccccttcctggctgctttcacaggctggcattgagtgtctgtggcttttccaggtgcatggtgcaagccaggtggatctaccattctggggtctggaggatggtggccgtcttctcacagttccactaggcagtgccccaatggcaactctgtgtgagggctgcaaccccacatttcccttccacattgccctagcagaagttcttcatgggatctccacccctgcagtagacttctgcctggacatccaggcatttccatacatactctgaaatctaggttcccaaacctcaattcttgtctcctgtgcacccacaggcccaacaccatgtggaagctgccaaggcttggggcttgcaccctctgaagcaatggcctgagctgtaccttggccccttttagccatggcttaagctggaggggttggaacgtaggacaccaagtccagaggctgcacagagcaggcgggccctaggcctgcccacaaaaccatttttccctgttaggcctccaggcctgtgatgggagggactgtggtgaaggtctctgacatgccctggagccattttcctcattgtcttggtgattaacatttggcttctcattacttatgcaaatttgtgcagctggcttgaatttctcccaagaaaatggttcttttattttctaccccatcatcaggatgcaaatattcaaaacttttatgctttgcttcctttttaaacataagttcctatttcagatcatttctctcaagttcaaagttccccagatcctaaaggcaggggtaaaatgctgccagtttctttgctaaagcatagcaagagtgacatttgctccagttcccaataagttccttatttccatctgagaccacttcagactgaacttcattgtcaacatctctatcaacattttggtcaaaaccattcaacaagtctctaggaagttctaaactttcccacatcttcctgtcttctgagccctccaaactgttccaacctctgcctgctacccagttcaaaagttacttccacattttttgatatctttacagcagtgtcccactctctgcagtactaatttacttgcctaaaaatgctgcaaatagtactcacacaatagtgcagagataggcaatcatttgtactagtttaaataactctatatgccaagcaaaaacccaggtgttttctatcattctttgcttgctggcttttggtccttacacttgttgcctcatggttgcaaaatggctgttttgtctccagacattacatcaacatttgaaggaggaagaagggttagtgtctgagccactgactctgctgataaaaggacagagaaagctctcccaaaagccctcaacagatttctgtttgtctcattggtcagaactatcttacttgaccactaatggttacagaggagagcagtgatgcaggatttttctcggtccctttgccagtcagggacctcttgctggtgatgctggaggccttgcttggggcacactacctgtccctggaggtgcctcacccaatcggcccccctgggccaggtctggcttgtataccagttcctgagttcttgtcccacgcccagaatgaatgaggctgtgctgaactctgaagggtgagggtgatggagaagaatttttttgaccaatgaaacagctttcagtggggaggggatgcaggggtggtctccctaccagaaggtgggaaagtcccccatgtggctgggtctggggctttttatggactcagaatggggagtatgtgttgattggtttatgagtatgcaaaaaaggttaaagcaaagactctactcaaaggtgggcatgacagtgtagaaaatcagttaggaaaggacaggtatatgtaaaataggtgaagggtggggatcaatcagaggaaagcacaccaaacaggaagacaagttctcaatctggtccaaggatttaagttgtagcttggctttcaggctttaaactgtcttcagcttggaggcgggatttcaccggggacccgccccatctgcctaggcatttggctgcctcctcctgcccttagtaggaaagtgagtgtttcattttctcaatttatctgtggatgtggacaaaggggaaaggggtagggaatagggggttgtagtcagcctccaacagattattaccactgaaattgtgccctaaaggattaaagaagtcagtaactaacagaaattcttgagtttgcaaggtaagaaacaacttgctcaaatgctgaaactccctctgcttgtgagataaaactagcagaaatcagttggaaccaccatggccaactggagtttgcacacagtgagcttggtgaagtcacggcccgaatttctaccacgtgtttcatattaaccccttgaaattttctcatgggacccctgaggcaacatgaagagataactgtgcaaacctgaggattttctaggcctcccctttcctttcactaatcacctacaatcccaatatccaccccctaaaccttttctaataaaattatgccctgccttaaagccatctcagggagacaggtttgagcttgacccctgttgcaatagaaagcttttcttttgtcaaaaacccagagtcatagcactggcttctagtgcatcaggcagcaatcactttttgcttgataacaccatttttcataagtacctgaaactggattcactaattatgtcagctaatgttgacattgatagaggtaaacgactcatttcttatttataaatctgcaactgttgacaagaaaagccattggcttggttataacttttgaaatgtgctgcaattagccaggtgtggtggctcatgcctgtaattccggcactttgggaggccgaggcaggcggatcacgaggtcaggagatcgagaccaccctggctaacacggtgaaaccccgtctctactaaaaatacaaaaaattagctgggcatggtggcgggcacctgtagtcccagctcctagggaggctgagacaggagaatggcgtgaacccaggaggcggagcttgcagtgagccgagatcccgccactgcactccagcctgggcgacagagcgagactccgtctcaaaaaaaaaaaaaaaaaaacataatgataataataataataaaaaatagaaagaaatgtgctgcaatcacaaaatttcccagaactttatcttatatgaccgttttcctccagctcatggacagctatgtctgaatcaacagcagtccatagggcactgcccgaggtatcaggtgatgtctaaaaggattcaataggaggagcaggcatctgggaatataccaaaggaaataaagtcaacacctagtaacaatatctgcactcccaagttcaatgcagcattattcacaatagccaggatatggaaacgacttctgtctgtcaacagatgaatagataaagatttggcatactgtatacacacaatgggatactattcagccttcaaaaaagcataaaggccaggcatggtggctcatgtcttaatcccagcactccgggaggctgaggtgggcagatcacttgagctcaggagtttgagaccagcctgggcaccatggtgaaaccctgtctctacaaaaaagtacaaaaaagttggccaggtgtcatggcatgcatctgtagtcccaactaccagggaggcagaggtgggtggatcacttaagccagggaggtcaagactgcagtgagccatgattgtgccactgcactccagcctgggtgacaaagtgagaccctgtctcacaaaaaaaaaaaaaaaaaaagcggtgtggaactgtgccgcttgctacaacatggatgactggaagacattatgctaaatgaaacaaggcagatacagaaaggaaaatactgcatgacctcatttatatgtggaatcttttttttttttaaagtgaatatacagagatagagaatacaacagtggttaccaggagttgaggaggagagcagggaggagatgtaggtcaaatgatacaaagcagcagatatatacgatgaacaagtctagagatccagcgtacaacatgaggactatacttaaaaatgtcacattgcacttgggatttttgctaaaagagtagactttacgtgtccttgccacctaaatgagtaactgtgagatgatggatatgttaattttcttgactaccataatcattttactatcttcatatcaaaatatcatgctgtacaccttatataaatacaataaaaattattattaaaaaagaaagaacaggctgagaatggtggcttatgcctgtaatcccaacactttgggaagttgaggtaggaggatcacttgagaccagtagttcaagatcagcctgggcaacatagcaagaccctatctctacaaaaaataaaaataaaaaaattagccagatatggtagcatgcacctgtagtcctagttactctggagactgaggcaggaggattctttgagacatgatcatgccactgcactccagtttggaggacagagcaagaccccaactctaaaacaacacacaaaaatacagccatttctcctgggtgaaatgggggagctcagactattttgttttatttgtcaggagtctaaagcccatgtgagccaacttaagaaaattctgtttgtatttgagtgttctttattggaaagaaatggacttttccctaatgagcccattttacaaatcgagagctccaaatgcccggttccagcccccatgatatagatgggcagcaatgaggaccagaaatgctgggaagtgtccttgtcatgcaaggaaaacttggaacttcaaggatccatcatgggatgcaggacagtaggctccacccctcttcctgctttccagtaaatgatgttcagggctcacatgcctcttccaaggcatgatgtggggttgcagttctgatcccagcagagaacaaggctcctgtgaaacaattagttttggctcagaggcaaaaaatggaaaccccattcctgtatttacccttcattctttccttacctcatacagctggttccaggtttgattgcatcatatacataaataaataatctgctttcctggtttggtttagttttgctggagagaggagtttatgtgttcatcccatgactagctgggtggtcaagagattggaaagtaggagttctagtttagaccaagtctcatctcagaaaccacagagtagaactgatcccaaacgtcatcatccctgtagggaaaagaaagagagatcagactgttactgtgtctatatagaaagggaagacataagagactccattttgaaaaagagctatactttaaacaattgctttgctgagatgttgttaatttgtagctttgccccagccactttgacccaacctggagctcacaaaaacatgtgttgtataaaatcaaggtttaagggatctagggctgtgtcaaagtggctggggcaaagctacaaattaacaacatctcagcaaagcaattgtttaaagtacagctctttttcaaaatggagtctcttatgtcttccctttctatatagacacagtaacagtctgatctctcttttttttccctacatatcccaatagcacctgcctgactaatacatcatgctctgctgactccatatgtggctggtttcctggatccgattctgactgatggtatctgttctcacgctgctaataaagacataccagagactgggtaatttataaagaaaaagaggtttaatggacttatgtgttccacgtggctggggaggcctcacaatcatggtggaaggtgaaggaggagcaaaagcacatcttacatggtggctggcaaaagagagaaagcatgttcaggggaactcccctttataaaaccatcagatctcatgagacttatgcactatcacaagaacagcatcagaaagatccaccctcatgattcaattacctcccactgggtccctcccatgacacatgggaattatgggagctacaattggagatttgggtggggacacagccaaaccatatcagatggcttatttggtttctatgtagaacctctgcttttcattcaacagtcttcatttagccacagataagctctgtccctaacttccactgatggaatgtacacataagaaacttccactgatggaatgaacacagaaggtgcctactgggaagaaaactggcctgaatctgagctgggtcaaatgtctgcagtcagtttgaatggctgctccttatgggaataatttacattctcaataaaattctctagcaattttctgattgattttaatgagctttaaagccttacgtagaagatcccccagctgatagtcagccttgggcatggattaagggcttttaaccaatcttgcaacaagtttaagcagatattctttattgggtccaatctaaccaaaattattttcttatgttctccccagtaacgtgtcattattaagagaagtttggcttgcttagaggccaaatttagagggtcctgaaattttattttcttttacaccactttccagcatgttacctgatcagttgtttattatctttgctgttgaatggagtgatcattccaagggcccgaggcaggaggcccaggcacagtggaaactctcccaaagaccaggatctttgttttgttccctgacatatgctgagcaccaggaatagtgagtgaatgaaacaaattgtgaggctttaaagagccgaaatatttaaacactgggcacaaggttgttgcttaatcagtgctagatccttacctcccccttgtgtccaggtggacttgttactgcagttaaaccacttgctgatcctcaaacaactagttagtggcacagccaggcctaggaccccagtctctactgttccaactaacccattcgcaggcaggagcactttgaatggtctcttattttaaaaaaattaaattaaaattgtctatttatttagagacagagtcttactctgtagcccaggctcgagtgcagtggtgcaatcatagctcactgtaacctccatctcctggcctcaaaaagtgtttgaattacagatgcgaggcactgtacctggcccgaatgttctgttcagacaaagccacctctaagtcgctgtggggccccagacaagtgatttttgaggagtccctatctataggaacaaagtaattaaaaaaatgtatttcagaatttacaggcccatgtgagatatgatttttttaaatgaagatttagagtaatgggtaaaaaagaggtatttgtgtgtttgttgattgttcagtcagtgaatgtacagcttctgcctcatatccaggcaccatctcttcctgctctttgttgttaaatgttccattcctgggtaatttcatgtctgccatcgtggatatgccgtggctccttgaacctgcttgtgttgaagcaggatcttccttcctgtcccttcagtgccctaataccatgtatttaaggctggacacatcaccactcccaacctgcctcacccactgcgtcacttgtgatcactggcttctggcgactctcaccaaggtctctgtcatgccctgttataatgactacaaaagcaagtcttacctataggaaaataagaattataacccttttactggtcatgtgaaacttaccatttgcaatttgtacagcataaacacagaacagcacatctttcaatgcctgcatcctgaaggcattttgtttgtgtctttcaatctggctgtgctattgttggtgtttaacagtctccccagctacactggaaacttccagaaggcacttttcacttgcttgtgtgttttccccagtgtctattagaggcctttgcacagggtaggctctttggagcagctgaaggtcacacatcccatgagcgggcagcagggtcagaagtggcccccgtgttgcctaagcaagactctcccctgccctctgccctctgcacctccggcctgcatgtccctgtggcctcttgggggtacatctcccggggctgggtcagaaggcctgggtggttggcctcaggctgtcacacacctagggagatgctcccgtttctgggaaccttggccccgactcctgcaaacttcggtaaatgtgtaactcgaccctgcaccggctcactctgttcagcagtgaaactctgcatcgatcactaagacttcctggaagaggtcccagcgtgagtgtcgcttctggcatctgtccttctggccagcctgtggtctggccaagtgatgtaaccctcctctccagcctgtgcacaggcagcctgggaacagctccatccccacccctcagctataaatagggcatcgtgacccggccgggggaagaagctgccgttgttctgggtactacagcagaaggtaagccgggggccccctcagctccttctcggtcttgtctctctcagatgtaactgagctgtgggctaggaggaaaaggccgggaggaggcacggtgatgactgaaaaacctctcccctctcataagaccagtcatccggacgcgggctttcccccactcggtgcccacctggggtcttacaggaggagctgctcctcctcagcaataggacaagatggtcaggtcttcctgcttccgctgagaaaagttagggtcctcaggaacggagcagactggtacaggaacagagtcatcatggccaagagtccaccgggtcctcttgccatcaggaggaatagcagggcttgtgcaggaattggggctggagggaagggccgggctcggtcagtctccagctgggatccccagagtggtcaccctacccctccctcgagacagactgcctgactgtgtgtcatcaggctggtcaccatctccctgaacctcgatttgctcacctataaaatggaactaataacgatgcctgggctccctgtctcaggggctctggtatagctgaagagaactaatataacatgaaagtgctttctaagctttgggataagctaaaaggcagattccaattttattcgagggcagcgtagattggtgcttcagctcgtggatgacagagtcagggggcctggttctgagtcctagttctgtctcttcccagctgtgtgacgttgaacaagtcactggacctctctgttcctctgcaaaacagcatgaaccaattcattaactacttctccaggatgcagtaggtcccagggactatcctaggaatgtgggctgtattagtaaacacaacagcgggaaccctgttccggggctcacattcacatcagagcaaacagacaaagacgctggacagaataagtgcataactacatggtacagagggttataaggagggaaaaggggagctggatgagagagttgagagtgcccggtgtggtggggaaagctgcagggtgaaatactgcatcagggaaacctcagggaaggtgaggactatggtgaggtcagaggggttgatatgagaacagtgccctgcaaatggcaggcaccacaggagcatgagccgtcatcttcacctttagcattcagcccgggagaagtagggagacatagaaggggcaggtgctggccaagaggcaggggcaggagaggagaaggcggaggggcactcagggcgagggtgtcaggcccgccaccccagagcaccattactcccaggacgcggctgcgtgcagacctggaaccagcctagggagcagccgcagatcacaactgagaacaaacgacagtctctgcctcaaaaatggcccatggaattgcgtctctggagacgctgcctgagcaggagcagcacagtgagcgggctgcatcgaccagcgccatccaaaccccgaacagttggcgcttgtcaggcaggacttcccagcagtcggttcccacaggtttcccctgttgacctgatttgatgtgactgtctagattaggtgtgaactggtggcttaggcttctctgcacagaaaggcctgcaagcagcagagagagttttctgttccatttttccatgtcatgtggctcttcctgagaacagcggatggagtcaaatgcatggggagtggggtgagatggtagctgaggtcagaatttggcatttgaatgactgaagcagaacaaaacacaccaggtacttcagcagctgcaccgtgttgagggcaggtgctggttacgggtctgggtgagggaagccagctgccaatgtaagaagaatgactgggtatgcttagatgaagcagaaaaatctaggcatcaaggtggccttgagtcagtgatgacacgctacagctccaaggaagcctggcctagccctggggggacagaaaaggccaagaagtgacgatattgcagtacacccccctccacaagaaatgagtgagatgtggtacaaaatgttagaattgaatgaatcaatagaataaacgttcatcccttcaatcaagaagagtcagatgaaatgaattagcagggccagcccaagaacctcttctgggggtctcagggtagctttcatttgtagcagctgaggctgaagcccagctgcaaggcctttgagagaacgtggtgctggacccgtgtctagggcaggggttctaaaccctgcttacatatcagagtcacctgagaattttctatttttttttttttttttttatacgtggtcccagcacagactaaggaatccaactatcattgggcaagccatgctaggtatgcatgcctttggggctctgcaggggatagcgctatgcagggatggttgagagctggttttggggttgagacacgtgggaaatacttggactttgggctgagcctgtggtgctcaatcccggctgcatgttgggaccacagggagatgacaaaaccatccccagccctcaccctagggccctcgaatgagcatctcaggggtctaggaggcctccacaaagacctactgattggcacacacttgtttctctaggaagagaacttacagctgcaggcaggagcatgtcttaatctgcttgggctgccataagtaccacagactgggagggtttaacaacagaaatgtgttatctcacagttctggaagctagaagcctgggagccagccatcagcagagttggtttcctctgggtcctctatccttggcttgtagatggccgtcttctctctgtgtccccacatggtcttccctctgtgtccccacatggtcttccctctgtgtgtgtccatgtcctcatctcctcttctcataaggacacaggtcatattagatcagggctcaccctcatggcctcattttaacttaatcatctctttaaagatcctgtctccaaataatggtcacattctgaggtcctggggttgaggacttcaacacgggcattatggccgttgggggaggtaggacataattcagctgatattggtgcattttgcacttggatcatgtagatattttccatggagctttgaatccatttcttcttttttttgtagacatgaatggatttattctgggctaaatggtgacagggaatattgagacaatgaaagatctggttagatggcacttaaaggtcagttaataaccacctttcaccctttgcaaaatgatatttcagggtatgcggaagcgagcaccccagtctgagatggctcctgccggtgtgagcctgagggccaccatcctctgcctcctggcctgggctggcctggctgcaggtgaccgggtgtacatacaccccttccacctcgtcatccacaatgagagtacctgtgagcagctggcaaaggccaatgccgggaagcccaaagaccccaccttcatacctgctccaattcaggccaagacatcccctgtggatgaaaaggccctacaggaccagctggtgctagtcgctgcaaaacttgacaccgaagacaagttgagggccgcaatggtcgggatgctggccaacttcttgggcttccgtatatatggcatgcacagtgagctatggggcgtggtccatggggccaccgtcctctccccaacggctgtctttggcaccctggcctctctctatctgggagccttggaccacacagctgacaggctacaggcaatcctgggtgttccttggaaggacaagaactgcacctcccggctggatgcgcacaaggtcctgtctgccctgcaggctgtacagggcctgctagtggcccagggcagggctgatagccaggcccagctgctgctgtccacggtggtgggcgtgttcacagccccaggcctgcacctgaagcagccgtttgtgcagggcctggctctctatacccctgtggtcctcccacgctctctggacttcacagaactggatgttgctgctgagaagattgacaggttcatgcaggctgtgacaggatggaagactggctgctccctgatgggagccagtgtggacagcaccctggctttcaacacctacgtccacttccaaggtaaggcaaacctctctgctggctctggccctaggacttagtatccaatgtgtagctgagatcagccagtcaggccttggagatgggcagggggcagccctgcggacatacctggtgaccacccttgagaagtggggaagtggctgctccgctgggtccctggatgggccgtccacctcctggacctgctgccctactatgtgcacgactatacaacatcctttttcttacatcatttaatccccttatgatgtggtgaagaggtatttgtgcctttgtttaccagtgaagaaatagagactcggagaaacaaagtgccttgctcaagatggcacagccaccagtgggggtcctgggattgaaacccacatctcctggccccacagcccagttctacactcagaagggtcaggttcatatctcttgagaaggtcaggaactggggtccctggcccatgcagaaataagcaattggcttgcttaaatccctttcatgttaggaggggcattactgaaaaccctctactacaaagattgttgatttttttttttttttttattgagacagggtcttgttctgtcacccaggctgcagtgtagtggtgccatcattgctcactgtagccttgaactcctggcctcaagcgatcctcccacctctgccttccaaagtgttgggattaaaggtgtgagccactgcacccagccacagattgcttaaagcattcatttaacaaatacttgttgaggatttgctacttgtaagactttaagcctggcatctcagaggaggccagaggagggctgtataggccctgcctccaggcttttaaaggtcaatgggcaaatgcctaggatttggagctgcagggaaacgtgctccacaaggtaactcagggaagcctcggggctctcagaggacagaggtcactggggagcggagagcaggccttgcctggcagtgagggcaacagggctggtgaagctaggagcaagcatgatgagcccagcctgcagagtttggggcaaggaacgaggatggggcggttggcttggcatgagtgttgaaccagaaaatgggcctggggagggcagagctggagacactttgaacgccatgcttggtaggtgtgggaatggggacgcgttctgttcagaggtcatcccggaagcctgccgtgtgcagactggaggcagggaggattgtttgaaggttacgcaagagtccaggcacacagtcacgggaacacgtgctcagggagcagctcggcaaatccatgggtggggtggggctgaggggtgtgtctaagagacactgaggaggctctgtcaagatgttaacctcgtgagggacagagagccaggcgggaggtgaaagacaagactgtggagaaagaggttcagtggcgcatagtgatttttcttaccacaacaacctccttgaggtctttcccttcgggttcagggagaggtgatagatggggggattgctcagccctggcactgactggtcacaggggcagaggccagcccgagggttgcccggttgagggtggcagcacactgtgcagggcagagcagggacacatggacttagcctgctgtccctaggagaagtgctgggaggagcgctcactgagaaggagggtcctgcagaaggcaaaggcaagaaagccagtggcatctgaaatgggtctcccttcgaaagagagcacatccacctgacccagaccgcagagccaggccaggaggaagaggaggaagaataaaaaagccaaccacatcgggactcaaaggaagcccaggatcctcgccggcctccaccgcatgctgccctgaccctgccccacttcctaactttgctggcctcagtttccgtcaaaggaggcagccacttcctgcccacatggtctgtccagtgaggagatcgggggctgtctcgggacctctaggtttccctttagcaatgatgttctatttacatgacctcagcaggcagctagatgtgtcccactagagaggacctgaggatctggggcctgatgggctccagggtaccgtctgcccagtgcttgctgtgctcctgagcatggggcgctggccctggtggtttccatgacaccaggtcctgacttgacctcgacagatttacctagcctccggatgagaatggtgagctgtgcatgtcagacgagcagagggaagacggcagccactctcatgtcaaatcccagcgtcttttgggaggcagcttcccttttttagtttagtttgttggaagaaaagaattgtccctttcccccctctaaactaaaagccttgccagcccaggtgggcagcaccgaggtccctgcagggaacgtgcaaggggaaccctgcagtttcccgctcacatgcccttccgagactgagtgctccgaggactgaggacgagaaatatgccaggtctgccactgccttcttacgagacccggacccaggggaggcacagccatgcccagctcctgcctgccagttctgtcctcccagctgccctactttcatgctgggacctccaattcagtacaaagggagacctcactgtttctgaaccatctctactcagactcccaagtgccacgtgcccaggggactgttctgtgacaaacttatacacaacttcaccctattctcctaagaacaaccgcagaataggcctttcaggatgagtgggaggacagccgagggcagggatgtgctagtgtaaggtcgaggcagagggtgggctgctgtcatggaaagaccccaggtaactgcgtcacacacaaatttgtgtccttctcccacaacgggctctcccgagttctctgtcatctgcacggccctgtgagcaggaggggaaacagagggctcacccctgcccccaaggcccagtgtgcaaatccattcatcacaacgaggttgtgtgagtctccccagtagcaagggctgctgaggaatggagccctcgtttccggggcctgcgtggcccactctgtattctatgactgtgatgggggagggtgggggccacaggacagctggtgggctctgccatggctggggctagacatggattaaaaagtgagtatgagcaggggcctctaggagtggtgggatagtgcggtggtggccacatgtcattctacgtgcgtccaaacctacagaatgtaaaacaccaggagggagactcaaagaaaactatcaactttgagtgctgaggacgtgtcagtgtaggttcgtcagttgcaacaaatgggccacgctggtgtgagatgttgatcacgggggaggctgtgtagtgggggacaagagttatatgggaactttctgtactttctgctcgattttgctgtgaacctaaagtcactctaaaaaataacatctcttaaattttttaaaaagtgagtgtgtcaaaccacagcctttgggtcaggacagttctaggtttgagttgacctggcaggtaccagtggcttatgtcccttaaggtgacagatgcaaaacccccggtttggtgcctggcatgttgtgtgtcttgcaggtggcggttagggctgcctcagtgaactcaaatggctgcattttacaggagaaatatttgagccacacttgcggtcctgtggccaggagaatgcagagtggcctggggggggccaaggaaggaggctgaggcagggcgaggggcaggatctgggcctttggtgtctgccagccctcattcctgcccctgtcttgggtgactcttccctccctgtctcctgtctggatttcagggaagatgaagggcttctccctgctggccgagccccaggagttctgggtggacaacagcacctcagtgtctgttcccatgctctctggcatgggcaccttccagcactggagtgacatccaggacaacttctcggtgactcaagtgcccttcactgagagcgcctgcctgctgctgatccagcctcactatgcctctgacctggacaaggtggagggtctcactttccagcaaaactccctcaactggatgaagaaactatctccccggtaggagcctcccggtctcccctggaatgtgggagccacactgtcctgcccaggctgggggcggggtggggagtagacacacctgagctgagccttgggtgcagagcagggcagggccgcggtggcacggggctgggcaggcggcctgtgtgtctgtctaccagtcctctatccagccagcacccagctctccagttagtgtctgtctttcaagtgcaggcaaggtaaaggaggagaggaagaatgctttttctacacttacacttgcctggtagttttggagggggagaaaacattgcaatccgccctctgagagaggaccattttggtcccacacctgacacacagcacacctgtgacatccaagagcttcttggaactgacttgccaggagggttcggacttcgcgtgagcgggggtggggccttctcagggagcgtcccttgactccagaacgcccttgctggcggctggcggctgggtggggataggtgttgttagctcctctttcctgctgcaattcctttccacagagccctggactcaaactacacatcaccccagatcatcgaggcctggaaatctgctcccagaggcaggcattgagtgacacgatggcttgacatcaactctgggtgttttttatgttttaaaaattgtgatggtaaaatatacgtaacaaaatttgccatcgtaaccattttcgagtgcacagttcagtggtactaggcccattcacactgttgtgcagccatcacccccgtccatctccatttatcttctcaacttcccaaactgaagctctgtcctgctgaaacactaactctccatttccccttccccttggccccggcaaccaccacgatgtcctcgaggttcacccatgttgtagcacatgtcagaatgtccttccttttgaaggctgaataatattccattgcatgtggttaccaccttttgtgtatccactcatccatcgatggacacgtgggttgcttccacctttgagctgctgtgaatagtgcagtgtaccctgtaaacatgggtgtactgtcagctcttataagtgcttgatacatcactggaaatgtccatgggctctgaaggatgccaaaagatggaagaggctctatacgaagatcaatcgagttgacatagcaacgtgtccagcacgaggttgacactgtaccctcctgcctctctccttttcatgggtgtcatgtcatcaagaacactgctgtggcagtagtaagacacagtgcattatttcagagaatagcatttaaaaattacccaagtaacacaccttcaatgcagccaacctaaaaacagaatgcaccaaaggacaaccattcctaggtcctcatcggtaaatcttctatgtccctcacatagtattgcaaatgacatgaaggatttttattgtaggttttgctgaaattttccccaagggggaggatgacttagttgggtgatggggggagcaaacatccctgtcgtcagggttgggtgcaaggagcataagcctgcctggcctctgggagagccctcactgtgtggcctggagccttcctaactgtgcatcatctccccaggaccatccacctgaccatgccccaactggtgctgcaaggatcttatgacctgcaggacctgctcgcccaggctgagctgcccgccattctgcacaccgagctgaacctgcaaaaattgagcaatgaccgcatcagggtgggggaggtatgtgtgagcctgtgtctgtgcctgacctgggttccaagtgtgcacagggtgggaggcatggatgtaagggacacagaggaggctatgggtggggccagcagggcaagagggagcggagagtagggccaaaggtgggagagaagtagccagagcattctggggccttccaggtgcagagcagcaaatccctccccatccctgctgtgcctcctcctgctaggtgtgtgttccatggtcctgcttggccttgccttgcctcagggtcctccagggttcctatagtggagttgaaaccgggatgaagacagcaagcacccctggacctggtgccctgggcccagccccttcttcagggaaatgctgagcagcagacagaatgtccccctgccatgtggcaccatgcacatctgcagctaccaaggatgtgccttgatgttctgggccctgtgctcagtgctggggagaaagtgggagttcttacgggggccagcgggaagagccctctgtgctaagttagctaagccctggcactggtgggccatggccaagggagccaggaattctgcctgggacatcagggcagaatgtgaagatgggaggatgtaaggggtgtgttagggaggagccggcatgtgagtttggccattgtggccaattaacggtcatctacacacagacacacccttgcctacactgaggggcaggcatacactgtgcatcctcctggcaggctggaaaatgtccccctccaggacagtgcacagcacagaggtcctgagcccaccccggccctctagccctcagcaccctgggtcacccagtgcgccctcagaatgatcctgatgtctgctgctttgcaggtgctgaacagcattttttttgagcttgaagcggatgagagagagcccacagagtctacccaacagcttaacaagcctgaggtcttggaggtgaccctgaaccgcccattcctgtttgctgtgtatgatcaaagcgccactgccctgcacttcctgggccgcgtggccaacccgctgagcacagcatgaggccagggccccagaacacagtgcctggcaaggcctctgcccctggcctttgaggcaaaggccagcagcagataacaaccccggacaaatcagcgatgtgtcacccccagtctcccaccttttcttctaatgagtcgactttgagctggaaagcagccgtttctccttggtctaagtgtgctgcatggagtgagcagtagaagcctgcagcggcacaaatgcacctcccagtttgctgggtttattttagagaatgggggtggggaggcaagaaccagtgtttagcgcgggactactgttccaaaaagaattccaaccgaccagcttgtttgtgaaacaaaaaagtgttcccttttcaagttgagaacaaaaattgggttttaaaattaaagtatacatttttgcattgccttcggtttgtatttagtgtcttgaatgtaagaacatgacctccgtgtagtgtctgtaataccttagttttttccacagatgcttgtgatttttgaacaatacgtgaaagatgcaagcacctgaatttctgtttgaatgcggaaccatagctggttatttctcccttgtgttagtaataaacgtcttgccacaataagcctccaaaaa

SEQ ID NO: 3 - DNA, 2148 bp, Homo sapiens
  source 1..2148: mol_type = genomic DNA
agaaagtagaccctcaccaggcatggaatctgccagtgccttgatcttggacttccagcttccagaactggtatgcggaagcgagcaccccagtctgagatggctcctgccggtgtgagcctgagggccaccatcctctgcctcctggcctgggctggcctggctgcaggtgaccgggtgtacatacaccccttccacctcgtcatccacaatgagagtacctgtgagcagctggcaaaggccaatgccgggaagcccaaagaccccaccttcatacctgctccaattcaggccaagacatcccctgtggatgaaaaggccctacaggaccagctggtgctagtcgctgcaaaacttgacaccgaagacaagttgagggccgcaatggtcgggatgctggccaacttcttgggcttccgtatatatggcatgcacagtgagctatggggcgtggtccatggggccaccgtcctctccccaacggctgtctttggcaccctggcctctctctatctgggagccttggaccacacagctgacaggctacaggcaatcctgggtgttccttggaaggacaagaactgcacctcccggctggatgcgcacaaggtcctgtctgccctgcaggctgtacagggcctgctagtggcccagggcagggctgatagccaggcccagctgctgctgtccacggtggtgggcgtgttcacagccccaggcctgcacctgaagcagccgtttgtgcagggcctggctctctatacccctgtggtcctcccacgctctctggacttcacagaactggatgttgctgctgagaagattgacaggttcatgcaggctgtgacaggatggaagactggctgctccctgatgggagccagtgtggacagcaccctggctttcaacacctacgtccacttccaagggaagatgaagggcttctccctgctggccgagccccaggagttctgggtggacaacagcacctcagtgtctgttcccatgctctctggcatgggcaccttccagcactggagtgacatccaggacaacttctcggtgactcaagtgcccttcactgagagcgcctgcctgctgctgatccagcctcactatgcctctgacctggacaaggtggagggtctcactttccagcaaaactccctcaactggatgaagaaactatctccccggaccatccacctgaccatgccccaactggtgctgcaaggatcttatgacctgcaggacctgctcgcccaggctgagctgcccgccattctgcacaccgagctgaacctgcaaaaattgagcaatgaccgcatcagggtgggggaggtgctgaacagcattttttttgagcttgaagcggatgagagagagcccacagagtctacccaacagcttaacaagcctgaggtcttggaggtgaccctgaaccgcccattcctgtttgctgtgtatgatcaaagcgccactgccctgcacttcctgggccgcgtggccaacccgctgagcacagcatgaggccagggccccagaacacagtgcctggcaaggcctctgcccctggcctttgaggcaaaggccagcagcagataacaaccccggacaaatcagcgatgtgtcacccccagtctcccaccttttcttctaatgagtcgactttgagctggaaagcagccgtttctccttggtctaagtgtgctgcatggagtgagcagtagaagcctgcagcggcacaaatgcacctcccagtttgctgggtttattttagagaatgggggtggggaggcaagaaccagtgtttagcgcgggactactgttccaaaaagaattccaaccgaccagcttgtttgtgaaacaaaaaagtgttcccttttcaagttgagaacaaaaattgggttttaaaattaaagtatacatttttgcattgccttcggtttgtatttagtgtcttgaatgtaagaacatgacctccgtgtagtgtctgtaataccttagttttttccacagatgcttgtgatttttgaacaatacgtgaaagatgcaagcacctgaatttctgtttgaatgcggaaccatagctggttatttctcccttgtgttagtaataaacgtcttgccacaataagcctccaaaaa

SEQ ID NO: 4 - DNA, 19068 bp, Homo sapiens
  source 1..19068: mol_type = genomic DNA
ctaaaaatacaaaaaattagctgggcatggtggcgggcacctgtagtcccagctcctagggaggctgagacaggagaatggcgtgaacccaggaggcggagcttgcagtgagccgagatcccgccactgcactccagcctgggcgacagagcgagactccgtctcaaaaaaaaaaaaaaaaaaacataatgataataataataataaaaaatagaaagaaatgtgctgcaatcacaaaatttcccagaactttatcttatatgaccgttttcctccagctcatggacagctatgtctgaatcaacagcagtccatagggcactgcccgaggtatcaggtgatgtctaaaaggattcaataggaggagcaggcatctgggaatataccaaaggaaataaagtcaacacctagtaacaatatctgcactcccaagttcaatgcagcattattcacaatagccaggatatggaaacgacttctgtctgtcaacagatgaatagataaagatttggcatactgtatacacacaatgggatactattcagccttcaaaaaagcataaaggccaggcatggtggctcatgtcttaatcccagcactccgggaggctgaggtgggcagatcacttgagctcaggagtttgagaccagcctgggcaccatggtgaaaccctgtctctacaaaaaagtacaaaaaagttggccaggtgtcatggcatgcatctgtagtcccaactaccagggaggcagaggtgggtggatcacttaagccagggaggtcaagactgcagtgagccatgattgtgccactgcactccagcctgggtgacaaagtgagaccctgtctcacaaaaaaaaaaaaaaaaaaagcggtgtggaactgtgccgcttgctacaacatggatgactggaagacattatgctaaatgaaacaaggcagatacagaaaggaaaatactgcatgacctcatttatatgtggaatcttttttttttttaaagtgaatatacagagatagagaatacaacagtggttaccaggagttgaggaggagagcagggaggagatgtaggtcaaatgatacaaagcagcagatatatacgatgaacaagtctagagatccagcgtacaacatgaggactatacttaaaaatgtcacattgcacttgggatttttgctaaaagagtagactttacgtgtccttgccacctaaatgagtaactgtgagatgatggatatgttaattttcttgactaccataatcattttactatcttcatatcaaaatatcatgctgtacaccttatataaatacaataaaaattattattaaaaaagaaagaacaggctgagaatggtggcttatgcctgtaatcccaacactttgggaagttgaggtaggaggatcacttgagaccagtagttcaagatcagcctgggcaacatagcaagaccctatctctacaaaaaataaaaataaaaaaattagccagatatggtagcatgcacctgtagtcctagttactctggagactgaggcaggaggattctttgagacatgatcatgccactgcactccagtttggaggacagagcaagaccccaactctaaaacaacacacaaaaatacagccatttctcctgggtgaaatgggggagctcagactattttgttttatttgtcaggagtctaaagcccatgtgagccaacttaagaaaattctgtttgtatttgagtgttctttattggaaagaaatggacttttccctaatgagcccattttacaaatcgagagctccaaatgcccggttccagcccccatgatatagatgggcagcaatgaggaccagaaatgctgggaagtgtccttgtcatgcaaggaaaacttggaacttcaaggatccatcatgggatgcaggacagtaggctccacccctcttcctgctttccagtaaatgatgttcagggctcacatgcctcttccaaggcatgatgtggggttgcagttctgatcccagcagagaacaaggctcctgtgaaacaattagttttggctcagaggcaaaaaatggaaaccccattcctgtatttacccttcattctttccttacctcatacagctggttccaggtttgattgcatcatatacataaataaataatctgctttcctggtttggtttagttttgctggagagaggagtttatgtgttcatcccatgactagctgggtggtcaagagattggaaagtaggagttctagtttagaccaagtctcatctcagaaaccacagagtagaactgatcccaaacgtcatcatccctgtagggaaaagaaagagagatcagactgttactgtgtctatatagaaagggaagacataagagactccattttgaaaaagagctatactttaaacaattgctttgctgagatgttgttaatttgtagctttgccccagccactttgacccaacctggagctcacaaaaacatgtgttgtataaaatcaaggtttaagggatctagggctgtgtcaaagtggctggggcaaagctacaaattaacaacatctcagcaaagcaattgtttaaagtacagctctttttcaaaatggagtctcttatgtcttccctttctatatagacacagtaacagtctgatctctcttttttttccctacatatcccaatagcacctgcctgactaatacatcatgctctgctgactccatatgtggctggtttcctggatccgattctgactgatggtatctgttctcacgctgctaataaagacataccagagactgggtaatttataaagaaaaagaggtttaatggacttatgtgttccacgtggctggggaggcctcacaatcatggtggaaggtgaaggaggagcaaaagcacatcttacatggtggctggcaaaagagagaaagcatgttcaggggaactcccctttataaaaccatcagatctcatgagacttatgcactatcacaagaacagcatcagaaagatccaccctcatgattcaattacctcccactgggtccctcccatgacacatgggaattatgggagctacaattggagatttgggtggggacacagccaaaccatatcagatggcttatttggtttctatgtagaacctctgcttttcattcaacagtcttcatttagccacagataagctctgtccctaacttccactgatggaatgtacacataagaaacttccactgatggaatgaacacagaaggtgcctactgggaagaaaactggcctgaatctgagctgggtcaaatgtctgcagtcagtttgaatggctgctccttatgggaataatttacattctcaataaaattctctagcaattttctgattgattttaatgagctttaaagccttacgtagaagatcccccagctgatagtcagccttgggcatggattaagggcttttaaccaatcttgcaacaagtttaagcagatattctttattgggtccaatctaaccaaaattattttcttatgttctccccagtaacgtgtcattattaagagaagtttggcttgcttagaggccaaatttagagggtcctgaaattttattttcttttacaccactttccagcatgttacctgatcagttgtttattatctttgctgttgaatggagtgatcattccaagggcccgaggcaggaggcccaggcacagtggaaactctcccaaagaccaggatctttgttttgttccctgacatatgctgagcaccaggaatagtgagtgaatgaaacaaattgtgaggctttaaagagccgaaatatttaaacactgggcacaaggttgttgcttaatcagtgctagatccttacctcccccttgtgtccaggtggacttgttactgcagttaaaccacttgctgatcctcaaacaactagttagtggcacagccaggcctaggaccccagtctctactgttccaactaacccattcgcaggcaggagcactttgaatggtctcttattttaaaaaaattaaattaaaattgtctatttatttagagacagagtcttactctgtagcccaggctcgagtgcagtggtgcaatcatagctcactgtaacctccatctcctggcctcaaaaagtgtttgaattacagatgcgaggcactgtacctggcccgaatgttctgttcagacaaagccacctctaagtcgctgtggggccccagacaagtgatttttgaggagtccctatctataggaacaaagtaattaaaaaaatgtatttcagaatttacaggcccatgtgagatatgatttttttaaatgaagatttagagtaatgggtaaaaaagaggtatttgtgtgtttgttgattgttcagtcagtgaatgtacagcttctgcctcatatccaggcaccatctcttcctgctctttgttgttaaatgttccattcctgggtaatttcatgtctgccatcgtggatatgccgtggctccttgaacctgcttgtgttgaagcaggatcttccttcctgtcccttcagtgccctaataccatgtatttaaggctggacacatcaccactcccaacctgcctcacccactgcgtcacttgtgatcactggcttctggcgactctcaccaaggtctctgtcatgccctgttataatgactacaaaagcaagtcttacctataggaaaataagaattataacccttttactggtcatgtgaaacttaccatttgcaatttgtacagcataaacacagaacagcacatctttcaatgcctgcatcctgaaggcattttgtttgtgtctttcaatctggctgtgctattgttggtgtttaacagtctccccagctacactggaaacttccagaaggcacttttcacttgcttgtgtgttttccccagtgtctattagaggcctttgcacagggtaggctctttggagcagctgaaggtcacacatcccatgagcgggcagcagggtcagaagtggcccccgtgttgcctaagcaagactctcccctgccctctgccctctgcacctccggcctgcatgtccctgtggcctcttgggggtacatctcccggggctgggtcagaaggcctgggtggttggcctcaggctgtcacacacctagggagatgctcccgtttctgggaaccttggccccgactcctgcaaacttcggtaaatgtgtaactcgaccctgcaccggctcactctgttcagcagtgaaactctgcatcgatcactaagacttcctggaagaggtcccagcgtgagtgtcgcttctggcatctgtccttctggccagcctgtggtctggccaagtgatgtaaccctcctctccagcctgtgcacaggcagcctgggaacagctccatccccacccctcagctataaatagggcatcgtgacccggccgggggaagaagctgccgttgttctgggtactacagcagaaggtaagccgggggccccctcagctccttctcggtcttgtctctctcagatgtaactgagctgtgggctaggaggaaaaggccgggaggaggcacggtgatgactgaaaaacctctcccctctcataagaccagtcatccggacgcgggctttcccccactcggtgcccacctggggtcttacaggaggagctgctcctcctcagcaataggacaagatggtcaggtcttcctgcttccgctgagaaaagttagggtcctcaggaacggagcagactggtacaggaacagagtcatcatggccaagagtccaccgggtcctcttgccatcaggaggaatagcagggcttgtgcaggaattggggctggagggaagggccgggctcggtcagtctccagctgggatccccagagtggtcaccctacccctccctcgagacagactgcctgactgtgtgtcatcaggctggtcaccatctccctgaacctcgatttgctcacctataaaatggaactaataacgatgcctgggctccctgtctcaggggctctggtatagctgaagagaactaatataacatgaaagtgctttctaagctttgggataagctaaaaggcagattccaattttattcgagggcagcgtagattggtgcttcagctcgtggatgacagagtcagggggcctggttctgagtcctagttctgtctcttcccagctgtgtgacgttgaacaagtcactggacctctctgttcctctgcaaaacagcatgaaccaattcattaactacttctccaggatgcagtaggtcccagggactatcctaggaatgtgggctgtattagtaaacacaacagcgggaaccctgttccggggctcacattcacatcagagcaaacagacaaagacgctggacagaataagtgcataactacatggtacagagggttataaggagggaaaaggggagctggatgagagagttgagagtgcccggtgtggtggggaaagctgcagggtgaaatactgcatcagggaaacctcagggaaggtgaggactatggtgaggtcagaggggttgatatgagaacagtgccctgcaaatggcaggcaccacaggagcatgagccgtcatcttcacctttagcattcagcccgggagaagtagggagacatagaaggggcaggtgctggccaagaggcaggggcaggagaggagaaggcggaggggcactcagggcgagggtgtcaggcccgccaccccagagcaccattactcccaggacgcggctgcgtgcagacctggaaccagcctagggagcagccgcagatcacaactgagaacaaacgacagtctctgcctcaaaaatggcccatggaattgcgtctctggagacgctgcctgagcaggagcagcacagtgagcgggctgcatcgaccagcgccatccaaaccccgaacagttggcgcttgtcaggcaggacttcccagcagtcggttcccacaggtttcccctgttgacctgatttgatgtgactgtctagattaggtgtgaactggtggcttaggcttctctgcacagaaaggcctgcaagcagcagagagagttttctgttccatttttccatgtcatgtggctcttcctgagaacagcggatggagtcaaatgcatggggagtggggtgagatggtagctgaggtcagaatttggcatttgaatgactgaagcagaacaaaacacaccaggtacttcagcagctgcaccgtgttgagggcaggtgctggttacgggtctgggtgagggaagccagctgccaatgtaagaagaatgactgggtatgcttagatgaagcagaaaaatctaggcatcaaggtggccttgagtcagtgatgacacgctacagctccaaggaagcctggcctagccctggggggacagaaaaggccaagaagtgacgatattgcagtacacccccctccacaagaaatgagtgagatgtggtacaaaatgttagaattgaatgaatcaatagaataaacgttcatcccttcaatcaagaagagtcagatgaaatgaattagcagggccagcccaagaacctcttctgggggtctcagggtagctttcatttgtagcagctgaggctgaagcccagctgcaaggcctttgagagaacgtggtgctggacccgtgtctagggcaggggttctaaaccctgcttacatatcagagtcacctgagaattttctatttttttttttttttttttatacgtggtcccagcacagactaaggaatccaactatcattgggcaagccatgctaggtatgcatgcctttggggctctgcaggggatagcgctatgcagggatggttgagagctggttttggggttgagacacgtgggaaatacttggactttgggctgagcctgtggtgctcaatcccggctgcatgttgggaccacagggagatgacaaaaccatccccagccctcaccctagggccctcgaatgagcatctcaggggtctaggaggcctccacaaagacctactgattggcacacacttgtttctctaggaagagaacttacagctgcaggcaggagcatgtcttaatctgcttgggctgccataagtaccacagactgggagggtttaacaacagaaatgtgttatctcacagttctggaagctagaagcctgggagccagccatcagcagagttggtttcctctgggtcctctatccttggcttgtagatggccgtcttctctctgtgtccccacatggtcttccctctgtgtccccacatggtcttccctctgtgtgtgtccatgtcctcatctcctcttctcataaggacacaggtcatattagatcagggctcaccctcatggcctcattttaacttaatcatctctttaaagatcctgtctccaaataatggtcacattctgaggtcctggggttgaggacttcaacacgggcattatggccgttgggggaggtaggacataattcagctgatattggtgcattttgcacttggatcatgtagatattttccatggagctttgaatccatttcttcttttttttgtagacatgaatggatttattctgggctaaatggtgacagggaatattgagacaatgaaagatctggttagatggcacttaaaggtcagttaataaccacctttcaccctttgcaaaatgatatttcagggtatgcggaagcgagcaccccagtctgagatggctcctgccggtgtgagcctgagggccaccatcctctgcctcctggcctgggctggcctggctgcaggtgaccgggtgtacatacaccccttccacctcgtcatccacaatgagagtacctgtgagcagctggcaaaggccaatgccgggaagcccaaagaccccaccttcatacctgctccaattcaggccaagacatcccctgtggatgaaaaggccctacaggaccagctggtgctagtcgctgcaaaacttgacaccgaagacaagttgagggccgcaatggtcgggatgctggccaacttcttgggcttccgtatatatggcatgcacagtgagctatggggcgtggtccatggggccaccgtcctctccccaacggctgtctttggcaccctggcctctctctatctgggagccttggaccacacagctgacaggctacaggcaatcctgggtgttccttggaaggacaagaactgcacctcccggctggatgcgcacaaggtcctgtctgccctgcaggctgtacagggcctgctagtggcccagggcagggctgatagccaggcccagctgctgctgtccacggtggtgggcgtgttcacagccccaggcctgcacctgaagcagccgtttgtgcagggcctggctctctatacccctgtggtcctcccacgctctctggacttcacagaactggatgttgctgctgagaagattgacaggttcatgcaggctgtgacaggatggaagactggctgctccctgatgggagccagtgtggacagcaccctggctttcaacacctacgtccacttccaaggtaaggcaaacctctctgctggctctggccctaggacttagtatccaatgtgtagctgagatcagccagtcaggccttggagatgggcagggggcagccctgcggacatacctggtgaccacccttgagaagtggggaagtggctgctccgctgggtccctggatgggccgtccacctcctggacctgctgccctactatgtgcacgactatacaacatcctttttcttacatcatttaatccccttatgatgtggtgaagaggtatttgtgcctttgtttaccagtgaagaaatagagactcggagaaacaaagtgccttgctcaagatggcacagccaccagtgggggtcctgggattgaaacccacatctcctggccccacagcccagttctacactcagaagggtcaggttcatatctcttgagaaggtcaggaactggggtccctggcccatgcagaaataagcaattggcttgcttaaatccctttcatgttaggaggggcattactgaaaaccctctactacaaagattgttgatttttttttttttttttattgagacagggtcttgttctgtcacccaggctgcagtgtagtggtgccatcattgctcactgtagccttgaactcctggcctcaagcgatcctcccacctctgccttccaaagtgttgggattaaaggtgtgagccactgcacccagccacagattgcttaaagcattcatttaacaaatacttgttgaggatttgctacttgtaagactttaagcctggcatctcagaggaggccagaggagggctgtataggccctgcctccaggcttttaaaggtcaatgggcaaatgcctaggatttggagctgcagggaaacgtgctccacaaggtaactcagggaagcctcggggctctcagaggacagaggtcactggggagcggagagcaggccttgcctggcagtgagggcaacagggctggtgaagctaggagcaagcatgatgagcccagcctgcagagtttggggcaaggaacgaggatggggcggttggcttggcatgagtgttgaaccagaaaatgggcctggggagggcagagctggagacactttgaacgccatgcttggtaggtgtgggaatggggacgcgttctgttcagaggtcatcccggaagcctgccgtgtgcagactggaggcagggaggattgtttgaaggttacgcaagagtccaggcacacagtcacgggaacacgtgctcagggagcagctcggcaaatccatgggtggggtggggctgaggggtgtgtctaagagacactgaggaggctctgtcaagatgttaacctcgtgagggacagagagccaggcgggaggtgaaagacaagactgtggagaaagaggttcagtggcgcatagtgatttttcttaccacaacaacctccttgaggtctttcccttcgggttcagggagaggtgatagatggggggattgctcagccctggcactgactggtcacaggggcagaggccagcccgagggttgcccggttgagggtggcagcacactgtgcagggcagagcagggacacatggacttagcctgctgtccctaggagaagtgctgggaggagcgctcactgagaaggagggtcctgcagaaggcaaaggcaagaaagccagtggcatctgaaatgggtctcccttcgaaagagagcacatccacctgacccagaccgcagagccaggccaggaggaagaggaggaagaataaaaaagccaaccacatcgggactcaaaggaagcccaggatcctcgccggcctccaccgcatgctgccctgaccctgccccacttcctaactttgctggcctcagtttccgtcaaaggaggcagccacttcctgcccacatggtctgtccagtgaggagatcgggggctgtctcgggacctctaggtttccctttagcaatgatgttctatttacatgacctcagcaggcagctagatgtgtcccactagagaggacctgaggatctggggcctgatgggctccagggtaccgtctgcccagtgcttgctgtgctcctgagcatggggcgctggccctggtggtttccatgacaccaggtcctgacttgacctcgacagatttacctagcctccggatgagaatggtgagctgtgcatgtcagacgagcagagggaagacggcagccactctcatgtcaaatcccagcgtcttttgggaggcagcttcccttttttagtttagtttgttggaagaaaagaattgtccctttcccccctctaaactaaaagccttgccagcccaggtgggcagcaccgaggtccctgcagggaacgtgcaaggggaaccctgcagtttcccgctcacatgcccttccgagactgagtgctccgaggactgaggacgagaaatatgccaggtctgccactgccttcttacgagacccggacccaggggaggcacagccatgcccagctcctgcctgccagttctgtcctcccagctgccctactttcatgctgggacctccaattcagtacaaagggagacctcactgtttctgaaccatctctactcagactcccaagtgccacgtgcccaggggactgttctgtgacaaacttatacacaacttcaccctattctcctaagaacaaccgcagaataggcctttcaggatgagtgggaggacagccgagggcagggatgtgctagtgtaaggtcgaggcagagggtgggctgctgtcatggaaagaccccaggtaactgcgtcacacacaaatttgtgtccttctcccacaacgggctctcccgagttctctgtcatctgcacggccctgtgagcaggaggggaaacagagggctcacccctgcccccaaggcccagtgtgcaaatccattcatcacaacgaggttgtgtgagtctccccagtagcaagggctgctgaggaatggagccctcgtttccggggcctgcgtggcccactctgtattctatgactgtgatgggggagggtgggggccacaggacagctggtgggctctgccatggctggggctagacatggattaaaaagtgagtatgagcaggggcctctaggagtggtgggatagtgcggtggtggccacatgtcattctacgtgcgtccaaacctacagaatgtaaaacaccaggagggagactcaaagaaaactatcaactttgagtgctgaggacgtgtcagtgtaggttcgtcagttgcaacaaatgggccacgctggtgtgagatgttgatcacgggggaggctgtgtagtgggggacaagagttatatgggaactttctgtactttctgctcgattttgctgtgaacctaaagtcactctaaaaaataacatctcttaaattttttaaaaagtgagtgtgtcaaaccacagcctttgggtcaggacagttctaggtttgagttgacctggcaggtaccagtggcttatgtcccttaaggtgacagatgcaaaacccccggtttggtgcctggcatgttgtgtgtcttgcaggtggcggttagggctgcctcagtgaactcaaatggctgcattttacaggagaaatatttgagccacacttgcggtcctgtggccaggagaatgcagagtggcctggggggggccaaggaaggaggctgaggcagggcgaggggcaggatctgggcctttggtgtctgccagccctcattcctgcccctgtcttgggtgactcttccctccctgtctcctgtctggatttcagggaagatgaagggcttctccctgctggccgagccccaggagttctgggtggacaacagcacctcagtgtctgttcccatgctctctggcatgggcaccttccagcactggagtgacatccaggacaacttctcggtgactcaagtgcccttcactgagagcgcctgcctgctgctgatccagcctcactatgcctctgacctggacaaggtggagggtctcactttccagcaaaactccctcaactggatgaagaaactatctccccggtaggagcctcccggtctcccctggaatgtgggagccacactgtcctgcccaggctgggggcggggtggggagtagacacacctgagctgagccttgggtgcagagcagggcagggccgcggtggcacggggctgggcaggcggcctgtgtgtctgtctaccagtcctctatccagccagcacccagctctccagttagtgtctgtctttcaagtgcaggcaaggtaaaggaggagaggaagaatgctttttctacacttacacttgcctggtagttttggagggggagaaaacattgcaatccgccctctgagagaggaccattttggtcccacacctgacacacagcacacctgtgacatccaagagcttcttggaactgacttgccaggagggttcggacttcgcgtgagcgggggtggggccttctcagggagcgtcccttgactccagaacgcccttgctggcggctggcggctgggtggggataggtgttgttagctcctctttcctgctgcaattcctttccacagagccctggactcaaactacacatcaccccagatcatcgaggcctggaaatctgctcccagaggcaggcattgagtgacacgatggcttgacatcaactctgggtgttttttatgttttaaaaattgtgatggtaaaatatacgtaacaaaatttgccatcgtaaccattttcgagtgcacagttcagtggtactaggcccattcacactgttgtgcagccatcacccccgtccatctccatttatcttctcaacttcccaaactgaagctctgtcctgctgaaacactaactctccatttccccttccccttggccccggcaaccaccacgatgtcctcgaggttcacccatgttgtagcacatgtcagaatgtccttccttttgaaggctgaataatattccattgcatgtggttaccaccttttgtgtatccactcatccatcgatggacacgtgggttgcttccacctttgagctgctgtgaatagtgcagtgtaccctgtaaacatgggtgtactgtcagctcttataagtgcttgatacatcactggaaatgtccatgggctctgaaggatgccaaaagatggaagaggctctatacgaagatcaatcgagttgacatagcaacgtgtccagcacgaggttgacactgtaccctcctgcctctctccttttcatgggtgtcatgtcatcaagaacactgctgtggcagtagtaagacacagtgcattatttcagagaatagcatttaaaaattacccaagtaacacaccttcaatgcagccaacctaaaaacagaatgcaccaaaggacaaccattcctaggtcctcatcggtaaatcttctatgtccctcacatagtattgcaaatgacatgaaggatttttattgtaggttttgctgaaattttccccaagggggaggatgacttagttgggtgatggggggagcaaacatccctgtcgtcagggttgggtgcaaggagcataagcctgcctggcctctgggagagccctcactgtgtggcctggagccttcctaactgtgcatcatctccccaggaccatccacctgaccatgccccaactggtgctgcaaggatcttatgacctgcaggacctgctcgcccaggctgagctgcccgccattctgcacaccgagctgaacctgcaaaaattgagcaatgaccgcatcagggtgggggaggtatgtgtgagcctgtgtctgtgcctgacctgggttccaagtgtgcacagggtgggaggcatggatgtaagggacacagaggaggctatgggtggggccagcagggcaagagggagcggagagtagggccaaaggtgggagagaagtagccagagcattctggggccttccaggtgcagagcagcaaatccctccccatccctgctgtgcctcctcctgctaggtgtgtgttccatggtcctgcttggccttgccttgcctcagggtcctccagggttcctatagtggagttgaaaccgggatgaagacagcaagcacccctggacctggtgccctgggcccagccccttcttcagggaaatgctgagcagcagacagaatgtccccctgccatgtggcaccatgcacatctgcagctaccaaggatgtgccttgatgttctgggccctgtgctcagtgctggggagaaagtgggagttcttacgggggccagcgggaagagccctctgtgctaagttagctaagccctggcactggtgggccatggccaagggagccaggaattctgcctgggacatcagggcagaatgtgaagatgggaggatgtaaggggtgtgttagggaggagccggcatgtgagtttggccattgtggccaattaacggtcatctacacacagacacacccttgcctacactgaggggcaggcatacactgtgcatcctcctggcaggctggaaaatgtccccctccaggacagtgcacagcacagaggtcctgagcccaccccggccctctagccctcagcaccctgggtcacccagtgcgccctcagaatgatcctgatgtctgctgctttgcaggtgctgaacagcattttttttgagcttgaagcggatgagagagagcccacagagtctacccaacagcttaacaagcctgaggtcttggaggtgaccctgaaccgcccattcctgtttgctgtgtatgatcaaagcgccactgccctgcacttcctgggccgcgtggccaacccgctgagcacagcatgaggccagggccccagaacacagtgcctggcaaggcctctgcccctggcctttgaggcaaaggccagcagcagataacaaccccggacaaatcagcgatgtgtcacccccagtctcccaccttttcttctaatgagtcgactttgagctggaaagcagccgtttctccttggtctaagtgtgctgcatggagtgagcagtagaagcctgcagcggcacaaatgcacctcccagtttgctgggtttattttagagaatgggggtggggaggcaagaaccagtgtttagcgcgggactactgttccaaaaagaattccaaccgaccagcttgtttgtgaaacaaaaaagtgttcccttttcaagttgagaacaaaaattgggttttaaaattaaagtatacatttttgcattgccttcggtttgtatttagtgtcttgaatgtaagaacatgacctccgtgtagtgtctgtaataccttagttttttccacagatgcttgtgatttttgaacaatacgtgaaagatgcaagcacctgaatttctgtttgaatgcggaaccatagctggttatttctcccttgtgttagtaataaacgtcttgccacaataagcctccaaaaattttatctttcatttagcagccaaacagatgtatacaattcagcagatagactgtgcaaacgaaagtgctttcctggactttggatggaatttccatgggaggtctgagccagtacttagcagtcctttgaagttttaggtgatgcttttctctggacacttccattggtaagcagtggtggccatctgtgtgatggacagggggcgggaagagggtgacagggaaggccccataccccatgtggcacctgggaaaggaaccaggcagatgggacttcttccgtcctggtgacacagggccagactgctgctggtattgtgccccgggagtggaaggtagagaaataaatcttcacaaataaatatttgcaattttcccccatctgttgagtgcctctgcctgctcctcctcgatgggattaggcccacagttcggaatcttggggagagccaaggaagcggtaggcacccagtaggcccacggccgtcggctgatagcaatggtgatgctgtcctacctacttgtgtaaggcattcgatcttcctcccttccatacatattgaaataaataagccgcgcaatgtgttagctattgatcagaactaaagtgaagtcagccacggggattacaaatctcggcttctcccctcatgttcctgagagtcttcccctggttttgaacacatctccctagctcgatgtcaaggtgagggattctgtcggcaacagcagtgcccttagttgcttcgtcgtaactccccgtcaccggttttattcagttaccttccagtcccactctcagagcttcctggcttgttctgctctcaaagcgggtagagctggcacacatggactctccgaaacggctgcaagatgccaagtttctcggaagaactggaagcacagagaccagaagtgccttaaggtctcgctattcagtgtggcgcttagaccggcagtggcggcagctgccctgggagcttgttagaatgtggcttctcacgcccctcctggacctacagagtcagaatctgcagttttacaggaggtccaggcttggaagttgctcgtagagacctgagacagcgcagccacgtgctggaaacaaagcatttaagtttgtgactttattttaaaaggcagcaggcagtcgacaaaccaatttcttctacttagaggcggcttcggcttctggaagtcgctaggagtataaagttgccaaccagcgctgttctcccgctgttttctgtgcacttataaatgggaagttaggtcaggatagatctctcagctattacaaggatacaaaatacgaacattctacaagttacttaacacacacacacacacacacacacacacacacacacacaaaattaattccacaggtcagtttctctgaaacattttttcactaaattctaagtcttcctggagttgcaagtgcctatctcctagacaaggcaattactcaccaactaaaatcactgtcaatctgagatttcggctgggcatgagaccatggtcaggggatgctttgaacagcctctgaggaaattagtgagtttgaaaaatggaaagatttttattactcacttggcagtaaaacctgatggggacagacgtcaggctgtttaagatcctcagaagaaaaagttgatagtgtgaatattcctaaatttgccacacgaagatgtacatgtgattataaggtgctgttgcagaagcccctgggggtgttatgggatatacactatatgggccactttaccttcctaaaatctgaaaaacttcaactactgaaacatggactgaaggttttgaatagtggatggtgaatttgaataccatcccgtgtgatttttttttctagcagactttagttttttagagcagttttaagcccacaccaaaactgagaggaagatacagcaatttctcatataccccctactaccttccagtctcccccattattgacatcccccacccagagtgg

SEQ ID NO: 5-9 - skipped (5 entries)

SEQ ID NO: 10 - RNA, 21 nt, synthetic construct
  source 1..21: mol_type = other RNA
tatgtacacccggtcacctgc

SEQ ID NO: 11 - RNA, 21 nt, synthetic construct
  source 1..21: mol_type = other RNA
ttatgtacacccggtcacctg

SEQ ID NO: 12 - RNA, 21 nt, synthetic construct
  source 1..21: mol_type = other RNA
  modified_base 14: mod_base = i
tgtatgtacacccngtcacct

SEQ ID NO: 13 - RNA, 21 nt, synthetic construct
  source 1..21: mol_type = other RNA
aaggggtgtatgtacacccgg

SEQ ID NO: 14 - RNA, 21 nt, synthetic construct
  source 1..21: mol_type = other RNA
taaggggtgtatgtacacccg

SEQ ID NO: 15 - RNA, 21 nt, synthetic construct
  source 1..21: mol_type = other RNA
tgaaggggtgtatgtacaccc

SEQ ID NO: 16 - RNA, 21 nt, synthetic construct
  source 1..21: mol_type = other RNA
tggaaggggtgtatgtacacc

SEQ ID NO: 17 - RNA, 21 nt, synthetic construct
  source 1..21: mol_type = other RNA
ttggaaggggtgtatgtacac

SEQ ID NO: 18 - RNA, 21 nt, synthetic construct
  source 1..21: mol_type = other RNA
acgaggtggaaggggtgtatg

SEQ ID NO: 19 - RNA, 21 nt, synthetic construct
  source 1..21: mol_type = other RNA
tacgaggtggaaggggtgtat

SEQ ID NO: 20 - RNA, 21 nt, synthetic construct
  source 1..21: mol_type = other RNA
tgacgaggtggaaggggtgta

SEQ ID NO: 21 - RNA, 21 nt, synthetic construct
  source 1..21: mol_type = other RNA
atgacgaggtggaaggggtgt

SEQ ID NO: 22 - RNA, 21 nt, synthetic construct
  source 1..21: mol_type = other RNA
tatgacgaggtggaaggggtg

SEQ ID NO: 23 - RNA, 21 nt, synthetic construct
  source 1..21: mol_type = other RNA
tgatgacgaggtggaaggggt

SEQ ID NO: 24 - RNA, 22 nt, synthetic construct
  source 1..22: mol_type = other RNA
tcattgtggatgatgaggtgga

SEQ ID NO: 25 - RNA, 21 nt, synthetic construct
  source 1..21: mol_type = other RNA
tcattgtggatgatgaggtgg

SEQ ID NO: 26 - RNA, 21 nt, synthetic construct
  source 1..21: mol_type = other RNA
tgccagctgctcacaggtact

SEQ ID NO: 27 - RNA, 21 nt, synthetic construct
  source 1..21: mol_type = other RNA
ttgccagctgctcacaggtac

SEQ ID NO: 28 - RNA, 21 nt, synthetic construct
  source 1..21: mol_type = other RNA
tttgccagctgctcacaggta

SEQ ID NO: 29 - RNA, 21 nt, synthetic construct
  source 1..21: mol_type = other RNA
  modified_base 14: mod_base = i
ttcatccacagggnatgtctc

SEQ ID NO: 30 - RNA, 21 nt, synthetic construct
  source 1..21: mol_type = other RNA
tgtagggccttttcatccaca

SEQ ID NO: 31 - RNA, 22 nt, synthetic construct
  source 1..22: mol_type = other RNA
aagttttgcagcgactagcaca

SEQ ID NO: 32 - RNA, 23 nt, synthetic construct
  source 1..23: mol_type = other RNA
tcaagaagttggccagcatccgc

SEQ ID NO: 33 - RNA, 21 nt, synthetic construct
  source 1..21: mol_type = other RNA
tccaagaagttggccagcatc

SEQ ID NO: 34 - RNA, 21 nt, synthetic construct
  source 1..21: mol_type = other RNA
tcccaagaagttggccagcat

SEQ ID NO: 35 - RNA, 21 nt, synthetic construct
  source 1..21: mol_type = other RNA
tggaagcccaagaagttggcc

SEQ ID NO: 36 - RNA, 22 nt, synthetic construct
  source 1..22: mol_type = other RNA
tccatatatacggaagcccgta

SEQ ID NO: 37 - RNA, 21 nt, synthetic construct
  source 1..21: mol_type = other RNA
tccatatatacggaagcccaa

SEQ ID NO: 38 - RNA, 21 nt, synthetic construct
  source 1..21: mol_type = other RNA
tccatatatacggaagcccag

SEQ ID NO: 39 - RNA, 21 nt, synthetic construct
  source 1..21: mol_type = other RNA
tgcatgccatatatacggaag

SEQ ID NO: 40 - RNA, 22 nt, synthetic construct
  source 1..22: mol_type = other RNA
atgcatgccatatatacggtga

SEQ ID NO: 41 - RNA, 21 nt, synthetic construct
  source 1..21: mol_type = other RNA
ttgcatgccatatatacggaa

SEQ ID NO: 42 - RNA, 21 nt, synthetic construct
  source 1..21: mol_type = other RNA
tgtgcatgccatatatacgga

SEQ ID NO: 43 - RNA, 21 nt, synthetic construct
  source 1..21: mol_type = other RNA
tcactgtgcatgccatatata

SEQ ID NO: 44 - RNA, 21 nt, synthetic construct
  source 1..21: mol_type = other RNA
tccatagctcactgtgcatgc

SEQ ID NO: 45 - RNA, 21 nt, synthetic construct
  source 1..21: mol_type = other RNA
tcccatagctcactgtgcatg

SEQ ID NO: 46 - RNA, 21 nt, synthetic construct
  source 1..21: mol_type = other RNA
taccacgccccatagctcact

SEQ ID NO: 47 - RNA, 21 nt, synthetic construct
  source 1..21: mol_type = other RNA
agggtgccaaagacagccgtt

SEQ ID NO: 48 - RNA, 21 nt, synthetic construct
  source 1..21: mol_type = other RNA
tagggtgccaaagacagccgt

SEQ ID NO: 49 - RNA, 21 nt, synthetic construct
  source 1..21: mol_type = other RNA
taggccagggtgccaaagaca

SEQ ID NO: 50 - RNA, 21 nt, synthetic construct
  source 1..21: mol_type = other RNA
tatagagagaggccagggtgc

SEQ ID NO: 51 - RNA, 21 nt, synthetic construct
  source 1..21: mol_type = other RNA
agatagagagaggccagggtg

SEQ ID NO: 52 - RNA, 21 nt, synthetic construct
  source 1..21: mol_type = other RNA
  modified_base 14: mod_base = i
tagatagagagagnccagggt

SEQ ID NO: 53 - RNA, 22 nt, synthetic construct
  source 1..22: mol_type = other RNA
tctgtgtggtccaaggctccca

SEQ ID NO: 54 - RNA, 21 nt, synthetic construct
  source 1..21: mol_type = other RNA
aggattgcctgtagcctgtca

SEQ ID NO: 55 - RNA, 21 nt, synthetic construct
  source 1..21: mol_type = other RNA
taggattgcctgtagcctgtc

SEQ ID NO: 56 - RNA, 21 nt, synthetic construct
  source 1..21: mol_type = other RNA
tcaggattgcctgtagcctgt

SEQ ID NO: 57 - RNA, 21 nt, synthetic construct
  source 1..21: mol_type = other RNA
tccaggattgcctgtagcctg

SEQ ID NO: 58 - RNA, 21 nt, synthetic construct
  source 1..21: mol_type = other RNA
acccaggattgcctgtagcct

SEQ ID NO: 59 - RNA, 21 nt, synthetic construct
  source 1..21: mol_type = other RNA
tacccaggattgcctgtagcc

SEQ ID NO: 60 - RNA, 21 nt, synthetic construct
  source 1..21: mol_type = other RNA
aggtgcagttcttgtccttcc

SEQ ID NO: 61 - RNA, 21 nt, synthetic construct
  source 1..21: mol_type = other RNA
taggtgcagttcttgtccttc

SEQ ID NO: 62 - RNA, 21 nt, synthetic construct
  source 1..21: mol_type = other RNA
tgaggtgcagttcttgtcctt

SEQ ID NO: 63 - RNA, 21 nt, synthetic construct
  source 1..21: mol_type = other RNA
tggaggtgcagttcttgtcct

SEQ ID NO: 64 - RNA, 22 nt, synthetic construct
  source 1..22: mol_type = other RNA
tttgtgcgcatccagccgggaa

SEQ ID NO: 65 - RNA, 21 nt, synthetic construct
  source 1..21: mol_type = other RNA
taggaccttgtgcgcatccag

SEQ ID NO: 66 - RNA, 21 nt, synthetic construct
  source 1..21: mol_type = other RNA
acaggaccttgtgcgcatcca

SEQ ID NO: 67 - RNA, 21 nt, synthetic construct
  source 1..21: mol_type = other RNA
tacaggaccttgtgcgcatcc

SEQ ID NO: 68 - RNA, 21 nt, synthetic construct
  source 1..21: mol_type = other RNA
ttgtacagcctgcagggcaga

SEQ ID NO: 69 - RNA, 21 nt, synthetic construct
  source 1..21: mol_type = other RNA
ttagcaggccctgtacagcct

SEQ ID NO: 70 - RNA, 21 nt, synthetic construct
  source 1..21: mol_type = other RNA
acaaacggctgcttcaggtgc

SEQ ID NO: 71 - RNA, 21 nt, synthetic construct
  source 1..21: mol_type = other RNA
tacaaacggctgcttcaggtg

SEQ ID NO: 72 - RNA, 21 nt, synthetic construct
  source 1..21: mol_type = other RNA
tcacaaacggctgcttcaggt

SEQ ID NO: 73 - RNA, 21 nt, synthetic construct
  source 1..21: mol_type = other RNA
tgcacaaacggctgcttcagg

SEQ ID NO: 74 - RNA, 21 nt, synthetic construct
  source 1..21: mol_type = other RNA
ttgcacaaacggctgcttcag

SEQ ID NO: 75 - RNA, 21 nt, synthetic construct
  source 1..21: mol_type = other RNA
tctgcacaaacggctgcttca

SEQ ID NO: 76 - RNA, 21 nt, synthetic construct
  source 1..21: mol_type = other RNA
tgtatagagagccaggccctg

SEQ ID NO: 77 - RNA, 22 nt, synthetic construct
  source 1..22: mol_type = other RNA
tagagagcgtgggaggaccaca

SEQ ID NO: 78 - RNA, 21 nt, synthetic construct
  source 1..21: mol_type = other RNA
ttgtgaagtccagagagcgtg

SEQ ID NO: 79 - RNA, 22 nt, synthetic construct
  source 1..22: mol_type = other RNA
tctgtgaagtccagagagcgta

SEQ ID NO: 80 - RNA, 21 nt, synthetic construct
  source 1..21: mol_type = other RNA
tctgtgaagtccagagagcgt

SEQ ID NO: 81 - RNA, 21 nt, synthetic construct
  source 1..21: mol_type = other RNA
ttctgtgaagtccagagagcg

SEQ ID NO: 82 - RNA, 21 nt, synthetic construct
  source 1..21: mol_type = other RNA
tttctgtgaagtccagagagc

SEQ ID NO: 83 - RNA, 21 nt, synthetic construct
  source 1..21: mol_type = other RNA
agttctgtgaagtccagagag

SEQ ID NO: 84 - RNA, 22 nt, synthetic construct
  source 1..22: mol_type = other RNA
tttctcagcagcaacatccaga

SEQ ID NO: 85 - RNA, 21 nt, synthetic construct
  source 1..21: mol_type = other RNA
acagcctgcatgaacctgtca

SEQ ID NO: 86 - RNA, 21 nt, synthetic construct
  source 1..21: mol_type = other RNA
tacagcctgcatgaacctgtc

SEQ ID NO: 87 - RNA, 21 nt, synthetic construct
  source 1..21: mol_type = other RNA
atcctgtcacagcctgcatga

SEQ ID NO: 88 - RNA, 21 nt, synthetic construct
  source 1..21: mol_type = other RNA
tatcctgtcacagcctgcatg

SEQ ID NO: 89 - RNA, 21 nt, synthetic construct
  source 1..21: mol_type = other RNA
tcatcctgtcacagcctgcat

SEQ ID NO: 90 - RNA, 21 nt, synthetic construct
  source 1..21: mol_type = other RNA
tccatcctgtcacagcctgca

SEQ ID NO: 91 - RNA, 21 nt, synthetic construct
  source 1..21: mol_type = other RNA
ttccatcctgtcacagcctgc

SEQ ID NO: 92 - RNA, 22 nt, synthetic construct
  source 1..22: mol_type = other RNA
tttccatcctgtcacagcctca

SEQ ID NO: 93 - RNA, 21 nt, synthetic construct
  source 1..21: mol_type = other RNA
tttccatcctgtcacagcctg

SEQ ID NO: 94 - RNA, 21 nt, synthetic construct
  source 1..21: mol_type = other RNA
taggtgttgaaagtcagggtg

SEQ ID NO: 95 - RNA, 21 nt, synthetic construct
  source 1..21: mol_type = other RNA
aagtggacgtaggtgttgaaa

SEQ ID NO: 96 - RNA, 21 nt, synthetic construct
  source 1..21: mol_type = other RNA
tgctgttgtccacccagaact

SEQ ID NO: 97 - RNA, 21 nt, synthetic construct
  source 1..21: mol_type = other RNA
ttgctgttgtccacccagaac

SEQ ID NO: 98 - RNA, 21 nt, synthetic construct
  source 1..21: mol_type = other RNA
tacactgaggtgctgttgtcc

SEQ ID NO: 99 - RNA, 21 nt, synthetic construct
  source 1..21: mol_type = other RNA
tactccagtgctggaaggtgc

SEQ ID NO: 100 - RNA, 21 nt, synthetic construct
  source 1..21: mol_type = other RNA
tcactccagtgctggaaggtg

SEQ ID NO: 101 - RNA, 21 nt, synthetic construct
  source 1..21: mol_type = other RNA
tgtcactccagtgttggaagg

SEQ ID NO: 102 - RNA, 23 nt, synthetic construct
  source 1..23: mol_type = other RNA
acttgagtcaccgagaagttgga

SEQ ID NO: 103 - RNA, 21 nt, synthetic construct
  source 1..21: mol_type = other RNA
tctcagtgaagggtacttgag

SEQ ID NO: 104 - RNA, 21 nt, synthetic construct
  source 1..21: mol_type = other RNA
accttgtccaggtcagaggca

SEQ ID NO: 105 - RNA, 21 nt, synthetic construct
  source 1..21: mol_type = other RNA
accttgtccaggttagaggca

SEQ ID NO: 106 - RNA, 21 nt, synthetic construct
  source 1..21: mol_type = other RNA
taccttgtccaggtcagaggc

SEQ ID NO: 107 - RNA, 21 nt, synthetic construct
  source 1..21: mol_type = other RNA
tcaccttgtccaggtcagagg

SEQ ID NO: 108 - RNA, 21 nt, synthetic construct
  source 1..21: mol_type = other RNA
tccaccttgtccaggtcagag

SEQ ID NO: 109 - RNA, 21 nt, synthetic construct
  source 1..21: mol_type = other RNA
ttccaccttgtccaggtcaga

SEQ ID NO: 110 - RNA, 21 nt, synthetic construct
  source 1..21: mol_type = other RNA
ttgagaccctccaccttgtcc

SEQ ID NO: 111 - RNA, 22 nt, synthetic construct
  source 1..22: mol_type = other RNA
tggaaagtgagactctccacca

SEQ ID NO: 112 - RNA, 21 nt, synthetic construct
  source 1..21: mol_type = other RNA
tggaaagtgagactctccacc

SEQ ID NO: 113 - RNA, 21 nt, synthetic construct
  source 1..21: mol_type = other RNA
ttgctggaaagtgagaccctc

SEQ ID NO: 114 - RNA, 23 nt, synthetic construct
  source 1..23: mol_type = other RNA
ttttgctggaaagtgagaccctc

SEQ ID NO: 115 - RNA, 23 nt, synthetic construct
  source 1..23: mol_type = other RNA
agtttcttcatccagttgaggga

SEQ ID NO: 116 - RNA, 21 nt, synthetic construct
  source 1..21: mol_type = other RNA
tggcatggtcaggtggatggt

SEQ ID NO: 117 - RNA, 21 nt, synthetic construct
  source 1..21: mol_type = other RNA
ttggggcatggtcaggtggat

SEQ ID NO: 118 - RNA, 21 nt, synthetic construct
  source 1..21: mol_type = other RNA
tttggggcatggtcaggtgga

SEQ ID NO: 119 - RNA, 21 nt, synthetic construct
  source 1..21: mol_type = other RNA
tctgcaggtcataagatcctt

SEQ ID NO: 120 - RNA, 21 nt, synthetic construct
  source 1..21: mol_type = other RNA
tcctgcaggtcataagatcct

SEQ ID NO: 121 - RNA, 21 nt, synthetic construct
  source 1..21: mol_type = other RNA
ttcctgcaggtcataagatcc

SEQ ID NO: 122 - RNA, 21 nt, synthetic construct
  source 1..21: mol_type = other RNA
tgagcaggtcctgcaggtcat

SEQ ID NO: 123 - RNA, 21 nt, synthetic construct
  source 1..21: mol_type = other RNA
tcaatttttgcaggttcagct

SEQ ID NO: 124 - RNA, 21 nt, synthetic construct
  source 1..21: mol_type = other RNA
ttcaatttttgcaggttcagc

SEQ ID NO: 125 - RNA, 21 nt, synthetic construct
  source 1..21: mol_type = other RNA
ttctgtgggctctctctcatc

SEQ ID NO: 126 - RNA, 21 nt, synthetic construct
  source 1..21: mol_type = other RNA
tggtagactctgtgggctctc

SEQ ID NO: 127 - RNA, 21 nt, synthetic construct
  source 1..21: mol_type = other RNA
tgggtagactctgtgggctct

SEQ ID NO: 128 - RNA, 21 nt, synthetic construct
  source 1..21: mol_type = other RNA
ttgggtagactctgtgggctc

SEQ ID NO: 129 - RNA, 21 nt, synthetic construct
  source 1..21: mol_type = other RNA
tttgggtagactctgtgggct

SEQ ID NO: 130 - RNA, 21 nt, synthetic construct
  source 1..21: mol_type = other RNA
  modified_base 14: mod_base = i
taaacaggaatggncggttca

SEQ ID NO: 131 - RNA, 21 nt, synthetic construct
  source 1..21: mol_type = other RNA
  modified_base 14: mod_base = i
tcaaacaggaatgngcggttc

SEQ ID NO: 132 - RNA, 21 nt, synthetic construct
  source 1..21: mol_type = other RNA
  modified_base 14: mod_base = i
acacagcaaacagnaatgggc

SEQ ID NO: 133 - RNA, 23 nt, synthetic construct
  source 1..23: mol_type = other RNA
tgatcatacacagtaaacaggaa

SEQ ID NO: 134 - RNA, 21 nt, synthetic construct
  source 1..21: mol_type = other RNA
tgatcatacacagtaaacagg

SEQ ID NO: 135 - RNA, 23 nt, synthetic construct
  source 1..23: mol_type = other RNA
tgatcatacacagtaaacaggga

SEQ ID NO: 136 - RNA, 23 nt, synthetic construct
  source 1..23: mol_type = other RNA
tgatcatacacagtaaacaccgg

SEQ ID NO: 137 - RNA, 23 nt, synthetic construct
  source 1..23: mol_type = other RNA
tgatcatacacagtaaacagcgg

SEQ ID NO: 138 - RNA, 21 nt, synthetic construct
  source 1..21: mol_type = other RNA
agaaaaggtgggagactgggg

SEQ ID NO: 139 - RNA, 23 nt, synthetic construct
  source 1..23: mol_type = other RNA
attagaagaaaaggtgggagact

SEQ ID NO: 140 - RNA, 21 nt, synthetic construct
  source 1..21: mol_type = other RNA
ttcgactcattagaagaaaag

SEQ ID NO: 141 - RNA, 21 nt, synthetic construct
  source 1..21: mol_type = other RNA
  modified_base 14: mod_base = i
accaaggagaaacngctgctt

SEQ ID NO: 142 - RNA, 21 nt, synthetic construct
  source 1..21: mol_type = other RNA
ctactgctcactctatgcagc

SEQ ID NO: 143 - RNA, 21 nt, synthetic construct
  source 1..21: mol_type = other RNA
tactggttcttgcctccccac

SEQ ID NO: 144 - RNA, 21 nt, synthetic construct
  source 1..21: mol_type = other RNA
acactggttcttgcctcccca

SEQ ID NO: 145 - RNA, 21 nt, synthetic construct
  source 1..21: mol_type = other RNA
aacactggttcttgcctcccc

SEQ ID NO: 146 - RNA, 22 nt, synthetic construct
  source 1..22: mol_type = other RNA
taacactggttcttgcctcgga

SEQ ID NO: 147 - RNA, 21 nt, synthetic construct
  source 1..21: mol_type = other RNA
aaacactggttcttgcctccc

SEQ ID NO: 148 - RNA, 21 nt, synthetic construct
  source 1..21: mol_type = other RNA
taacactggttcttgcctccc

SEQ ID NO: 149 - RNA, 21 nt, synthetic construct
  source 1..21: mol_type = other RNA
taacactggttcttgcctccg

SEQ ID NO: 150 - skipped

SEQ ID NO: 151 - RNA, 23 nt, synthetic construct
  source 1..23: mol_type = other RNA
tctaaacactggttcttgcctcc

SEQ ID NO: 152 - RNA, 21 nt, synthetic construct
  source 1..21: mol_type = other RNA
ttcccgcgctaaacactggtt

SEQ ID NO: 153 - RNA, 21 nt, synthetic construct
  source 1..21: mol_type = other RNA
agtcccgcgctaaacactggt

SEQ ID NO: 154 - RNA, 21 nt, synthetic construct
  source 1..21: mol_type = other RNA
tagtcccgcgctaaacactgg

SEQ ID NO: 155 - RNA, 21 nt, synthetic construct
  source 1..21: mol_type = other RNA
ttagtcccgcgctaaacactg

SEQ ID NO: 156 - RNA, 21 nt, synthetic construct
  source 1..21: mol_type = other RNA
tggaacagtagtctcgcgcta

SEQ ID NO: 157 - RNA, 21 nt, synthetic construct
  source 1..21: mol_type = other RNA
tttggaattctttttggaaca

SEQ ID NO: 158 - RNA, 21 nt, synthetic construct
  source 1..21: mol_type = other RNA
tgtcggttggaattctttttg

SEQ ID NO: 159 - RNA, 21 nt, synthetic construct
  source 1..21: mol_type = other RNA
  modified_base 14: mod_base = i
caacttgaaaaggnaacactg

SEQ ID NO: 160 - RNA, 21 nt, synthetic construct
  source 1..21: mol_type = other RNA
  modified_base 14: mod_base = i
tcaacttgaaaagngaacact

SEQ ID NO: 161 - RNA, 21 nt, synthetic construct
  source 1..21: mol_type = other RNA
  modified_base 14: mod_base = i
tcaacttgaaaagngaacacg

SEQ ID NO: 162 - RNA, 21 nt, synthetic construct
  source 1..21: mol_type = other RNA
tacaaaccgaaggtaatgcaa

SEQ ID NO: 163 - RNA, 21 nt, synthetic construct
  source 1..21: mol_type = other RNA
tacaaaccgaaggtaatgcag

SEQ ID NO: 164 - RNA, 21 nt, synthetic construct
  source 1..21: mol_type = other RNA
actaaatacaaactgaaggcg

SEQ ID NO: 165 - RNA, 21 nt, synthetic construct
  source 1..21: mol_type = other RNA
  modified_base 14: mod_base = i
acagacactacacngaggtca

SEQ ID NO: 166 - RNA, 21 nt, synthetic construct
  source 1..21: mol_type = other RNA
  modified_base 14: mod_base = i
acagacactacacngaggtcg

SEQ ID NO: 167 - RNA, 23 nt, synthetic construct
  source 1..23: mol_type = other RNA
tgcaggtgaccgggtgtacatac

SEQ ID NO: 168 - RNA, 23 nt, synthetic construct
  source 1..23: mol_type = other RNA
gcaggtgaccgggtgtacataaa

SEQ ID NO: 169 - RNA, 23 nt, synthetic construct
  source 1..23: mol_type = other RNA
taggtgaccgggtgtacatacat

SEQ ID NO: 170 - RNA, 22 nt, synthetic construct
  source 1..22: mol_type = other RNA
taggtgaccgggtgtacataca

SEQ ID NO: 171 - RNA, 23 nt, synthetic construct
  source 1..23: mol_type = other RNA
accgggtgtacatacaccccttc

SEQ ID NO: 172 - RNA, 23 nt, synthetic construct
  source 1..23: mol_type = other RNA
ccgggtgtacatacaccccttac

SEQ ID NO: 173 - RNA, 23 nt, synthetic construct
  source 1..23: mol_type = other RNA
cgggtgtacatacaccccttcaa

SEQ ID NO: 174 - RNA, 23 nt, synthetic construct
  source 1..23: mol_type = other RNA
gggtgtacatacaccccttccac

SEQ ID NO: 175 - RNA, 23 nt, synthetic construct
  source 1..23: mol_type = other RNA
ggtgtacatacaccccttccaac

SEQ ID NO: 176 - RNA, 23 nt, synthetic construct
  source 1..23: mol_type = other RNA
acatacaccccttccacctcgtc

SEQ ID NO: 177 - RNA, 23 nt, synthetic construct
  source 1..23: mol_type = other RNA
catacaccccttccacctcgtaa

SEQ ID NO: 178 - RNA, 23 nt, synthetic construct
  source 1..23: mol_type = other RNA
atacaccccttccacctcgtcat

SEQ ID NO: 179 - RNA, 23 nt, synthetic construct
  source 1..23: mol_type = other RNA
tacaccccttccacctcgtcatc

SEQ ID NO: 180 - RNA, 23 nt, synthetic construct
  source 1..23: mol_type = other RNA
acaccccttccacctcgtcatac

SEQ ID NO: 181 - RNA, 23 nt, synthetic construct
  source 1..23: mol_type = other RNA
caccccttccacctcgtcatcaa

SEQ ID NO: 182 - RNA, 23 nt, synthetic construct
  source 1..23: mol_type = other RNA
tccacctcatcatccacaatgat

SEQ ID NO: 183 - RNA, 22 nt, synthetic construct
  source 1..22: mol_type = other RNA
tccacctcatcatccacaatga

SEQ ID NO: 184 - RNA, 23 nt, synthetic construct
  source 1..23: mol_type = other RNA
gagtacctgtgagcagctggcaa

SEQ ID NO: 185 - RNA, 23 nt, synthetic construct
  source 1..23: mol_type = other RNA
agtacctgtgagcagctggcaaa

SEQ ID NO: 186 - RNA, 23 nt, synthetic construct
  source 1..23: mol_type = other RNA
gtacctgtgagcagctggcaaag

SEQ ID NO: 187 - RNA, 23 nt, synthetic construct
  source 1..23: mol_type = other RNA
tgagacatcccctgtggatgaat

SEQ ID NO: 188 - RNA, 23 nt, synthetic construct
  source 1..23: mol_type = other RNA
ctgtggatgaaaaggccctacag

SEQ ID NO: 189 - RNA, 23 nt, synthetic construct
  source 1..23: mol_type = other RNA
tgtgctagtcgctgcaaaacttt

SEQ ID NO: 190 - RNA, 21 nt, synthetic construct
  source 1..21: mol_type = other RNA
ggatgctggccaacttcttga

SEQ ID NO: 191 - RNA, 23 nt, synthetic construct
  source 1..23: mol_type = other RNA
ggatgctggccaacttcttggac

SEQ ID NO: 192 - RNA, 23 nt, synthetic construct
  source 1..23: mol_type = other RNA
gatgctggccaacttcttgggat

SEQ ID NO: 193 - RNA, 23 nt, synthetic construct
  source 1..23: mol_type = other RNA
tggccaacttcttgggcttccat

SEQ ID NO: 194 - RNA, 23 nt, synthetic construct
  source 1..23: mol_type = other RNA
tacgggcttccgtatatatggat

SEQ ID NO: 195 - RNA, 23 nt, synthetic construct
  source 1..23: mol_type = other RNA
cttgggcttccgtatatatggaa

SEQ ID NO: 196 - RNA, 22 nt, synthetic construct
  source 1..22: mol_type = other RNA
tctgggcttccgtatatatgga

SEQ ID NO: 197 - RNA, 23 nt, synthetic construct
  source 1..23: mol_type = other RNA
gcttccgtatatatggcatgcac

SEQ ID NO: 198 - RNA, 23 nt, synthetic construct
  source 1..23: mol_type = other RNA
tcaccgtatatatggcatgcatt

SEQ ID NO: 199 - RNA, 23 nt, synthetic construct
  source 1..23: mol_type = other RNA
cttccgtatatatggcatgcaaa

SEQ ID NO: 200 - RNA, 23 nt, synthetic construct
  source 1..23: mol_type = other RNA
ttccgtatatatggcatgcacag

SEQ ID NO: 201 - RNA, 23 nt, synthetic construct
  source 1..23: mol_type = other RNA
gtatatatggcatgcacagtgag

SEQ ID NO: 202 - RNA, 23 nt, synthetic construct
  source 1..23: mol_type = other RNA
ggcatgcacagtgagctatggag

SEQ ID NO: 203 - RNA, 23 nt, synthetic construct
  source 1..23: mol_type = other RNA
gcatgcacagtgagctatgggac

SEQ ID NO: 204 - RNA, 23 nt, synthetic construct
  source 1..23: mol_type = other RNA
cagtgagctatggggcgtggtac

SEQ ID NO: 205 - RNA, 23 nt, synthetic construct
  source 1..23: mol_type = other RNA
caacggctgtctttggcaccctg

SEQ ID NO: 206 - RNA, 23 nt, synthetic construct
  source 1..23: mol_type = other RNA
aacggctgtctttggcaccctag

SEQ ID NO: 207 - RNA, 23 nt, synthetic construct
  source 1..23: mol_type = other RNA
ctgtctttggcaccctggcctat

SEQ ID NO: 208 - RNA, 23 nt, synthetic construct
  source 1..23: mol_type = other RNA
ggcaccctggcctctctctatat

SEQ ID NO: 209 - RNA, 23 nt, synthetic construct
  source 1..23: mol_type = other RNA
gcaccctggcctctctctatctg

SEQ ID NO: 210 - RNA, 23 nt, synthetic construct
  source 1..23: mol_type = other RNA
taccctggcctctctctatctat

SEQ ID NO: 211 - RNA, 23 nt, synthetic construct
  source 1..23: mol_type = other RNA
tgggagccttggaccacacagat

SEQ ID NO: 212 - RNA, 23 nt, synthetic construct
  source 1..23: mol_type = other RNA
ctgacaggctacaggcaatcctg

SEQ ID NO: 213 - RNA, 23 nt, synthetic construct
  source 1..23: mol_type = other RNA
tgacaggctacaggcaatcctag

SEQ ID NO: 214 - RNA, 23 nt, synthetic construct
  source 1..23: mol_type = other RNA
gacaggctacaggcaatcctgag

SEQ ID NO: 215 - RNA, 23 nt, synthetic construct
  source 1..23: mol_type = other RNA
acaggctacaggcaatcctggat

SEQ ID NO: 216 - RNA, 23 nt, synthetic construct
  source 1..23: mol_type = other RNA
caggctacaggcaatcctgggtg

SEQ ID NO: 217 - RNA, 23 nt, synthetic construct
  source 1..23: mol_type = other RNA
aggctacaggcaatcctgggtat

SEQ ID NO: 218 - RNA, 23 nt, synthetic construct
  source 1..23: mol_type = other RNA
tggaaggacaagaactgcacctc

SEQ ID NO: 219 - RNA, 23 nt, synthetic construct
  source 1..23: mol_type = other RNA
ggaaggacaagaactgcacctac

SEQ ID NO: 220 - RNA, 23 nt, synthetic construct
  source 1..23: mol_type = other RNA
gaaggacaagaactgcacctcac

SEQ ID NO: 221 - RNA, 23 nt, synthetic construct
  source 1..23: mol_type = other RNA
aaggacaagaactgcacctccag

SEQ ID NO: 222 - RNA, 23 nt, synthetic construct
  source 1..23: mol_type = other RNA
ttcccggctggatgcgcacaaat

SEQ ID NO: 223 - RNA, 23 nt, synthetic construct
  source 1..23: mol_type = other RNA
gctggatgcgcacaaggtcctat

SEQ ID NO: 224 - RNA, 23 nt, synthetic construct
  source 1..23: mol_type = other RNA
ctggatgcgcacaaggtcctgtc

SEQ ID NO: 225 - RNA, 23 nt, synthetic construct
  source 1..23: mol_type = other RNA
tggatgcgcacaaggtcctgtat

SEQ ID NO: 226 - RNA, 23 nt, synthetic construct
  source 1..23: mol_type = other RNA
gtctgccctgcaggctgtacaag

SEQ ID NO: 227 - RNA, 23 nt, synthetic construct
  source 1..23: mol_type = other RNA
caggctgtacagggcctgctaat

SEQ ID NO: 228 - RNA, 23 nt, synthetic construct
  source 1..23: mol_type = other RNA
tgcacctgaagcagccgtttgtg

SEQ ID NO: 229 - RNA, 23 nt, synthetic construct
  source 1..23: mol_type = other RNA
gcacctgaagcagccgtttgtac

SEQ ID NO: 230 - RNA, 23 nt, synthetic construct
  source 1..23: mol_type = other RNA
cacctgaagcagccgtttgtgaa

SEQ ID NO: 231 - RNA, 23 nt, synthetic construct
  source 1..23: mol_type = other RNA
acctgaagcagccgtttgtgcag

SEQ ID NO: 232 - RNA, 23 nt, synthetic construct
  source 1..23: mol_type = other RNA
cctgaagcagccgtttgtgcaag

SEQ ID NO: 233 - RNA, 23 nt, synthetic construct
  source 1..23: mol_type = other RNA
ctgaagcagccgtttgtgcagag

SEQ ID NO: 234 - RNA, 23 nt, synthetic construct
  source 1..23: mol_type = other RNA
gcagggcctggctctctatacac

SEQ ID NO: 235 - RNA, 23 nt, synthetic construct
  source 1..23: mol_type = other RNA
tgtggtcctcccacgctctctat

SEQ ID NO: 236 - RNA, 23 nt, synthetic construct
  source 1..23: mol_type = other RNA
ccacgctctctggacttcacaaa

SEQ ID NO: 237 - RNA, 23 nt, synthetic construct
  source 1..23: mol_type = other RNA
tacgctctctggacttcacagat

SEQ ID NO: 238 - RNA, 23 nt, synthetic construct
  source 1..23: mol_type = other RNA
cacgctctctggacttcacagaa

SEQ ID NO: 239 - RNA, 23 nt, synthetic construct
  source 1..23: mol_type = other RNA
acgctctctggacttcacagaac

SEQ ID NO: 240 - RNA, 23 nt, synthetic construct
  source 1..23: mol_type = other RNA
cgctctctggacttcacagaaat

SEQ ID NO: 241 - RNA, 23 nt, synthetic construct
  source 1..23: mol_type = other RNA
gctctctggacttcacagaactg

SEQ ID NO: 242 - RNA, 23 nt, synthetic construct
  source 1..23: mol_type = other RNA
tctggatgttgctgctgagaaat

SEQ ID NO: 243 - RNA, 23 nt, synthetic construct
  source 1..23: mol_type = other RNA
ttgacaggttcatgcaggctgtg

SEQ ID NO: 244 - RNA, 22 nt, synthetic construct
  source 1..22: mol_type = other RNA
ttgacaggttcatgcagactgt

SEQ ID NO: 245 - RNA, 23 nt, synthetic construct
  source 1..23: mol_type = other RNA
tgacaggttcatgcaggctgtaa

SEQ ID NO: 246 - RNA, 23 nt, synthetic construct
  source 1..23: mol_type = other RNA
ttcatgcaggctgtgacaggatg

SEQ ID NO: 247 - RNA, 23 nt, synthetic construct
  source 1..23: mol_type = other RNA
tcatgcaggctgtgacaggatag

SEQ ID NO: 248 - RNA, 23 nt, synthetic construct
  source 1..23: mol_type = other RNA
catgcaggctgtgacaggatgaa

SEQ ID NO: 249 - RNA, 23 nt, synthetic construct
  source 1..23: mol_type = other RNA
atgcaggctgtgacaggatggaa

SEQ ID NO: 250 - RNA, 23 nt, synthetic construct
  source 1..23: mol_type = other RNA
tgcaggctgtgacaggatggaag

SEQ ID NO: 251 - RNA, 23 nt, synthetic construct
  source 1..23: mol_type = other RNA
tgaggctgtgacaggatggaaat

SEQ ID NO: 252 - RNA, 23 nt, synthetic construct
  source 1..23: mol_type = other RNA
gcaggctgtgacaggatggaaaa

SEQ ID NO: 253 - RNA, 23 nt, synthetic construct
  source 1..23: mol_type = other RNA
tcaccctgactttcaacacctat

SEQ ID NO: 254 - RNA, 23 nt, synthetic construct
  source 1..23: mol_type = other RNA
ctttcaacacctacgtccacttc

SEQ ID NO: 255 - RNA, 23 nt, synthetic construct
  source 1..23: mol_type = other RNA
gagttctgggtggacaacagcac

SEQ ID NO: 256 - RNA, 23 nt, synthetic construct
  source 1..23: mol_type = other RNA
agttctgggtggacaacagcaac

SEQ ID NO: 257 - RNA, 23 nt, synthetic construct
  source 1..23: mol_type = other RNA
tggacaacagcacctcagtgtat

SEQ ID NO: 258 - RNA, 23 nt, synthetic construct
  source 1..23: mol_type = other RNA
ggcaccttccagcactggagtaa

SEQ ID NO: 259 - RNA, 23 nt, synthetic construct
  source 1..23: mol_type = other RNA
gcaccttccagcactggagtgac

SEQ ID NO: 260 - RNA, 23 nt, synthetic construct
  source 1..23: mol_type = other RNA
tccttccaacactggagtgacat

SEQ ID NO: 261 - RNA, 24 nt, synthetic construct
  source 1..24: mol_type = other RNA
tccaacttctcggtgactcaagtt

SEQ ID NO: 262 - RNA, 23 nt, synthetic construct
  source 1..23: mol_type = other RNA
tctcaagtacccttcactgagat

SEQ ID NO: 263 - RNA, 23 nt, synthetic construct
  source 1..23: mol_type = other RNA
atgcctctgacctggacaaggtg

SEQ ID NO: 264 - RNA, 22 nt, synthetic construct
  source 1..22: mol_type = other RNA
ttgcctctaacctggataaggt

SEQ ID NO: 265 - RNA, 23 nt, synthetic construct
  source 1..23: mol_type = other RNA
tgcctctgacctggacaaggtag

SEQ ID NO: 266 - RNA, 23 nt, synthetic construct
  source 1..23: mol_type = other RNA
gcctctgacctggacaaggtgaa

SEQ ID NO: 267 - RNA, 23 nt, synthetic construct
  source 1..23: mol_type = other RNA
cctctgacctggacaaggtggag

SEQ ID NO: 268 - RNA, 23 nt, synthetic construct
  source 1..23: mol_type = other RNA
ctctgacctggacaaggtggaag

SEQ ID NO: 269 - RNA, 23 nt, synthetic construct
  source 1..23: mol_type = other RNA
tggacaaggtggagggtctcaat

SEQ ID NO: 270 - RNA, 23 nt, synthetic construct
  source 1..23: mol_type = other RNA
tggtggagagtctcactttccat

SEQ ID NO: 271 - skipped

SEQ ID NO: 272 - RNA, 23 nt, synthetic construct
  source 1..23: mol_type = other RNA
ggagggtctcactttccagcaaa

SEQ ID NO: 273 - RNA, 21 nt, synthetic construct
  source 1..21: mol_type = other RNA
gggtctcactttccagcaaaa

SEQ ID NO: 274 - RNA, 21 nt, synthetic construct
  source 1..21: mol_type = other RNA
cctcaactggatgaagaaact

SEQ ID NO: 275 - RNA, 23 nt, synthetic construct
  source 1..23: mol_type = other RNA
gaccatccacctgaccatgccac

SEQ ID NO: 276 - RNA, 23 nt, synthetic construct
  source 1..23: mol_type = other RNA
catccacctgaccatgccccaac

SEQ ID NO: 277 - RNA, 23 nt, synthetic construct
  source 1..23: mol_type = other RNA
atccacctgaccatgccccaaat

SEQ ID NO: 278 - RNA, 23 nt, synthetic construct
  source 1..23: mol_type = other RNA
caaggatcttatgacctgcagaa

SEQ ID NO: 279 - RNA, 23 nt, synthetic construct
  source 1..23: mol_type = other RNA
aaggatcttatgacctgcaggac

SEQ ID NO: 280 - RNA, 23 nt, synthetic construct
  source 1..23: mol_type = other RNA
aggatcttatgacctgcaggaac

SEQ ID NO: 281 - RNA, 23 nt, synthetic construct
  source 1..23: mol_type = other RNA
tatgacctgcaggacctgctcac

SEQ ID NO: 282 - RNA, 23 nt, synthetic construct
  source 1..23: mol_type = other RNA
gagctgaacctgcaaaaattgag

SEQ ID NO: 283 - RNA, 23 nt, synthetic construct
  source 1..23: mol_type = other RNA
agctgaacctgcaaaaattgaac

SEQ ID NO: 284 - RNA, 23 nt, synthetic construct
  source 1..23: mol_type = other RNA
ggatgagagagagcccacagaat

SEQ ID NO: 285 - RNA, 23 nt, synthetic construct
  source 1..23: mol_type = other RNA
agagagcccacagagtctaccaa

SEQ ID NO: 286 - RNA, 23 nt, synthetic construct
  source 1..23: mol_type = other RNA
gagagcccacagagtctacccaa

SEQ ID NO: 287 - RNA, 23 nt, synthetic construct
  source 1..23: mol_type = other RNA
agagcccacagagtctacccaac

SEQ ID NO: 288 - RNA, 23 nt, synthetic construct
  source 1..23: mol_type = other RNA
gagcccacagagtctacccaaaa

SEQ ID NO: 289 - RNA, 23 nt, synthetic construct
  source 1..23: mol_type = other RNA
ttgaaccgcccattcctgtttat

SEQ ID NO: 290 - RNA, 23 nt, synthetic construct
  source 1..23: mol_type = other RNA
tgaaccgcccattcctgtttgat

SEQ ID NO: 291 - RNA, 23 nt, synthetic construct
  source 1..23: mol_type = other RNA
tgcccattcctgtttgctgtgtt

SEQ ID NO: 292 - RNA, 21 nt, synthetic construct
  source 1..21: mol_type = other RNA
cctgtttactgtgtatgatca

SEQ ID NO: 293 - RNA, 23 nt, synthetic construct
  source 1..23: mol_type = other RNA
tcctgtttactgtgtatgatcat

SEQ ID NO: 294 - RNA, 22 nt, synthetic construct
  source 1..22: mol_type = other RNA
tcctgtttactgtgtatgatca

SEQ ID NO: 295 - RNA, 21 nt, synthetic construct
  source 1..21: mol_type = other RNA
cctgtttactgtgtatgatca

SEQ ID NO: 296 - RNA, 22 nt, synthetic construct
  source 1..22: mol_type = other RNA
ccctgtttactgtgtatgatca

SEQ ID NO: 297 - RNA, 23 nt, synthetic construct
  source 1..23: mol_type = other RNA
ccggtgtttactgtgtatgatca

SEQ ID NO: 298 - RNA, 23 nt, synthetic construct
  source 1..23: mol_type = other RNA
ccgctgtttactgtgtatgatca

SEQ ID NO: 299 - RNA, 23 nt, synthetic construct
  source 1..23: mol_type = other RNA
cccccagtctcccaccttttctt

SEQ ID NO: 300 - RNA, 22 nt, synthetic construct
  source 1..22: mol_type = other RNA
ttctcccaccttttcttctaat

SEQ ID NO: 301 - RNA, 23 nt, synthetic construct
  source 1..23: mol_type = other RNA
ccttttcttctaatgagtcgaat

SEQ ID NO: 302 - RNA, 23 nt, synthetic construct
  source 1..23: mol_type = other RNA
taagcagccgtttctccttggtt

SEQ ID NO: 303 - RNA, 23 nt, synthetic construct
  source 1..23: mol_type = other RNA
tgctgcatagagtgagcagtagt

SEQ ID NO: 304 - RNA, 23 nt, synthetic construct
  source 1..23: mol_type = other RNA
ggtggggaggcaagaaccagtat

SEQ ID NO: 305 - RNA, 23 nt, synthetic construct
  source 1..23: mol_type = other RNA
gtggggaggcaagaaccagtgtt

SEQ ID NO: 306 - RNA, 23 nt, synthetic construct
  source 1..23: mol_type = other RNA
tggggaggcaagaaccagtgttt

SEQ ID NO: 307 - RNA, 23 nt, synthetic construct
  source 1..23: mol_type = other RNA
tccgaggcaagaaccagtgttat

SEQ ID NO: 308 - RNA, 23 nt, synthetic construct
  source 1..23: mol_type = other RNA
ggggaggcaagaaccagtgttta

SEQ ID NO: 309 - RNA, 23 nt, synthetic construct
  source 1..23: mol_type = other RNA
tgggaggcaagaaccagtgttat

SEQ ID NO: 310 - RNA, 23 nt, synthetic construct
  source 1..23: mol_type = other RNA
tcggaggcaagaaccagtgttat

SEQ ID NO: 311 - RNA, 22 nt, synthetic construct
  source 1..22: mol_type = other RNA
tgggaggcaagaaccagtgttt

SEQ ID NO: 312 - skipped

SEQ ID NO: 313 - RNA, 23 nt, synthetic construct
  source 1..23: mol_type = other RNA
gaaccagtgtttagcgcgggaat

SEQ ID NO: 314 - RNA, 23 nt, synthetic construct
  source 1..23: mol_type = other RNA
aaccagtgtttagcgcgggacta

SEQ ID NO: 315 - RNA, 23 nt, synthetic construct
  source 1..23: mol_type = other RNA
accagtgtttagcgcgggactac

SEQ ID NO: 316 - RNA, 23 nt, synthetic construct
  source 1..23: mol_type = other RNA
ccagtgtttagcgcgggactaat

SEQ ID NO: 317 - RNA, 23 nt, synthetic construct
  source 1..23: mol_type = other RNA
ttagcgcgagactactgttccat

SEQ ID NO: 318 - RNA, 23 nt, synthetic construct
  source 1..23: mol_type = other RNA
ctgttccaaaaagaattccaaac

SEQ ID NO: 319 - RNA, 23 nt, synthetic construct
  source 1..23: mol_type = other RNA
ccaaaaagaattccaaccgacaa

SEQ ID NO: 320 - RNA, 23 nt, synthetic construct
  source 1..23: mol_type = other RNA
tcagtgttcccttttcaagttgt

SEQ ID NO: 321 - RNA, 23 nt, synthetic construct
  source 1..23: mol_type = other RNA
tagtgttcccttttcaagttgat

SEQ ID NO: 322 - RNA, 23 nt, synthetic construct
  source 1..23: mol_type = other RNA
tcgtgttcccttttcaagttgat

SEQ ID NO: 323 - RNA, 23 nt, synthetic construct
  source 1..23: mol_type = other RNA
tttgcattaccttcggtttgtat

SEQ ID NO: 324 - RNA, 23 nt, synthetic construct
  source 1..23: mol_type = other RNA
tctgcattaccttcggtttgtat

SEQ ID NO: 325 - RNA, 23 nt, synthetic construct
  source 1..23: mol_type = other RNA
tcgccttcagtttgtatttagtt

SEQ ID NO: 326 - RNA, 23 nt, synthetic construct
  source 1..23: mol_type = other RNA
ttgacctccgtgtagtgtctgtt

SEQ ID NO: 327 - RNA, 23 nt, synthetic construct
  source 1..23: mol_type = other RNA
tcgacctccgtgtagtgtctgtt

(19) United States
(12) Patent Application Publication
(10) Pub. No.: US 2024/0401045 A1
(43) Pub. Date: Dec. 5, 2024

(54) ANGIOTENSINOGEN-MODULATING COMPOSITIONS AND METHODS OF USE THEREOF

(71) Applicant: ADARx Pharmaceuticals, Inc., an Diego, CA (US)

(72) Inventors: Zhen Li, San Diego, CA (US); Rui Zhu, San Diego, CA (US); Zhiqing (Joel) Zhou, San Diego, CA (US); Kimberly Fultz, San Diego, CA (US)

(73) Assignee: ADARx Pharmaceuticals, Inc., San Diego, CA (US)

(21) Appl. No.: 18/697,165

(22) PCT Filed: Sep. 30, 2022
(86) PCT No.: PCT/US2022/077389

§ 371 Date: Mar. 29, 2024

Related U.S. Application Data
(60) Provisional application No. 63/287,960, filed on Dec. 9, 2021.
(60) Provisional application No. 63/251,562, filed on Oct. 1, 2021.

(51) Int. Cl. C12N 15/113

(52) U.S. Cl. CPC C12N 15/113; C12N 2310/11; C12N 2310/14; C12N 2310/3125; C12N 2310/315; C12N 2310/321; C12N 2310/322; C12N 2310/351

(57) ABSTRACT

Aspects of the disclosure provide compounds, compositions, and methods for modulating the expression or activity of angiotensinogen (AGT). In some aspects, the compounds, compositions, and methods of the disclosure can be used to reduce the expression of AGT mRNA in a cell or animal. In some aspects, the compounds, compositions, and methods of the disclosure can be used to reduce the expression of AGT protein in a cell or animal.

### CROSS-REFERENCE TO RELATED APPLICATIONS

This application claims the benefit of U.S. Provisional Application No. 63/251,562, filed Oct. 1, 2021; and U.S. Provisional Application No. 63/287,960, filed Dec. 9, 2021. The disclosure of each of the prior applications is considered part of and is incorporated by reference in its entirety in the disclosure of this application.

### BACKGROUND

Angiotensinogen (AGT), also known as SERPINA8, is a member of the serpin family. The encoded protein is angiotensinogen precursor which is largely expressed in the liver and is cleaved by the enzyme renin in response to lowered blood pressure. The product, angiotensin I, is subsequently cleaved by angiotensin converting enzyme (ACE) to generate the physiologically active angiotensin II. Angiotensin II is the active peptide of the renin-angiotensin-aldosterone system (RAAS). Angiotensin II interacts with receptors to mediate vasoconstriction, thirst, release of vasopressin and aldosterone, renal sodium reabsorption, fibrosis, inflammation, angiogenesis, vascular aging, and atherosclerosis. Release of aldosterone causes the kidneys to increase reabsorption of sodium and water, leading to an increase of the fluid volume in a body which, in turn, can increase blood pressure. Therefore, overstimulation or activity of the RAAS pathway can lead to high blood pressure. High levels of angiotensin II are associated with chronic high blood pressure (systemic arterial hypertension, essential hypertension or hypertension), renal failure and cardiac fibrosis.

Hypertension is the most common risk factor for cardiovascular disease (CVD; including coronary heart disease, heart failure, stroke, myocardial infarction, atrial fibrillation and peripheral artery disease), chronic kidney disease (CKD) and cognitive impairment, and is the leading single contributor to all-cause death and disability worldwide (Forouzanfar et al., Lancet, 2016, 388:1659-1724). The World Health Organization estimates that 1.28 billion adults aged 30-79 years worldwide have hypertension. Less than half that population is diagnosed and treated and of those only about 20% are able to control their hypertension through pharmacological treatment, diet and lifestyle changes.

The American Heart Association has defined resistant hypertension as uncontrolled blood pressure (BP) ≥130/80 mmHg, despite concurrent use of 3 anti-hypertension drug classes comprising a calcium channel blocker, a blocker of renin-angiotensin system, and a thiazide diuretic, preferably chlorthalidone. Resistant hypertension may also be defined as treatment with ≥4 classes of anti-hypertension medication regardless of BP. The global prevalence of resistant hypertension is estimated to be approximately 14.7% among the treated population. Therapies currently approved for treating hypertension have significant limitations. Drugs such as ACE inhibitors and angiotensin receptor blockers are the primary treatments for hypertension. Such drugs are limited in their ability to inhibit the RAAS pathway and have considerable adverse effects and contraindications in certain patient populations (Momoniat et al., Cleveland Clinic Journal of Medicine, 2019, 86:601-607).

Factors such as increased age and obesity predispose individuals to risk of resistant hypertension. With an aging and increasingly overweight population and lack of effective treatments, the prevalence of hypertension, resistant hypertension and related disease is expected to continue to rise. Accordingly, there is a need to find effective treatments for RAAS related diseases.

### SUMMARY

The present disclosure provides compounds, compositions, and methods for modulating the expression or activity of AGT. In certain embodiments, the compounds, compositions, and methods can be used to reduce the expression of AGT mRNA in a cell or animal. In certain embodiments, the compounds, compositions, and methods can be used to reduce the amount of AGT protein in a cell or animal.

In certain embodiments, the animal has a RAAS related disease, disorder or condition. In certain embodiments, the disease, disorder, or condition is hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment. Certain compounds, compositions and methods provided herein are directed to reducing a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment in an animal. In certain embodiments, the compounds and compositions provided herein are potent and tolerable and inhibit AGT expression, which can be used to treat, prevent, ameliorate, or slow progression of a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment.

In certain embodiments, the compounds and compositions comprise one or more features that are effective for increasing potency. In certain embodiments, the compounds and compositions comprise one or more features that are effective for increasing tolerability. In certain embodiments, compounds and compositions comprise one or more features that are effective for targeting the compound or composition to a cell or tissue. In certain embodiments, the compounds and compositions are more potent, have greater duration of action or have greater therapeutic value than compounds publicly disclosed.

### DETAILED DESCRIPTION

It is to be understood that both the foregoing summary and the following detailed description are exemplary and explanatory only and are not restrictive of the embodiments, as claimed. The section headings used herein are for organizational purposes only and are not to be construed as limiting the subject matter described.

All documents, or portions of documents, cited in this application, including, but not limited to, patents, patent applications, articles, books, treatises, and GenBank, NCBI and other sequence reference records are hereby expressly incorporated by reference for the portions of the document discussed herein, as well as in their entirety as of the date of filing this application.

It is understood that the sequence set forth in each SEQ ID NO contained herein is independent of any modification to a sugar moiety, an internucleoside linkage, or a nucleobase even if shown in context with a modified compound. As such, compounds defined by a SEQ ID NO may comprise, independently, one or more modifications to a sugar moiety, an internucleoside linkage, or a nucleobase. Oligomeric compounds referenced by Compound Number or Ref ID NO indicate a combination of nucleobase sequence, chemical modification, and motif.

Herein, the use of the singular includes the plural unless specifically stated otherwise. For example, the articles “a” and “an” are used herein to refer to one or to more than one (i.e., to at least one) of the grammatical object of the article. By way of example, “an element” means one element or more than one element, e.g., a plurality of elements. As used herein, the use of “or” means “and/or” unless stated otherwise. Furthermore, the use of the term “including” as well as other forms, such as “includes” and “included” is not limiting and is used interchangeably with the phrase “including but not limited to”.

### Definitions

Unless otherwise indicated, the following terms have the following meanings:

“Angiotensinogen,” used interchangeably with the term “AGT,” refers to any nucleic acid or protein of AGT. Exemplary nucleotide and amino acid sequences of AGT can be found, for example, at GenBank Accession No. NM_000029.4 (incorporated herein as SEQ ID NO: 1), the complement of nucleotides 230702523 to 230745583 of NC_000001.11 (incorporated herein as SEQ ID NO: 2), NM_001382817.3 (incorporated herein as SEQ ID NO: 3) and nucleotides 5469 to 17068 of NG_008836.2 (incorporated herein as SEQ ID NO: 4). Additional examples of AGT sequences are readily available through publicly available databases, e.g., GenBank, UniProt, and OMIM. Further information on AGT can be found, for example, at ncbi.nlm.nih.gov/gene/?term=AGT. AGT, as used herein, also refers to variations of the AGT gene including variants provided in the SNP database. Numerous sequence variations within the AGT gene have been identified and may be found at, for example, NCBI dbSNP and UniProt (see, e.g., ncbi.nlm.nih.gov/snp/?term=AGT). “AGT mRNA” means an mRNA encoding a AGT protein. AGT may be referred to in either upper or lower case.

“AGT specific inhibitor” refers to any agent capable of specifically inhibiting AGT RNA and/or AGT protein expression or activity at the molecular level. For example, AGT specific inhibitors include nucleic acids (including oligonucleotide compounds), peptides, antibodies, small molecules, and other agents capable of inhibiting the expression of AGT RNA and/or AGT protein.

“2′-O-methoxyethyl” or “2′-MOE” means a 2′-O(CH₂)₂—OCH₃ modification. A 2′-O-methoxyethyl modified sugar is a modified sugar with 2′-O(CH₂)₂—OCH₃ in the place of the 2′-OH group of a ribosyl ring.

“5′ start site” means the nucleotide of the target nucleic acid or region which is aligned to the 3′-most nucleoside of an antisense oligonucleotide.

“3′ stop site” means the nucleotide of the target nucleic acid or region which is aligned to the 5′-most nucleoside of an antisense oligonucleotide.

“About” means within ±10% of a value. For example, if it is stated, “a compound achieved about 70% inhibition of AGT”, it is implied that AGT levels are inhibited within a range of 60% and 80%. When about is present before a series of numbers or a range, it is understood that “about” can modify each of the numbers in the series or range.

“Administer” or “administering” refers to routes of introducing a compound or composition provided herein to an individual to perform its intended function. An example, routes of administration that can be used include, but are not limited to, parenteral administration, such as subcutaneous, intravenous, or intramuscular injection or infusion.

“Ameliorate” refers to an improvement or lessening of at least one indicator, sign, or symptom of an associated disease, disorder, or condition. In certain embodiments, amelioration includes a delay or slowing in the progression or severity of one or more indicators of a condition or disease. The progression or severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antisense oligonucleotide” or “antisense strand” means an oligonucleotide which includes a region that is complementary to a target nucleic acid, e.g., a AGT RNA or a region thereof.

“Complementarity” in reference to an oligonucleotide means the nucleobase sequence of such oligonucleotide or one or more regions thereof that is complementary to the nucleobase sequence of another oligonucleotide or nucleic acid or one or more regions thereof when the two nucleobase sequences are aligned in opposing directions. Complementary nucleobases, as described herein, are limited to the following pairs: adenine (A) and thymine (T), adenine (A) and uracil (U), and cytosine (C) and guanine (G) unless otherwise specified. Complementary oligonucleotides and/or nucleic acids need not have nucleobase complementarity at each nucleoside and may include one or more nucleobase mismatches. By contrast, “fully complementary” or “100% complementary” in reference to oligonucleotides means that such oligonucleotides have nucleobase matches at each nucleoside without any nucleobase mismatches.

“Composition” or “pharmaceutical composition” means a mixture of substances suitable for administering to an individual. For example, a composition may comprise one or more compounds or salt thereof and a sterile aqueous solution.

“Co-administration” means administration of two or more compounds in any manner in which the pharmacological effects of both are manifest in the patient. Co-administration does not require both compounds to be administered in a single pharmaceutical composition, in the same dosage form, by the same route of administration, or at the same time. The effects of both compounds need not manifest themselves at the same time. The effects need only be overlapping for a period of time and need not be coextensive. Co-administration includes parallel or sequential administration of the one or more compounds.

“Conjugate group” means a group of atoms that is attached to an oligonucleotide. A conjugate group is optionally attached to an oligonucleotide through a conjugate linker. A conjugate group may, for example, alter the distribution, targeting, or half-life of a compound into which it is incorporated. Conjugate groups include targeting moieties.

“Conjugate linker” means a group of atoms comprising at least one bond that connects a linked moiety to an oligonucleotide.

“Identity” in reference to an oligonucleotide means the nucleobase sequence of such oligonucleotide or one or more regions thereof that matches the nucleobase sequence of another oligonucleotide or nucleic acid or one or more regions thereof. Identity of an oligonucleotide to another oligonucleotide or nucleic acid need not require each nucleobase to match and may include one or more different nucleobases. By contrast, “fully identical” or “100% identity” in reference to oligonucleotides means that such oligonucleotides have the same nucleobase at each relative position over its length as the other oligonucleotide or nucleic acid.

“Individual” means a human or non-human animal selected for treatment or therapy.

“Inhibiting the expression or activity” with reference to a target nucleic acid or protein means to reduce or block the expression or activity of such target relative to the expression or activity in an untreated or control sample and does not necessarily indicate a total elimination of expression or activity.

As used herein, the term “internucleoside linkage” is the covalent linkage between adjacent nucleosides in an oligonucleotide. As used herein, “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations as further described below. Unless otherwise indicated, chiral internucleoside linkages of modified oligonucleotides described herein can be stereorandom or in a particular stereochemical configuration.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

The term “isotopic variant” refers to a therapeutic agent (e.g., a compound and/or modified oligonucleotide disclosed herein) that contains an unnatural proportion of an isotope at one or more of the atoms that constitute such a therapeutic agent. In certain embodiments, an “isotopic variant” of a therapeutic agent contains unnatural proportions of one or more isotopes, including, but not limited to, hydrogen (H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine 123 (¹²³I), iodine-125 (¹²⁵I), iodine-127 (¹²⁷I), iodine-129 (¹²⁹I), and iodine-131 (¹³¹I). In certain embodiments, an “isotopic variant” of a therapeutic agent contains unnatural proportions of one or more isotopes, including, but not limited to, hydrogen (H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine 123 (¹²³I), iodine-125 (¹²⁵I), iodine-127 (¹²⁷I), iodine-129 (¹²⁹I), and iodine-131 (¹³¹I).

It will be understood that, in a therapeutic agent (e.g., a compound and/or modified oligonucleotide disclosed herein), any hydrogen can be ²H, for example, or any carbon can be ¹³C, for example, or any nitrogen can be ¹⁵N, for example, or any oxygen can be ¹⁸O, for example, where feasible according to the judgment of one of skill. In certain embodiments, an “isotopic variant” of a therapeutic agent contains unnatural proportions of deuterium (D).

“Mismatch” or “non-complementary” means a nucleobase of a first oligonucleotide or nucleic acid that is not complementary to the corresponding nucleobase of a second oligonucleotide or nucleic acid when the first oligonucleotide/nucleic acid and second oligonucleotide/nucleic acid are aligned in an antiparallel orientation. For example, nucleobases including, but not limited to, a universal nucleobase, inosine, and hypoxanthine, are capable of hybridizing with at least one nucleobase but are still mismatched or non-complementary with respect to the nucleobase to which they are hybridized. As another example, a nucleobase of a first oligonucleotide/nucleic acid that is not capable of hybridizing to the corresponding nucleobase of a second oligonucleotide/nucleic acid when the first and second oligonucleotides are aligned in an antiparallel orientation is a mismatch or non-complementary nucleobase.

“Modified oligonucleotide” means an oligonucleotide, wherein at least one sugar, nucleobase, or internucleoside linkage is modified.

“Modulating” refers to changing or adjusting a feature in a cell, tissue, organ or organism. For example, modulating AGT RNA can mean to increase or decrease the level of AGT RNA and/or AGT protein in a cell, tissue, organ, or organism. A “modulator” effects the change in the cell, tissue, organ or organism. For example, a AGT compound can be a modulator that decreases the amount of AGT RNA and/or AGT protein in a cell, tissue, organ or organism.

“Motif” means the pattern of unmodified and modified sugar moieties, nucleobases, and/or internucleoside linkages, in an oligonucleotide.

“Nucleic acid” refers to molecules composed of monomeric nucleotides. A nucleic acid includes, but is not limited to, ribonucleic acids (RNA), deoxyribonucleic acids (DNA), single-stranded nucleic acids, and double-stranded nucleic acids.

“Nucleobase” means a heterocyclic moiety capable of pairing with a base of another nucleic acid. As used herein a “naturally occurring nucleobase” is adenine (A), thymine (T), cytosine (C), uracil (U), and guanine (G). A “modified nucleobase” is a naturally occurring nucleobase that is chemically modified. A “universal base” or “universal nucleobase” is a nucleobase other than a naturally occurring nucleobase and modified nucleobase and is capable of pairing with any nucleobase.

“Nucleobase sequence” means the order of contiguous nucleobases in a nucleic acid or oligonucleotide independent of any sugar or internucleoside linkage.

“Nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase.

“Oligomeric Compound” means a compound comprising one or more oligonucleotides and optionally one or more additional features, such as a conjugate group or terminal group. Examples of oligomeric compounds include single-stranded and double-stranded compounds, such as, oligonucleotides, antisense oligonucleotides, interfering RNA compounds (RNAi compounds), microRNA targeting oligonucleotides, occupancy-based compounds (e.g., mRNA processing or translation blocking compounds and splicing compounds). RNAi compounds include double-stranded compounds (e.g., short-interfering RNA (siRNA) and double-stranded RNA (dsRNA)) and single-stranded compounds (e.g., single-stranded siRNA (ssRNA), single-stranded RNAi (ssRNAi), short hairpin RNA (shRNA) and microRNA mimics) which work at least in part through the RNA-induced silencing complex (RISC) pathway resulting in sequence specific degradation and/or sequestration of a target nucleic acid through a process known as RNA interference (RNAi). The term “RNAi compound” is meant to be equivalent to other terms used to describe nucleic acid compounds that are capable of mediating sequence-specific RNA interference, for example, interfering RNA (iRNA), iRNA agent, RNAi agent, short interfering oligonucleotide, short interfering nucleic acid, short interfering modified oligonucleotide, chemically modified siRNA, and others. Additionally, the term “RNAi” is meant to be equivalent to other terms used to describe sequence-specific RNA interference.

“Oligonucleotide” means a polymer of linked nucleosides, each of which can be modified or unmodified, independent from one another.

The term “oligomeric duplex” means a duplex formed by two oligomeric compounds having complementary nucleobase sequences. Each oligomeric compound of an oligomeric duplex may be referred to as a “duplexed oligomeric compound.” The oligonucleotides of each oligomeric compound of an oligomeric duplex may include non-complementary overhanging nucleosides. In some embodiments, the terms “duplexed oligomeric compound” and “modified oligonucleotide” are used interchangeably. In other embodiments, the terms “oligomeric duplex” and “compound” are used interchangeably.

“Parenteral administration” means administration through injection or infusion. Parenteral administration includes subcutaneous administration, intravenous administration, intramuscular administration, intraarterial administration, intraperitoneal administration, or intracranial administration, e.g., intrathecal or intracerebroventricular administration.

“Pharmaceutically acceptable carrier or diluent” means any substance suitable for use in administering to an individual. In certain embodiments, a pharmaceutically acceptable carrier or diluent aids the administration of a compound to and absorption by an individual and can be included in the compositions of the present disclosure without causing a significant adverse toxicological effect on the patient. Non-limiting examples of pharmaceutically acceptable excipients include water, NaCl, normal saline solutions, and the like. For example, a pharmaceutically acceptable carrier can be a sterile aqueous solution, such as PBS or water-for-injection. One of skill in the art will recognize that other pharmaceutical excipients are useful in the present disclosure.

“Pharmaceutically acceptable salts” means or refers to physiologically and pharmaceutically acceptable salts of compounds, such as oligomeric compounds or oligonucleotides, i.e., salts that retain the desired biological activity of the parent compound and do not impart undesired toxicological effects thereto. As used herein, a pharmaceutically acceptable salt is any salt of a compound provided herein which retains its biological properties and which is not toxic or otherwise undesirable for pharmaceutical use. The pharmaceutically acceptable salts of the therapeutic agents disclosed herein include salts that are prepared with relatively nontoxic acids or bases, depending on the particular substituents found on the compounds or modified oligonucleotides described herein.

When compounds of the present disclosure contain relatively acidic functionalities, base addition salts can be obtained by contacting the neutral form of such compounds with a sufficient amount of the desired base, either neat or in a suitable inert solvent.

When compounds of the present disclosure contain relatively basic functionalities, acid addition salts can be obtained by contacting the neutral form of such compounds with a sufficient amount of the desired acid, either neat or in a suitable inert solvent.

Thus, the compounds of the present disclosure may exist as salts, such as with pharmaceutically acceptable acids. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia, or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like (see, for example, Berge et al., “Pharmaceutical Salts,” Journal of Pharmaceutical Science, 1977, 66, 1-19).

Pharmaceutically acceptable salts further include, by way of example only and without limitation, sodium, potassium, calcium, magnesium, ammonium, tetraalkylammonium, and the like, and when the compound contains a basic functionality, salts of non-toxic organic or inorganic acids, such as hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate, and the like. In some embodiments, the pharmaceutically acceptable salt of the compounds and modified oligonucleotides disclosed herein is a sodium or a potassium salt. In some embodiments, the pharmaceutically acceptable salt of the compounds and modified oligonucleotides disclosed herein is a sodium salt.

The neutral forms of the compounds are preferably regenerated by contacting the salt with a base or acid and isolating the parent compound in the conventional manner. The parent form of the compound may differ from the various salt forms in certain physical properties, such as solubility in polar solvents. In embodiments, compounds of the present disclosure contain both basic and acidic functionalities that allow the compounds to be converted into either base or acid addition salts. The neutral forms of the compounds may be regenerated by contacting the salt with a base or acid and isolating the parent compound in a conventional manner. The parent form of the compounds differs from the various salt forms in certain physical properties, such as solubility in polar solvents, but, unless specifically indicated, the salts disclosed herein are equivalent to the parent form of the compound for the purposes of the present disclosure.

“Pharmaceutical agent” means a compound that provides a therapeutic benefit when administered to an individual.

“Phosphorothioate linkage” means a modified phosphate linkage in which one of the non-bridging oxygen atoms is replaced with a sulfur atom.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an oligonucleotide.

“Prevent” refers to delaying or forestalling the onset, development or progression of a disease, disorder, or condition for a period of time.

“RNA interference compound” or “RNAi compound” means a compound that acts, at least in part, through an RNA-induced silencing complex (RISC) pathway or Ago2, but not through RNase H, to modulate a target nucleic acid and/or protein encoded by a target nucleic acid. RNAi compounds include, but are not limited to double-stranded siRNA, single-stranded siRNA, and microRNA, including microRNA mimics.

“Sense oligonucleotide” or “sense strand” means the strand of a double-stranded compound that includes a region that is substantially complementary to a region of the antisense strand of the compound.

“Specifically inhibit” with reference to a target nucleic acid or protein means to reduce or block expression or activity of the target nucleic acid or protein while minimizing or eliminating effects on non-target nucleic acids or proteins.

“Subunit” with reference to an oligonucleotide means a nucleotide, nucleoside, nucleobase or sugar or a modified nucleotide, nucleoside, nucleobase or sugar as provided herein.

“Target nucleic acid,” “target RNA,” and “nucleic acid target” all mean a nucleic acid capable of being targeted by compounds described herein.

“Target region” means a portion of a target nucleic acid to which one or more compounds is targeted.

“Targeting moiety” means a conjugate group that provides an enhanced affinity for a selected target, e.g., molecule, cell or cell type, compartment, e.g., a cellular or organ compartment, tissue, organ or region of the body, as, e.g., compared to a compound absent such a moiety.

“Terminal group” means a chemical group or group of atoms that is covalently linked to a terminus of an oligonucleotide.

“Therapeutically effective amount” or “effective amount” means an amount of a compound, pharmaceutical agent, or composition that provides a therapeutic benefit to an individual. A “therapeutically effective amount” or “effective amount” is an amount sufficient for a compound to accomplish a stated purpose relative to the absence of the compound (e.g., achieve the effect for which it is administered, treat, prevent or ameliorate a disease or reduce one or more symptoms of a disease or condition). An example of a “therapeutically effective amount” or “effective amount” is an amount sufficient to contribute to the treatment, prevention, amelioration, or reduction of a symptom or symptoms of a disease. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The term “therapeutically effective amount,” as used herein, refers to that amount of the therapeutic agent sufficient to provide a therapeutic benefit to an individual, such as treating, preventing or ameliorating the disease or disorder or symptom thereof, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

The terms “treating” or “treatment” refer to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters, including the results of a physical examination. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of a compound described herein. The administering step may consist of a single administration or may include a series of administrations. The length of the treatment period depends on a variety of factors, such as the severity of the condition, the age of the patient, the concentration of the compound, the activity of the compositions used in the treatment, or a combination thereof. It will also be appreciated that the effective dosage of an agent used for the treatment or prophylaxis may increase or decrease over the course of a particular treatment or prophylaxis regime. In some instances, chronic administration may be required. For example, the compositions are administered to the subject in an amount and for a duration sufficient to treat the patient.

“Treat” refers to administering a compound or pharmaceutical composition to an animal in order to effect an alteration or improvement of a disease, disorder, or condition in the animal.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium, and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure (i.e., the R and S configurations for each asymmetric center). Therefore, single stereochemical isomers as well as enantiomeric and diastereomeric mixtures of the present compounds are within the scope of the disclosure.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the results of a synthetic method that is not designed to control the stereochemical configuration. In certain embodiments, a stereorandom chiral center is a stereorandom phosphorothioate internucleoside linkage.

### Certain Embodiments

In certain aspects, the disclosure relates to methods, compounds, and compositions for inhibiting AGT. In certain embodiments, AGT is specifically inhibited. In certain embodiments, AGT is specifically degraded. In certain embodiments, AGT expression is inhibited. In certain embodiments, AGT translation is inhibited. In certain embodiments, AGT activity is inhibited. In certain embodiments, AGT expression, translation, or activity is reduced by at least 10% relative to the expression, translation, or activity in an untreated or control sample. For example, in certain embodiments, AGT expression, translation, or activity is reduced by at least 10%, at least 20%, at least 30%, at least 40%, at least 50%, at least 60%, at least 70%, at least 80%, at least 90%, at least 95%, 10-50%, 25-50%, 25-75%, 50-75%, 50-99%, or 75-99% relative to the expression, translation, or activity in an untreated or control sample. In certain embodiments, AGT expression, translation, or activity is reduced as measured by any suitable assay, including but not limited to, an immunoassay, a hybridization-based assay, or a sequencing-based assay (e.g., RNA-Seq).

In certain aspects, the disclosure relates to compounds targeted to a AGT nucleic acid. In certain embodiments, the AGT nucleic acid has the sequence set forth in GENBANK Accession No. NM_000029.4 (incorporated herein as SEQ ID NO: 1), the complement of nucleotides 230702523 to 230745583 of NC_000001.11 (incorporated herein as SEQ ID NO: 2), NM_001382817.3 (incorporated herein as SEQ ID NO: 3) and nucleotides 5469 to 17068 of NG_008836.2 (incorporated herein as SEQ ID NO: 4).

In certain embodiments, the compound is an oligomeric compound. In certain embodiments, the compound is single-stranded. In certain embodiments, the compound is double-stranded.

Certain embodiments provide a compound comprising a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166.

Certain embodiments provide a compound comprising a modified oligonucleotide (e.g., of 14 to 30, for example, 14 to 23, linked nucleosides in length) having a nucleobase sequence comprising the nucleobase sequence of any one of SEQ ID NOs: 10-166.

Certain embodiments provide a compound comprising a modified oligonucleotide having a nucleobase sequence selected from any one of the nucleobase sequences of SEQ ID NOs: 10-166.

In certain embodiments, the modified oligonucleotide has a nucleobase sequence that is at least 80%, at least 85%, at least 90%, or at least 95% complementary to the nucleobase sequence of SEQ ID NO: 1 or 3. In certain embodiments, the modified oligonucleotide comprises at least one modification selected from a modified internucleoside linkage, a modified sugar, and a modified nucleobase. In certain embodiments, the compound is double-stranded.

Certain embodiments provide a compound comprising a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166 or 167-327 and a second modified oligonucleotide (e.g., of 14 to 30, for example, 14 to 23, linked nucleosides in length) having a region of complementarity to the first modified oligonucleotide.

In certain embodiments, the compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences provided in Tables 2-25, 42, 45, 50, and 51, and a second modified oligonucleotide (e.g., of 14 to 30, for example, 14 to 23, linked nucleosides in length) having a region of complementarity to the first modified oligonucleotide.

Certain embodiments provide a compound comprising a first modified oligonucleotide (e.g., of 14 to 30, for example, 14 to 23, linked nucleosides in length) having a nucleobase sequence comprising the nucleobase sequences of any one of SEQ ID NOs: 10-166 or 167-327 and a second modified oligonucleotide (e.g., of 14 to 30, for example, 14 to 23, linked nucleosides in length) having a region of complementarity to the first modified oligonucleotide.

Certain embodiments provide a compound comprising a first modified oligonucleotide having a nucleobase sequence selected from the nucleobase sequences of any one of SEQ ID NOs: 10-166 and 167-327 and a second modified oligonucleotide 19 to 23 linked nucleosides in length having a region of complementarity to the first modified oligonucleotide.

In certain embodiments, the first modified oligonucleotide has a nucleobase sequence that has at least 80%, at least 85%, at least 90%, or at least 95% complementarity or identity to the nucleobase sequence of SEQ ID NO: 1 or 3 over its length. In certain embodiments, the first modified oligonucleotide having a nucleobase sequence has at least 1, at least 2, at least 3 mismatches to a region of the nucleobase sequence of SEQ ID NO: 1 or 3. In certain embodiments, the region of complementarity between the first strand and the second strand is 14 to 30 linked nucleosides in length. In certain embodiments, the region of complementarity between the first strand and the second strand is 14 to 23 linked nucleosides in length. In certain embodiments, the region of complementarity between the first strand and the second strand is 19 to 23 linked nucleosides in length. In certain embodiments, the region of complementarity between the first strand and the second strand is 21 to 23 linked nucleosides in length. In certain embodiments, the first modified oligonucleotide is fully complementary to the second modified oligonucleotide.

In certain embodiments, the first modified oligonucleotide of any preceding compound comprises at least one modification selected from a modified internucleoside linkage, a modified sugar, and a modified nucleobase. In certain embodiments, the second modified oligonucleotide of any preceding compound comprises at least one modification selected from the group consisting of a modified internucleoside linkage, a modified sugar, and a modified nucleobase. In certain embodiments, the modified internucleoside linkage is a phosphorothioate internucleoside linkage or a methylphosphonate internucleoside linkage. In certain embodiments, the phosphorothioate internucleoside linkage or methylphosphonate internucleoside linkage is at the 3′ terminus of the first or second modified oligonucleotide or at the 5′ terminus of the first modified oligonucleotide. In certain embodiments, the modified sugar comprises a modification selected from the group consisting of a halogen, an alkoxy group and a bicyclic sugar. In certain embodiments, the modified sugar comprises a 2′-F modification. In certain embodiments, the modified sugar comprises a 2′-OMe modification. In certain embodiments, each nucleoside of the first modified oligonucleotide comprises a modified sugar. In certain embodiments, each nucleoside of the second modified oligonucleotide comprises a modified sugar. In certain embodiments, the modified sugar comprises a modification selected from the group consisting of a halogen, an alkoxy group and a bicyclic sugar or a combination thereof. In certain embodiments, the modified sugar comprises a modification selected from the group consisting of 2′-MOE, 2′-F, and 2′-OMe or a combination thereof. In certain embodiments, the first modified oligonucleotide comprises no more than ten 2′-F sugar modifications. In certain embodiments, the second modified oligonucleotide comprises no more than five 2′-F sugar modifications.

In certain embodiments, the compound of any preceding embodiment comprises a conjugate group. In certain embodiments, the conjugate group is attached to the 5′ end of the modified oligonucleotide. In certain embodiments, the conjugate group is a targeting moiety. In certain embodiments, the targeting moiety comprises one or more GalNAc. In certain embodiments, the modified oligonucleotide is the second modified oligonucleotide or sense oligonucleotide. In certain embodiments, the one or more GalNAc is attached to the 2′ or 3′ position of the ribosyl ring. In certain embodiments, the one or more GalNAc is attached to the 5′ nucleoside of the modified oligonucleotide. In certain embodiments, the 5′ nucleoside of a modified oligonucleotide is selected from the following Formulae or a salt, solvate, or hydrate thereof, wherein R is the portion of the modified oligonucleotide other than the 5′ nucleoside:

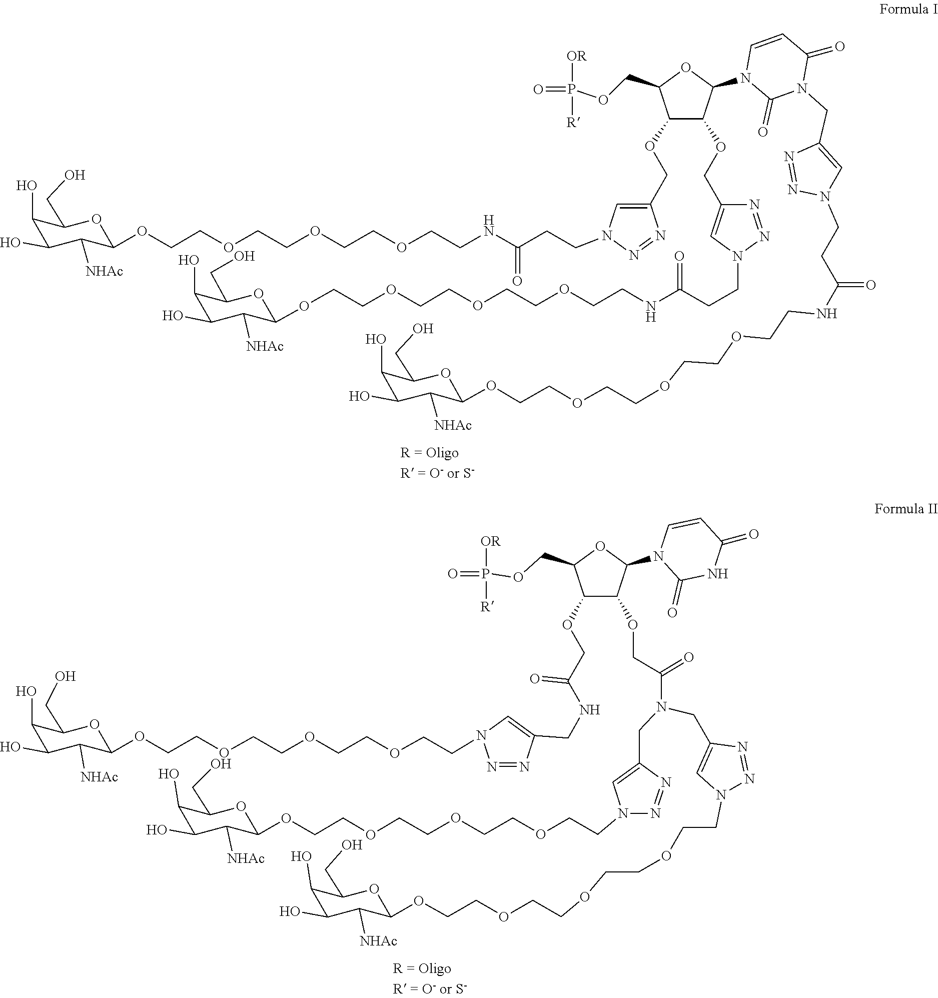

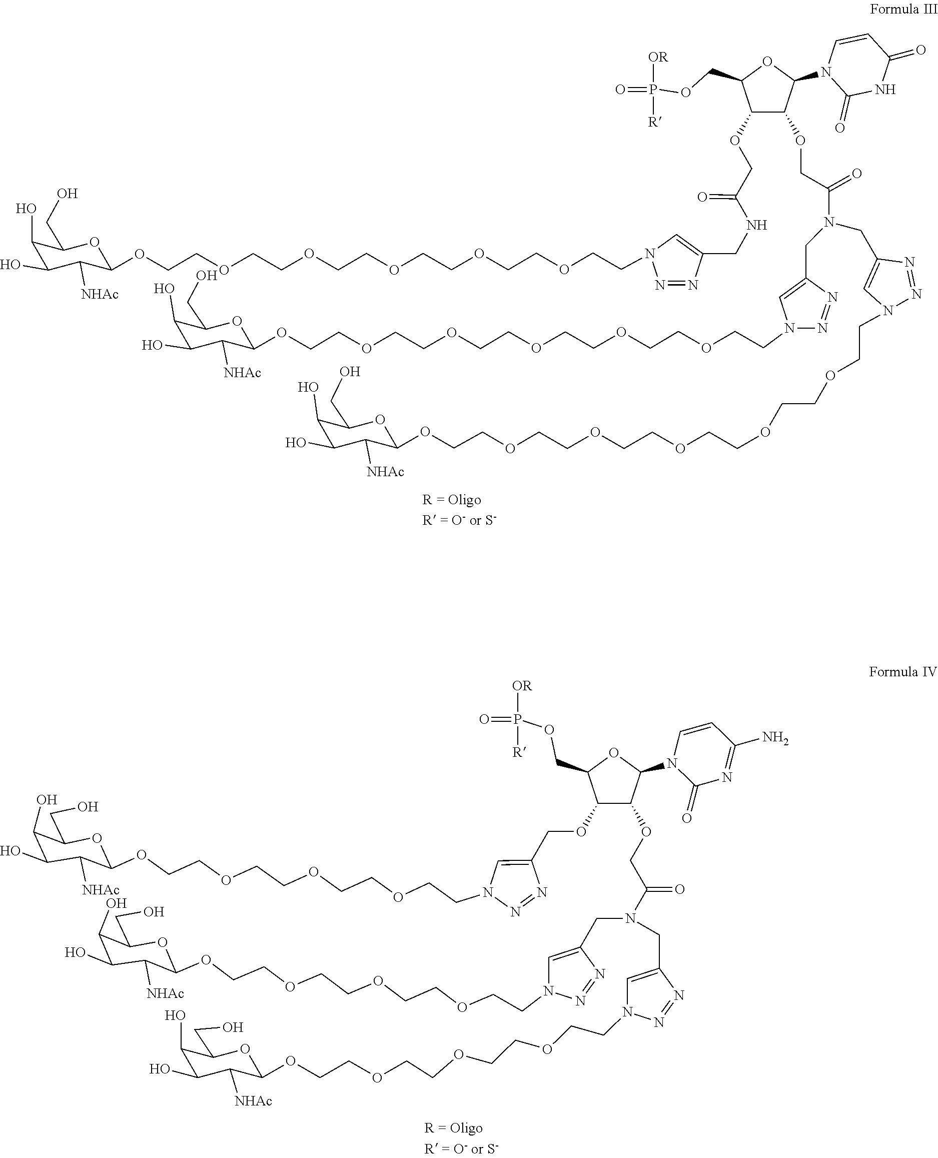

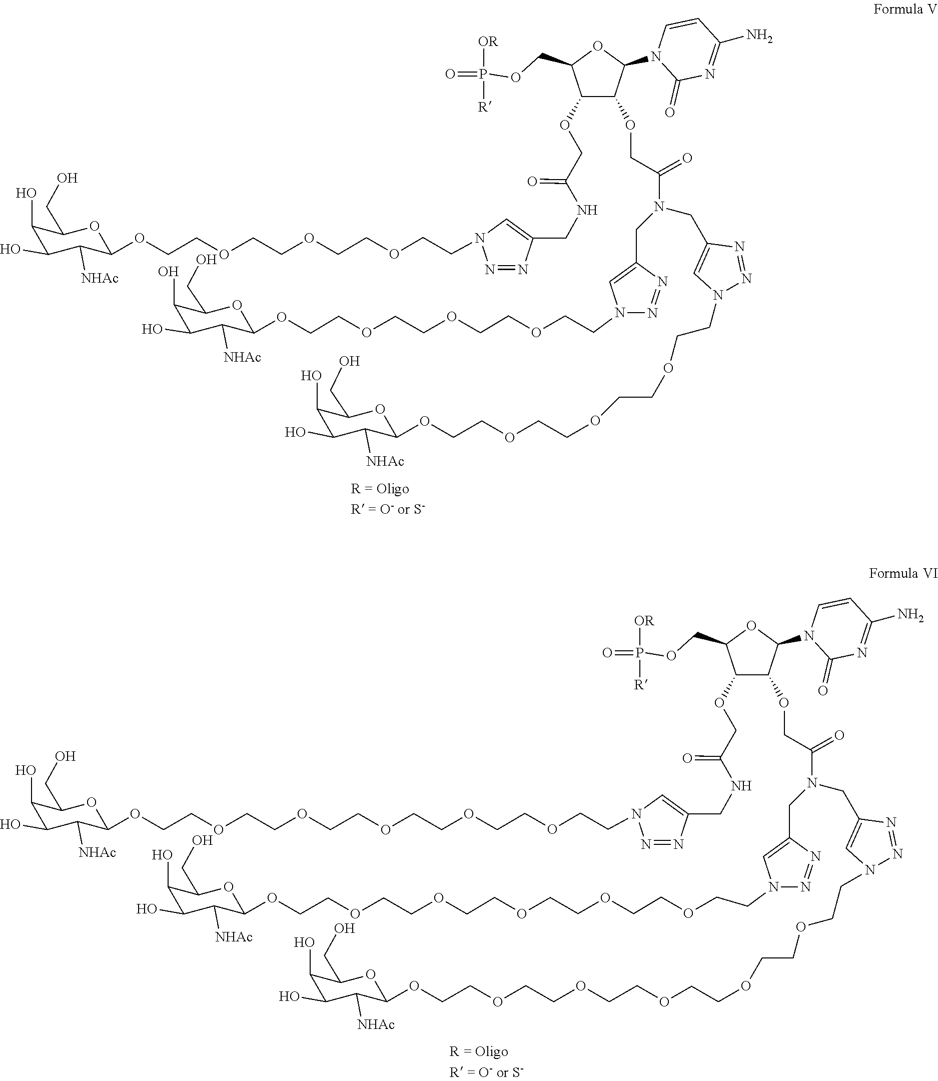

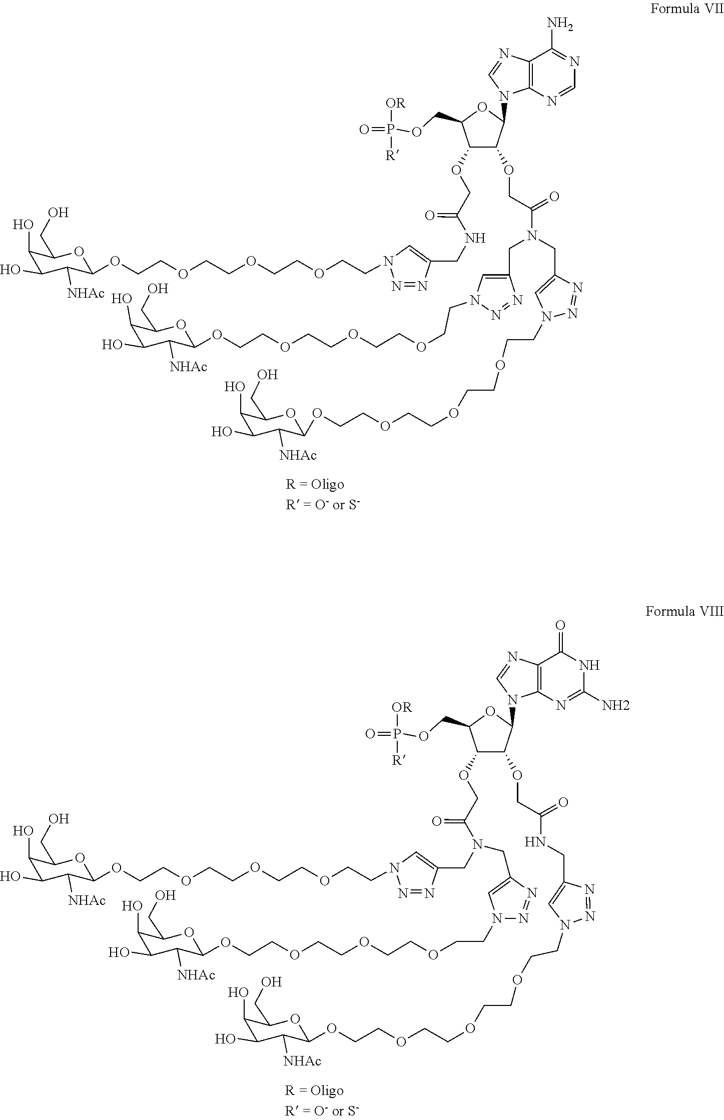

In certain embodiments, R′ is O. In certain embodiments, R′ is S. In certain embodiments, the 5′ nucleoside of the modified oligonucleotide is Formula I and R′ is O. In certain embodiments, the 5′ nucleoside of the modified oligonucleotide is Formula I and R′ is S. In certain embodiments, the 5′ nucleoside of the modified oligonucleotide is Formula II and R′ is O. In certain embodiments, the 5′ nucleoside of the modified oligonucleotide is Formula II and R′ is S. In certain embodiments, the 5′ nucleoside of the modified oligonucleotide is Formula III and R′ is O. In certain embodiments, the 5′ nucleoside of the modified oligonucleotide is Formula III and R′ is S. In certain embodiments, the 5′ nucleoside of the modified oligonucleotide is Formula IV and R′ is O. In certain embodiments, the 5′ nucleoside of the modified oligonucleotide is Formula IV and R′ is S. In certain embodiments, the 5′ nucleoside of the modified oligonucleotide is Formula V and R′ is O. In certain embodiments, the 5′ nucleoside of the modified oligonucleotide is Formula V and R′ is S. In certain embodiments, the 5′ nucleoside of the modified oligonucleotide is Formula VI and R′ is O. In certain embodiments, the 5′ nucleoside of the modified oligonucleotide is Formula VI and R′ is S. In certain embodiments, the 5′ nucleoside of the modified oligonucleotide is Formula VII and R′ is O. In certain embodiments, the 5′ nucleoside of the modified oligonucleotide is Formula VII and R′ is S. In certain embodiments, the 5′ nucleoside of the modified oligonucleotide is Formula VIII and R′ is O. In certain embodiments, the 5′ nucleoside of the modified oligonucleotide is Formula VIII and R′ is S.

Certain embodiments provide a compound comprising a first modified oligonucleotide selected from any one of Ref ID NOs: IA0297, IA0300, IA0301, IA0304, IA0305, IA0335-338, IA0343-359, IA0431-432, IA0435, IA440-446, IA0727-728, IA0500-501, and IA0868, and a second modified oligonucleotide 14 to 21 linked nucleosides in length fully complementary to the first modified oligonucleotide.

Certain embodiments provide a compound comprising a first modified oligonucleotide consisting of IA0443 and a second modified oligonucleotide consisting of IS0505. In certain embodiments, the compound comprises a first modified oligonucleotide consisting of IA0445 and a second modified oligonucleotide consisting of IS0509.

In certain embodiments, the compound of any foregoing embodiment is in a pharmaceutically acceptable salt form. In certain embodiments, the pharmaceutically acceptable salt is a sodium salt. In certain embodiments, the pharmaceutically acceptable salt is a potassium salt.

Certain embodiments provide a composition comprising the compound of any one of the foregoing embodiments and a pharmaceutically acceptable carrier.

Certain embodiments provide a composition comprising a compound of any preceding embodiment, for use in therapy.

Certain embodiments provide a method of treating, preventing, or ameliorating a disease, disorder or condition associated with AGT in an individual comprising administering to the individual a compound targeted to AGT, thereby treating, preventing, or ameliorating the disease.

In certain embodiments, the compound or composition of any foregoing embodiment is administered to an individual. In certain embodiments, the disease, disorder, or condition is a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment.

In certain embodiments, administering the compound inhibits or reduces or improves a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment.

In certain embodiments, a compound or composition comprising a compound of any preceding embodiment is administered to an individual in a therapeutically effective amount. In certain embodiments, a compound or composition comprising a compound of any preceding embodiment is administered to an individual at a dosage level sufficient to deliver about 1 to 100 mg/kg of body weight of the individual. In certain embodiments, a compound or composition comprising a compound of any preceding embodiment is administered to an individual at a fixed dose of about 25 mg to about 1,000 mg. In certain embodiments, the compound or composition is administered to the individual one or more times in a day up to the dosage level or fixed dose.

In certain embodiments, a compound or composition comprising a compound of any preceding embodiment is administered to an individual daily, weekly, monthly, quarterly or yearly. In certain embodiments, a compound or composition comprising a compound of any preceding embodiment is administered to an individual about once per quarter (i.e., once every three months) to about once per year. In certain embodiments, a compound or composition comprising a compound of any preceding embodiment is administered to an individual about once per quarter, about once every six months or about once per year.

Certain embodiments provide a method of inhibiting expression of AGT in a cell comprising contacting the cell with a compound targeted to AGT, thereby inhibiting expression of AGT in the cell. In certain embodiments, the cell is in the liver of an individual. In certain embodiments, the individual has, or is at risk of having, a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm, and peripheral artery disease), organ damage (e.g., heart, liver or kidney), inflammatory bowel disease or cognitive impairment.

Certain embodiments provide a method of reducing or inhibiting a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment in an individual, comprising administering a compound targeted to AGT to the individual, thereby reducing or inhibiting a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment in the individual. In certain embodiments, the individual has, or is at risk of having, a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm, and peripheral artery disease), organ damage (e.g., heart, liver or kidney), inflammatory bowel disease or cognitive impairment. In certain embodiments, the compound is a compound targeted to AGT. In certain embodiments, the compound is any of the foregoing compounds. In certain embodiments, the compound or composition is administered parenterally.

Certain embodiments provide use of a compound targeted to AGT for treating, preventing, or ameliorating a disease, disorder or condition associated with AGT. In certain embodiments, the disease, disorder, or condition is a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment. In certain embodiments, the compound is a compound targeted to AGT. In certain embodiments, the compound is any of the foregoing compounds.

Certain embodiments provide use of a compound targeted to AGT in the manufacture of a medicament for treating, preventing, or ameliorating a disease, disorder or condition associated with AGT. In certain embodiments, the disease, disorder, or condition is a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment. In certain embodiments, the compound is a compound targeted to AGT. In certain embodiments, the compound is any of the foregoing compounds.

### Certain Indications

In certain aspects, the disclosure relates to methods of inhibiting AGT expression, which can be useful for treating, preventing, or ameliorating a disease associated with AGT in an individual, by administration of a compound that targets AGT. In certain embodiments, the compound can be a AGT specific inhibitor. In certain embodiments, the compound can be an antisense oligonucleotide, an oligomeric compound, or an oligonucleotide targeted to AGT.

In certain aspects, the disclosure relates to treating, preventing, or ameliorating a disease, disorder or condition associated with AGT. In certain embodiments, diseases, disorders or conditions associated with AGT treatable, preventable, and/or ameliorable with the methods provided herein include a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment. Certain compounds provided herein are directed to compounds and compositions that reduce a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment in an animal.

In certain embodiments, a method of treating, preventing, or ameliorating a disease associated with AGT in an individual comprises administering to the individual a compound comprising a AGT specific inhibitor, thereby treating, preventing, or ameliorating the disease. In certain embodiments, the individual is identified as having, or at risk of having, a disease associated with AGT. In certain embodiments, the disease is a RAAS related disease. In certain embodiments, the compound comprises an antisense oligonucleotide targeted to AGT. In certain embodiments, the compound comprises an oligonucleotide targeted to AGT. In certain embodiments, a compound comprises a modified oligonucleotide (e.g., of 14 to 30, for example, 14 to 23, linked nucleosides) in length having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166. In certain embodiments, a compound comprises a modified oligonucleotide (e.g., of 14 to 30, for example, 14 to 23, linked nucleosides in length) and having a nucleobase sequence comprising the nucleobase sequence of any one of SEQ ID NOs: 10-166. In certain embodiments, a compound comprises a modified oligonucleotide having a nucleobase sequence selected from the nucleobase sequences of any one of SEQ ID NOs: 10-166. In certain embodiments, a compound comprises a modified oligonucleotide (e.g., of 14 to 30, for example, 14 to 23, linked nucleosides in length) having a nucleobase sequence comprising the nucleobase sequence of any one of SEQ ID NOs: 85 and 134. In certain embodiments, a compound comprises a modified oligonucleotide having a nucleobase sequence selected from the nucleobase sequences of any one of SEQ ID NOs: 85 and 134. In any of the foregoing embodiments, the compound can be single-stranded or double-stranded. In certain embodiments, a single-stranded compound can be 14 to 30, 14 to 23, 14 to 20, 16 to 20, or 14 to 16, linked nucleosides in length. In certain embodiments, a single-stranded compound can be 14, 15, 16, 17, 18, 19, 20, 21, 22, 23, 24, 25, 26, 27, 28, 29, or 30, linked nucleosides in length. In certain embodiments, a double-stranded compound can comprise two oligonucleotides of the same or different lengths, as described elsewhere herein. In any of the foregoing embodiments, the compound can be an antisense oligonucleotide or oligomeric compound. In certain embodiments, a compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166 or 167-327 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide (e.g., of 14 to 30, for example, 14 to 23, linked nucleosides in length) having a nucleobase sequence comprising the nucleobase sequence of any one of SEQ ID NOs: 10-166 or 167-327 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide having a nucleobase sequence selected from the nucleobase sequences of any one of SEQ ID NOs: 10-166 and 167-327 and a second modified oligonucleotide 19 to 23 linked nucleosides in length having a region of complementarity to the first modified oligonucleotide. In certain embodiments, a compound comprises a first modified oligonucleotide having a nucleobase sequence selected from the nucleobase sequences of any one of SEQ ID NOs: 85 and 134 and a second modified oligonucleotide having a nucleobase sequence selected from the nucleobase sequences of any one of SEQ ID NOs: 244 and 294. In certain embodiments, the compound is administered to the individual parenterally. In certain embodiments, administering the compound improves, preserves, or prevents a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment in an animal.

In certain embodiments, a method of treating, preventing, or ameliorating a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment in an animal comprises administering to the individual a compound comprising a AGT specific inhibitor, thereby treating, preventing, or ameliorating a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment. In certain embodiments, the compound comprises an antisense oligonucleotide targeted to AGT. In certain embodiments, the compound comprises an oligonucleotide targeted to AGT. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166. In certain embodiments, a compound comprises a modified oligonucleotide (e.g., of 14 to 30, for example, 14 to 23, linked nucleosides in length) and having a nucleobase sequence comprising the nucleobase sequence of any one of SEQ ID NOs: 10-166. In certain embodiments, a compound comprises a modified oligonucleotide having a nucleobase sequence selected from the nucleobase sequences of any one of SEQ ID NOs: 10-166. In certain embodiments, a compound comprises a modified oligonucleotide (e.g., of 14 to 30, for example, 14 to 23, linked nucleosides in length) having a nucleobase sequence comprising the nucleobase sequence of any one of SEQ ID NOs: 85 and 134. In certain embodiments, a compound comprises a modified oligonucleotide having a nucleobase sequence selected from the nucleobase sequences of any one of SEQ ID NOs: 85 and 134. In any of the foregoing embodiments, the compound can be single-stranded or double-stranded. In any of the foregoing embodiments, the compound can be an antisense oligonucleotide or oligomeric compound. In certain embodiments, a compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166 or 167-327 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide (e.g., of 14 to 30, for example, 14 to 23, linked nucleosides in length) having a nucleobase sequence comprising the nucleobase sequence of any one of SEQ ID NOs: 10-166 or 167-327 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide having a nucleobase sequence selected from the nucleobase sequences of any one of SEQ ID NOs: 10-166 and 167-327 and a second modified oligonucleotide 19 to 23 linked nucleosides in length having a region of complementarity to the first modified oligonucleotide. In certain embodiments, a compound comprises a first modified oligonucleotide having a nucleobase sequence selected from the nucleobase sequences of any one of SEQ ID NOs: 85 and 134 and a second modified oligonucleotide having a nucleobase sequence selected from the nucleobase sequences of any one of SEQ ID NOs: 244 and 294. In certain embodiments, administering the compound improves, preserves, or prevents a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment in an animal. In certain embodiments, the individual is identified as having, or at risk of having, a disease associated with AGT.

In certain embodiments, a method of inhibiting expression of AGT in an individual having, or at risk of having, a disease associated with AGT comprises administering to the individual a compound comprising a AGT specific inhibitor, thereby inhibiting expression of AGT in the individual. In certain embodiments, administering the compound inhibits expression of AGT in the liver. In certain embodiments, the disease is a RAAS related disease. In certain embodiments, the individual has, or is at risk of having, a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm, and peripheral artery disease), organ damage (e.g., heart, liver or kidney), inflammatory bowel disease or cognitive impairment. In certain embodiments, the compound comprises an antisense oligonucleotide targeted to AGT. In certain embodiments, the compound comprises an oligonucleotide targeted to AGT. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166. In certain embodiments, a compound comprises a modified oligonucleotide (e.g., of 14 to 30, for example, 14 to 23, linked nucleosides in length) and having a nucleobase sequence comprising the nucleobase sequence of any one of SEQ ID NOs: 10-166. In certain embodiments, a compound comprises a modified oligonucleotide having a nucleobase sequence selected from the nucleobase sequences of any one of SEQ ID NOs: 10-166. In certain embodiments, a compound comprises a modified oligonucleotide (e.g., of 14 to 30, for example, 14 to 23, linked nucleosides in length) having a nucleobase sequence comprising any one of the nucleobase sequences of SEQ ID NOs: 85 and 134. In certain embodiments, a compound comprises a modified oligonucleotide having a nucleobase sequence selected from any one of the nucleobase sequences of SEQ ID NOs: 85 and 134. In any of the foregoing embodiments, the compound can be single-stranded or double-stranded. In any of the foregoing embodiments, the compound can be an antisense oligonucleotide or oligomeric compound. In certain embodiments, a compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166 or 167-327 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide (e.g., of 14 to 30, for example, 14 to 23, linked nucleosides in length) having a nucleobase sequence comprising the nucleobase sequence of any one of SEQ ID NOs: 10-166 or 167-327 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide having a nucleobase sequence selected from the nucleobase sequences of any one of SEQ ID NOs: 10-166 and 167-327 and a second modified oligonucleotide 19 to 23 linked nucleosides in length having a region of complementarity to the first modified oligonucleotide. In certain embodiments, a compound comprises a first modified oligonucleotide having a nucleobase sequence selected from the nucleobase sequences of any one of SEQ ID NOs: 85 and 134 and a second modified oligonucleotide having a nucleobase sequence selected from the nucleobase sequences of any one of SEQ ID NOs: 244 and 294. In certain embodiments, the compound is administered to the individual parenterally. In certain embodiments, administering the compound improves, preserves, or prevents a RAAS related disease, disorder or condition or a symptom thereof, hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment.

In certain embodiments, a method of inhibiting expression of AGT in a cell comprises contacting the cell with a compound comprising a AGT specific inhibitor, thereby inhibiting expression of AGT in the cell. In certain embodiments, the cell is a hepatocyte. In certain embodiments, the cell is in the liver. In certain embodiments, the cell is in the liver of an individual who has, or is at risk of having, a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment. In certain embodiments, the compound comprises an antisense oligonucleotide targeted to AGT. In certain embodiments, the compound comprises an oligonucleotide targeted to AGT. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166. In certain embodiments, a compound comprises a modified oligonucleotide (e.g., of 14 to 30, for example, 14 to 23, linked nucleosides in length) and having a nucleobase sequence comprising the nucleobase sequence of any one of SEQ ID NOs: 10-166. In certain embodiments, a compound comprises a modified oligonucleotide having a nucleobase sequence selected from the nucleobase sequences of any one of SEQ ID NOs: 10-166. In certain embodiments, a compound comprises a modified oligonucleotide (e.g., of 14 to 30, for example, 14 to 23, linked nucleosides in length) having a nucleobase sequence comprising the nucleobase sequence of any one of SEQ ID NOs: 85 and 134. In certain embodiments, a compound comprises a modified oligonucleotide having a nucleobase sequence selected from any one of the nucleobase sequences of SEQ ID NOs: 85 and 134. In any of the foregoing embodiments, the compound can be single-stranded or double-stranded. In any of the foregoing embodiments, the compound can be an antisense oligonucleotide or oligomeric compound. In certain embodiments, a compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166 or 167-327 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide (e.g., of 14 to 30, for example, 14 to 23, linked nucleosides in length) having a nucleobase sequence comprising the nucleobase sequence of any one of SEQ ID NOs: 10-166 or 167-327 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide having a nucleobase sequence selected from the nucleobase sequences of any one of SEQ ID NOs: 10-166 and 167-327 and a second modified oligonucleotide 19 to 23 linked nucleosides in length having a region of complementarity to the first modified oligonucleotide. In certain embodiments, a compound comprises a first modified oligonucleotide having a nucleobase sequence selected from the nucleobase sequences of any one of SEQ ID NOs: 85 and 134 and a second modified oligonucleotide having a nucleobase sequence selected from the nucleobase sequences of any one of SEQ ID NOs: 244 and 294.

In certain embodiments, a method of reducing or inhibiting a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment in an individual having, or at risk of having, a disease associated with AGT comprises administering to the individual a compound comprising a AGT specific inhibitor, thereby reducing or inhibiting a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment in the individual. In certain embodiments, the individual has, or is at risk of having, a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm, and peripheral artery disease), organ damage (e.g., heart, liver or kidney), inflammatory bowel disease or cognitive impairment. In certain embodiments, the compound comprises an antisense oligonucleotide targeted to AGT. In certain embodiments, the compound comprises an oligonucleotide targeted to AGT. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166. In certain embodiments, a compound comprises a modified oligonucleotide (e.g., of 14 to 30, for example, 14 to 23, linked nucleosides in length) and having a nucleobase sequence comprising the nucleobase sequence of any one of SEQ ID NOs: 10-166. In certain embodiments, a compound comprises a modified oligonucleotide having a nucleobase sequence selected from the nucleobase sequences of any one of SEQ ID NOs: 10-166. In certain embodiments, a compound comprises a modified oligonucleotide (e.g., of 14 to 30, for example, 14 to 23, linked nucleosides in length) having a nucleobase sequence comprising the nucleobase sequence of any one of SEQ ID NOs: 85 and 134. In certain embodiments, a compound comprises a modified oligonucleotide having a nucleobase sequence selected from the nucleobase sequences of any one of SEQ ID NOs: 85 and 134. In any of the foregoing embodiments, the compound can be single-stranded or double-stranded. In any of the foregoing embodiments, the compound can be an antisense oligonucleotide or oligomeric compound. In certain embodiments, a compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166 or 167-327 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide (e.g., of 14 to 30, for example, 14 to 23, linked nucleosides in length) having a nucleobase sequence comprising the nucleobase sequence of any one of SEQ ID NOs: 10-166 or 167-327 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide having a nucleobase sequence selected from the nucleobase sequences of any one of SEQ ID NOs: 10-166 and 167-327 and a second modified oligonucleotide 19 to 23 linked nucleosides in length having a region of complementarity to the first modified oligonucleotide. In certain embodiments, a compound comprises a first modified oligonucleotide having a nucleobase sequence selected from the nucleobase sequences of any one of SEQ ID NOs: 85 and 134 and a second modified oligonucleotide having a nucleobase sequence selected from the nucleobase sequences of any one of SEQ ID NOs: 244 and 294. In certain embodiments, the compound is administered to the individual parenterally. In certain embodiments, the individual is identified as having, or at risk of having, a disease associated with AGT.

Certain embodiments are drawn to a compound comprising a AGT specific inhibitor for use in treating a disease, disorder or condition associated with AGT. In certain embodiments, the disease, disorder or condition is a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment. In certain embodiments, the compound comprises an antisense oligonucleotide targeted to AGT. In certain embodiments, the compound comprises an oligonucleotide targeted to AGT. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166. In certain embodiments, a compound comprises a modified oligonucleotide (e.g., of 14 to 30, for example, 14 to 23, linked nucleosides in length) and having a nucleobase sequence comprising the nucleobase sequence of any one of SEQ ID NOs: 10-166. In certain embodiments, a compound comprises a modified oligonucleotide having a nucleobase sequence selected from the nucleobase sequences of any one of SEQ ID NOs: 10-166. In certain embodiments, a compound comprises a modified oligonucleotide (e.g., of 14 to 30, for example, 14 to 23, linked nucleosides in length) having a nucleobase sequence comprising any one of the nucleobase sequences of SEQ ID NOs: 85 and 134. In certain embodiments, a compound comprises a modified oligonucleotide having a nucleobase sequence selected from the nucleobase sequences of any one of SEQ ID NOs: 85 and 134. In any of the foregoing embodiments, the compound can be single-stranded or double-stranded. In any of the foregoing embodiments, the compound can be an antisense oligonucleotide or oligomeric compound. In certain embodiments, a compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166 or 167-327 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide (e.g., of 14 to 30, for example, 14 to 23, linked nucleosides in length) having a nucleobase sequence comprising the nucleobase sequence of any one of SEQ ID NOs: 10-166 or 167-327 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide having a nucleobase sequence selected from the nucleobase sequences of any one of SEQ ID NOs: 10-166 and 167-327 and a second modified oligonucleotide 19 to 23 linked nucleosides in length having a region of complementarity to the first modified oligonucleotide. In certain embodiments, a compound comprises a first modified oligonucleotide having a nucleobase sequence selected from the nucleobase sequences of any one of SEQ ID NOs: 85 and 134 and a second modified oligonucleotide having a nucleobase sequence selected from the nucleobase sequences of any one of SEQ ID NOs: 244 and 294. In certain embodiments, the compound is administered to the individual parenterally.

Certain embodiments are drawn to a compound comprising a AGT specific inhibitor for use in reducing or inhibiting a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment. In certain embodiments, the compound comprises an antisense oligonucleotide targeted to AGT. In certain embodiments, the compound comprises an oligonucleotide targeted to AGT. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166. In certain embodiments, a compound comprises a modified oligonucleotide (e.g., of 14 to 30, for example, 14 to 23, linked nucleosides in length) and having a nucleobase sequence comprising the nucleobase sequence of any one of SEQ ID NOs: 10-166. In certain embodiments, a compound comprises a modified oligonucleotide having a nucleobase sequence selected from the nucleobase sequences of any one of SEQ ID NOs: 10-166. In certain embodiments, a compound comprises a modified oligonucleotide (e.g., of 14 to 30, for example, 14 to 23, linked nucleosides in length) having a nucleobase sequence comprising any one of the nucleobase sequences of SEQ ID NOs: 85 and 134. In certain embodiments, a compound comprises a modified oligonucleotide having a nucleobase sequence selected from any one of the nucleobase sequences of SEQ ID NOs: 85 and 134. In any of the foregoing embodiments, the compound can be single-stranded or double-stranded. In any of the foregoing embodiments, the compound can be an antisense oligonucleotide or oligomeric compound. In certain embodiments, a compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166 or 167-327 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide (e.g., of 14 to 30, for example, 14 to 23, linked nucleosides in length) having a nucleobase sequence comprising the nucleobase sequence of any one of SEQ ID NOs: 10-166 or 167-327 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide having a nucleobase sequence selected from any one of the nucleobase sequences of SEQ ID NOs: 10-166 and 167-327 and a second modified oligonucleotide 19 to 23 linked nucleosides in length having a region of complementarity to the first modified oligonucleotide. In certain embodiments, a compound comprises a first modified oligonucleotide having a nucleobase sequence selected from the nucleobase sequences of any one of SEQ ID NOs: 85 and 134 and a second modified oligonucleotide having a nucleobase sequence selected from the nucleobase sequences of any one of SEQ ID NOs: 244 and 294.

Certain embodiments are drawn to the use of a compound comprising a AGT specific inhibitor for the manufacture or preparation of a medicament for treating a disease associated with AGT. Certain embodiments are drawn to the use of a compound comprising a AGT specific inhibitor for the preparation of a medicament for treating a disease, disorder or condition associated with AGT. In certain embodiments, the disease, disorder or condition is a RAAS related disease, disorder or condition or a symptom thereof. In certain embodiments, the disease, disorder or condition is hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment. In certain embodiments, the compound comprises an antisense oligonucleotide targeted to AGT. In certain embodiments, the compound comprises an oligonucleotide targeted to AGT. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166. In certain embodiments, a compound comprises a modified oligonucleotide (e.g., of 14 to 30, for example, 14 to 23, linked nucleosides in length) and having a nucleobase sequence comprising the nucleobase sequence of any one of SEQ ID NOs: 10-166. In certain embodiments, a compound comprises a modified oligonucleotide having a nucleobase sequence selected from the nucleobase sequences of any one of SEQ ID NOs: 10-166. In certain embodiments, a compound comprises a modified oligonucleotide (e.g., of 14 to 30, for example, 14 to 23, linked nucleosides in length) having a nucleobase sequence comprising any one of the nucleobase sequences of SEQ ID NOs: 85 and 134. In certain embodiments, a compound comprises a modified oligonucleotide having a nucleobase sequence selected from any one of the nucleobase sequences of SEQ ID NOs: 85 and 134. In any of the foregoing embodiments, the compound can be single-stranded or double-stranded. In any of the foregoing embodiments, the compound can be an antisense oligonucleotide or oligomeric compound. In certain embodiments, a compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166 or 167-327 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide (e.g., of 14 to 30, for example, 14 to 23, linked nucleosides in length) having a nucleobase sequence comprising the nucleobase sequence of any one of SEQ ID NOs: 10-166 or 167-327 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide having a nucleobase sequence selected from the nucleobase sequences of any one of SEQ ID NOs: 10-166 and 167-327 and a second modified oligonucleotide 19 to 23 linked nucleosides in length having a region of complementarity to the first modified oligonucleotide. In certain embodiments, a compound comprises a first modified oligonucleotide having a nucleobase sequence selected from the nucleobase sequences of any one of SEQ ID NOs: 85 and 134 and a second modified oligonucleotide having a nucleobase sequence selected from the nucleobase sequences of any one of SEQ ID NOs: 244 and 294.

Certain embodiments are drawn to the use of a compound comprising a AGT specific inhibitor for the manufacture or preparation of a medicament for reducing or inhibiting a RAAS related disease, disorder or condition or a symptom thereof in an individual having, or at risk of having, a RAAS related disease, disorder or condition or a symptom thereof associated with AGT. In certain embodiments, the RAAS related disease, disorder or condition is hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment. Certain embodiments are drawn to use of a compound comprising a AGT specific inhibitor for the preparation of a medicament for treating a disease, disorder or condition associated with AGT. In certain embodiments, the disease, disorder or condition is a RAAS related disease, disorder or condition or a symptom thereof or hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment. In certain embodiments, the compound comprises an antisense oligonucleotide targeted to AGT. In certain embodiments, the compound comprises an oligonucleotide targeted to AGT. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166. In certain embodiments, a compound comprises a modified oligonucleotide (e.g., of 14 to 30, for example, 14 to 23, linked nucleosides in length) and having a nucleobase sequence comprising the nucleobase sequence of any one of SEQ ID NOs: 10-166. In certain embodiments, a compound comprises a modified oligonucleotide having a nucleobase sequence selected from the nucleobase sequences of any one of SEQ ID NOs: 10-166. In certain embodiments, a compound comprises a modified oligonucleotide (e.g., of 14 to 30, for example, 14 to 23, linked nucleosides in length) having a nucleobase sequence comprising the nucleobase sequence of any one of SEQ ID NOs: 85 and 134. In certain embodiments, a compound comprises a modified oligonucleotide having a nucleobase sequence selected from the nucleobase sequences of any one of SEQ ID NOs: 85 and 134. In any of the foregoing embodiments, the compound can be single-stranded or double-stranded. In any of the foregoing embodiments, the compound can be an antisense oligonucleotide or oligomeric compound. In certain embodiments, a compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166 or 167-327 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide (e.g., of 14 to 30, for example, 14 to 23, linked nucleosides in length) having a nucleobase sequence comprising the nucleobase sequence of any one of SEQ ID NOs: 10-166 or 167-327 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide having a nucleobase sequence selected from the nucleobase sequences of any one of SEQ ID NOs: 10-166 and 167-327 and a second modified oligonucleotide 19 to 23 linked nucleosides in length having a region of complementarity to the first modified oligonucleotide. In certain embodiments, a compound comprises a first modified oligonucleotide having a nucleobase sequence selected from the nucleobase sequences of any one of SEQ ID NOs: 85 and 134 and a second modified oligonucleotide having a nucleobase sequence selected from the nucleobase sequences of any one of SEQ ID NOs: 244 and 294.

In any of the foregoing methods or uses, the compound can be an oligomeric compound. In any of the foregoing methods or uses, the compound can be single-stranded or double-stranded. In any of the foregoing methods or uses, the compound can be targeted to AGT. In certain embodiments, the compound comprises or consists of a modified oligonucleotide. In certain embodiments, the compound comprises one or more modified oligonucleotides. In certain embodiments, the compound comprises a first modified oligonucleotide and a second modified oligonucleotide. In certain embodiments, a modified oligonucleotide is 8 to 80 linked nucleosides in length, 10 to 30 linked nucleosides in length, 14 to 30 linked nucleosides in length, 14 to 23 linked nucleosides in length, or 19 to 23 linked nucleosides in length. In certain embodiments, a modified oligonucleotide having a nucleobase sequence that is at least 80%, at least 85%, at least 90%, at least 95% or 100% complementary to any of the nucleobase sequences recited in SEQ ID NOs: 1 and 3 over its length. In certain embodiments, a modified oligonucleotide comprises at least one modified internucleoside linkage, at least one modified sugar and/or at least one modified nucleobase. In certain embodiments, the modified internucleoside linkage is a phosphorothioate internucleoside linkage. In certain embodiments, the modified sugar is a bicyclic sugar, 2′-MOE, 2′-F, or 2′-OMe. In certain embodiments, the modified nucleobase is a 5-methylcytosine. In any of the foregoing embodiments, each modified oligonucleotide is independently 12 to 30, 14 to 30, 14 to 25, 14 to 24, 14 to 23, 16 to 23, 17 to 23, 18 to 23, 19 to 23, 19 to 22, or 19 to 20 linked nucleosides in length. In certain embodiments, a modified oligonucleotide having a nucleobase sequence has at least 1, at least 2, at least 3 mismatches to a region of the nucleobases of SEQ ID NO: 1 and 3.

In any of the forgoing methods or uses, the compound comprises a first and second modified oligonucleotide, wherein there is a region of complementarity between a first modified oligonucleotide and a second modified oligonucleotide. In certain embodiments, the region of complementarity between the first oligonucleotide and the second oligonucleotide is 14 to 23, 19 to 23, or 21 to 23 linked nucleosides in length. In certain embodiments, the first modified oligonucleotide is fully complementary to the second modified oligonucleotide. In certain embodiments, the first modified oligonucleotide comprises at least one modification selected from a modified internucleoside linkage, a modified sugar, and a modified nucleobase. In certain embodiments, the second modified oligonucleotide comprises at least one modification selected from the group consisting of a modified internucleoside linkage, a modified sugar, and a modified nucleobase. In certain embodiments, the modified internucleoside linkage is a phosphorothioate internucleoside linkage or a methylphosphonate internucleoside linkage. In certain embodiments, the modified internucleoside linkage is at the 3′ terminus of the first or second modified oligonucleotide or at the 5′ terminus of the first or second modified oligonucleotide. In certain embodiments, the first or second modified oligonucleotide comprises one or more modified sugars. In certain embodiments, each nucleoside of the first or second modified oligonucleotide comprises a modified sugar. In certain embodiments, the modified sugar comprises a modification selected from the group consisting of a halogen, an alkoxy group and a bicyclic sugar. In certain embodiments, the modified sugar comprises a modification selected from group consisting of 2′-MOE, 2′-F, and 2′-OMe or a combination thereof. In certain embodiments, the first or second modified oligonucleotide comprises no more than ten 2′-F sugar modifications. In certain embodiments, the first or second modified oligonucleotide comprises no more than five 2′-F sugar modifications.

In any of the forgoing methods or uses, a compound comprises a conjugate group. In certain embodiments, the conjugate group is attached to the 5′ end of a modified oligonucleotide. In certain embodiments, the conjugate group is a targeting moiety. In certain embodiments, the targeting moiety comprises one or more GalNAc. In certain embodiments, the one or more GalNAc is attached to the 2′ or 3′ position of the ribosyl ring. In certain embodiments, the one or more GalNAc is attached to the 5′ nucleoside of the modified oligonucleotide. In certain embodiments, the 5′ nucleoside of a modified oligonucleotide is selected from Formulae I-VIII, or a salt, solvate, or hydrate thereof, wherein R is the modified oligonucleotide other than the 5′ nucleoside. In certain embodiments, the 5′ nucleoside of the modified oligonucleotide is Formula I and R′ is O. In certain embodiments, the 5′ nucleoside of the modified oligonucleotide is Formula I and R′ is S. In certain embodiments, the 5′ nucleoside of the modified oligonucleotide is Formula II and R′ is O. In certain embodiments, the 5′ nucleoside of the modified oligonucleotide is Formula II and R′ is S. In certain embodiments, the 5′ nucleoside of the modified oligonucleotide is Formula III and R′ is O. In certain embodiments, the 5′ nucleoside of the modified oligonucleotide is Formula III and R′ is S. In certain embodiments, the 5′ nucleoside of the modified oligonucleotide is Formula IV and R′ is O. In certain embodiments, the 5′ nucleoside of the modified oligonucleotide is Formula IV and R′ is S. In certain embodiments, the 5′ nucleoside of the modified oligonucleotide is Formula V and R′ is O. In certain embodiments, the 5′ nucleoside of the modified oligonucleotide is Formula V and R′ is S. In certain embodiments, the 5′ nucleoside of the modified oligonucleotide is Formula VI and R′ is O. In certain embodiments, the 5′ nucleoside of the modified oligonucleotide is Formula VI and R′ is S. In certain embodiments, the 5′ nucleoside of the modified oligonucleotide is Formula VII and R′ is O. In certain embodiments, the 5′ nucleoside of the modified oligonucleotide is Formula VII and R′ is S. In certain embodiments, the 5′ nucleoside of the modified oligonucleotide is Formula VIII and R′ is O. In certain embodiments, the 5′ nucleoside of the modified oligonucleotide is Formula VIII and R′ is S.

In any of the foregoing methods or uses, the compound comprises a first modified oligonucleotide selected from any one of Ref ID NOs: IA0297, IA0300, IA0301, IA0304, IA0305, IA0335-338, IA0343-359, IA0431-432, IA0435, IA440-446, IA0727-728, IA0500-501, and IA0868, and a second modified oligonucleotide 14 to 23 linked nucleosides in length fully complementary to the first modified oligonucleotide. In certain embodiments, the compound comprises a first modified oligonucleotide which is Ref ID NO: IA0443 and a second modified oligonucleotide which is Ref ID NO: IS0505. In certain embodiments, the compound comprises a first modified oligonucleotide which is Ref ID NO: IA0445 and a second modified oligonucleotide which is Ref ID NO: IS0509. In certain embodiments, the compound is in a pharmaceutically acceptable salt form. In certain embodiments, the pharmaceutically acceptable salt is a sodium salt. In certain embodiments, the pharmaceutically acceptable salt is a potassium salt. In certain embodiments, a composition comprises the compound of any one of the foregoing embodiments and a pharmaceutically acceptable carrier.

In any of the foregoing methods or uses, a compound or composition comprising a compound of any preceding embodiment is administered to an individual in a therapeutically effective amount. In certain embodiments, a compound or composition comprising a compound of any preceding embodiment is administered to an individual at a dosage level sufficient to deliver about 1 to 100 mg/kg of body weight of the individual. In certain embodiments, a compound or composition comprising a compound of any preceding embodiment is administered to an individual at a fixed dose of about 25 mg to about 1,000 mg. In certain embodiments, the composition is administered to the individual one or more times in a day up to the dosage level or fixed dose.

In any of the foregoing methods or uses, a compound or composition comprising a compound of any preceding embodiment is administered to an individual daily, weekly, monthly, quarterly, or yearly. In certain embodiments, a compound or composition comprising a compound of any preceding embodiment is administered to an individual about once per quarter (i.e., once every three months) to about once per year. In certain embodiments, a compound or composition comprising a compound of any preceding embodiment is administered to an individual about once per quarter, about once every six months or about once per year.

### Certain Compounds

In certain aspects, the disclosure relates to a compound that comprises or consists of an oligomeric compound. In certain embodiments, the oligomeric compound comprises a nucleobase sequence complementary to that of a target nucleic acid.

In certain aspects, the disclosure relates to a compound that comprises or consists of a modified oligonucleotide. In certain embodiments, the modified oligonucleotide has a nucleobase sequence complementary to that of a target nucleic acid.

In certain aspects, the disclosure relates to a compound that comprises or consists of an antisense oligonucleotide. In certain embodiments, the antisense oligonucleotide has a nucleobase sequence complementary to that of a target nucleic acid.

In certain aspects, the disclosure relates to a compound that is a single-stranded compound. In certain embodiments, the single-stranded compound comprises or consists of an oligomeric compound. In certain embodiments, such an oligomeric compound comprises or consists of an oligonucleotide and optionally a conjugate group. In certain embodiments, the oligonucleotide is a modified oligonucleotide. In certain embodiments, the oligonucleotide is an antisense oligonucleotide. In certain embodiments, the oligonucleotide or modified oligonucleotide of a single-stranded compound comprises a self-complementary nucleobase sequence.

In certain aspects, the disclosure relates to a compound that is a double-stranded compound. In certain embodiments, the double-stranded compound comprises or consists of an oligomeric compound. In certain embodiments, the double-stranded compound comprises a first oligonucleotide and a second oligonucleotide. In certain embodiments, the first oligonucleotide has a region complementarity to a target nucleic acid and the second oligonucleotide has a region complementarity to the first modified oligonucleotide. In certain embodiments, the double-stranded compound comprises a modified oligonucleotide. In certain embodiments, the modified oligonucleotide has a region complementarity to a target nucleic acid. In certain embodiments, the double-stranded compound comprises a first modified oligonucleotide and a second modified oligonucleotide. In certain embodiments, the first modified oligonucleotide has a region complementarity to a target nucleic acid and the second modified oligonucleotide has a region complementarity to the first modified oligonucleotide. In certain embodiments, an oligonucleotide or modified oligonucleotide of a double-stranded compound is an RNA oligonucleotide. In such embodiments, the thymine nucleobase in the modified oligonucleotide is replaced by a uracil nucleobase.

In certain embodiments, a compound described herein comprises a conjugate group. In certain embodiments, the first oligonucleotide or first modified oligonucleotide of a double-stranded compound comprises a conjugate group. In certain embodiments, the second oligonucleotide or second modified oligonucleotide of a double-stranded compound comprises a conjugate group. In certain embodiments, a first oligonucleotide or first modified oligonucleotide and a second oligonucleotide or second modified oligonucleotide of a double-stranded compound each comprises a conjugate group.

In certain embodiments, a compound is 14-30 linked nucleosides in length. In certain embodiments, the first oligonucleotide or first modified oligonucleotide of a double-stranded compound is 14-30 linked nucleosides in length. In certain embodiments, the second oligonucleotide or second modified oligonucleotide is 14-30 linked nucleosides in length. In certain embodiments, the oligonucleotides or modified oligonucleotides of a double-stranded compound are blunt ended at one or both ends of the compound. In certain embodiments, the oligonucleotides or modified oligonucleotides of a double-stranded compound include non-complementary overhanging nucleosides at one or both ends of the compound.

In certain embodiments, a compound has a nucleobase sequence comprising at least 14 contiguous nucleobases of any of the nucleobase sequences of SEQ ID NOs: 10-166. In certain embodiments, one of the oligonucleotides or modified oligonucleotides of a double-stranded compound has a nucleobase sequence comprising at least 14 contiguous nucleobases of any of the nucleobase sequences of SEQ ID NOs: 10-166.

Examples of single-stranded and double-stranded compounds include, but are not limited to, oligonucleotides, antisense oligonucleotides, siRNAs, microRNA targeting oligonucleotides, occupancy-based compounds (e.g., mRNA processing or translation blocking compounds and splicing compounds), and single-stranded RNAi compounds (e.g., small hairpin RNAs (shRNAs), single stranded siRNAs (ssRNAs) and microRNA mimics).

In certain embodiments, a compound described herein has a nucleobase sequence that, when written in the 5′ to 3′ direction, comprises the reverse complement of the target region of a target nucleic acid to which it is targeted.

In certain embodiments, a compound described herein comprises an oligonucleotide 12 to 30 linked subunits in length. In certain embodiments, a compound described herein comprises an oligonucleotide 12 to 23 linked subunits in length. In certain embodiments, compound described herein comprises an oligonucleotide 14 to 30 linked subunits in length. In certain embodiments, compound described herein comprises an oligonucleotide 14 to 23 linked subunits in length. In certain embodiments, a compound described herein comprises an oligonucleotide 15 to 30 linked subunits in length. In certain embodiments, a compound described herein comprises an oligonucleotide 15 to 23 linked subunits in length. In certain embodiments, a compound described herein comprises an oligonucleotide 16 to 30 linked subunits in length. In certain embodiments, a compound described herein comprises an oligonucleotide 16 to 23 linked subunits in length. In certain embodiments, a compound described herein comprises an oligonucleotide 17 to 30 linked subunits in length. In certain embodiments, a compound described herein comprises an oligonucleotide 17 to 23 linked subunits in length. In certain embodiments, a compound described herein comprises an oligonucleotide 18 to 30 linked subunits in length. In certain embodiments, a compound described herein comprises an oligonucleotide 18 to 23 linked subunits in length. In certain embodiments, a compound described herein comprises an oligonucleotide 19 to 30 linked subunits in length. In certain embodiments, a compound described herein comprises an oligonucleotide 19 to 23 linked subunits in length. In other words, such oligonucleotides are 12 to 30 linked subunits, 12 to 23 linked subunits, 14 to 30 linked subunits, 14 to 23 linked subunits, 15 to 30 linked subunits, 15 to 23 linked subunits, 16 to 30 linked subunits, 16 to 23 linked subunits, 17 to 30 linked subunits, 17 to 23 linked subunits, 18 to 30 linked subunits, 18 to 23 linked subunits, 19 to 30 linked subunits or 19 to 23 linked subunits, respectively. In certain embodiments, a compound described herein comprises an oligonucleotide 14 linked subunits in length. In certain embodiments, a compound described herein comprises an oligonucleotide 16 linked subunits in length. In certain embodiments, a compound described herein comprises an oligonucleotide 17 linked subunits in length. In certain embodiments, compound described herein comprises an oligonucleotide 18 linked subunits in length. In certain embodiments, a compound described herein comprises an oligonucleotide 19 linked subunits in length. In certain embodiments, a compound described herein comprises an oligonucleotide 20 linked subunits in length. In certain embodiments, a compound described herein comprises an oligonucleotide 21 linked subunits in length. In certain embodiments, a compound described herein comprises an oligonucleotide 22 linked subunits in length. In certain embodiments, a compound described herein comprises an oligonucleotide 23 linked subunits in length. In other embodiments, a compound described herein comprises an oligonucleotide 8 to 80, 12 to 50, 13 to 30, 13 to 50, 14 to 30, 14 to 50, 15 to 30, 15 to 50, 16 to 30, 16 to 50, 17 to 30, 17 to 50, 18 to 23, 18 to 24, 18 to 25, 18 to 50, 19 to 23, 19 to 30, 19 to 50, 20 to 23 or 20 to 30 linked subunits. In certain such embodiments, the compound described herein comprises an oligonucleotide 8, 9, 10, 11, 12, 13, 14, 15, 16, 17, 18, 19, 20, 21, 22, 23, 24, 25, 26, 27, 28, 29, or 30 linked subunits in length, or a range defined by any two of the above values.

In certain embodiments, the compound may further comprise an additional moiety, such as a conjugate group or delivery moiety. In certain embodiments, such compounds are oligomeric compounds, and the additional moiety is attached to an oligonucleotide. In certain embodiments, a conjugate group is attached to a nucleoside of an oligonucleotide.

In certain embodiments, compounds may be shortened or truncated. For example, one or more subunits may be deleted from the 5′ end (5′ truncation), or alternatively from the 3′ end (3′ truncation) of an oligonucleotide.

In certain embodiments, compounds may be lengthened. For example, one or more subunits may be attached to the 3′ end or 5′ end of an oligonucleotide. In certain embodiments, at least one subunit (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more subunits) is attached to the 5′ end of an oligonucleotide. In certain embodiments, at least one subunit (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more subunits) is attached to the 3′ end of an oligonucleotide. In certain embodiments, at least one or more subunits may be attached to the 3′ end or 5′ end of an oligonucleotide of a double-stranded compound creating a 3′ and/or 5′ end overhang. In certain embodiments, at least one subunit (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more subunits) is attached to the 5′ end of both oligonucleotides of a double-stranded compound. In certain embodiments, at least one subunit (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more subunit) is attached to the 3′ end of both oligonucleotides of a double-stranded compound. In certain embodiments, subunits are attached to both oligonucleotides of a double-stranded compound at the same end (e.g., that subunits are attached to the 3′ end of one of the oligonucleotides and subunits are attached to the 5′ end of the other oligonucleotide). In certain embodiments, when subunits are attached to both oligonucleotides of a double-stranded compound at the same end, the number of subunits attached to each oligonucleotide may be the same or may be different. In certain embodiments, when subunits are attached to both oligonucleotides of a double-stranded compound at the same end, the number of subunits attached to each oligonucleotide is the same. In certain embodiments, when subunits are attached to both oligonucleotides of a double-stranded compound at the same end, the number of subunits attached to each oligonucleotide is different. This scenario, where subunits are attached to both oligonucleotides of a double-stranded compound at the same end, may occur at one or both ends of a double-stranded compound. In certain embodiments, the subunits attached to the 3′ and/or 5′ end are modified.

In certain embodiments, compounds described herein are oligonucleotides. In certain embodiments, compounds described herein are modified oligonucleotides. In certain embodiments, compounds described herein are antisense oligonucleotides. In certain embodiments, compounds described herein are oligomeric compounds. In certain embodiments, compounds described herein are RNAi compounds. In certain embodiments, compounds described herein are siRNA compounds.

In certain embodiments, a compound described herein can comprise any of the oligonucleotide sequences targeted to AGT described herein. In certain embodiments, the compound can be double-stranded.

In certain embodiments, the compound comprises an oligonucleotide having a nucleobase sequence comprising at least an 8, 9, 10, 11, 12, 13, 14, 15, 16, 17, 18, 19, 20, 21, 22 or 23 contiguous nucleobase portion of any one of the nucleobase sequences of SEQ ID NOs: 10-166. In certain embodiments, the compound comprises a second oligonucleotide.

In certain embodiments, the compound comprises ribonucleotides in which the oligonucleotide has uracil (U) in place of thymine (T) for any of the sequences provided here. In certain embodiments, the compound comprises deoxyribonucleotides in which the oligonucleotide has thymine (T) in place of uracil (U) for any of the sequences provided here.

### Certain Mechanisms

In certain embodiments, compounds described herein comprise or consist of modified oligonucleotides. In certain embodiments, compounds described herein comprise or consist of antisense oligonucleotides. In certain embodiments, compounds comprise or consist of oligomeric compounds. In certain embodiments, compounds described herein are capable of hybridizing to a target nucleic acid. In certain embodiments, compounds described herein selectively affect one or more target nucleic acid. Such compounds comprise a nucleobase sequence that hybridizes to one or more target nucleic acid, resulting in one or more desired activity and does not hybridize to one or more non-target nucleic acid or does not hybridize to one or more non-target nucleic acid in such a way that results in a significant undesired activity.

In certain embodiments, hybridization of a compound described herein to a target nucleic acid results in recruitment of one or more proteins that cause the cleavage of the target nucleic acid. For example, certain compounds described herein or a portion of the compound is loaded into an RNA-induced silencing complex (RISC), ultimately resulting in cleavage of the target nucleic acid. For example, certain compounds described herein result in cleavage of the target nucleic acid by Argonaute. Compounds that are loaded into RISC are RNAi compounds. RNAi compounds may be double-stranded (siRNA) or single-stranded (ssRNA).

In certain embodiments, hybridization of compounds described herein to a target nucleic acid does not result in recruitment of a protein that cleaves that target nucleic acid. In certain such embodiments, hybridization of the compound to the target nucleic acid results in the alteration of splicing of the target nucleic acid. In certain embodiments, hybridization of the compound to a target nucleic acid results in inhibition of a binding interaction between the target nucleic acid and a protein or other nucleic acid. In certain such embodiments, hybridization of the compound to the target nucleic acid results in the alteration of RNA processing. In certain such embodiments, hybridization of the compound to a target nucleic acid results in alteration of translation of the target nucleic acid.

Activities resulting from the hybridization of a compound to a target nucleic acid may be observed directly or indirectly. In certain embodiments, observation or detection of an activity involves observation or detection of a change in an amount of a target nucleic acid or protein encoded by such target nucleic acid, a change in the ratio of splice variants of a nucleic acid or protein, and/or a phenotypic change in a cell or animal.

### Certain Modifications

In certain aspects, the disclosure relates to compounds that comprise or consist of oligonucleotides. Oligonucleotides consist of linked nucleosides. In certain embodiments, oligonucleotides may be unmodified RNA or DNA or may be modified. In certain embodiments, the oligonucleotides are modified oligonucleotides. In certain embodiments, the modified oligonucleotides comprise at least one modified sugar, modified nucleobase or modified internucleoside linkage relative to an unmodified RNA or DNA. In certain embodiments, an oligonucleotide has a modified nucleoside. A modified nucleoside may comprise a modified sugar, a modified nucleobase or both a modified sugar and a modified nucleobase. Modified oligonucleotides may also include end modifications, e.g., 5′-end modifications and 3′-end modifications.

### Sugar Modifications and Motifs

In certain embodiments, a modified sugar is a substituted furanosyl sugar or non-bicyclic modified sugar. In certain embodiments, a modified sugar is a bicyclic or tricyclic modified sugar. In certain embodiments, a modified sugar is a sugar surrogate. A sugar surrogate may comprise one or more substitutions described herein.

In certain embodiments, a modified sugar is a substituted furanosyl or non-bicyclic modified sugar. In certain embodiments, the furanosyl sugar is a ribosyl sugar. In certain embodiments, the furanosyl sugar comprises one or more substituent groups, including, but not limited to, substituent groups at the 2′, 3′, 4′, and 5′ positions.

In certain embodiments, substituents at the 2′ position include, but are not limited to, F and OCH₃ (“OMe”, “O-methyl” or “methoxy”). In certain embodiments, substituent groups at the 2′ position suitable for non-bicyclic modified sugars include, but are not limited to, halo, allyl, amino, azido, SH, CN, OCN, CF₃, OCF₃, F, Cl, Br, SCH₃, SOCH₃, SO₂CH₃, ONO₂, NO₂, N₃, and NH₂. In certain embodiments, substituent groups at the 2′ position include, but are not limited to, O—(C₁-C₁₀) alkoxy, alkoxyalkyl, O-alkyl, S-alkyl, N-alkyl, O-alkenyl, S-alkenyl, N-alkenyl, O-alkynyl, S-alkynyl, N-alkynyl, O-alkyl-O-alkyl, alkynyl, wherein the alkyl, alkenyl and alkynyl can be substituted or unsubstituted C₁ to C₁₀ alkyl or C₂ to C₁₀ alkenyl and alkynyl. In certain embodiments, substituent groups at the 2′ position include, but are not limited to, alkaryl, aralkyl, O-alkaryl, and O-aralkyl. In certain embodiments, these 2′ substituent groups can be further substituted with one or more substituent groups independently selected from hydroxyl, alkoxy, carboxy, benzyl, phenyl, nitro (NO₂), thiol, thioalkoxy, thioalkyl, halogen, alkyl, aryl, alkenyl, and alkynyl. In certain embodiments, substituent groups at the 2′ position include, but are not limited to, O[(CH₂)ₙO]ₘCH₃, O(CH₂)ₙOCH₃, O(CH₂)ₙCH₃, O(CH₂)ₙONH₂, O(CH₂)ₙNH₂, O(CH₂)ₙSCH₃, and O(CH₂)ₙON[(CH₂)ₙCH₃)]₂, where n and m are independently from 1 to about 10. In certain embodiments, substituent groups at the 2′ position include, but are not limited to, OCH₂CH₂OCH₃ (“MOE”), O(CH₂)₂ON(CH₃)₂ (“DMAOE”), O(CH₂)₂O(CH₂)₂N(CH₃)₂ (“DMAEOE”), and OCH₂C(═O)—N(H)CH₃ (“NMA”).

In certain embodiments, substituent groups at the 4′ position suitable for non-bicyclic modified sugars include, but are not limited to, alkoxy (e.g., methoxy), alkyl, and those described in Manoharan et al., WO 2015/106128. In certain embodiments, substituent groups at the 5′ position suitable for non-bicyclic modified sugars include, but are not limited to, methyl (“Me”) (R or S), vinyl, and methoxy. In certain embodiments, substituents described herein for the 2′, 4′ and 5′ position can be added to other specific positions on the sugar. In certain embodiments, such substituents may be added to the 3′ position of the sugar on the 3′ terminal nucleoside or the 5′ position of the 5′ terminal nucleoside. In certain embodiments, a non-bicyclic modified sugar may comprise more than one non-bridging sugar substituent. In certain such embodiments, non-bicyclic modified sugars substituents include, but are not limited to, 5′-Me-2′-F, 5′-Me-2′-OMe (including both R and S isomers). In certain embodiments, modified sugar substituents include those described in Migawa et al., WO 2008/101157 and Rajeev et al., US2013/0203836.

In certain embodiments, a modified sugar is a bicyclic sugar. A bicyclic sugar is a modified sugar comprising two rings, wherein the second ring is formed via a bridge connecting two of the atoms in the first ring thereby forming a bicyclic structure. In certain embodiments, a bicyclic sugar comprises a bridging substituent that bridges two atoms of the furanosyl ring to form a second ring. In certain embodiments, a bicyclic sugar does not comprise a furanosyl moiety. A “bicyclic nucleoside” (“BNA”) is a nucleoside having a bicyclic sugar. In certain embodiments, the bicyclic sugar comprises a bridge between the 4′ and 2′ furanose ring atoms. In certain embodiments, the bicyclic sugar comprises a bridge between the 5′ and 3′ furanose ring atoms. In certain such embodiments, the furanose ring is a ribose ring. In certain embodiments, 4′ to 2′ bridging substituents include, but are not limited to, 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (“constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (e.g., U.S. Pat. No. 7,399,845), 4′-C(CH₃)(CH₃)—O-2′ and analogs thereof (e.g., U.S. Pat. No. 8,278,283), 4′-CH₂—N(OCH₃)-2′ and analogs thereof (e.g., U.S. Pat. No. 8,278,425), 4′-CH₂—O—N(CH₃)-2′ (e.g., U.S. Patent Publication No. 2004/0171570), 4′-CH₂—N(R)—O-2′, wherein R is H, C₁-C₁₂ alkyl, or a protecting group (e.g., U.S. Pat. No. 7,427,672), 4′-CH₂—C(H)(CH₃)-2′ (e.g., Chattopadhyaya el al., J. Org. Chem., 2009, 74, 118-134), and 4′-CH₂—C(═CH₂)-2′ and analogs thereof (e.g., U.S. Pat. No. 8,278,426). The entire contents of each of the foregoing are hereby incorporated herein by reference. Additional representative U.S. patents and U.S. patent Publications that teach the preparation of bicyclic nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, US 2013/0190383; and WO 2013/036868, the entire contents of each of which are hereby incorporated herein by reference. Any of the foregoing bicyclic nucleosides can be prepared having one or more stereochemical sugar configurations including for example α-L-ribofuranose and β-D-ribofuranose (see e.g., WO 99/14226). Specified bicyclic nucleosides herein are in the 3-D configuration, unless otherwise specified.

In certain embodiments, a modified sugar is a sugar surrogate. In certain embodiments, a sugar surrogate has the oxygen atom replaced, e.g., with a sulfur, carbon or nitrogen atom. In certain such embodiments, the sugar surrogate may also comprise bridging and/or non-bridging substituents as described herein. In certain embodiments, sugar surrogates comprise rings having other than 5 atoms. In certain such embodiments, the sugar surrogate comprises a cyclobutyl moiety in place of the pentofuranosyl sugar. In certain embodiments, the sugar surrogate comprises a six membered ring in place of the pentofuranosyl sugar. In certain embodiments, the sugar surrogate comprises a tetrahydropyran (“THP”) in place of the pentofuranosyl sugar. In certain embodiments, the sugar surrogate comprises a morpholino in place of the pentofuranosyl sugar. Representative US patents that teach the preparation of such modified sugar structures include, but are not limited to, U.S. Pat. Nos. 4,981,957; 5,118,800; 5,166,315; 5,185,444; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,700,920; 7,875,733; 7,939,677, 8,088,904; 8,440,803; and 9,005,906, the entire contents of each of the foregoing are hereby incorporated herein by reference.

In some embodiments, sugar surrogates comprise acyclic moieties. In certain embodiments, the sugar surrogate is an unlocked nucleic acid (“UNA”). A UNA is unlocked acyclic nucleic acid, wherein any of the bonds of the sugar has been removed, forming an unlocked “sugar” residue. In one example, UNA also encompasses a monomer where the bonds between C1′-C4′ have been removed (i.e. the covalent carbon-oxygen-carbon bond between the C1′ and C4′ carbons). In another example, the C2′-C3′ bond (i.e., the covalent carbon-carbon bond between the C2′ and C3′ carbons) of the sugar has been removed. Representative U.S. publications that teach the preparation of UNA include, but are not limited to, U.S. Pat. No. 8,314,227; and U.S. Patent Publication Nos. 2013/0096289; 2013/0011922; and 2011/0313020, the entire contents of each of which are hereby incorporated herein by reference. In certain embodiments, sugar surrogates comprise peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., US2013/130378, the entire contents of which is hereby incorporated herein by reference. Many other bicyclic and tricyclic sugar and sugar surrogate ring systems are known in the art that can be used in modified nucleosides.

In certain aspects, the disclosure relates to compounds comprising at least one oligonucleotide wherein the nucleosides of such oligonucleotide comprise one or more types of modified sugars and/or unmodified sugars arranged along the oligonucleotide or region thereof in a defined pattern or “sugar motif”. In certain instances, such sugar motifs include, but are not limited to, any of the patterns of sugar modifications described herein.

In certain embodiments, an oligonucleotide comprises a gapmer sugar motif A gapmer oligonucleotide comprises or consists of a region having two external “wing” regions and a central or internal “gap” region. The gap and wing regions form a contiguous sequence of nucleosides, wherein the majority of nucleoside sugars of each of the wings differ from the majority of nucleoside sugars of the gap. In certain embodiments, the wing regions comprise a majority of modified sugars and the gap comprises a majority of unmodified sugars. In certain embodiments, the nucleosides of the gap are deoxynucleosides. Compounds with a gapmer sugar motif are described in, for example U.S. Pat. No. 8,790,919, the entire contents of which is hereby incorporated herein by reference.

In certain embodiments, one or both oligonucleotides of a double-stranded compound comprise a triplet sugar motif. An oligonucleotide with a triplet sugar motif comprises three identical sugar modifications on three consecutive nucleosides. In certain embodiments, the triplet is at or near the cleavage site of the oligonucleotide. In certain embodiments, an oligonucleotide of a double-stranded compound may contain more than one triplet sugar motif. In certain embodiments, the identical sugar modification of the triplet sugar motif is a 2′-F modification. Compounds with a triplet sugar motif are disclosed, for example, in U.S. Pat. No. 10,668,170, the entire contents of which is incorporated herein by reference.

In certain embodiments, one or both oligonucleotides of a double-stranded compound comprise a quadruplet sugar motif. An oligonucleotide with a quadruplet sugar motif comprises four identical sugar modifications on four consecutive nucleosides. In certain embodiments, the quadruplet is at or near the cleavage site. In certain embodiments, an oligonucleotide of a double-stranded compound may contain more than one quadruplet sugar motif. In certain embodiments, the identical sugar modification of the quadruplet sugar motif is a 2′-F modification. For a double-stranded compound having a duplex region of 19-23 nucleotides in length, the cleavage site of the antisense oligonucleotide is typically around the 10, 11, and 12 positions from the 5′-end. In certain embodiments, the quadruplet sugar motif is at the 8, 9, 10, 11 positions; the 9, 10, 11, 12 positions; the 10, 11, 12, 13 positions; the 11, 12, 13, 14 positions; or the 12, 13, 14, 15 positions of the sense oligonucleotide, counting from the first nucleoside of the 5′-end of the sense oligonucleotide, or, the count starting from the first paired nucleotide within the duplex region from the 5′-end of the sense oligonucleotide. In certain embodiments, the quadruplet sugar motif is at the 8, 9, 10, 11 positions; the 9, 10, 11, 12 positions; the 10, 11, 12, 13 positions; the 11, 12, 13, 14 positions; or the 12, 13, 14, 15 positions of the antisense oligonucleotide, counting from the first nucleoside of the 5′-end of the antisense oligonucleotide, or, the count starting from the first paired nucleotide within the duplex region from the 5′-end of the antisense oligonucleotide. The cleavage site may change according to the length of the duplex region of the double-stranded compound and may change the position of the quadruplet accordingly.

In certain embodiments, an oligonucleotide comprises an alternating sugar motif. In certain embodiments, one or both oligonucleotides of a double-stranded compound comprise an alternating sugar motif. An oligonucleotide with an alternating sugar motif comprises at least two different sugar modifications wherein one or more consecutive nucleosides comprising a first sugar modification alternates with one or more consecutive nucleosides comprising a second sugar modification and one or more consecutive nucleosides comprising a third sugar modification, etc. For example, if A, B and C each represent one type of modification to the nucleoside, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ” etc. In certain embodiments, the alternating sugar motif is repeated for at least 2, 3, 4, 5, 6, 7, 8, 9, 10, 11, 12, 13, 14, 15, 16, 17, 18, 19, 20, 21, 22, or 23 contiguous nucleobases along an oligonucleotide. In certain embodiments, the alternating sugar motif is comprised of two different sugar modifications. In certain embodiments, the alternating sugar motif comprises 2′-OMe and 2′-F sugar modifications.

In certain embodiments, each nucleoside of an oligonucleotide is independently modified with one or more sugar modifications provided herein. In certain embodiments, each oligonucleotide of a double-stranded compound independently has one or more sugar motifs provided herein. In certain embodiments, an oligonucleotide containing a sugar motif, is fully modified in that each nucleoside other than the nucleosides comprising the sugar motif comprises a sugar modification.

### Nucleobase Modifications and Motifs

In certain embodiments, compounds described herein comprise modified oligonucleotides. In certain embodiments, modified oligonucleotides comprise one or more nucleosides comprising a modified nucleobase. In certain embodiments, modified oligonucleotides comprise one or more nucleosides that do not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly, 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size expanded bases, and fluorinated bases. Further modified nucleobases include tricyclic pyrimidines, such as 1,3-diazaphenoxazine-2-one, 1,3-diazaphenothiazine-2-one, and 9-(2-aminoethoxy)-1,3-diazaphenoxazine-2-one (G-clamp). Modified nucleobases may also include those in which the purine or pyrimidine base is replaced with other heterocycles, for example, 7-deaza-adenine, 7-deazaguanosine, 2-aminopyridine and 2-pyridone.

Further nucleobases include those disclosed in U.S. Pat. No. 3,687,808; Modified Nucleosides in Biochemistry, Biotechnology and Medicine, Herdewijn, P. ed. Wiley-VCH, 2008; The Concise Encyclopedia Of Polymer Science And Engineering, pages 858-859; Kroschwitz, J. L., Ed., John Wiley & Sons, 1990, 858-859; Englisch et al., Angewandte Chemie, International Edition, 1991, 30, 613; Sanghvi, Y. S., Chapter 15, dsRNA Research and Applications, pages 289-302; Antisense Research and Applications, Crooke, S. T. and Lebleu, B., Eds., CRC Press, 1993, 273-288; Antisense Drug Technology, Crooke S. T., Ed., CRC Press, 2008, 163-166 and 442-443 (Chapters 6 and 15), each of which are hereby incorporated herein by reference.

Publications that teach the preparation of certain of the above noted modified nucleobases, as well as other modified nucleobases include without limitation, US Applications 2003/0158403 and 2003/0175906; U.S. Pat. Nos. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121; 5,596,091; 5,614,617; 5,645,985; 5,681,941; 5,811,534; 5,750,692; 5,948,903; 5,587,470; 5,457,191; 5,763,588; 5,830,653; 5,808,027; 6,005,096, 6,015,886; 6,147,200; 6,166,197; 6,166,199; 6,222,025; 6,235,887; 6,380,368; 6,528,640; 6,639,062; 6,617,438; 7,045,610; 7,427,672; and 7,495,088, the entire contents of each of which are hereby incorporated herein by reference.

In certain embodiments, compounds described herein comprise oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments, the block is within 3 nucleosides of the 3′-end of the oligonucleotide. In certain embodiments, the block is at the 5′-end of the oligonucleotide. In certain embodiments, the block is within 3 nucleosides of the 5′-end of the oligonucleotide.

### Internucleoside Linkage Modifications and Motifs

A 3′ to 5′ phosphodiester linkage is the naturally occurring internucleoside linkage of RNA and DNA. In certain embodiments, compounds described herein have one or more modified, i.e. non-naturally occurring, internucleoside linkages. Certain non-naturally occurring internucleoside linkages may impart desirable properties such as, for example, enhanced cellular uptake, enhanced affinity for target nucleic acids, and increased stability in the presence of nucleases. Representative phosphorus-containing modified internucleoside linkages include, but are not limited to, phosphotriesters, alkylphosphonates (e.g. methylphosphonates), phosphoramidates, and phosphorothioates (“P═S”), and phosphorodithioates (“HS—P═S”). Representative non-phosphorus containing internucleoside linking groups include, but are not limited to, methylenemethylimino (—CH₂—N(CH₃)—O—CH₂), thiodiester, thionocarbamate (—O—C(═O)(NH)—S—); siloxane (—O—SiH₂—O—); and N,N′-dimethylhydrazine (—CH₂—N((CH₃)—N((CH₃)—). Methods of preparation of phosphorous-containing and non-phosphorous-containing internucleoside linkages are well known to those skilled in the art. Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See, for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, compounds provided herein comprise at least one modified internucleoside linkage. A modified internucleoside linkage may be placed at any position of an oligonucleotide. For double-stranded compounds, a modified internucleoside linkage may be placed within the sense oligonucleotide, antisense oligonucleotide, or both oligonucleotides of the double-stranded compound.

In certain embodiments, the internucleoside linkage modification may occur on every nucleoside of an oligonucleotide. In certain embodiments, internucleoside linkage modifications may occur in an alternating pattern along an oligonucleotide. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the pattern of the internucleoside linkage modification on each oligonucleotide of a double-stranded compound is the same. In certain embodiments, the pattern of the internucleoside linkage modification on each oligonucleotide of a double-stranded compound is different. In certain embodiments, a double-stranded compound comprises 6-8 modified internucleoside linkages. In certain embodiments, the 6-8 modified internucleoside linkages are phosphorothioate internucleoside linkages or alkylphosphonate internucleoside linkages. In certain embodiments, the sense oligonucleotide comprises at least two modified internucleoside linkages at either or both the 5′-end and the 3′-end. In certain such embodiments, the modified internucleoside linkages are phosphorothioate internucleoside linkages or alkylphosphonate internucleoside linkages. In certain embodiments, the antisense oligonucleotide comprises at least two modified internucleoside linkages at either or both the 5′-end and the 3′-end. In certain such embodiments, the modified internucleoside linkages are phosphorothioate internucleoside linkages or alkylphosphonate internucleoside linkages.

In certain embodiments, a double-stranded compound comprises an overhang region. In certain embodiments, a double-stranded compound comprises a phosphorothioate or alkylphosphonate internucleoside linkage modification in the overhang region. In certain embodiments, a double-stranded compound comprises a phosphorothioate or alkylphosphonate internucleotide linkage linking the overhang nucleotide with a paired nucleotide that is next to the overhang nucleotide. For instance, there may be at least two phosphorothioate internucleoside linkages between the terminal three nucleosides, in which two of the three nucleosides are overhang nucleosides, and the third is a paired nucleoside next to the overhang nucleoside. These terminal three nucleosides may be at the 3′-end of the antisense oligonucleotide, the 3′-end of the sense oligonucleotide, the 5′-end of the antisense oligonucleotide, or the 5′end of the antisense oligonucleotide.

In certain embodiments, modified oligonucleotides comprise one or more internucleoside linkages having chiral centers. Representative chiral internucleoside linkages include, but are not limited to, alkylphosphonates and phosphorothioates. Modified oligonucleotides comprising internucleoside linkages having chiral centers can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. As is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration.

### Conjugate Groups

In certain embodiments, the compounds described herein comprise or consist of one or more oligonucleotides and, optionally, one or more conjugate groups. Conjugate groups may be attached to either or both ends of an oligonucleotide and/or at any internal position. In certain embodiments, a conjugate group is attached at the 3′ end of an oligonucleotide. In certain embodiments, a conjugate group is attached at the 5′ end of an oligonucleotide. In certain embodiments, oligonucleotides are covalently attached to one or more conjugate groups.

In certain embodiments, conjugate groups are terminal groups attached to either or both ends of an oligonucleotide. In certain such embodiments, terminal groups are attached at the 3′ end of an oligonucleotide. In certain such embodiments, terminal groups are attached at the 5′ end of an oligonucleotide. In certain embodiments, terminal groups include, but are not limited to, capping groups, phosphate moieties, protecting groups, modified or unmodified nucleosides, and two or more nucleosides that are independently modified or unmodified, such as an overhang.

In certain embodiments, conjugate groups modify one or more properties of the attached oligonucleotide, including, but not limited to, pharmacodynamics, pharmacokinetics, stability, activity, half-life, binding, absorption, tissue distribution, cellular distribution, cellular uptake, charge and clearance. In certain embodiments, conjugate groups enhance the affinity of a compound for a selected target, e.g., molecule, cell or cell type, compartment, e.g., a cellular or organ compartment, tissue, organ or region of the body, as, e.g., compared to a compound absent such a conjugate group. In certain embodiments, conjugate groups impart a new property on the attached oligonucleotide, e.g., fluorophores or reporter groups that enable detection of the oligonucleotide.

In certain embodiments, conjugate groups include, but are not limited to, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, conjugate groups include an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial, or an antibiotic.

In certain embodiments, conjugate groups are targeting moieties. In certain embodiments, a targeting moiety includes, but is not limited to, a lectin, glycoprotein, lipid, protein, peptide, peptide mimetic, receptor ligand, antibody, thyrotropin, melanotropin, surfactant protein A, carbohydrate, carbohydrate derivative, modified carbohydrate, carbohydrate cluster, polysaccharide, modified polysaccharide, or polysaccharide derivative, mucin carbohydrate, multivalent lactose, multivalent galactose, N-acetyl-galactosamine (GalNAc), N-acetylglucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

In certain embodiments, conjugate groups may include, but are not limited to, the conjugate groups described in the following references such as cholesterol (e.g., Letsinger et al., Proc. Natl. Acid. Sci. USA, 1989, 86: 6553-6556), cholic acid (e.g., Manoharan et al., Biorg. Med. Chem. Let., 1994, 4:1053-1060), thioether, e.g., hexyl-S-tritylthiol (e.g., Manoharan et al., Ann. NY. Acad. Sci., 1992, 660:306-309; Manoharan et al., Biorg. Med. Chem. Let., 1993, 3:2765-2770), thiocholesterol (e.g., Oberhauser et al., Nucl. Acids Res., 1992, 20:533-538), aliphatic chains, e.g., do-decan-diol or undecyl residues (e.g., Saison-Behmoaras et al., EMBO J, 1991, 10:1111-1118; Kabanov et al., FEBS Lett., 1990, 259:327-330; Svinarchuk et al., Biochimie, 1993, 75:49-54), phospholipids, e.g., di-hexadecyl-rac-glycerol or triethyl-ammonium 1,2-di-O-hexadecyl-rac-glycero-3-H-phosphonate (e.g, Manoharan et al., Tetrahedron Lett., 1995, 36:3651-3654; Shea et al., Nucl. Acids Res., 1990, 18:3777-3783), polyamines or a polyethylene glycol chains (e.g., Manoharan et al., Nucleosides & Nucleotides, 1995, 14:969-973), adamantane acetic acid (e.g., Manoharan et al., Tetrahedron Lett., 1995, 36:3651-3654), palmityl (e.g., Mishra et al., Biochim. Biophys. Acta, 1995, 1264:229-237), octadecylamine or hexylamino-carbonyloxychole sterol moiety (e.g., Crooke et al. J. Pharmacol. Exp. Ther., 1996, 277:923-937), tocopherol (e.g., Nishina et al., Molecular Therapy Nucleic Acids, 2015, 4, e220 and Nishina et al., Molecular Therapy, 2008, 16:734-740), GalNAc and other carbohydrates (e.g., Maier et al., Bioconjugate Chemistry, 2003, 14, 18-29; Rensen et al., J. Med. Chem. 2004, 47, 5798-5808; WO2009/073809 and U.S. Pat. Nos. 8,106,022; 8,450,467 and 8,828,957; and WO2014/179445; WO2014/179620 and U.S. Pat. Nos. 9,127,276; 9,181,549 and 10,844,379) each of which is incorporated herein by reference in its entirety.

Conjugate groups may be attached to oligonucleotides through conjugate linkers. In certain embodiments, a conjugate linker comprises a chain structure, such as a hydrocarbyl chain, or an oligomer of repeating units or combination of such repeating units. In certain embodiments, a conjugate linker comprises one or more groups selected from alkyl, amino, oxo, amide, disulfide, polyethylene glycol, ether, thioether, and hydroxylamino. In certain embodiments, a conjugate linker comprises at least one phosphorus group. In certain embodiments, a conjugate linker comprises at least one phosphate group. In certain embodiments, a conjugate linker includes at least one neutral linking group. In certain embodiments, conjugate linkers include, but are not limited to, pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include, but are not limited to, substituted or unsubstituted C₁-C₁₀ alkyl, substituted or unsubstituted C₂-C₁₀ alkenyl, or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl, and alkynyl. In certain embodiments, conjugate linkers comprise 1-10 linker-nucleosides. In certain embodiments, such linker-nucleosides may be modified or unmodified nucleosides. It is typically desirable for linker-nucleosides to be cleaved from the compound after it reaches a target tissue. Accordingly, linker-nucleosides herein can be linked to one another and to the remainder of the compound through cleavable bonds. Herein, linker-nucleosides are not considered to be part of the oligonucleotide. Accordingly, in embodiments in which a compound comprises an oligonucleotide consisting of a specified number or range of linked nucleosides and/or a specified percent complementarity to a reference nucleic acid and the compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid.

In certain embodiments, conjugate groups and conjugate linkers as well as other modifications include, without limitation, those described in the following references: U.S. Pat. Nos. 5,994,517; 6,300,319; 6,660,720; 6,906,182; 7,262,177; 7,491,805; 8,106,022; 7,723,509; 9,127,276; US 2006/0148740; US 2011/0123520; WO2013/033230; WO2012/037254, Biessen et al., J. Med. Chem. 1995, 38, 1846-1852; Lee et al., Bioorganic & Medicinal Chemistry 2011, 19, 2494-2500; Rensen et al., J. Biol. Chem. 2001, 276, 37577-37584; Rensen et al., J. Med. Chem. 2004, 47, 5798-5808; Sliedregt et al., J. Med. Chem. 1999, 42, 609-618; Valentijn et al., Tetrahedron, 1997, 53, 759-770; Lee, Carhohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycohiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromh Vase Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 7,582,744; 8,552,163; 8,137,695; 6,908,903; 6,383,812; 7,262,177; 6,525,031; 5,994,517; 6,660,720; 6,300,319; 7,723,509; 8,106,022; 7,491,805; 7,491,805; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2003/0119724; US2011/0207799; US2012/0035115; US2012/0230938; US2005/0164235; US2006/0183886; US2012/0136042; US2012/0095075; US2013/0109817; US2006/0148740; US2008/0206869; US2012/0165393; US2012/0101148; US2013/0121954; US2011/0123520; US2003/0077829; US2008/0108801; and US2009/0203132; each of which is incorporated herein by reference in its entirety.

### Certain Targeting Moieties

In certain embodiments, a compound provided herein comprises a conjugate group. In certain embodiments, an oligonucleotide provided herein comprises a conjugate group. In certain embodiments, the conjugate group is a targeting moiety. In certain embodiments, the targeting moiety comprises one or more GalNAc. In certain embodiments, the one or more GalNAc are attached to one or more positions on a furanose ring. In certain embodiments, the one or more GalNAc are attached to the 2′ or 3′ position on a furanose ring. In certain embodiments, the furanose ring is a subunit of the oligonucleotide. In certain embodiments, the furanose ring is the 5′ nucleoside sugar of an oligonucleotide. In certain embodiments, the furanose ring is the 5′ nucleoside sugar of a sense oligonucleotide. In certain embodiments, a compound or oligonucleotide comprises one or more subunits with the following formula or a salt, solvate, or hydrate thereof:

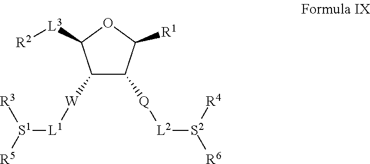

wherein:
R¹ is H, adenine, guanine, thymine, cytosine, uracil, carbocyclyl, heterocyclyl, aryl, heteroaryl, or a nucleobase isostere;
R² is the oligonucleotide sequence;
L¹ is alkyl, or alkyl-C(═O)—NH-alkyl;
L² is alkyl, or alkyl-C(═O)—NH-alkyl;
L³ is a bond, a phosphodiester bond, a phosphorothioate bond, a triazole, a tetrazole, an amide, a reverse-amide, a carbamate, a carbonate, urea, O, S, S(═O), S(═O)₂, NH, substituted N group, alkyl, alkenyl, dienyl, alkynyl, heteroalkyl, phosphate;
R³ is H, —C═(O)—NH—(CH₂CH₂O)ⱼ-GalNAc, or —C(═O)—NH-alkyl-NH—C(═O)-alkyl-O-GalNAc;
R⁴ is H, —C═(O)—NH—(CH₂CH₂O)ₖ-GalNAc, or —C(═O)—NH-alkyl-NH—C(═O)-alkyl-O-GalNAc;
R⁵ is —C═(O)—NH—(CH₂CH₂O)ₘ-GalNAc, or —C(═O)—NH-alkyl-NH—C(═O)-alkyl-O-GalNAc;
R⁶ is —C═(O)—NH—(CH₂CH₂O)ₙ-GalNAc, or —C(═O)—NH-alkyl-NH—C(═O)-alkyl-O-GalNAc;
W and Q are each independently O, NH, CH₂, or CH₂O;
S¹ and S² are each independently C(R⁷) or N, wherein each instance of R⁷ is independently H, alkyl, heteroalkyl, or halogen;
j is an integer 1-10, inclusive;
k is an integer 1-10, inclusive;
m is an integer 1-10, inclusive; and
n is an integer 1-10, inclusive.

In certain embodiments, R³, R⁴, R⁵, and R⁶ are the same. In certain embodiments, R³, R⁵, and R⁶ are the same. In certain embodiments, R³ or R⁴ is H.

In certain embodiments, L¹ and L² are the same.

In certain embodiments, L¹ and L² are each independently alkyl; R³ is H, —C═(O)—NH—(CH₂CH₂O)ⱼ-GalNAc, or —C(═O)—NH-alkyl-NH—C(═O)-alkyl-O-GalNAc; R⁴ is H, —C═(O)—NH—(CH₂CH₂O)ₖ-GalNAc, or —C(═O)—NH-alkyl-NH—C(═O)-alkyl-O-GalNAc; R⁵ is —C═(O)—NH—(CH₂CH₂O)ₘ-GalNAc, or —C(═O)—NH-alkyl-NH—C(═O)-alkyl-O-GalNAc; and R⁶ is —C═(O)—NH—(CH₂CH₂O)ₙ-GalNAc, or —C(═O)—NH-alkyl-NH—C(═O)-alkyl-O-GalNAc.

In certain embodiments, L¹ and L² are each independently alkyl-C(═O)—NH-alkyl; R³ is H, —C═(O)—NH—(CH₂CH₂O)ⱼ-GalNAc, or —C(═O)—NH-alkyl-NH—C(═O)-alkyl-O-GalNAc; R⁴ is H, —C═(O)—NH—(CH₂CH₂O)ₖ-GalNAc, or —C(═O)—NH-alkyl-NH—C(═O)-alkyl-O-GalNAc; R⁵ is —C═(O)—NH—(CH₂CH₂O)ₘ-GalNAc, or —C(═O)—NH-alkyl-NH—C(═O)-alkyl-O-GalNAc; and R⁶ is —C═(O)—NH—(CH₂CH₂O)ₙ-GalNAc, or —C(═O)—NH-alkyl-NH—C(═O)-alkyl-O-GalNAc.

In certain embodiments, R⁴ is H.

In certain embodiments, L¹ and L² are each independently alkyl; R³ is —C═(O)—NH—(CH₂CH₂O)ⱼ-GalNAc, or —C(═O)—NH-alkyl-NH—C(═O)-alkyl-O-GalNAc; R⁴ is H; R⁵ is —C═(O)—NH—(CH₂CH₂O)ₘ-GalNAc, or —C(═O)—NH-alkyl-NH—C(═O)-alkyl-O-GalNAc; and R⁶ is —C═(O)—NH—(CH₂CH₂O)ₙ-GalNAc, or —C(═O)—NH-alkyl-NH—C(═O)-alkyl-O-GalNAc.

In certain embodiments, L¹ and L² are each independently alkyl-C(═O)—NH-alkyl; R³ is —C═(O)—NH—(CH₂CH₂O)ⱼ-GalNAc, or —C(═O)—NH-alkyl-NH—C(═O)-alkyl-O-GalNAc; R⁴ is H; R⁵ is —C═(O)—NH—(CH₂CH₂O)ₘ-GalNAc, or —C(═O)—NH-alkyl-NH—C(═O)-alkyl-O-GalNAc; and R⁶ is —C═(O)—NH—(CH₂CH₂O)ₙ-GalNAc, or —C(═O)—NH-alkyl-NH—C(═O)-alkyl-O-GalNAc.

In certain embodiments, R³ is —C═(O)—NH—(CH₂CH₂O)ⱼ-GalNAc; R⁴ is H; R⁵ is —C═(O)—NH—(CH₂CH₂O)ₘ-GalNAc; and R⁶ is —C═(O)—NH—(CH₂CH₂O)ₙ-GalNAc.

In certain embodiments, R³ is —C(═O)—NH-alkyl-NH—C(═O)-alkyl-O-GalNAc; R⁴ is H; R⁵ is —C(═O)—NH-alkyl-NH—C(═O)-alkyl-O-GalNAc; and R⁶ is —C(═O)—NH-alkyl-NH—C(═O)-alkyl-O-GalNAc.

In certain embodiments, a compound or oligonucleotide comprises one or more subunits with the following formula or a salt, solvate, or hydrate thereof:

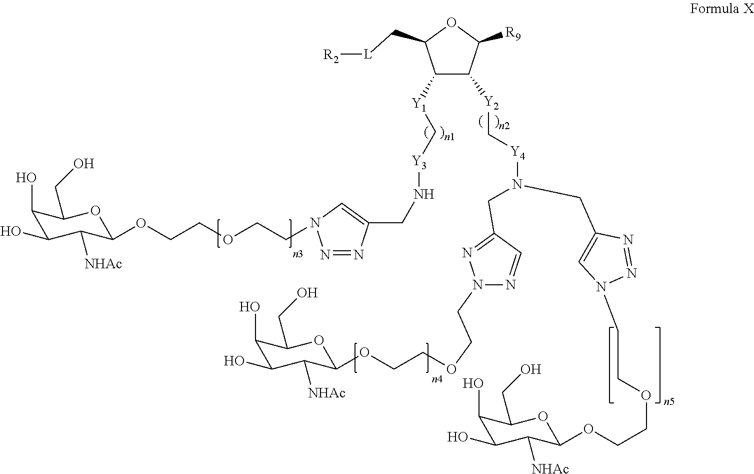

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
L is a bond, a phosphodiester bond, a phosphorothioate bond, a triazole, a tetrazole, an amide, a reverse-amide, a carbamate, a carbonate, urea, alkyl, or heteroalkyl;
R² is the oligonucleotide sequence;
Y₁ is O, CH₂, CH₂O, or optionally substituted NH;
Y₂ is O, CH₂, CH₂O, or optionally substituted NH;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6; and
each n₁, n₃, n₄ and n₅ is independently 0, 1, 2, 3, 4, 5, 6, 7, 8, 9, or 10.

In certain embodiments, a compound or oligonucleotide comprises one or more subunits with the following formula or a salt, solvate, or hydrate thereof:

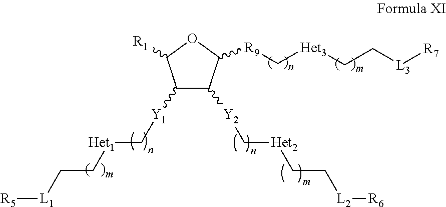

wherein:
each n is independently 1, 2, 3, 4, or 5;
each m is independently 0, 1, 2, 3, 4, 5, or 6;
each o is independently 0, 1, 2, 3, 4, 5, or 6;
each of L₁, L₂, and L₃ is independently absent, C(═O), or C(═O)NH;
each Y₁ is independently O, CH(R^{a}), S, S(═O), S(═O)₂, NH, substituted N group, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)₂—, —O—P(═O)(═S)—, —O—P(═S)₂—;
each Y₂ is independently O, CH(R^{b}), S, S(═O), S(═O)₂, NH, substituted N group, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)₂—, —O—P(═O)(═S)—, —O—P(═S)₂—;
each of Het₁, Het₂, and Het₃ is independently optionally substituted heteroaryl or optionally substituted heterocyclyl;
R¹ is the oligonucleotide sequence linked by a bond, a phosphodiester bond, a phosphorothioate bond, a triazole, a tetrazole, an amide, a reverse-amide, a carbamate, a carbonate, urea, alkyl, or heteroalky;

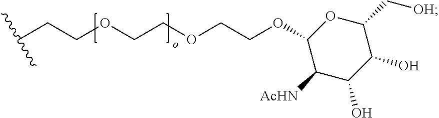

each R₅, R₆, and R₇ is independently OH
R₉ is optionally substituted heterocyclyl;
each R^{a} is independently H, alkyl, halo, OR^{c}, or SR^{c};
each R^{b} is independently H, alkyl, halo, OR^{c}, or SR^{c}; and
each R^{c} is independently H or alkyl.

In certain embodiments, the subunit is selected from Formulae I through VIII or a salt, solvate, or hydrate thereof, wherein R is the modified oligonucleotide other than the 5′ nucleoside. In certain embodiments, the 5′ nucleoside of the modified oligonucleotide is Formula I and R′ is O. In certain embodiments, the 5′ nucleoside of the modified oligonucleotide is Formula I and R′ is S. In certain embodiments, the 5′ nucleoside of the modified oligonucleotide is Formula II and R′ is O. In certain embodiments, the 5′ nucleoside of the modified oligonucleotide is Formula II and R′ is S. In certain embodiments, the 5′ nucleoside of the modified oligonucleotide is Formula III and R′ is O. In certain embodiments, the 5′ nucleoside of the modified oligonucleotide is Formula III and R′ is S. In certain embodiments, the 5′ nucleoside of the modified oligonucleotide is Formula IV and R′ is O. In certain embodiments, the 5′ nucleoside of the modified oligonucleotide is Formula IV and R′ is S. In certain embodiments, the 5′ nucleoside of the modified oligonucleotide is Formula V and R′ is O. In certain embodiments, the 5′ nucleoside of the modified oligonucleotide is Formula V and R′ is S. In certain embodiments, the 5′ nucleoside of the modified oligonucleotide is Formula VI and R′ is O. In certain embodiments, the 5′ nucleoside of the modified oligonucleotide is Formula VI and R′ is S. In certain embodiments, the 5′ nucleoside of the modified oligonucleotide is Formula VII and R′ is O. In certain embodiments, the 5′ nucleoside of the modified oligonucleotide is Formula VII and R′ is S. In certain embodiments, the 5′ nucleoside of the modified oligonucleotide is Formula VIII and R′ is O. In certain embodiments, the 5′ nucleoside of the modified oligonucleotide is Formula VIII and R′ is S.

### Target Nucleic Acids and Target Regions

In certain embodiments, compounds described herein comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain embodiments, the target nucleic acid is non-coding. In certain such embodiments, the target nucleic acid is selected from an mRNA and a pre-mRNA, including intronic, exonic and untranslated regions. In certain embodiments, the target RNA is an mRNA. In certain embodiments, the target nucleic acid is a pre-mRNA. In certain such embodiments, the target region is entirely within an exon. In certain such embodiments, the target region is entirely within an intron. In certain embodiments, the target region spans an intron/exon junction. In certain embodiments, the target region is at least 50% within an intron.

In certain embodiments, compounds disclosed herein hybridize with a AGT nucleic acid. The most common mechanism of hybridization involves hydrogen bonding between complementary nucleobases of the nucleic acid molecules. Hybridization can occur under varying conditions. Hybridization conditions are sequence-dependent and are determined by the nature and composition of the nucleic acid molecules to be hybridized. Methods of determining whether a sequence hybridizes specifically to a target nucleic acid are well known in the art. In certain embodiments, the compounds provided herein specifically hybridize with a AGT nucleic acid.

Nucleotide sequences that encode AGT include, without limitation, the following: GENBANK Accession Nos. NM_000029.4 (incorporated herein as SEQ ID NO: 1), the complement of nucleotides 230702523 to 230745583 of NC_000001.11 (incorporated herein as SEQ ID NO: 2), NM_001382817.3 (incorporated herein as SEQ ID NO: 3) and nucleotides 5469 to 17068 of NG_008836.2 (incorporated herein as SEQ ID NO: 4).

### Complementarity

Oligonucleotides provided herein may have a defined percent complementarity to a particular nucleic acid, target region, oligonucleotide, or portion thereof. Non-complementary nucleobases may be tolerated provided that the oligonucleotide remains able to specifically hybridize to the nucleic acid, oligonucleotide, or portion thereof. In certain embodiments, the oligonucleotides provided herein, or a specified portion thereof are at least, or are up to 70%, 80%, 85%, 86%, 87%, 88%, 89%, 90%, 91%, 92%, 93%, 94%, 95%, 96%, 97%, 98%, 99%, or 100% complementary to a target nucleic acid, a target region, an oligonucleotide or specified portion thereof. In certain embodiments, the oligonucleotides provided herein, or a specified portion thereof, are 70% to 75%, 75% to 80%, 80% to 85%, 85% to 90%, 90% to 95%, 95% to 100%, or any number in between these ranges, complementary to a target nucleic acid, a target region, an oligonucleotide or specified portion thereof. Percent complementarity of an oligonucleotide with a target nucleic acid, a target region, an oligonucleotide or specified portion thereof can be determined using routine methods. For example, an oligonucleotide in which 18 of 20 nucleobases of the oligonucleotide are complementary to a target region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, an oligonucleotide which is 18 nucleobases in length having four non-complementary nucleobases which are flanked by two regions of complete complementarity with the target nucleic acid would have 77.8% overall complementarity with the target nucleic acid. Percent complementarity of an oligonucleotide with a region of a target nucleic acid, a target region, an oligonucleotide or specified portion thereof can be determined routinely using BLAST programs (basic local alignment search tools) known in the art. In certain embodiments, oligonucleotides described herein, or specified portions thereof, are fully complementary (i.e. 100% complementary) to a target nucleic acid, a target region, an oligonucleotide or specified portion thereof. For example, an oligonucleotide may be fully complementary to a target nucleic acid, a target region, an oligonucleotide, or specified portion thereof. As used herein, “fully complementary” means each nucleobase of an oligonucleotide is complementary to the corresponding nucleobase of a target nucleic acid, a target region, an oligonucleotide, or a specified portion thereof. For example, a 20 nucleobase oligonucleotide is fully complementary to a target sequence that is 400 nucleobases long, so long as there is a corresponding 20 nucleobase portion of the target nucleic acid that is fully complementary to the compound. “Fully complementary” can also be used in reference to a specified portion of the first and/or the second nucleic acid. For example, a 20 nucleobase portion of a 30 nucleobase oligonucleotide can be “fully complementary” to a 20 nucleobase region of a target sequence that is 400 nucleobases long. The 20 nucleobase portion of the 30 nucleobase compound is fully complementary to the target sequence if the target sequence has a corresponding 20 nucleobase portion wherein each nucleobase is complementary to the 20 nucleobase portion of the compound. At the same time, the entire 30 nucleobase compound may or may not be fully complementary to the target sequence, depending on whether the remaining 10 nucleobases of the compound are also complementary to the target sequence. In certain embodiments, oligonucleotides described herein comprise one or more mismatched nucleobases relative to a target nucleic acid, a target region, an oligonucleotide or a specified portion thereof. In certain embodiments, oligonucleotides described herein that are, or are up to 12, 13, 14, 15, 16, 17, 18, 19, 20, 21, 22, or 23 nucleobases in length comprise no more than 4, no more than 3, no more than 2, or no more than 1 non-complementary nucleobase(s) relative to a target nucleic acid, or specified portion thereof. In certain embodiments, oligonucleotides described herein that are, or are up to 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, a target region, an oligonucleotide, or specified portion thereof. In certain embodiments, the mismatch is at position 1, 2, 3, 4, 5, 6, 7, 8, 9, 10, 11, 12, 13 or 14 from the 5′-end of the oligonucleotide. In certain embodiments, the mismatch is at position 1, 2, 3, 4, 5, 6, 7, 8, 9, 10, 11, or 12, 13 or 14 from the 3′-end of the oligonucleotide. In certain embodiments, the mismatch forms a wobble base pair with a corresponding nucleobase on the target nucleic acid. For example, in certain embodiments, the mismatch forms a wobble base pair selected from hypoxanthine (nucleobase of inosine) and uracil (I:U base pair); guanine and uracil (G:U base pair); hypoxanthine and adenine (I:A base pair); and hypoxanthine and cytosine (I:C base pair). Accordingly, in certain embodiments, a mismatched nucleobase on an oligonucleotide comprises hypoxanthine, guanine, or uracil.

In certain embodiments, oligonucleotides described herein may be complementary to a portion of a nucleic acid. As used herein, “portion” refers to a defined number of contiguous nucleobases within a region of a nucleic acid. A “portion” can also refer to a defined number of contiguous nucleobases of an oligonucleotide. In certain embodiments, the oligonucleotides are complementary to at least an 8 nucleobase portion of a nucleic acid. In certain embodiments, the oligonucleotides are complementary to at least a 9 nucleobase portion of a nucleic acid. In certain embodiments, the oligonucleotides are complementary to at least a 10 nucleobase portion of a nucleic acid. In certain embodiments, the oligonucleotides are complementary to at least an 11 nucleobase portion of a nucleic acid. In certain embodiments, the oligonucleotides are complementary to at least a 12 nucleobase portion of a nucleic acid. In certain embodiments, the oligonucleotides are complementary to at least a 13 nucleobase portion of a nucleic acid. In certain embodiments, the oligonucleotides are complementary to at least a 14 nucleobase portion of a nucleic acid. In certain embodiments, the oligonucleotides are complementary to at least a 15 nucleobase portion of a nucleic acid. In certain embodiments, the oligonucleotides are complementary to at least a 16 nucleobase portion of a nucleic acid. Also contemplated are oligonucleotides that are complementary to at least a 9, 10, 17, 18, 19, 20, 21, 22, 23 or more nucleobase portion of a nucleic acid, or a range defined by any two of these values. In certain embodiments, the oligonucleotide is an antisense oligonucleotide. In certain embodiments, a portion of the antisense oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid. In certain embodiments, the oligonucleotide is a sense oligonucleotide. In certain embodiments, a portion of the sense oligonucleotide is compared to an equal length portion of an antisense oligonucleotide. In certain embodiments, an 8, 9, 10, 11, 12, 13, 14, 15, 16, 17, 18, 19, 20, 21, 22, 23, 24, or 25 nucleobase portion of a sense oligonucleotide is compared to an equal length portion of an antisense oligonucleotide.

### Identity

The oligonucleotides provided herein may also have a defined percent identity to a particular nucleic acid, target region, oligonucleotide, or specified portion thereof. As used herein, an oligonucleotide is identical to a sequence disclosed herein if it has the same nucleobase pairing ability. For example, a DNA which contains thymidine in place of uracil in a disclosed RNA sequence would be considered identical to the RNA sequence since both uracil and thymidine pair with adenine. Shortened and lengthened versions of the compounds described herein as well as compounds having non-identical bases relative to the compounds provided herein also are contemplated. The non-identical bases may be adjacent to each other or dispersed throughout the compound. Percent identity of an oligonucleotide is calculated according to the number of bases that have identical base pairing relative to the sequence to which it is being compared. In certain embodiments, oligonucleotides described herein, or portions thereof, are, or are at least, 70%, 75%, 80%, 85%, 90%, 91%, 92%, 93%, 94%, 95%, 96%, 97%, 98%, 99% or 100% identical to one or more of the nucleic acids, oligonucleotides, or a portion thereof, disclosed herein. In certain embodiments, oligonucleotides described herein are about 70%, 75%, 80%, 85%, 90%, 91%, 92%, 93%, 94%, 95%, 96%, 97%, 98%, or 99% identical, or any percentage between such values, to a particular nucleic acid or oligonucleotide, or portion thereof.

In certain embodiments, an oligonucleotide may have one or more mismatched nucleobases. In certain such embodiments, the mismatch is at position 1, 2, 3, 4, 5, 6, 7, 8, 9, 10, 11, 12, 13 or 14 from the 5′-end of the oligonucleotide. In certain such embodiments, the mismatch is at position 1, 2, 3, 4, 5, 6, 7, 8, 9, 10, 11, or 12, 13 or 14 from the 3′-end of the oligonucleotide. In certain embodiments, a portion of the oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid. In certain embodiments, the oligonucleotide is a sense oligonucleotides. In certain embodiments, a portion of the sense oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Pharmaceutical Compositions and Formulations

Compounds described herein may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered. Certain embodiments provide pharmaceutical compositions comprising one or more compounds or a salt thereof. In certain embodiments, the compounds are antisense oligonucleotides. In certain embodiments, the compounds are oligomeric compounds. In certain embodiments, the compounds comprise or consist of one or more modified oligonucleotides. In certain such embodiments, the pharmaceutical composition comprises one or more compound and a suitable pharmaceutically acceptable diluent or carrier. In certain embodiments, a pharmaceutical composition comprises one or more compound and a sterile saline solution. In certain embodiments, such pharmaceutical composition consists of one compound and a sterile saline solution. In certain embodiments, the sterile saline is pharmaceutical grade saline. In certain embodiments, a pharmaceutical composition comprises one or more compound and sterile water. In certain embodiments, a pharmaceutical composition consists of one compound and sterile water. In certain embodiments, the sterile water is pharmaceutical grade water. In certain embodiments, a pharmaceutical composition comprises one or more compounds and phosphate-buffered saline (PBS). In certain embodiments, a pharmaceutical composition consists of one compound and sterile PBS. In certain embodiments, the sterile PBS is pharmaceutical grade PBS.

A compound described herein targeted to AGT can be utilized in pharmaceutical compositions by combining the compound with a suitable pharmaceutically acceptable diluent or carrier. In certain embodiments, a pharmaceutically acceptable diluent is water, such as sterile water suitable for injection. Accordingly, in one embodiment, employed in the methods described herein is a pharmaceutical composition comprising a compound targeted to AGT and a pharmaceutically acceptable diluent. In certain embodiments, the pharmaceutically acceptable diluent is water. In certain embodiments, the compound comprises or consists of one or more modified oligonucleotide provided herein.

Pharmaceutical compositions comprising compounds provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the compounds are antisense oligonucleotides. In certain embodiments, the compounds are oligomeric compounds. In certain embodiments, the compound comprises or consists of one or more modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts. A prodrug can include the incorporation of additional nucleosides at one or both ends of a compound which are cleaved by endogenous nucleases within the body, to form the active compound. In certain embodiments, the compounds or compositions further comprise a pharmaceutically acceptable carrier or diluent.

### EXAMPLES

The following examples describe the process to identify lead compounds targeted to AGT. Certain compounds are distinguished as having high potency and tolerability.

The following examples serve only to illustrate the compounds described herein and are not intended to limit the same. The following examples and related sequence listing accompanying this filing may identify sequence as either “RNA” or “DNA”; however, as disclosed herein, those sequences may be modified with any combination of chemical modifications. One of skill in the art will readily appreciate that the designation of a sequence as “RNA” or “DNA” is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (methylated uracil for natural uracil of RNA). Accordingly, nucleic acid sequences provided herein, including, but not limited to, those in the sequence listing, are intended to encompass nucleic acids containing any combination of natural or modified RNA and/or DNA, including, but not limited to, such nucleic acids having modified nucleobases.

Each of the references recited in the present application is incorporated herein by reference in its entirety.

TABLE 1
Chemical Nomenclature

| Abbreviation | Structure |
|---|---|
| ‘m’ | 2′-O-methyl sugar modification (e.g., mA, mG, mC, mU) |
| ‘f’ | 2′-F sugar modification (e.g., fA, fG, fC, fU) |
| ‘*’ | phosphorothioate internucleoside linkage |
| ‘.’ | Phosphate internucleoside linkage |
| ‘dQ’ | inverted abasic deoxyribose |
| ‘H1’ | Formula I |
| ‘H2’ | Formula II |
| ‘H4’ | Formula III |
| ‘H9’ | Formula VI |

### Example 1—Inhibition of AGT in HepG2 Cells

HepG2 cells (ATCC Cat #HB-8065) were seeded in antibiotic-free media at 10,000 cells/well in a 96-well plate. The following day, test AGT compound was diluted to 1.0 μM (stock solution is 10 μM). Transfection mixes were prepared according to instructions for Dharmafect 4 transfection reagent (Dharmacon Cat #T-2004-0). Prepared transfection mixtures were incubated at room temperature for 20 minutes. During this incubation, the medium was replaced in the 96-well plates with 80 μl of antibiotic-free medium. 20 μl of the transfection mixture was then added to each well for a final concentration of 10 nM (tested in triplicate). AGT siRNA SMARTpool (Dharmacon Cat #L-010988-00-0005) was used as a positive control. The cells were then incubated at 37° C. in 5% CO₂ for 24 hours.

Cell lysis was performed according to the Cells-To-CT 1 Step TaqMan Kit instructions. Following cell lysis, the 96-well plate was placed on ice while the qRT-PCR reaction was prepared. 2 μl of cell lysate was added to the reaction mixture containing 5 μl TaqMan 1-Step qRT-PCR Mix, 1 μl AGT(FAM) TaqMan Gene Expression Assay (Hs01586213_m1), 1 μl GAPDH(VIC) TaqMan Gene Expression Assay (Hs02786624_g1) and 11 μl RT-PCR grade nuclease-free water in a MicroAmp Optical 96-well plate (0.2 mL). qPCR was performed using a QuantStudio3 qPCR machine with the following cycles: 50° C. for 1 minute, 95° C. for 20 seconds and 40 cycles at 95° C. for 15 seconds and 60° C. for 1 minute. Results are presented in the tables below as percent inhibition of AGT, relative to untreated control cells. Unless otherwise indicated in a separate compound chemistry table below, compounds are unmodified. Abbreviations for chemical modifications are provided in Table 1 above. IA and IS in a Ref ID NO:, identifies an antisense strand and sense strand of a compound, respectively.

TABLE 2
Inhibition of AGT mRNA by double-stranded compounds targeting SEQ ID NO: 1

| | Seq | SEQ | | | | | |
|---|---|---|---|---|---|---|---|
| | ID | ID | | | | | |
| | NO: | NO: | | | | | AGT |
| | 1 | 1 | | SEQ | | SEQ | % |
| Compound | Start | Stop | Antisense Sequence | ID | Sense Sequence | ID | Inhi- |
| Number | Site | Site | (5′-3′) | NO: | (5′-3′) | NO: | bition |
| RD0588 | 134 | 154 | UAUGUACACCCGGUCACCUGC | 10 | UGCAGGUGACCGGGUGUACAUAC | 167 | 43 |
| RD0592 | 144 | 164 | UGAAGGGGUGUAUGUACACCC | 15 | CGGGUGUACAUACACCCCUUCAA | 173 | 26 |
| RD0594 | 146 | 166 | UUGGAAGGGGUGUAUGUACAC | 17 | GGUGUACAUACACCCCUUCCAAC | 175 | 62 |
| RD0602 | 186 | 206 | UUGCCAGCUGCUCACAGGUAC | 27 | AGUACCUGUGAGCAGCUGGCAAA | 185 | 61 |
| RD0604 | 274 | 294 | UGUAGGGCCUUUUCAUCCACA | 30 | CUGUGGAUGAAAAGGCCCUACAG | 188 | 69 |
| RD0605 | 361 | 381 | UCCAAGAAGUUGGCCAGCAUC | 33 | GGAUGCUGGCCAACUUCUUGGAC | 191 | 39 |
| RD0607 | 367 | 387 | UGGAAGCCCAAGAAGUUGGCC | 35 | UGGCCAACUUCUUGGGCUUCCAU | 193 | 55 |
| RD0608 | 377 | 397 | UCCAUAUAUACGGAAGCCCAA | 37 | CUUGGGCUUCCGUAUAUAUGGAA | 195 | 68 |
| RD0610 | 383 | 403 | UUGCAUGCCAUAUAUACGGAA | 41 | CUUCCGUAUAUAUGGCAUGCAAA | 199 | 76 |
| RD0611 | 384 | 404 | UGUGCAUGCCAUAUAUACGGA | 42 | UUCCGUAUAUAUGGCAUGCACAG | 200 | 60 |
| RD0612 | 388 | 408 | UCACUGUGCAUGCCAUAUAUA | 43 | GUAUAUAUGGCAUGCACAGUGAG | 201 | 51 |
| RD0613 | 396 | 416 | UCCAUAGCUCACUGUGCAUGC | 44 | GGCAUGCACAGUGAGCUAUGGAG | 202 | 44 |
| RD0618 | 454 | 474 | UAGGCCAGGGUGCCAAAGACA | 49 | CUGUCUUUGGCACCCUGGCCUAU | 207 | 48 |
| RD0622 | 506 | 526 | UAGGAUUGCCUGUAGCCUGUC | 55 | UGACAGGCUACAGGCAAUCCUAG | 213 | 60 |
| RD0627 | 537 | 557 | AGGUGCAGUUCUUGUCCUUCC | 60 | UGGAAGGACAAGAACUGCACCUC | 218 | 17 |
| RD0628 | 538 | 558 | UAGGUGCAGUUCUUGUCCUUC | 61 | GGAAGGACAAGAACUGCACCUAC | 219 | 52 |
| RD0632 | 564 | 584 | ACAGGACCUUGUGCGCAUCCA | 66 | CUGGAUGCGCACAAGGUCCUGUC | 224 | 44 |
| RD0634 | 584 | 604 | UUGUACAGCCUGCAGGGCAGA | 68 | GUCUGCCCUGCAGGCUGUACAAG | 226 | 22 |
| RD0638 | 693 | 713 | UCACAAACGGCUGCUUCAGGU | 72 | CACCUGAAGCAGCCGUUUGUGAA | 230 | 46 |
| RD0641 | 696 | 716 | UCUGCACAAACGGCUGCUUCA | 75 | CUGAAGCAGCCGUUUGUGCAGAG | 233 | 53 |
| RD0648 | 793 | 813 | ACAGCCUGCAUGAACCUGUCA | 85 | UUGACAGGUUCAUGCAGGCUGUG | 243 | 76 |
| RD0650 | 801 | 821 | AUCCUGUCACAGCCUGCAUGA | 87 | UUCAUGCAGGCUGUGACAGGAUG | 246 | 48 |

TABLE 3
Inhibition of AGT mRNA by double-stranded compounds targeting SEQ ID NO: 1

| | Seq | SEQ | | | | | |
|---|---|---|---|---|---|---|---|
| | ID | ID | | | | | |
| | NO: | NO: | | | | | AGT |
| | 1 | 1 | | SEQ | | SEQ | % |
| Compound | Start | Stop | Antisense Sequence | ID | Sense Sequence | ID | Inhi- |
| Number | Site | Site | (5′-3′) | NO: | (5′-3′) | NO: | bition |
| RD0589 | 135 | 155 | UUAUGUACACCCGGUCACCUG | 11 | GCAGGUGACCGGGUGUACAUAAA | 168 | 49 |
| RD0595 | 151 | 171 | ACGAGGUGGAAGGGGUGUAUG | 18 | ACAUACACCCCUUCCACCUCGUC | 176 | 65 |
| RD0596 | 152 | 172 | UACGAGGUGGAAGGGGUGUAU | 19 | CAUACACCCCUUCCACCUCGUAA | 177 | 47 |
| RD0597 | 153 | 173 | UGACGAGGUGGAAGGGGUGUA | 20 | AUACACCCCUUCCACCUCGUCAU | 178 | 74 |
| RD0598 | 154 | 174 | AUGACGAGGUGGAAGGGGUGU | 21 | UACACCCCUUCCACCUCGUCAUC | 179 | 88 |
| RD0599 | 155 | 175 | UAUGACGAGGUGGAAGGGGUG | 22 | ACACCCCUUCCACCUCGUCAUAC | 180 | 60 |
| RD0600 | 156 | 176 | UGAUGACGAGGUGGAAGGGGU | 23 | CACCCCUUCCACCUCGUCAUCAA | 181 | 80 |
| RD0601 | 185 | 205 | UGCCAGCUGCUCACAGGUACU | 26 | GAGUACCUGUGAGCAGCUGGCAA | 184 | 77 |
| RD0603 | 187 | 207 | UUUGCCAGCUGCUCACAGGUA | 28 | GUACCUGUGAGCAGCUGGCAAAG | 186 | 93 |
| RD0606 | 362 | 382 | UCCCAAGAAGUUGGCCAGCAU | 34 | GAUGCUGGCCAACUUCUUGGGAU | 192 | 22 |
| RD0614 | 397 | 417 | UCCCAUAGCUCACUGUGCAUG | 45 | GCAUGCACAGUGAGCUAUGGGAC | 203 | 85 |
| RD0615 | 404 | 424 | UACCACGCCCCAUAGCUCACU | 46 | CAGUGAGCUAUGGGGCGUGGUAC | 204 | 73 |
| RD0616 | 448 | 468 | AGGGUGCCAAAGACAGCCGUU | 47 | CAACGGCUGUCUUUGGCACCCUG | 205 | 63 |
| RD0617 | 449 | 469 | UAGGGUGCCAAAGACAGCCGU | 48 | AACGGCUGUCUUUGGCACCCUAG | 206 | 95 |
| RD0619 | 462 | 482 | UAUAGAGAGAGGCCAGGGUGC | 50 | GGCACCCUGGCCUCUCUCUAUAU | 208 | 86 |
| RD0620 | 463 | 483 | AGAUAGAGAGAGGCCAGGGUG | 51 | GCACCCUGGCCUCUCUCUAUCUG | 209 | 80 |
| RD0621 | 505 | 525 | AGGAUUGCCUGUAGCCUGUCA | 54 | CUGACAGGCUACAGGCAAUCCUG | 212 | 74 |
| RD0623 | 507 | 527 | UCAGGAUUGCCUGUAGCCUGU | 56 | GACAGGCUACAGGCAAUCCUGAG | 214 | 81 |
| RD0624 | 508 | 528 | UCCAGGAUUGCCUGUAGCCUG | 57 | ACAGGCUACAGGCAAUCCUGGAU | 215 | 66 |
| RD0625 | 509 | 529 | ACCCAGGAUUGCCUGUAGCCU | 58 | CAGGCUACAGGCAAUCCUGGGUG | 216 | 77 |
| RD0626 | 510 | 530 | UACCCAGGAUUGCCUGUAGCC | 59 | AGGCUACAGGCAAUCCUGGGUAU | 217 | 51 |
| RD0630 | 540 | 560 | UGGAGGUGCAGUUCUUGUCCU | 63 | AAGGACAAGAACUGCACCUCCAG | 221 | 68 |
| RD0631 | 563 | 583 | UAGGACCUUGUGCGCAUCCAG | 65 | GCUGGAUGCGCACAAGGUCCUAU | 223 | 67 |
| RD0633 | 565 | 585 | UACAGGACCUUGUGCGCAUCC | 67 | UGGAUGCGCACAAGGUCCUGUAU | 225 | 65 |
| RD0635 | 594 | 614 | UUAGCAGGCCCUGUACAGCCU | 69 | CAGGCUGUACAGGGCCUGCUAAU | 227 | 34 |
| RD0636 | 691 | 711 | ACAAACGGCUGCUUCAGGUGC | 70 | UGCACCUGAAGCAGCCGUUUGUG | 228 | 62 |
| RD0637 | 692 | 712 | UACAAACGGCUGCUUCAGGUG | 71 | GCACCUGAAGCAGCCGUUUGUAC | 229 | 84 |
| RD0639 | 694 | 714 | UGCACAAACGGCUGCUUCAGG | 73 | ACCUGAAGCAGCCGUUUGUGCAG | 231 | 82 |
| RD0640 | 695 | 715 | UUGCACAAACGGCUGCUUCAG | 74 | CCUGAAGCAGCCGUUUGUGCAAG | 232 | 89 |
| RD0642 | 713 | 733 | UGUAUAGAGAGCCAGGCCCUG | 76 | GCAGGGCCUGGCUCUCUAUACAC | 234 | 92 |
| RD0643 | 747 | 767 | UUGUGAAGUCCAGAGAGCGUG | 78 | CCACGCUCUCUGGACUUCACAAA | 236 | 75 |
| RD0644 | 748 | 768 | UCUGUGAAGUCCAGAGAGCGU | 80 | CACGCUCUCUGGACUUCACAGAA | 238 | 78 |
| RD0645 | 749 | 769 | UUCUGUGAAGUCCAGAGAGCG | 81 | ACGCUCUCUGGACUUCACAGAAC | 239 | 65 |
| RD0646 | 750 | 770 | UUUCUGUGAAGUCCAGAGAGC | 82 | CGCUCUCUGGACUUCACAGAAAU | 240 | 89 |
| RD0649 | 794 | 814 | UACAGCCUGCAUGAACCUGUC | 86 | UGACAGGUUCAUGCAGGCUGUAA | 245 | 78 |
| RD0650 | 801 | 821 | AUCCUGUCACAGCCUGCAUGA | 87 | UUCAUGCAGGCUGUGACAGGAUG | 246 | 35 |
| RD0651 | 802 | 822 | UAUCCUGUCACAGCCUGCAUG | 88 | UCAUGCAGGCUGUGACAGGAUAG | 247 | 82 |
| RD0652 | 803 | 823 | UCAUCCUGUCACAGCCUGCAU | 89 | CAUGCAGGCUGUGACAGGAUGAA | 248 | 76 |
| RD0653 | 804 | 824 | UCCAUCCUGUCACAGCCUGCA | 90 | AUGCAGGCUGUGACAGGAUGGAA | 249 | 91 |
| RD0654 | 805 | 825 | UUCCAUCCUGUCACAGCCUGC | 91 | UGCAGGCUGUGACAGGAUGGAAG | 250 | 79 |
| RD0655 | 806 | 826 | UUUCCAUCCUGUCACAGCCUG | 93 | GCAGGCUGUGACAGGAUGGAAAA | 252 | 94 |
| RD0656 | 871 | 891 | AAGUGGACGUAGGUGUUGAAA | 95 | CUUUCAACACCUACGUCCACUUC | 254 | 62 |
| RD0657 | 936 | 956 | UGCUGUUGUCCACCCAGAACU | 96 | GAGUUCUGGGUGGACAACAGCAC | 255 | 25 |
| RD0658 | 937 | 957 | UUGCUGUUGUCCACCCAGAAC | 97 | AGUUCUGGGUGGACAACAGCAAC | 256 | 71 |

TABLE 4
Inhibition of AGT mRNA by double-stranded compounds targeting SEQ ID NO: 1

| | Seq | SEQ | | | | | |
|---|---|---|---|---|---|---|---|
| | ID | ID | | | | | |
| | NO: | NO: | | | | | AGT |
| Com- | 1 | 1 | | SEQ | | SEQ | % |
| pound | Start | Stop | Antisense Sequence | ID | Sense Sequence | ID | Inhi- |
| Number | Site | Site | (5′-3′) | NO: | (5′-3′) | NO: | bition |
| RD0659 | 946 | 966 | UACACUGAGGUGCUGUUGUCC | 98 | UGGACAACAGCACCUCAGUGUAU | 257 | 76 |
| RD0660 | 990 | 1010 | UACUCCAGUGCUGGAAGGUGC | 99 | GGCACCUUCCAGCACUGGAGUAA | 258 | 56 |
| RD0661 | 991 | 1011 | UCACUCCAGUGCUGGAAGGUG | 100 | GCACCUUCCAGCACUGGAGUGAC | 259 | 55 |
| RD0662 | 1087 | 1107 | ACCUUGUCCAGGUCAGAGGCA | 104 | AUGCCUCUGACCUGGACAAGGUG | 263 | 54 |
| RD0663 | 1088 | 1108 | UACCUUGUCCAGGUCAGAGGC | 106 | UGCCUCUGACCUGGACAAGGUAG | 265 | 20 |
| RD0664 | 1089 | 1109 | UCACCUUGUCCAGGUCAGAGG | 107 | GCCUCUGACCUGGACAAGGUGAA | 266 | 15 |
| RD0665 | 1090 | 1110 | UCCACCUUGUCCAGGUCAGAG | 108 | CCUCUGACCUGGACAAGGUGGAG | 267 | 27 |
| RD0666 | 1091 | 1111 | UUCCACCUUGUCCAGGUCAGA | 109 | CUCUGACCUGGACAAGGUGGAAG | 268 | 13 |
| RD0667 | 1099 | 1119 | UUGAGACCCUCCACCUUGUCC | 110 | UGGACAAGGUGGAGGGUCUCAAU | 269 | 69 |
| RD0668 | 1109 | 1129 | UUGCUGGAAAGUGAGACCCUC | 113 | GGAGGGUCUCACUUUCCAGCAAA | 272 | 73 |
| RD0669 | 1166 | 1186 | UGGCAUGGUCAGGUGGAUGGU | 116 | GACCAUCCACCUGACCAUGCCAC | 275 | 41 |
| RD0670 | 1169 | 1189 | UUGGGGCAUGGUCAGGUGGAU | 117 | CAUCCACCUGACCAUGCCCCAAC | 276 | 63 |
| RD0671 | 1170 | 1190 | UUUGGGGCAUGGUCAGGUGGA | 118 | AUCCACCUGACCAUGCCCCAAAU | 277 | 24 |
| RD0672 | 1200 | 1220 | UCUGCAGGUCAUAAGAUCCUU | 119 | CAAGGAUCUUAUGACCUGCAGAA | 278 | 53 |
| RD0673 | 1201 | 1221 | UCCUGCAGGUCAUAAGAUCCU | 120 | AAGGAUCUUAUGACCUGCAGGAC | 279 | 46 |
| RD0674 | 1202 | 1222 | UUCCUGCAGGUCAUAAGAUCC | 121 | AGGAUCUUAUGACCUGCAGGAAC | 280 | 0 |
| RD0675 | 1209 | 1229 | UGAGCAGGUCCUGCAGGUCAU | 122 | UAUGACCUGCAGGACCUGCUCAC | 281 | 11 |
| RD0676 | 1263 | 1283 | UCAAUUUUUGCAGGUUCAGCU | 123 | GAGCUGAACCUGCAAAAAUUGAG | 282 | 62 |
| RD0677 | 1264 | 1284 | UUCAAUUUUUGCAGGUUCAGC | 124 | AGCUGAACCUGCAAAAAUUGAAC | 283 | 64 |
| RD0678 | 1343 | 1363 | UUCUGUGGGCUCUCUCUCAUC | 125 | GGAUGAGAGAGAGCCCACAGAAU | 284 | 57 |
| RD0679 | 1350 | 1370 | UGGUAGACUCUGUGGGCUCUC | 126 | AGAGAGCCCACAGAGUCUACCAA | 285 | 61 |
| RD0680 | 1351 | 1371 | UGGGUAGACUCUGUGGGCUCU | 127 | GAGAGCCCACAGAGUCUACCCAA | 286 | 74 |
| RD0591 | 143 | 163 | UAAGGGGUGUAUGUACACCCG | 14 | CCGGGUGUACAUACACCCCUUAC | 172 | 45 |
| RD0593 | 145 | 165 | UGGAAGGGGUGUAUGUACACC | 16 | GGGUGUACAUACACCCCUUCCAC | 174 | 33 |
| RD0609 | 382 | 402 | UGCAUGCCAUAUAUACGGAAG | 39 | GCUUCCGUAUAUAUGGCAUGCAC | 197 | 41 |
| RD0681 | 1352 | 1372 | UUGGGUAGACUCUGUGGGCUC | 128 | AGAGCCCACAGAGUCUACCCAAC | 287 | 73 |
| RD0682 | 1353 | 1373 | UUUGGGUAGACUCUGUGGGCU | 129 | GAGCCCACAGAGUCUACCCAAAA | 288 | 88 |
| RD0683 | 1603 | 1623 | AGAAAAGGUGGGAGACUGGGG | 138 | CCCCCAGUCUCCCACCUUUUCUU | 299 | 89 |
| RD0684 | 1617 | 1637 | UUCGACUCAUUAGAAGAAAAG | 140 | CCUUUUCUUCUAAUGAGUCGAAU | 301 | 87 |
| RD0685 | 1758 | 1778 | UACUGGUUCUUGCCUCCCCAC | 143 | GGUGGGGAGGCAAGAACCAGUAU | 304 | 87 |
| RD0686 | 1759 | 1779 | ACACUGGUUCUUGCCUCCCCA | 144 | GUGGGGAGGCAAGAACCAGUGUU | 305 | 86 |
| RD0687 | 1760 | 1780 | AACACUGGUUCUUGCCUCCCC | 145 | UGGGGAGGCAAGAACCAGUGUUU | 306 | 83 |
| RD0688 | 1761 | 1781 | AAACACUGGUUCUUGCCUCCC | 147 | GGGGAGGCAAGAACCAGUGUUUA | 308 | 80 |
| RD0689 | 1771 | 1791 | UUCCCGCGCUAAACACUGGUU | 152 | GAACCAGUGUUUAGCGCGGGAAU | 313 | 86 |
| RD0690 | 1772 | 1792 | AGUCCCGCGCUAAACACUGGU | 153 | AACCAGUGUUUAGCGCGGGACUA | 314 | 71 |
| RD0691 | 1773 | 1793 | UAGUCCCGCGCUAAACACUGG | 154 | ACCAGUGUUUAGCGCGGGACUAC | 315 | 78 |
| RD0692 | 1774 | 1794 | UUAGUCCCGCGCUAAACACUG | 155 | CCAGUGUUUAGCGCGGGACUAAU | 316 | 79 |
| RD0693 | 1795 | 1815 | UUUGGAAUUCUUUUUGGAACA | 157 | CUGUUCCAAAAAGAAUUCCAAAC | 318 | 75 |
| RD0694 | 1800 | 1820 | UGUCGGUUGGAAUUCUUUUUG | 158 | CCAAAAAGAAUUCCAACCGACAA | 319 | 85 |

TABLE 5
Inhibition of AGT mRNA by double-stranded compounds targeting SEQ ID NO: 1

| | Seq | SEQ | | | | | |
|---|---|---|---|---|---|---|---|
| | ID | ID | | | | | |
| | NO: | NO | | | | | AGT |
| Com- | 1 | 1 | | SEQ | | SEQ | % |
| pound | Start | Stop | Antisense Sequence | ID | Sense Sequence | ID | Inhi- |
| Number | Site | Site | (5′-3′) | NO: | (5′-3′) | NO: | bition |
| RD0590 | 142 | 162 | AAGGGGUGUAUGUACACCCGG | 13 | ACCGGGUGUACAUACACCCCUUC | 171 | 61 |
| RD0598 | 154 | 174 | AUGACGAGGUGGAAGGGGUGU | 21 | UACACCCCUUCCACCUCGUCAUC | 179 | 67 |
| RD0608 | 377 | 397 | UCCAUAUAUACGGAAGCCCAA | 37 | CUUGGGCUUCCGUAUAUAUGGAA | 195 | 67 |
| RD0617 | 449 | 469 | UAGGGUGCCAAAGACAGCCGU | 48 | AACGGCUGUCUUUGGCACCCUAG | 206 | 75 |
| RD0629 | 539 | 559 | UGAGGUGCAGUUCUUGUCCUU | 62 | GAAGGACAAGAACUGCACCUCAC | 220 | 50 |
| RD0642 | 713 | 733 | UGUAUAGAGAGCCAGGCCCUG | 76 | GCAGGGCCUGGCUCUCUAUACAC | 234 | 75 |
| RD0646 | 750 | 770 | UUUCUGUGAAGUCCAGAGAGC | 82 | CGCUCUCUGGACUUCACAGAAAU | 240 | 75 |
| RD0647 | 751 | 771 | AGUUCUGUGAAGUCCAGAGAG | 83 | GCUCUCUGGACUUCACAGAACUG | 241 | 68 |
| RD0653 | 804 | 824 | UCCAUCCUGUCACAGCCUGCA | 90 | AUGCAGGCUGUGACAGGAUGGAA | 249 | 78 |
| RD0655 | 806 | 826 | UUUCCAUCCUGUCACAGCCUG | 93 | GCAGGCUGUGACAGGAUGGAAAA | 252 | 88 |

TABLE 6
Inhibition of AGT mRNA by double-stranded compounds targeting SEQ ID NO: 1

| | Seq | SEQ | | | | | |
|---|---|---|---|---|---|---|---|
| | ID | ID | | | | | |
| | NO: | NO: | | | | | AGT |
| Com- | 1 | 1 | | SEQ | | SEQ | % |
| pound | Start | Stop | Antisense Sequence | ID | Sense Sequence | ID | Inhi- |
| Number | Site | Site | (5′-3′) | NO: | (5′-3′) | NO: | bition |
| RD0608 | 377 | 397 | UCCAUAUAUACGGAAGCCCAA | 37 | CUUGGGCUUCCGUAUAUAUGGAA | 195 | 73 |
| RD0653 | 804 | 824 | UCCAUCCUGUCACAGCCUGCA | 90 | AUGCAGGCUGUGACAGGAUGGAA | 249 | 86 |
| RD0655 | 806 | 826 | UUUCCAUCCUGUCACAGCCUG | 93 | GCAGGCUGUGACAGGAUGGAAAA | 252 | 88 |
| RD1033 | 376 | 397 | UCCAUAUAUACGGAAGCCCGUA | 36 | UACGGGCUUCCGUAUAUAUGGAU | 194 | 21 |

TABLE 7
Compound Chemistry

| | | Ref | SEQ |
|---|---|---|---|
| Compound | | ID | ID |
| Number | Modified Strand (5′-3′) | NO: | NO: |
| RD1033 | mU*fC*mC.mA.mU.fA.mU.fA. | IA0297 | 36 |
| | mU.mA.mC.mG.mG.fA.mA.fG. | | |
| | mC.mC.mC*fG*mU.mA | | |
| | H1.mA*mC.mG.fG.mG.mC.fU. | IS0297 | 194 |
| | mU.mC.fC.fG.fU.mA.mU.fA. | | |
| | mU.mA.fU.mG.mG*mA*mU | | |

TABLE 8
Inhibition of AGT mRNA by double-stranded
compounds targeting SEQ ID NO: 1

| | Seq | SEQ | | | | | |
|---|---|---|---|---|---|---|---|
| | ID | ID | | | | | |
| | NO: | NO: | | | | | AGT |
| Com- | 1 | 1 | | SEQ | | SEQ | % |
| pound | Start | Stop | Antisense Sequence | ID | Sense Sequence | ID | Inhi- |
| Number | Site | Site | (5′-3′) | NO: | (5′-3′) | NO: | bition |
| RD0608 | 377 | 397 | UCCAUAUAUACGGAAGCCCAA | 37 | CUUGGGCUUCCGUAUAUAUGGAA | 195 | 79 |
| RD0653 | 804 | 824 | UCCAUCCUGUCACAGCCUGCA | 90 | AUGCAGGCUGUGACAGGAUGGAA | 249 | 82 |
| RD0655 | 806 | 826 | UUUCCAUCCUGUCACAGCCUG | 93 | GCAGGCUGUGACAGGAUGGAAAA | 252 | 88 |
| RD1033 | 376 | 397 | UCCAUAUAUACGGAAGCCCGUA | 36 | UACGGGCUUCCGUAUAUAUGGAU | 194 | 45 |
| RD1045 | 805 | 826 | UUUCCAUCCUGUCACAGCCUCA | 92 | UGAGGCUGUGACAGGAUGGAAAU | 251 | 66 |

TABLE 9
Compound Chemistry

| Compound | Modified Strand | Ref ID | SEQ ID |
|---|---|---|---|
| Number | (5′-3′) | NO: | NO: |
| RD1033 | mU*fC*mC.mA.mU.fA.mU.fA. | IA0297 | 36 |
| | mU.mA.mC.mG.mG.fA.mA.fG. | | |
| | mC.mC.mC*fG*mU.mA | | |
| | H1.mA*mC.mG.fG.mG.mC.fU. | IS0297 | 194 |
| | mU.mC.fC.fG.fU.mA.mU.fA. | | |
| | mU.mA.fU.mG.mG*mA*mU | | |
| RD1045 | mU*fU*mU.mC.mC.fA.mU.fC. | IA0304 | 92 |
| | mC.mU.mG.mU.mC.fA.mC.fA. | | |
| | mG.mC.mC*fU*mC.mA | | |
| | H1.mG*mA.mG.fG.mC.mU.fG. | IS0303 | 251 |
| | mU.mG.fA.fC.fA.mG.mG.fA. | | |
| | mU.mG.fG.mA.mA*mA*mU | | |

TABLE 10
Inhibition of AGT mRNA by double-stranded
compounds targeting SEQ ID NO: 1

| | Seq | SEQ | | | | | |
|---|---|---|---|---|---|---|---|
| | ID | ID | | | | | AGT |
| | NO: | NO: | | | | | % |
| Com- | 1 | 1 | Antisense | SEQ | Sense | SEQ | Inhi- |
| pound | Start | Stop | Sequence | ID | Sequence | ID | bi- |
| Number | Site | Site | (5′-3′) | NO: | (5′-3′) | NO: | tion |
| RD1033 | 376 | 397 | UCCAUAUAUAC | 36 | UACGGGCUUCCG | 194 | 35 |
| | | | GGAAGCCCGUA | | UAUAUAUGGAU | | |
| RD1036 | 747 | 768 | UCUGUGAAGUC | 79 | UACGCUCUCUGG | 237 | 29 |
| | | | CAGAGAGCGUA | | ACUUCACAGAU | | |
| RD1037 | 1760 | 1781 | UAACACUGGUU | 146 | UCCGAGGCAAGA | 307 | 83 |
| | | | CUUGCCUCGGA | | ACCAGUGUUAU | | |
| RD1045 | 805 | 826 | UUUCCAUCCUG | 92 | UGAGGCUGUGAC | 251 | 66 |
| | | | UCACAGCCUCA | | AGGAUGGAAAU | | |
| RD1046 | 382 | 403 | AUGCAUGCCAU | 40 | UCACCGUAUAUA | 198 | 22 |
| | | | AUAUACGGUGA | | UGGCAUGCAUU | | |

TABLE 11
Compound Chemistry

| Compound | Modified Strand | Ref ID | SEQ ID |
|---|---|---|---|
| Number | (5′-3′) | NO: | NO: |
| RD1033 | mU*fC*mC.mA.mU.fA.mU. | IA0297 | 36 |
| | fA.mU.mA.mC.mG.mG.fA. | | |
| | mA.fG.mC.mC.mC*fG*mU. | | |
| | mA | | |
| | H1.mA*mC.mG.fG.mG.mC. | IS0297 | 194 |
| | fU.mU.mC.fC.fG.fU.mA. | | |
| | mU.fA.mU.mA.fU.mG.mG* | | |
| | mA*mU | | |
| RD1036 | mU*fC*mU.mG.mU.fG.mA. | IA0300 | 79 |
| | fA.mG.mU.mC.mC.mA.fG. | | |
| | mA.fG.mA.mG.mC*fG*mU. | | |
| | mA | | |
| | H1.mA*mC.mG.fC.mU.mC. | IS0300 | 237 |
| | fU.mC.mU.fG.fG.fA.mC. | | |
| | mU.fU.mC.mA.fC.mA.mG* | | |
| | mA*mU | | |
| RD1037 | mU*fA*mA.mC.mA.fC.mU. | IA0301 | 146 |
| | fG.mG.mU.mU.mC.mU.fU. | | |
| | mG.fC.mC.mU.mC*fG*mG. | | |
| | mA | | |
| | H1.mC*mC.mG.fA.mG.mG. | IS0301 | 307 |
| | fC.mA.mA.fG.fA.fA.mC. | | |
| | mC.fA.mG.mU.fG.mU.mU* | | |
| | mA*mU | | |
| RD1045 | mU*fU*mU.mC.mC.fA.mU. | IA0304 | 92 |
| | fC.mC.mU.mG.mU.mC.fA. | | |
| | mC.fA.mG.mC.mC*fU*mC. | | |
| | mA | | |
| | H1.mG*mA.mG.fG.mC.mU. | IS0303 | 251 |
| | fG.mU.mG.fA.fC.fA.mG. | | |
| | mG.fA.mU.mG.fG.mA.mA* | | |
| | mA*mU | | |
| RD1046 | mA*fU*mG.mC.mA.fU.mG. | IA0305 | 40 |
| | fC.mC.mA.mU.mA.mU.fA. | | |
| | mU.fA.mC.mG.mG*fU*mG. | | |
| | mA | | |
| | H1.mC*mA.mC.fC.mG.mU. | IS0304 | 198 |
| | fA.mU.mA.fU.fA.fU.mG. | | |
| | mG.fC.mA.mU.fG.mC.mA* | | |
| | mU*mU | | |

TABLE 12
Inhibition of AGT mRNA by double-stranded
compounds targeting SEQ ID NO: 1

| | Seq | SEQ | | | | | |
|---|---|---|---|---|---|---|---|
| | ID | ID | Anti- | | | | AGT |
| | NO: | NO: | sense | | Sense | | % |
| Com- | 1 | 1 | Se- | SEQ | Se- | SEQ | Inhi- |
| pound | Start | Stop | quence | ID | quence | ID | bi- |
| Number | Site | Site | (5′-3′) | NO: | (5′-3′) | NO: | tion |
| RD1298 | 1104 | 1125 | UGGA | 111 | UGGU | 270 | 27 |
| | | | AAGU | | GGAG | | |
| | | | GAGA | | AGUC | | |
| | | | CUCU | | UCAC | | |
| | | | CCAC | | UUUC | | |
| | | | CA | | CAU | | |

TABLE 13
Compound Chemistry

| | | Ref | SEQ |
|---|---|---|---|
| Compound | | ID | ID |
| Number | Modified Strand (5′-3′) | NO: | NO: |
| RD1298 | mU*fG*mG.mA.mA.fA.mG.fU. | IA0335 | 111 |
| | mG.mA.mG.mA.mC.fU.mC.fU. | | |
| | mC.mC.mA*fC*mC.mA | | |
| | H1.mG*mG.mU.fG.mG.mA.fG. | IS0334 | 270 |
| | mA.mG.fU.fC.fU.mC.mA.fC. | | |
| | mU.mU.fU.mC.mC* mA*mU | | |

TABLE 14
Inhibition of AGT mRNA by double-stranded compounds targeting SEQ ID NO: 1

| | Seq | SEQ | | | | | |
|---|---|---|---|---|---|---|---|
| | ID | ID | | | | | AGT |
| | NO: 1 | NO: 1 | | SEQ | | SEQ | % |
| Compound | Start | Stop | Antisense Sequence | ID | Sense Sequence | ID | Inhi- |
| Number | Site | Site | (5′-3′) | NO: | (5′-3′) | NO: | bition |
| RD1270 | 136 | 156 | UGUAUGUACACCCIGUCACCU | 12 | UAGGUGACCGGGUGUACAUACAU | 169 | 66 |
| RD1271 | 263 | 283 | UUCAUCCACAGGGIAUGUCUC | 29 | UGAGACAUCCCCUGUGGAUGAAU | 187 | 39 |
| RD1272 | 464 | 484 | UAGAUAGAGAGAGICCAGGGU | 52 | UACCCUGGCCUCUCUCUAUCUAU | 210 | 59 |
| RD1273 | 862 | 882 | UAGGUGUUGAAAGUCAGGGUG | 94 | UCACCCUGACUUUCAACACCUAU | 253 | 83 |
| RD1274 | 993 | 1013 | UGUCACUCCAGUGUUGGAAGG | 101 | UCCUUCCAACACUGGAGUGACAU | 260 | 69 |
| RD1275 | 1035 | 1055 | UCUCAGUGAAGGGUACUUGAG | 103 | UCUCAAGUACCCUUCACUGAGAU | 262 | 71 |
| RD1276 | 1105 | 1125 | UGGAAAGUGAGACUCUCCACC | 112 | UGGUGGAGAGUCUCACUUUCCAU | 270 | 73 |
| RD1278 | 1408 | 1428 | UCAAACAGGAAUGIGCGGUUC | 131 | UGAACCGCCCAUUCCUGUUUGAU | 290 | 81 |
| RD1279 | 1413 | 1433 | ACACAGCAAACAGIAAUGGGC | 132 | UGCCCAUUCCUGUUUGCUGUGUU | 291 | 62 |
| RD1280 | 1420 | 1440 | UGAUCAUACACAGUAAACAGG | 134 | UCCUGUUUACUGUGUAUGAUCAU | 293 | 75 |
| RD1281 | 1649 | 1669 | ACCAAGGAGAAACIGCUGCUU | 141 | UAAGCAGCCGUUUCUCCUUGGUU | 302 | 86 |
| RD1282 | 1678 | 1698 | CUACUGCUCACUCUAUGCAGC | 142 | UGCUGCAUAGAGUGAGCAGUAGU | 303 | 40 |
| RD1283 | 1781 | 1801 | UGGAACAGUAGUCUCGCGCUA | 156 | UUAGCGCGAGACUACUGUUCCAU | 317 | 84 |
| RD1284 | 1841 | 1861 | CAACUUGAAAAGGIAACACUG | 159 | UCAGUGUUCCCUUUUCAAGUUGU | 320 | 78 |
| RD1285 | 1842 | 1862 | UCAACUUGAAAAGIGAACACU | 160 | UAGUGUUCCCUUUUCAAGUUGAU | 321 | 87 |
| RD1286 | 1842 | 1862 | UCAACUUGAAAAGIGAACACG | 161 | UCGUGUUCCCUUUUCAAGUUGAU | 322 | 87 |
| RD1287 | 1900 | 1920 | UACAAACCGAAGGUAAUGCAA | 162 | UUUGCAUUACCUUCGGUUUGUAU | 323 | 36 |
| RD1288 | 1900 | 1920 | UACAAACCGAAGGUAAUGCAG | 163 | UCUGCAUUACCUUCGGUUUGUAU | 324 | 39 |
| RD1289 | 1906 | 1926 | ACUAAAUACAAACUGAAGGCG | 164 | UCGCCUUCAGUUUGUAUUUAGUU | 325 | 22 |
| RD1290 | 1945 | 1965 | ACAGACACUACACIGAGGUCA | 165 | UUGACCUCCGUGUAGUGUCUGUU | 326 | 21 |
| RD1291 | 1945 | 1965 | ACAGACACUACACIGAGGUCG | 166 | UCGACCUCCGUGUAGUGUCUGUU | 327 | 27 |
| RD1292 | 163 | 183 | UCAUUGUGGAUGAUGAGGUGG | 25 | UCCACCUCAUCAUCCACAAUGAU | 182 | 70 |
| RD1298 | 1104 | 1125 | UGGAAAGUGAGACUCUCCACCA | 111 | UGGUGGAGAGUCUCACUUUCCAU | 270 | 9 |

TABLE 15
Compound Chemistry

| Compound | | Ref ID | SEQ ID |
|---|---|---|---|
| Number | Modified Strand (5′-3′) | NO: | NO: |
| RD1298 | mU*fG*mG.mA.mA.fA.mG.fU. | IA0335 | 111 |
| | mG.mA.mG.mA.mC.fU.mC.fU. | | |
| | mC.mC.mA*fC*mC.mA | | |
| | H1.mG*mG.mU.fG.mG.mA.fG. | IS0334 | 270 |
| | mA.mG.fU.fC.fU.mC.mA.fC. | | |
| | mU.mU.fU.mC.mC*mA*mU | | |

TABLE 16
Inhibition of AGT mRNA by double-stranded compounds targeting SEQ ID NO: 1

| | Seq | SEQ | | | | | |
|---|---|---|---|---|---|---|---|
| | ID | ID | | | | | |
| | NO: | NO: | | | | | AGT |
| | 1 | 1 | | SEQ | | SEQ | % |
| Compound | Start | Stop | Antisense Sequence | ID | Sense Sequence | ID | Inhi- |
| Number | Site | Site | (5′-3′) | NO: | (5′-3′) | NO: | bition |
| RD1277 | 1407 | 1427 | UAAACAGGAAUGGICGGUUCA | 130 | UUGAACCGCCCAUUCCUGUUUAU | 289 | 17 |
| RD1324 | 1104 | 1125 | UGGAAAGUGAGACUCUCCACCA | 111 | UGGUGGAGAGUCUCACUUUCCAU | 270 | 65 |
| RD1354 | 162 | 183 | UCAUUGUGGAUGAUGAGGUGGA | 24 | UCCACCUCAUCAUCCACAAUGAU | 182 | 44 |
| RD1355 | 736 | 757 | UAGAGAGCGUGGGAGGACCACA | 77 | UGUGGUCCUCCCACGCUCUCUAU | 235 | 83 |
| RD1356 | 769 | 790 | UUUCUCAGCAGCAACAUCCAGA | 84 | UCUGGAUGUUGCUGCUGAGAAAU | 242 | 55 |
| RD1377 | 1019 | 1041 | ACUUGAGUCACCGAGAAGUUGGA | 102 | UCCAACUUCUCGGUGACUCAAGUU | 261 | 85 |
| RD1378 | 304 | 325 | AAGUUUUGCAGCGACUAGCACA | 31 | UGUGCUAGUCGCUGCAAAACUUU | 189 | 12 |
| RD1379 | 483 | 504 | UCUGUGUGGUCCAAGGCUCCCA | 53 | UGGGAGCCUUGGACCACACAGAU | 211 | 34 |
| RD1380 | 556 | 577 | UUUGUGCGCAUCCAGCCGGGAA | 64 | UUCCCGGCUGGAUGCGCACAAAU | 222 | 66 |

TABLE 17
Compound Chemistry

| | | | SEQ |
|---|---|---|---|
| Compound | | Ref ID | ID |
| Number | Modified Strand (5′-3′) | NO: | NO: |
| RD1324 | mU*fG*mG.mA.mA.fA.mG.fU.mG.mA.mG.mA.mC.fU.mC.fU.mC.mC.mA*fC*mC.mA | IA0335 | 111 |
| | H2.mG*mG.mU.fG.mG.mA.fG.mA.mG.fU.fC.fU.mC.mA.fC.mU.mU.fU.mC.mC*mA*mU | IS0336 | 270 |
| RD1354 | mU*fC*mA.mU.mU.fG.mU.fG.mG.mA.mU.mG.mA.fU.mG.fA.mG.mG.mU*fG*mG.mA | IA0336 | 24 |
| | H2.mC*mC.mA.fC.mC.mU.fC.mA.mU.fC.fA.fU.mC.mC.fA.mC.mA.fA.mU.mG*mA*mU | IS0340 | 182 |
| RD1355 | mU*fA*mG.mA.mG.fA.mG.fC.mG.mU.mG.mG.mG.fA.mG.fG.mA.mC.mC*mA*mC.mA | IA0337 | 77 |
| | H2.mG*mU.mG.fG.mU.mC.fC.mU.mC.fC.fC.fA.mC.mG.fC.mU.mC.fU.mC.mU*mA*mU | IS0344 | 235 |
| RD1356 | mU*fU*mU.mC.mU.fC.mA.fG.mC.mA.mG.mC.mA.fA.mC.fA.mU.mC.mC*mA*mG.mA | IA0338 | 84 |
| | H2.mC*mU.mG.fG.mA.mU.fG.mU.mU.fG.fC.fU.mG.mC.fU.mG.mA.fG.mA.mA*mA*mU | IS0345 | 242 |
| RD1377 | mA*fC*mU.fU.mG.fA.mG.fU.mC.fA.mC.fC.mG.fA.mG.fA.mA.fG.mU.fU*mG*mG.mA | IA0343 | 102 |
| | H2.mC*mC.mA.mA.fC.mU.mU.fC.mU.mC.fG.fG.fU.mG.mA.fC.mU.mC.fA.mA.mG*mU* | IS0346 | 261 |
| | mU | | |
| RD1378 | mA*fA*mG.fU.mU.fU.mU.fG.mC.fA.mG.fC.mG.fA.mC.fU.mA.fG.mC*fA*mC.mA | IA0344 | 31 |
| | H2.mG*mU*mG.fC.mU.mA.fG.mU.mC.fG.fC.fU.mG.mC.fA.mA.mA.fA.mC.mU*mU*mU | IS0347 | 189 |
| RD1379 | mU*fC*mU.fG.mU.fG.mU.fG.mG.fU.mC.fC.mA.fA.mG.fG.mC.fU.mC*fC*mC.mA | IA0345 | 53 |
| | H2.mG*mG*mG.fA.mG.mC.fC.mU.mU.fG.fG.fA.mC.mC.fA.mC.mA.fC.mA.mG*mA*mU | IS0348 | 211 |
| RD1380 | mU*fU*mU.fG.mU.fG.mC.fG.mC.fA.mU.fC.mC.fA.mG.fC.mC.fG.mG*fG*mA.mA | IA0346 | 64 |
| | H2.mU*mC*mC.fC.mG.mG.fC.mU.mG.fG.fA.fU.mG.mC.fG.mC.mA.fC.mA.mA*mA*mU | IS0349 | 222 |

TABLE 18
Inhibition of AGT mRNA by double-stranded compounds targeting SEQ ID NO: 1

| | Seq | SEQ | | | | | |
|---|---|---|---|---|---|---|---|
| | ID | ID | | | | | AGT |
| | NO:1 | NO:1 | | SEQ | | SEQ | % |
| Compound | Start | Stop | Antisense Sequence | ID | Sense Sequence | ID | Inhi- |
| Number | Site | Site | (5′-3′) | NO: | (5′-3′) | NO: | bition |
| RD0608 | 377 | 397 | UCCAUAUAUACGGAAGCCCAA | 37 | CUUGGGCUUCCGUAUAUAUGGAA | 195 | 74 |
| RD0644 | 748 | 768 | UCUGUGAAGUCCAGAGAGCGU | 80 | CACGCUCUCUGGACUUCACAGAA | 238 | 66 |
| RD0655 | 806 | 826 | UUUCCAUCCUGUCACAGCCUG | 93 | GCAGGCUGUGACAGGAUGGAAAA | 252 | 94 |
| RD0688 | 1761 | 1781 | AAACACUGGUUCUUGCCUCCC | 147 | GGGGAGGCAAGAACCAGUGUUUA | 308 | 88 |
| RD1389 | 376 | 397 | UCCAUAUAUACGGAAGCCCGUA | 36 | UACGGGCUUCCGUAUAUAUGGAU | 194 | 40 |
| RD1390 | 747 | 768 | UCUGUGAAGUCCAGAGAGCGUA | 79 | UACGCUCUCUGGACUUCACAGAU | 237 | 18 |
| RD1392 | 805 | 826 | UUUCCAUCCUGUCACAGCCUCA | 92 | UGAGGCUGUGACAGGAUGGAAAU | 251 | 33 |
| RD1402 | 376 | 397 | UCCAUAUAUACGGAAGCCCGUA | 36 | UACGGGCUUCCGUAUAUAUGGAU | 194 | 81 |
| RD1403 | 747 | 768 | UCUGUGAAGUCCAGAGAGCGUA | 79 | UACGCUCUCUGGACUUCACAGAU | 237 | 61 |
| RD1405 | 805 | 826 | UUUCCAUCCUGUCACAGCCUCA | 92 | UGAGGCUGUGACAGGAUGGAAAU | 251 | 71 |
| RD1415 | 376 | 397 | UCCAUAUAUACGGAAGCCCGUA | 36 | UACGGGCUUCCGUAUAUAUGGAU | 194 | 77 |
| RD1416 | 747 | 768 | UCUGUGAAGUCCAGAGAGCGUA | 79 | UACGCUCUCUGGACUUCACAGAU | 237 | 65 |
| RD1418 | 805 | 826 | UUUCCAUCCUGUCACAGCCUCA | 92 | UGAGGCUGUGACAGGAUGGAAAU | 251 | 81 |
| RD1037 | 1760 | 1781 | UAACACUGGUUCUUGCCUCGGA | 146 | UCCGAGGCAAGAACCAGUGUUAU | 307 | 87 |
| RD1033 | 376 | 397 | UCCAUAUAUACGGAAGCCCGUA | 36 | UACGGGCUUCCGUAUAUAUGGAU | 194 | 30 |
| RD1045 | 805 | 826 | UUUCCAUCCUGUCACAGCCUCA | 92 | UGAGGCUGUGACAGGAUGGAAAU | 251 | 50 |

TABLE 19
Compound Chemistry

| | | | SEQ |
|---|---|---|---|
| Compound | | Ref ID | ID |
| Number | Modified Strand (5′-3′) | NO: | NO: |
| RD1389 | mU*fC*mC.fA.mU.fA.mU.fA.mU.fA.mC.fG.mG.fA.mA.fG.mC.fC.mC.fG*mU.mA | IA0347 | 36 |
| | mU.mA*mC*mG.fG.mG.mC.fU.mU.fC.mC.fG.fU.mA.mU.fA.mU.mA.fU.mG.mG*mA*mU | IS0350 | 194 |
| RD1390 | mU*fC*mU.fG.mU.fG.mA.fA.mG.fU.mC.fC.mA.fG.mA.fG.mA.fG.mC.fG*mU.mA | IA0348 | 79 |
| | mU.mA*mC*mG.fC.mU.mC.fU.mC.fU.mG.fG.fA.mC.mU.fU.mC.mA.fC.mA.mG*mA*mU | IS0351 | 237 |
| RD1392 | mU*fU*mU.fC.mC.fA.mU.fC.mC.fU.mG.fU.mC.fA.mC.fA.mG.fC.mC.fU*mC.mA | IA0350 | 92 |
| | mU.mG*mA*mG.fG.mC.mU.fG.mU.fG.mA.fC.fA.mG.mG.fA.mU.mG.fG.mA.mA*mA*mU | IS0353 | 251 |
| RD1402 | mU*fC*mC.fA.mU.fA.mU.fA.mU.fA.mC.fG.mG.fA.mA.fG.mC.fC.mC.fG*mU.mA | IA0347 | 36 |
| | mU.mA*mC*mG.fG.mG.mC.fU.mU.fC.fC.fG.fU.mA.mU.fA.mU.mA.fU.mG.mG*mA*mU | IS0363 | 194 |
| RD1403 | mU*fC*mU.fG.mU.fG.mA.fA.mG.fU.mC.fC.mA.fG.mA.fG.mA.fG.mC.fG*mU.mA | IA0348 | 79 |
| | mU.mA*mC*mG.fC.mU.mC.fU.mC.fU.fG.fG.fA.mC.mU.fU.mC.mA.fC.mA.mG*mA*mU | IS0364 | 237 |
| RD1405 | mU*fU*mU.fC.mC.fA.mU.fC.mC.fU.mG.fU.mC.fA.mC.fA.mG.fC.mC.fU*mC.mA | IA0350 | 92 |
| | mU.mG*mA*mG.fG.mC.mU.fG.mU.fG.fA.fC.fA.mG.mG.fA.mU.mG.fG.mA.mA*mA*mU | IS0366 | 251 |
| RD1415 | mU*fC*mC.fA.mU.fA.mU.fA.mU.fA.mC.fG.mG.fA.mA.fG.mC.fC.mC.fG*mU.mA | IA0347 | 36 |
| | mU.mA*mC*mG.mG.mG.mC.fU.mU.fC.fC.fG.fU.mA.mU.mA.mU.mA.mU.mG.mG*mA*mU | IS0376 | 194 |
| RD1416 | mU*fC*mU.fG.mU.fG.mA.fA.mG.fU.mC.fC.mA.fG.mA.fG.mA.fG.mC.fG*mU.mA | IA0348 | 79 |
| | mU.mA*mC*mG.mC.mU.mC.fU.mC.fU.fG.fG.fA.mC.mU.mU.mC.mA.mC.mA.mG*mA*mU | IS0377 | 237 |
| RD1418 | mU*fU*mU.fC.mC.fA.mU.fC.mC.fU.mG.fU.mC.fA.mC.fA.mG.fC.mC.fU*mC.mA | IA0350 | 92 |
| | mU.mG*mA*mG.mG.mC.mU.fG.mU.fG.fA.fC.fA.mG.mG.mA.mU.mG.mG.mA.mA*mA*mU | IS0379 | 251 |
| RD1037 | mU*fA*mA.mC.mA.fC.mU.fG.mG.mU.mU.mC.mU.fU.mG.fC.mC.mU.mC*fG*mG.mA | IA0301 | 146 |
| | H1.mC*mC.mG.fA.mG.mG.fC.mA.mA.fG.fA.fA.mC.mC.fA.mG.mU.fG.mU.mU*mA*mU | IS0301 | 307 |
| RD1033 | mU*fC*mC.mA.mU.fA.mU.fA.mU.mA.mC.mG.mG.fA.mA.fG.mC.mC.mC*fG*mU.mA | IA0297 | 36 |
| | H1.mA*mC.mG.fG.mG.mC.fU.mU.mC.fC.fG.fU.mA.mU.fA.mU.mA.fU.mG.mG*mA*mU | IS0297 | 194 |
| RD1045 | mU*fU*mU.mC.mC.fA.mU.fC.mC.mU.mG.mU.mC.fA.mC.fA.mG.mC.mC*fU*mC.mA | IA0304 | 92 |
| | H1.mG*mA.mG.fG.mC.mU.fG.mU.mG.fA.fC.fA.mG.mG.fA.mU.mG.fG.mA.mA*mA*mU | IS0303 | 251 |

TABLE 20
Inhibition of AGT mRNA by double-stranded compounds targeting SEQ ID NO: 1

| | Seq | SEQ | | | | | |
|---|---|---|---|---|---|---|---|
| | ID | ID | | | | | AGT |
| | NO:1 | NO:1 | | SEQ | | SEQ | % |
| Compound | Start | Stop | Antisense Sequence | ID | Sense Sequence | ID | Inhi- |
| Number | Site | Site | (5′-3′) | NO: | (5′-3′) | NO: | bition |
| RD1276 | 1105 | 1125 | UGGAAAGUGAGACUCUCCACC | 112 | UGGUGGAGAGUCUCACUUUCCAU | 270 | 61 |
| RD1292 | 163 | 183 | UCAUUGUGGAUGAUGAGGUGG | 25 | UCCACCUCAUCAUCCACAAUGAU | 182 | 62 |
| RD1393 | 382 | 403 | AUGCAUGCCAUAUAUACGGUGA | 40 | UCACCGUAUAUAUGGCAUGCAUU | 198 | 43 |
| RD1394 | 1104 | 1125 | UGGAAAGUGAGACUCUCCACCA | 111 | UGGUGGAGAGUCUCACUUUCCAU | 270 | 31 |
| RD1395 | 162 | 183 | UCAUUGUGGAUGAUGAGGUGGA | 24 | UCCACCUCAUCAUCCACAAUGAU | 182 | 51 |
| RD1396 | 736 | 757 | UAGAGAGCGUGGGAGGACCACA | 77 | UGUGGUCCUCCCACGCUCUCUAU | 235 | 22 |
| RD1397 | 769 | 790 | UUUCUCAGCAGCAACAUCCAGA | 84 | UCUGGAUGUUGCUGCUGAGAAAU | 242 | 77 |
| RD1398 | 1019 | 1041 | ACUUGAGUCACCGAGAAGUUGGA | 102 | UCCAACUUCUCGGUGACUCAAGUU | 261 | 29 |
| RD1400 | 483 | 504 | UCUGUGUGGUCCAAGGCUCCCA | 53 | UGGGAGCCUUGGACCACACAGAU | 211 | 22 |
| RD1401 | 556 | 577 | UUUGUGCGCAUCCAGCCGGGAA | 64 | UUCCCGGCUGGAUGCGCACAAAU | 222 | 25 |
| RD1406 | 382 | 403 | AUGCAUGCCAUAUAUACGGUGA | 40 | UCACCGUAUAUAUGGCAUGCAUU | 198 | 78 |
| RD1407 | 1104 | 1125 | UGGAAAGUGAGACUCUCCACCA | 111 | UGGUGGAGAGUCUCACUUUCCAU | 270 | 34 |
| RD1408 | 162 | 183 | UCAUUGUGGAUGAUGAGGUGGA | 24 | UCCACCUCAUCAUCCACAAUGAU | 182 | 77 |
| RD1409 | 736 | 757 | UAGAGAGCGUGGGAGGACCACA | 77 | UGUGGUCCUCCCACGCUCUCUAU | 235 | 43 |
| RD1410 | 769 | 790 | UUUCUCAGCAGCAACAUCCAGA | 84 | UCUGGAUGUUGCUGCUGAGAAAU | 242 | 86 |
| RD1411 | 1019 | 1041 | ACUUGAGUCACCGAGAAGUUGGA | 102 | UCCAACUUCUCGGUGACUCAAGUU | 261 | 63 |
| RD1413 | 483 | 504 | UCUGUGUGGUCCAAGGCUCCCA | 53 | UGGGAGCCUUGGACCACACAGAU | 211 | 32 |
| RD1414 | 556 | 577 | UUUGUGCGCAUCCAGCCGGGAA | 64 | UUCCCGGCUGGAUGCGCACAAAU | 222 | 32 |
| RD1419 | 382 | 403 | AUGCAUGCCAUAUAUACGGUGA | 40 | UCACCGUAUAUAUGGCAUGCAUU | 198 | 83 |
| RD1420 | 1104 | 1125 | UGGAAAGUGAGACUCUCCACCA | 111 | UGGUGGAGAGUCUCACUUUCCAU | 270 | 42 |
| RD1421 | 162 | 183 | UCAUUGUGGAUGAUGAGGUGGA | 24 | UCCACCUCAUCAUCCACAAUGAU | 182 | 80 |
| RD1422 | 736 | 757 | UAGAGAGCGUGGGAGGACCACA | 77 | UGUGGUCCUCCCACGCUCUCUAU | 235 | 44 |
| RD1423 | 769 | 790 | UUUCUCAGCAGCAACAUCCAGA | 84 | UCUGGAUGUUGCUGCUGAGAAAU | 242 | 89 |
| RD1424 | 1019 | 1041 | ACUUGAGUCACCGAGAAGUUGGA | 102 | UCCAACUUCUCGGUGACUCAAGUU | 261 | 72 |
| RD1426 | 483 | 504 | UCUGUGUGGUCCAAGGCUCCCA | 53 | UGGGAGCCUUGGACCACACAGAU | 211 | 27 |
| RD1427 | 556 | 577 | UUUGUGCGCAUCCAGCCGGGAA | 64 | UUCCCGGCUGGAUGCGCACAAAU | 222 | 51 |
| RD1037 | 1760 | 1781 | UAACACUGGUUCUUGCCUCGGA | 146 | UCCGAGGCAAGAACCAGUGUUAU | 307 | 77 |
| RD1046 | 382 | 403 | AUGCAUGCCAUAUAUACGGUGA | 40 | UCACCGUAUAUAUGGCAUGCAUU | 198 | 25 |
| RD1298 | 1104 | 1125 | UGGAAAGUGAGACUCUCCACCA | 111 | UGGUGGAGAGUCUCACUUUCCAU | 270 | 11 |

TABLE 21
Compound Chemistry

| | | | SEQ |
|---|---|---|---|
| Compound | | Ref ID | ID |
| Number | Modified Strand (5′-3′) | NO: | NO: |
| RD1393 | mA*fU*mG.fC.mA.fU.mG.fC.mC.fA.mU.fA.mU.fA.mU.fA.mC.fG.mG.fU*mG.mA | IA0351 | 40 |
| | mU.mC*mA*mC.fC.mG.mU.fA.mU.fA.mU.fA.fU.mG.mG.fC.mA.mU.fG.mC.mA*mU*mU | IS0354 | 198 |
| RD1394 | mU*fG*mG.fA.mA.fA.mG.fU.mG.fA.mG.fA.mC.fU.mC.fU.mC.fC.mA.fC*mC.mA | IA0352 | 111 |
| | mU.mG*mG*mU.fG.mG.mA.fG.mA.fG.mU.fC.fU.mC.mA.fC.mU.mU.fU.mC.mC*mA*mU | IS0355 | 270 |
| RD1395 | mU*fC*mA.fU.mU.fG.mU.fG.mG.fA.mU.fG.mA.fU.mG.fA.mG.fG.mU.fG*mG.mA | IA0353 | 24 |
| | mU.mC*mC*mA.fC.mC.mU.fC.mA.fU.mC.fA.fU.mC.mC.fA.mC.mA.fA.mU.mG*mA*mU | IS0356 | 182 |
| RD1396 | mU*fA*mG.fA.mG.fA.mG.fC.mG.fU.mG.fG.mG.fA.mG.fG.mA.fC.mC.fA*mC.mA | IA0354 | 77 |
| | mU.mG*mU*mG.fG.mU.mC.fC.mU.fC.mC.fC.fA.mC.mG.fC.mU.mC.fU.mC.mU*mA*mU | IS0357 | 235 |
| RD1397 | mU*fU*mU.fC.mU.fC.mA.fG.mC.fA.mG.fC.mA.fA.mC.fA.mU.fC.mC.fA*mG.mA | IA0355 | 84 |
| | mU.mC*mU*mG.fG.mA.mU.fG.mU.fU.mG.fC.fU.mG.mC.fU.mG.mA.fG.mA.mA*mA*mU | IS0358 | 242 |
| RD1398 | mA*fC*mU.fU.mG.fA.mG.fU.mC.fA.mC.fC.mG.fA.mG.fA.mA.fG.mU.fU.mG*mG.mA | IA0356 | 102 |
| | mU.mC*mC*mA.mA.fC.mU.mU.fC.mU.fC.mG.fG.fU.mG.mA.fC.mU.mC.fA.mA.mG*mU*mU | IS0359 | 261 |
| RD1400 | mU*fC*mU.fG.mU.fG.mU.fG.mG.fU.mC.fC.mA.fA.mG.fG.mC.fU.mC.fC*mC.mA | IA0358 | 53 |
| | mU.mG*mG*mG.fA.mG.mC.fC.mU.fU.mG.fG.fA.mC.mC.fA.mC.mA.fC.mA.mG*mA*mU | IS0361 | 211 |
| RD1401 | mU*fU*mU.fG.mU.fG.mC.fG.mC.fA.mU.fC.mC.fA.mG.fC.mC.fG.mG.fG*mA.mA | IA0359 | 64 |
| | mU.mU*mC*mC.fC.mG.mG.fC.mU.fG.mG.fA.fU.mG.mC.fG.mC.mA.fC.mA.mA*mA*mU | IS0362 | 222 |
| RD1406 | mA*fU*mG.fC.mA.fU.mG.fC.mC.fA.mU.fA.mU.fA.mU.fA.mC.fG.mG.fU*mG.mA | IA0351 | 40 |
| | mU.mC*mA*mC.fC.mG.mU.fA.mU.fA.fU.fA.fU.mG.mG.fC.mA.mU.fG.mC.mA*mU*mU | IS0367 | 198 |
| RD1407 | mU*fG*mG.fA.mA.fA.mG.fU.mG.fA.mG.fA.mC.fU.mC.fU.mC.fC.mA.fC*mC.mA | IA0352 | 111 |
| | mU.mG*mG*mU.fG.mG.mA.fG.mA.fG.fU.fC.fU.mC.mA.fC.mU.mU.fU.mC.mC*mA*mU | IS0368 | 270 |
| RD1408 | mU*fC*mA.fU.mU.fG.mU.fG.mG.fA.mU.fG.mA.fU.mG.fA.mG.fG.mU.fG*mG.mA | IA0353 | 24 |
| | mU.mC*mC*mA.fC.mC.mU.fC.mA.fU.fC.fA.fU.mC.mC.fA.mC.mA.fA.mU.mG*mA*mU | IS0369 | 182 |
| RD1409 | mU*fA*mG.fA.mG.fA.mG.fC.mG.fU.mG.fG.mG.fA.mG.fG.mA.fC.mC.fA*mC.mA | IA0354 | 77 |
| | mU.mG*mU*mG.fG.mU.mC.fC.mU.fC.fC.fC.fA.mC.mG.fC.mU.mC.fU.mC.mU*mA*mU | IS0370 | 235 |
| RD1410 | mU*fU*mU.fC.mU.fC.mA.fG.mC.fA.mG.fC.mA.fA.mC.fA.mU.fC.mC.fA*mG.mA | IA0355 | 84 |
| | mU.mC*mU*mG.fG.mA.mU.fG.mU.fU.fG.fC.fU.mG.mC.fU.mG.mA.fG.mA.mA*mA*mU | IS0371 | 242 |
| RD1411 | mA*fC*mU.fU.mG.fA.mG.fU.mC.fA.mC.fC.mG.fA.mG.fA.mA.fG.mU.fU.mG*mG.mA | IA0356 | 102 |
| | mU.mC*mC*mA.mA.fC.mU.mU.fC.mU.fC.fG.fG.fU.mG.mA.fC.mU.mC.fA.mA.mG*mU*mU | IS0372 | 261 |
| RD1413 | mU*fC*mU.fG.mU.fG.mU.fG.mG.fU.mC.fC.mA.fA.mG.fG.mC.fU.mC.fC*mC.mA | IA0358 | 53 |
| | mU.mG*mG*mG.fA.mG.mC.fC.mU.fU.fG.fG.fA.mC.mC.fA.mC.mA.fC.mA.mG*mA*mU | IS0374 | 211 |
| RD1414 | mU*fU*mU.fG.mU.fG.mC.fG.mC.fA.mU.fC.mC.fA.mG.fC.mC.fG.mG.fG*mA.mA | IA0359 | 64 |
| | mU.mU*mC*mC.fC.mG.mG.fC.mU.fG.fG.fA.fU.mG.mC.fG.mC.mA.fC.mA.mA*mA*mU | IS0375 | 222 |
| RD1419 | mA*fU*mG.fC.mA.fU.mG.fC.mC.fA.mU.fA.mU.fA.mU.fA.mC.fG.mG.fU*mG.mA | IA0351 | 40 |
| | mU.mC*mA*mC.mC.mG.mU.fA.mU.fA.fU.fA.fU.mG.mG.mC.mA.mU.mG.mC.mA*mU*mU | IS0380 | 198 |
| RD1420 | mU*fG*mG.fA.mA.fA.mG.fU.mG.fA.mG.fA.mC.fU.mC.fU.mC.fC.mA.fC*mC.mA | IA0352 | 111 |
| | mU.mG*mG*mU.mG.mG.mA.fG.mA.fG.fU.fC.fU.mC.mA.mC.mU.mU.mU.mC.mC*mA*mU | IS0381 | 270 |
| RD1421 | mU*fC*mA.fU.mU.fG.mU.fG.mG.fA.mU.fG.mA.fU.mG.fA.mG.fG.mU.fG*mG.mA | IA0353 | 24 |
| | mU.mC*mC*mA.mC.mC.mU.fC.mA.fU.fC.fA.fU.mC.mC.mA.mC.mA.mA.mU.mG*mA*mU | IS0382 | 182 |
| RD1422 | mU*fA*mG.fA.mG.fA.mG.fC.mG.fU.mG.fG.mG.fA.mG.fG.mA.fC.mC.fA*mC.mA | IA0354 | 77 |
| | mU.mG*mU*mG.mG.mU.mC.fC.mU.fC.fC.fC.fA.mC.mG.mC.mU.mC.mU.mC.mU*mA*mU | IS0383 | 235 |
| RD1423 | mU*fU*mU.fC.mU.fC.mA.fG.mC.fA.mG.fC.mA.fA.mC.fA.mU.fC.mC.fA*mG.mA | IA0355 | 84 |
| | mU.mC*mU*mG.mG.mA.mU.fG.mU.fU.fG.fC.fU.mG.mC.mU.mG.mA.mG.mA.mA*mA*mU | IS0384 | 242 |
| RD1424 | mA*fC*mU.fU.mG.fA.mG.fU.mC.fA.mC.fC.mG.fA.mG.fA.mA.fG.mU.fU.mG*mG.mA | IA0356 | 102 |
| | mU.mC*mC*mA.mA.mC.mU.mU.fC.mU.fC.fG.fG.fU.mG.mA.mC.mU.mC.mA.mA.mG*mU*mU | IS0385 | 261 |
| RD1426 | mU*fC*mU.fG.mU.fG.mU.fG.mG.fU.mC.fC.mA.fA.mG.fG.mC.fU.mC.fC*mC.mA | IA0358 | 53 |
| | mU.mG*mG*mG.mA.mG.mC.fC.mU.fU.fG.fG.fA.mC.mC.mA.mC.mA.mC.mA.mG*mA*mU | IS0387 | 211 |
| RD1427 | mU*fU*mU.fG.mU.fG.mC.fG.mC.fA.mU.fC.mC.fA.mG.fC.mC.fG.mG.fG*mA.mA | IA0359 | 64 |
| | mU.mU*mC*mC.mC.mG.mG.fC.mU.fG.fG.fA.fU.mG.mC.mG.mC.mA.mC.mA.mA*mA*mU | IS0388 | 222 |
| RD1037 | mU*fA*mA.mC.mA.fC.mU.fG.mG.mU.mU.mC.mU.fU.mG.fC.mC.mU.mC*fG*mG.mA | IA0301 | 146 |
| | H1.mC*mC.mG.fA.mG.mG.fC.mA.mA.fG.fA.fA.mC.mC.fA.mG.mU.fG.mU.mU*mA*mU | IS0301 | 307 |
| RD1046 | mA*fU*mG.mC.mA.fU.mG.fC.mC.mA.mU.mA.mU.fA.mU.fA.mC.mG.mG*fU*mG.mA | IA0305 | 40 |
| | H1.mC*mA.mC.fC.mG.mU.fA.mU.mA.fU.fA.fU.mG.mG.fC.mA.mU.fG.mC.mA*mU*mU | IS0304 | 198 |
| RD1298 | mU*fG*mG.mA.mA.fA.mG.fU.mG.mA.mG.mA.mC.fU.mC.fU.mC.mC.mA*fC*mC.mA | IA0335 | 111 |
| | H1.mG*mG.mU.fG.mG.mA.fG.mA.mG.fU.fC.fU.mC.mA.fC.mU.mU.fU.mC.mC*mA*mU | IS0334 | 270 |

TABLE 22
Inhibition of AGT mRNA by double-stranded compounds targeting SEQ ID NO: 1

| | Seq | SEQ | | | | | |
|---|---|---|---|---|---|---|---|
| | ID | ID | | | | | AGT |
| | NO:1 | NO:1 | | SEQ | | SEQ | % |
| Compound | Start | Stop | Antisense Sequence | ID | Sense Sequence | ID | Inhi- |
| Number | Site | Site | (5′-3′) | NO: | (5′-3′) | NO: | bition |
| RD1391 | 1760 | 1781 | UAACACUGGUUCUUGCCUCGGA | 146 | UCCGAGGCAAGAACCAGUGUUAU | 307 | 84 |
| RD1399 | 304 | 325 | AAGUUUUGCAGCGACUAGCACA | 31 | UGUGCUAGUCGCUGCAAAACUUU | 189 | 84 |
| RD1404 | 1760 | 1781 | UAACACUGGUUCUUGCCUCGGA | 146 | UCCGAGGCAAGAACCAGUGUUAU | 307 | 88 |
| RD1412 | 304 | 325 | AAGUUUUGCAGCGACUAGCACA | 31 | UGUGCUAGUCGCUGCAAAACUUU | 189 | 7 |
| RD1417 | 1760 | 1781 | UAACACUGGUUCUUGCCUCGGA | 146 | UCCGAGGCAAGAACCAGUGUUAU | 307 | 87 |
| RD1425 | 304 | 325 | AAGUUUUGCAGCGACUAGCACA | 31 | UGUGCUAGUCGCUGCAAAACUUU | 189 | 87 |
| RD1553 | 1761 | 1781 | UAACACUGGUUCUUGCCUCCC | 148 | UGGGAGGCAAGAACCAGUGUUAU | 309 | 81 |
| RD1554 | 1761 | 1781 | UAACACUGGUUCUUGCCUCCG | 149 | UCGGAGGCAAGAACCAGUGUUAU | 310 | 82 |
| RD1037 | 1760 | 1781 | UAACACUGGUUCUUGCCUCGGA | 146 | UCCGAGGCAAGAACCAGUGUUAU | 307 | 76 |

TABLE 23
Compound Chemistry

| Compound | | Ref ID | SEQ ID |
|---|---|---|---|
| Number | Modified Strand (5′-3′) | NO: | NO: |
| RD1391 | mU*fA*mA.fC.mA.fC.mU.fG.mG.fU.mU.fC.mU.fU.mG.fC.mC.fU.mC.fG*mG.mA | IA0349 | 146 |
| | mU.mC*mC*mG.fA.mG.mG.fC.mA.fA.mG.fA.fA.mC.mC.fA.mG.mU.fG.mU.mU*mA*mU | IS0352 | 307 |
| RD1399 | mA*fA*mG.fU.mU.fU.mU.fG.mC.fA.mG.fC.mG.fA.mC.fU.mA.fG.mC.fA*mC.mA | IA0357 | 31 |
| | mU.mG*mU*mG.fC.mU.mA.fG.mU.fC.mG.fC.fU.mG.mC.fA.mA.mA.fA.mC.mU*mU*mU | IS0360 | 189 |
| RD1404 | mU*fA*mA.fC.mA.fC.mU.fG.mG.fU.mU.fC.mU.fU.mG.fC.mC.fU.mC.fG*mG.mA | IA0349 | 146 |
| | mU.mC*mC*mG.fA.mG.mG.fC.mA.fA.fG.fA.fA.mC.mC.fA.mG.mU.fG.mU.mU*mA*mU | IS0365 | 307 |
| RD1412 | mA*fA*mG.fU.mU.fU.mU.fG.mC.fA.mG.fC.mG.fA.mC.fU.mA.fG.mC.fA*mC.mA | IA0357 | 31 |
| | mU.mG*mU*mG.fC.mU.mA.fG.mU.fC.fG.fC.fU.mG.mC.fA.mA.mA.fA.mC.mU*mU*mU | IS0373 | 189 |
| RD1417 | mU*fA*mA.fC.mA.fC.mU.fG.mG.fU.mU.fC.mU.fU.mG.fC.mC.fU.mC.fG*mG.mA | IA0349 | 146 |
| | mU.mC*mC*mG.mA.mG.mG.fC.mA.fA.fG.fA.fA.mC.mC.mA.mG.mU.mG.mU.mU*mA*mU | IS0378 | 307 |
| RD1425 | mA*fA*mG.fU.mU.fU.mU.fG.mC.fA.mG.fC.mG.fA.mC.fU.mA.fG.mC.fA*mC.mA | IA0357 | 31 |
| | mU.mG*mU*mG.mC.mU.mA.fG.mU.fC.fG.fC.fU.mG.mC.mA.mA.mA.mA.mC.mU*mU*mU | IS0386 | 189 |
| RD1553 | mU*fA*mA.fC.mA.fC.mU.fG.mG.fU.mU.fC.mU.fU.mG.fC.mC.fU.mC*fC*mC | IA0431 | 148 |
| | mU.mG*mG*mG.fA.mG.mG.fC.mA.fA.mG.fA.fA.mC.mC.fA.mG.mU.fG.mU.mU*mA*mU | IS0491 | 309 |
| RD1554 | mU*fA*mA.fC.mA.fC.mU.fG.mG.fU.mU.fC.mU.fU.mG.fC.mC.fU.mC*fC*mG | IA0432 | 149 |
| | mU.mC*mG*mG.fA.mG.mG.fC.mA.fA.mG.fA.fA.mC.mC.fA.mG.mU.fG.mU.mU*mA*mU | IS0492 | 310 |
| RD1037 | mU*fA*mA.mC.mA.fC.mU.fG.mG.mU.mU.mC.mU.fU.mG.fC.mC.mU.mC*fG*mG.mA | IA0301 | 146 |
| | H1.mC*mC.mG.fA.mG.mG.fC.mA.mA.fG.fA.fA.mC.mC.fA.mG.mU.fG.mU.mU*mA*mU | IS0301 | 307 |

TABLE 24
Inhibition of AGT mRNA by double-stranded compounds targeting SEQ ID NO: 1

| | Seq | SEQ | | | | | |
|---|---|---|---|---|---|---|---|
| | ID | ID | | | | | AGT |
| | NO:1 | NO:1 | | SEQ | | SEQ | % |
| Compound | Start | Stop | Antisense Sequence | ID | Sense Sequence | ID | Inhi- |
| Number | Site | Site | (5′-3′) | NO: | (5′-3′) | NO: | bition |
| RD1404 | 1760 | 1781 | UAACACUGGUUCUUGCCUCGGA | 146 | UCCGAGGCAAGAACCAGUGUUAU | 307 | 86 |
| RD1412 | 304 | 325 | AAGUUUUGCAGCGACUAGCACA | 31 | UGUGCUAGUCGCUGCAAAACUUU | 189 | 86 |
| RD1037 | 1760 | 1781 | UAACACUGGUUCUUGCCUCGGA | 146 | UCCGAGGCAAGAACCAGUGUUAU | 307 | 80 |
| RD1613 | 377 | 397 | UCCAUAUAUACGGAAGCCCAG | 38 | UCUGGGCUUCCGUAUAUAUGGA | 196 | 76 |
| RD1614 | 1761 | 1781 | AAACACUGGUUCUUGCCUCCC | 147 | UGGGAGGCAAGAACCAGUGUUU | 311 | 81 |
| RD1615 | 1607 | 1629 | AUUAGAAGAAAAGGUGGGAGACU | 139 | UUCUCCCACCUUUUCUUCUAAU | 300 | 85 |
| RD1622 | 136 | 156 | UGUAUGUACACCCIGUCACCU | 12 | UAGGUGACCGGGUGUACAUACA | 170 | 66 |
| RD1623 | 1420 | 1440 | UGAUCAUACACAGUAAACAGG | 134 | UCCUGUUUACUGUGUAUGAUCA | 294 | 74 |
| RD1624 | 163 | 183 | UCAUUGUGGAUGAUGAGGUGG | 25 | UCCACCUCAUCAUCCACAAUGA | 183 | 76 |
| RD1625 | 793 | 813 | ACAGCCUGCAUGAACCUGUCA | 85 | UUGACAGGUUCAUGCAGACUGU | 244 | 76 |
| RD1626 | 1087 | 1107 | ACCUUGUCCAGGUUAGAGGCA | 105 | UUGCCUCUAACCUGGAUAAGGU | 264 | 66 |

TABLE 25
Compound Chemistry

| | | | SEQ |
|---|---|---|---|
| Compound | | Ref ID | ID |
| Number | Modified Strand (5′-3′) | NO: | NO: |
| RD1404 | mU*fA*mA.fC.mA.fC.mU.fG.mG.fU.mU.fC.mU.fU.mG.fC.mC.fU.mC.fG*mG.mA | IA0349 | 146 |
| | mU.mC*mC*mG.fA.mG.mG.fC.mA.fA.fG.fA.fA.mC.mC.fA.mG.mU.fG.mU.mU*mA*mU | IS0365 | 307 |
| RD1412 | mA*fA*mG.fU.mU.fU.mU.fG.mC.fA.mG.fC.mG.fA.mC.fU.mA.fG.mC.fA*mC.mA | IA0357 | 31 |
| | mU.mG*mU*mG.fC.mU.mA.fG.mU.fC.fG.fC.fU.mG.mC.fA.mA.mA.fA.mC.mU*mU*mU | IS0373 | 189 |
| RD1037 | mU*fA*mA.mC.mA.fC.mU.fG.mG.mU.mU.mC.mU.fU.mG.fC.mC.mU.mC*fG*mG.mA | IA0301 | 146 |
| | H1.mC*mC.mG.fA.mG.mG.fC.mA.mA.fG.fA.fA.mC.mC.fA.mG.mU.fG.mU.mU*mA*mU | IS0301 | 307 |
| RD1613 | mU*fC*mC.fA.mU.fA.mU.fA.mU.fA.mC.fG.mG.fA.mA.fG.mC.fC.mC*fA*mG | IA0440 | 38 |
| | H2.mC*mU*mG.mG.mG.mC.fU.mU.fC.fC.fG.fU.mA.mU.mA.mU.mA.mU.mG.mG*mA*dQ | IS0499 | 196 |
| RD1614 | mA*fA*mA.fC.mA.fC.mU.fG.mG.fU.mU.fC.mU.fU.mG.fC.mC.fU.mC*fC*mC | IA0435 | 147 |
| | H2.mG*mG*mG.mA.mG.mG.fC.mA.fA.fG.fA.fA.mC.mC.mA.mG.mU.mG.mU.mU*mU*dQ | IS0500 | 311 |
| RD1615 | mA*fU*mU.mA.mG.fA.mA.mG.mA.mA.mA.mA.mG.fG.mU.fG.mG.mG.mA.mG.mA*mC*mU | IA0441 | 139 |
| | H2.mU*mC*mU.mC.mC.mC.fA.mC.fC.fU.fU.mU.mU.mC.mU.mU.mC.mU.mA.mA.mU*dQ | IS0501 | 300 |
| RD1622 | mU*fG*mU.fA.mU.fG.mU.fA.mC.fA.mC.fC.mC.fl.mG.fU.mC.fA.mC.fC*mU | IA0442 | 12 |
| | H2.mA*mG*mG.mU.mG.mA.fC.mC.fG.fG.fG.fU.mG.mU.mA.mC.mA.mU.mA.mC*mA*dQ | IS0504 | 170 |
| RD1623 | mU*fG*mA.fU.mC.fA.mU.fA.mC.fA.mC.fA.mG.fU.mA.fA.mA.fC.mA.fG*mG | IA0443 | 134 |
| | H2.mC*mC*mU.mG.mU.mU.fU.mA.fC.fU.fG.fU.mG.mU.mA.mU.mG.mA.mU.mC*mA*dQ | IS0505 | 294 |
| RD1624 | mU*fC*mA.fU.mU.fG.mU.fG.mG.fA.mU.fG.mA.fU.mG.fA.mG.fG.mU*fG*mG | IA0444 | 25 |
| | H2.mC*mC*mA.mC.mC.mU.fC.mA.fU.fC.fA.fU.mC.mC.mA.mC.mA.mA.mU.mG*mA*dQ | IS0508 | 183 |
| RD1625 | mA*fC*mA.fG.mC.fC.mU.fG.mC.fA.mU.fG.mA.fA.mC.fC.mU.fG.mU.fC*mA | IA0445 | 85 |
| | H2.mU*mG*mA.mC.mA.mG.fG.mU.fU.fC.fA.fU.mG.mC.mA.mG.mA.mC.mU.mG*mU*dQ | IS0509 | 244 |
| RD1626 | mA*fC*mC.fU.mU.fG.mU.fC.mC.fA.mG.fG.mU.fU.mA.fG.mA.fG.mG.fC*mA | IA0446 | 105 |
| | H2.mU*mG*mC.mC.mU.mC.fU.mA.fA.fC.fC.fU.mG.mG.mA.mU.mA.mA.mG.mG*mU*dQ | IS0510 | 264 |

### Example 2: Effect of Compounds Targeting Human AGT in Cynomolgus Monkeys

Compounds of interest, identified from in vitro gene expression screening, were evaluated in cynomolgus monkeys. Prior to the study the monkeys were kept in quarantine during which the animals were observed daily for general health. The monkeys were 6-8.5 years old and weighed 3.7-6.8 kg. 2 groups of 3 cynomolgus monkeys each were injected with a single 3 mg/kg subcutaneous dose of oligonucleotide (1 monkey) or two 3 mg/kg subcutaneous doses of oligonucleotide (5 monkeys). The first dose was on Day 1 of the study and the second dose was on Day 17. During the study period, the monkeys were observed daily for signs of illness or distress. Animals were bled on day −6 and on days 1 (prior to dosing), 4, 8, 15, 25, 32, 39, 53 and 60 for serum collection and analysis. The protocols described were approved by the Institutional Animal Care and Use Committee (IACUC). Circulating AGT levels were quantified using an ELISA specific for human angiotensinogen (and cross-reactive with cynomolgus), according to manufacturer's protocol (IBL America #27412). AGT inhibition data were expressed as percent of baseline value (Day 1 prior to dosing) and presented as a group average for each compound.

TABLE 26
Average AGT Inhibition

| | Days | | | | | | | | |
|---|---|---|---|---|---|---|---|---|---|
| Compound | 4 | 8 | 15 | 25 | 32 | 39 | 46 | 53 | 60 |
| RD1033 | 20 | 34 | 49 | 57 | 66 | 70 | 63 | 67 | 62 |
| RD1045 | 24 | 33 | 42 | 49 | 59 | 54 | 47 | 49 | 46 |

### Example 3: Effect of Compounds Targeting Human AGT in Cynomolgus Monkeys

Compounds of interest, identified from in vitro gene expression screening, were evaluated in cynomolgus monkeys. Prior to the study the monkeys were kept in quarantine during which the animals were observed daily for general health. The monkeys were 3.5-4 years old and weighed 3.1-4.6 kg. Three groups of 3 cynomolgus monkeys each were injected with a single 3 mg/kg subcutaneous dose of oligonucleotide on Day 1 of the study. During the study period, the monkeys were observed daily for signs of illness or distress. Animals were bled on day −6 and on days 1 (prior to dosing), 4, 8, 15, 22, 29, 36 and 43 for serum analysis. The protocols described were approved by the Institutional Animal Care and Use Committee (IACUC). Circulating AGT levels were quantified using an ELISA specific for human angiotensinogen (and cross-reactive with cynomolgus), according to manufacturer's protocol (IBL America #27412). AGT inhibition data were expressed as percent of baseline value (Day 1 prior to dosing) and presented as a group average for each compound.

TABLE 27
Average AGT Inhibition

| Compound | Day 15 | Day 22 |
|---|---|---|
| RD1036 | 12 | 0 |
| RD1046 | 15 | 0 |

TABLE 28
Average AGT Inhibition

Days

| Compound | 4 | 8 | 15 | 22 | 29 | 36 | 43 |
|---|---|---|---|---|---|---|---|
| RD1037 | 21 | 39 | 52 | 62 | 64 | 62 | 50 |

### Example 4: Effect of Compounds Targeting Human AGT in Cynomolgus Monkeys

Compounds of interest, identified from in vitro gene expression screening, were evaluated in cynomolgus monkeys. Prior to the study the monkeys were kept in quarantine during which the animals were observed daily for general health. The monkeys were 2.6-5.3 years old and weighed 2.2-4.4 kg. Eight groups of 2 cynomolgus monkeys each were injected with a single 3 mg/kg subcutaneous dose of oligonucleotide on Day 1 of the study. Animals were bled on day −6 and on days 1 (prior to dosing), 4, 8, 15, 22, 29, 36 and 43 for serum analysis. One group (RD1354) was injected with a 2^{nd} dose of 3 mg/kg on Day 43 and the study continued for a total of 92 days. These animals were subsequently bled on days 50, 57, 64, 71, 78, 85 and 92. The protocols described were approved by the Institutional Animal Care and Use Committee (IACUC). Circulating AGT levels were quantified using an ELISA specific for human angiotensinogen (and cross-reactive with cynomolgus), according to manufacturer's protocol (IBL America #27412). AGT inhibition data were expressed as percent of baseline value (Day 1 prior to dosing) and presented as a group average for each compound. Clinical chemistry was performed on Day 43 or 92 (RD1354 group). There were no test article-related effects on body weight and all serum chemistry values were within reference ranges.

TABLE 29
Average AGT Inhibition

| Compound | Day 15 |
|---|---|
| RD1324 | 0 |
| RD1355 | 0 |
| RD1356 | 16 |
| RD1377 | 15 |
| RD1378 | 25 |
| RD1379 | 7 |
| RD1380 | 24 |

TABLE 30
Average AGT Inhibition

| | Days | | | | | | | | | | | | | |
|---|---|---|---|---|---|---|---|---|---|---|---|---|---|---|
| Compound | 4 | 8 | 15 | 22 | 29 | 36 | 43 | 50 | 57 | 64 | 71 | 78 | 85 | 92 |
| RD1354 | 29 | 46 | 53 | 61 | 57 | 62 | 52 | 60 | 65 | 71 | 64 | 69 | 64 | 62 |

TABLE 31
Body Weight (kg)

| Compound | Day 1 | Day 35 | Day 92 |
|---|---|---|---|
| RD1324 Cyno#1 | 2.6 | 3.0 | |
| RD1324 Cyno#2 | 3.4 | 3.4 | |
| RD1354 Cyno#1 | 3.2 | 3.4 | 3.3 |
| RD1354 Cyno#2 | 2.7 | 2.8 | 2.8 |
| RD1355 Cyno#1 | 2.4 | 2.8 | |
| RD1355 Cyno#2 | 2.6 | 2.8 | |
| RD1356 Cyno#1 | 3.2 | 3.4 | |
| RD1356 Cyno#2 | 3.1 | 3.2 | |
| RD1377 Cyno#1 | 3.3 | 4.0 | |
| RD1377 Cyno#2 | 2.6 | 3.1 | |
| RD1378 Cyno#1 | 2.5 | 2.8 | |
| RD1378 Cyno#2 | 2.7 | 2.8 | |
| RD1379 Cyno#1 | 3.1 | 3.2 | |
| RD1379 Cyno#2 | 4.4 | 4.8 | |
| RD1380 Cyno#1 | 2.3 | 2.4 | |
| RD1380 Cyno#2 | 2.4 | 2.4 | |

TABLE 32
Liver Function Markers

| | ALT | ALT | AST | AST | Bilirubin | Bilirubin | Albumin | Albumin |
|---|---|---|---|---|---|---|---|---|
| | Day 43 | Day 92 | Day 43 | Day 92 | Day 43 | Day 92 | Day 43 | Day 92 |
| Compound | [U/L] | [U/L] | [U/L] | [U/L] | [mg/dL] | [mg/dL] | [g/dL] | [g/dL] |
| RD1324 | 61 | | 49 | | 0.35 | | 4.1 | |
| Cyno#1 | | | | | | | | |
| RD1324 | 66 | | 48 | | 0.18 | | 4.4 | |
| Cyno#2 | | | | | | | | |
| RD1354 | 57 | 47 | 48 | 32 | 0.29 | 0.26 | 4.9 | 4.9 |
| Cyno#1 | | | | | | | | |
| RD1354 | 40 | 31 | 40 | 28 | 0.24 | 0.14 | 4.2 | 4.0 |
| Cyno#2 | | | | | | | | |
| RD1355 | 56 | | 46 | | 0.25 | | 3.9 | |
| Cyno#1 | | | | | | | | |
| RD1355 | 62 | | 42 | | 0.22 | | 4.0 | |
| Cyno#2 | | | | | | | | |
| RD1356 | 48 | | 74 | | 0.31 | | 4.2 | |
| Cyno#1 | | | | | | | | |
| RD1356 | 48 | | 39 | | 0.24 | | 4.0 | |
| Cyno#2 | | | | | | | | |
| RD1377 | 55 | | 68 | | 0.22 | | 4.9 | |
| Cyno#1 | | | | | | | | |
| RD1377 | 33 | | 52 | | 0.36 | | 4.4 | |
| Cyno#2 | | | | | | | | |
| RD1378 | 50 | | 39 | | 0.19 | | 4.7 | |
| Cyno#1 | | | | | | | | |
| RD1378 | 34 | | 38 | | 0.23 | | 4.7 | |
| Cyno#2 | | | | | | | | |
| RD1379 | 32 | | 30 | | 0.15 | | 4.0 | |
| Cyno#1 | | | | | | | | |
| RD1379 | 50 | | 35 | | 0.15 | | 4.5 | |
| Cyno#2 | | | | | | | | |
| RD1380 | 64 | | 45 | | 0.37 | | 4.5 | |
| Cyno#1 | | | | | | | | |
| RD1380 | 39 | | 50 | | 0.16 | | 4.0 | |
| Cyno#2 | | | | | | | | |

TABLE 33
Kidney Function Markers

| | Bun | Bun | Creatinine | Creatinine |
|---|---|---|---|---|
| | Day 43 | Day 92 | Day 43 | Day 92 |
| Compound | [mg/dL] | [mg/dL] | [mg/dL] | [mg/dL] |
| RD1324 Cyno#1 | 19 | | 0.56 | |
| RD1324 Cyno#2 | 20 | | 0.73 | |
| RD1354 Cyno#1 | 28 | 25 | 0.72 | 0.65 |
| RD1354 Cyno#2 | 21 | 18 | 0.54 | 0.41 |
| RD1355 Cyno#1 | 22 | | 0.52 | |
| RD1355 Cyno#2 | 29 | | 0.90 | |
| RD1356 Cyno#1 | 22 | | 0.48 | |
| RD1356 Cyno#2 | 23 | | 0.59 | |
| RD1377 Cyno#1 | 34 | | 0.76 | |
| RD1377 Cyno#2 | 23 | | 0.50 | |
| RD1378 Cyno#1 | 27 | | 0.58 | |
| RD1378 Cyno#2 | 19 | | 0.50 | |
| RD1379 Cyno#1 | 28 | | 0.59 | |
| RD1379 Cyno#2 | 24 | | 0.62 | |
| RD1380 Cyno#1 | 24 | | 0.46 | |
| RD1380 Cyno#2 | 27 | | 0.49 | |

### Example 5: Effect of Compounds Targeting Human AGT in Cynomolgus Monkeys

Compounds of interest, identified from in vitro gene expression screening, were evaluated in cynomolgus monkeys. Prior to the study the monkeys were kept in quarantine during which the animals were observed daily for general health. The monkeys were 2.3-3.1 years old and weighed 1.8-2.4 kg. Eight groups of 2 cynomolgus monkeys each were injected with a 3 mg/kg subcutaneous dose of oligonucleotide on Day 1 and Day 22 of the study. All animals were bled on day −6 and on days 1 (prior to dosing), 4, 8, 14, 22, 29, 36, 43, 50, and 57 for serum analysis. Four of the groups (RD1614, RD1615, RD1623 and RD1625) were kept on the study an additional 35 days (for a total of 92 days) and were subsequently bled on days 64, 71, 78, 85 and 92. The protocols described were approved by the Institutional Animal Care and Use Committee (IACUC). Circulating AGT levels were quantified using an ELISA specific for human angiotensinogen (and cross-reactive with cynomolgus), according to manufacturer's protocol (IBL America #27412). AGT inhibition data were expressed as percent of baseline value (Day 1 prior to dosing) and presented as a group average for each compound. Clinical chemistry was performed on Day −6 and 57 (RD1613, RD1622, RD1624 and RD1626) or 92 (RD1614, RD1615, RD1623 and RD1625). There were no test article-related effects on body weight and all serum chemistry values were generally within reference ranges.

TABLE 34
Average AGT Inhibition

| Compound | Day 14 | Day 22 |
|---|---|---|
| RD1613 | 42 | 51 |
| RD1614 | 66 | 70 |
| RD1615 | 81 | 88 |
| RD1622 | 39 | 44 |
| RD1624 | 50 | 48 |
| RD1626 | 14 | 36 |

TABLE 35
Average AGT Inhibition

| | Days | | | | | | | | | | | | | |
|---|---|---|---|---|---|---|---|---|---|---|---|---|---|---|
| Compound | 4 | 8 | 14 | 22 | 29 | 36 | 43 | 50 | 57 | 64 | 71 | 78 | 85 | 92 |
| RD1623 | 25 | 46 | 60 | 72 | 76 | 78 | 78 | 73 | 81 | 68 | 66 | 66 | 62 | 55 |
| RD1625 | 20 | 47 | 63 | 63 | 68 | 75 | 77 | 73 | 75 | 65 | 69 | 63 | 61 | 61 |

TABLE 36
Body Weight (kg)

| Compound | Day 1 | Day 57 | Day 92 |
|---|---|---|---|
| RD1613 Cyno #1 | 2.1 | 2.3 | |
| RD1613 Cyno #2 | 2.2 | 2.4 | |
| RD1614 Cyno #1 | 2.6 | 2.8 | 2.8 |
| RD1614 Cyno #2 | 1.9 | 2.1 | 1.9 |
| RD1615 Cyno #1 | 2.1 | 2.6 | 2.1 |
| RD1615 Cyno #2 | 2.1 | 2.4 | 2.1 |
| RD1622 Cyno #1 | 1.8 | 2.0 | |
| RD1622 Cyno #2 | 2.1 | 2.3 | |
| RD1623 Cyno #1 | 2.4 | 2.6 | 2.5 |
| RD1623 Cyno #2 | 2.1 | 2.3 | 2.1 |
| RD1624 Cyno #1 | 2.1 | 2.2 | |
| RD1624 Cyno #2 | 1.7 | 1.9 | |
| RD1625 Cyno #1 | 2.0 | 2.2 | 1.9 |
| RD1625 Cyno #2 | 2.1 | 2.3 | 2.1 |
| RD1626 Cyno #1 | 2.1 | 2.3 | |
| RD1626 Cyno #2 | 1.7 | 1.0 | |

TABLE 37
Liver Function Markers

| | ALT | ALT | ALT | AST | AST | AST |
|---|---|---|---|---|---|---|
| | Day −6 | Day 57 | Day 92 | Day −6 | Day 57 | Day 92 |
| Compound | [U/L] | [U/L] | [U/L] | [U/L] | [U/L] | [U/L] |
| RD1613 Cyno #1 | 86 | 70 | | 55 | 43 | |
| RD1613 Cyno #2 | 70 | 61 | | 53 | 54 | |
| RD1614 Cyno #1 | 42 | | 51 | 48 | | 44 |
| RD1614 Cyno #2 | 82 | | 87 | 73 | | 57 |
| RD1615 Cyno #1 | 41 | | 42 | 62 | | 38 |
| RD1615 Cyno #2 | 125 | | 128 | 66 | | 71 |
| RD1622 Cyno #1 | 88 | 71 | | 69 | 80 | |
| RD1622 Cyno #2 | 105 | 74 | | 58 | 44 | |
| RD1623 Cyno #1 | 25 | | 26 | | | |
| RD1623 Cyno #2 | 49 | | 49 | | | |
| RD1624 Cyno #1 | 66 | 57 | | 67 | 55 | |
| RD1624 Cyno #2 | 29 | 34 | | 43 | 62 | |
| RD1625 Cyno #1 | 62 | | 53 | | | |
| RD1625 Cyno #2 | 38 | | 29 | | | |
| RD1626 Cyno #1 | 57 | 42 | | 90 | 40 | |
| RD1626 Cyno #2 | 64 | 57 | | 59 | 45 | |

TABLE 38
Liver Function Markers

| | Bilirubin | Bilirubin | Bilirubin | Albumin | Albumin | Albumin |
|---|---|---|---|---|---|---|
| | Day −6 | Day 57 | Day 92 | Day −6 | Day 57 | Day 92 |
| Compound | [mg/dL] | [mg/dL] | [mg/dL] | [g/dL] | [g/dL] | [g/dL] |
| RD1613 | 0.33 | 0.15 | | 4.1 | 4.2 | |
| Cyno #1 | | | | | | |
| RD1613 | 0.35 | | 0.22 | 4.2 | 3.9 | |
| Cyno #2 | | | | | | |
| RD1614 | 0.39 | | 0.35 | 4.2 | | 4.5 |
| Cyno #1 | | | | | | |
| RD1614 | 0.34 | | 0.27 | 4.3 | | 4.9 |
| Cyno #2 | | | | | | |
| RD1615 | 0.26 | | 0.31 | 4.2 | | 4.4 |
| Cyno #1 | | | | | | |
| RD1615 | 0.50 | | 0.33 | 3.8 | | 3.9 |
| Cyno #2 | | | | | | |
| RD1622 | 0.41 | 0.17 | | 4.6 | 4.5 | |
| Cyno #1 | | | | | | |
| RD1622 | 0.29 | 0.18 | | 4.6 | 4.3 | |
| Cyno #2 | | | | | | |
| RD1623 | 0.34 | | 0.31 | 4.5 | | 4.4 |
| Cyno #1 | | | | | | |
| RD1623 | 0.34 | | 0.29 | 4.6 | | 4.8 |
| Cyno #2 | | | | | | |
| RD1624 | 0.33 | 0.20 | | 4.7 | 4.7 | |
| Cyno #1 | | | | | | |
| RD1624 | 0.50 | 0.29 | | 4.2 | 4.5 | |
| Cyno #2 | | | | | | |
| RD1625 | 0.19 | | 0.21 | 4.4 | | 4.5 |
| Cyno #1 | | | | | | |
| RD1625 | 0.27 | | 0.46 | 4.4 | | 4.7 |
| Cyno #2 | | | | | | |
| RD1626 | 0.39 | 0.12 | | 3.8 | 3.9 | |
| Cyno #1 | | | | | | |
| RD1626 | 0.30 | 0.18 | | 4.7 | 4.6 | |
| Cyno #2 | | | | | | |

TABLE 39
Kidney Function Markers

| | Bun | Bun | Bun | Creatinine | Creatinine | Creatinine |
|---|---|---|---|---|---|---|
| | Day −6 | Day 57 | Day 92 | Day −6 | Day 57 | Day 92 |
| Compound | [mg/dL] | [mg/dL] | [mg/dL] | [mg/dL] | [mg/dL] | [mg/dL] |
| RD1613 | 34 | 27 | | 0.44 | 0.49 | |
| Cyno #1 | | | | | | |
| RD1613 | 33 | 34 | | 0.55 | 0.54 | |
| Cyno #2 | | | | | | |
| RD1614 | 27 | | 55 | 0.61 | | 0.71 |
| Cyno #1 | | | | | | |
| RD1614 | 29 | | 26 | 0.55 | | 0.66 |
| Cyno #2 | | | | | | |
| RD1615 | 20 | | 31 | 0.44 | | 0.55 |
| Cyno #1 | | | | | | |
| RD1615 | 35 | | 26 | 0.33 | | 0.32 |
| Cyno #2 | | | | | | |
| RD1622 | 23 | 25 | | 0.70 | 0.63 | |
| Cyno #1 | | | | | | |
| RD1622 | 29 | 27 | | 0.53 | 0.38 | |
| Cyno #2 | | | | | | |
| RD1623 | 28 | | 28 | 0.49 | | 0.48 |
| Cyno #1 | | | | | | |
| RD1623 | 23 | | 25 | 0.47 | | 0.46 |
| Cyno #2 | | | | | | |
| RD1624 | 36 | 29 | | 0.71 | 0.55 | |
| Cyno #1 | | | | | | |
| RD1624 | 29 | 27 | | 0.42 | 0.48 | |
| Cyno #2 | | | | | | |
| RD1625 | 32 | | 40 | 0.61 | | 0.76 |
| Cyno #1 | | | | | | |
| RD1625 | 31 | | 24 | 0.55 | | 0.54 |
| Cyno #2 | | | | | | |
| RD1626 | 38 | 29 | | 0.44 | 0.52 | |
| Cyno #1 | | | | | | |
| RD1626 | 26 | 24 | | 0.46 | 0.50 | |
| Cyno #2 | | | | | | |

### Example 6: Effect of Compounds Targeting Human AGT in Cynomolgus Monkeys

Compounds of interest were evaluated in cynomolgus monkeys. Prior to the study the monkeys were kept in quarantine during which the animals were observed daily for general health. The monkeys were 3.86-4.28 years old and weighed 2.98-3.92 kg. Two groups of 2 cynomolgus monkeys each were injected with a single 5 mg/kg subcutaneous dose of oligonucleotide on Day 1 of the study. All animals were bled on day −8 and on days 1 (prior to dosing), 4, 8, 15, 22, 27, 36, 43, 50, and 57 for serum analysis. The protocols described were approved by the Institutional Animal Care and Use Committee (IACUC). Circulating AGT levels were quantified using an ELISA specific for human angiotensinogen (and cross-reactive with cynomolgus), according to manufacturer's protocol (IBL America #27412). AGT inhibition data were expressed as percent of baseline value (Day 1 prior to dosing) and presented as a group average for each compound. There were no test article-related effects on body weight.

TABLE 40
Average AGT Inhibition

| | Days | | | | | | | | |
|---|---|---|---|---|---|---|---|---|---|
| Compound | 4 | 8 | 15 | 22 | 27 | 36 | 43 | 50 | 57 |
| RD1623 | 21 | 40 | 73 | 83 | 84 | 81 | 81 | 81 | 85 |
| RD1625 | 38 | 63 | 77 | 80 | 81 | 80 | 80 | 76 | 79 |

TABLE 41
Body Weight (kg)

| Compound | Day 1 | Day 57 |
|---|---|---|
| RD1623 Cyno#1 | 3.76 | 3.83 |
| RD1623 Cyno#2 | 2.98 | 3.15 |
| RD1623 Cyno#3 | 3.92 | 4.54 |
| RD1625 Cyno#1 | 3.40 | 3.44 |
| RD1625 Cyno#2 | 3.22 | 3.32 |
| RD1625 Cyno#3 | 2.98 | 3.16 |

### Example 7: Effect of Compounds Targeting Human AGT in Cynomolgus Monkeys

Compounds of interest were evaluated in cynomolgus monkeys. Prior to the study the monkeys were kept in quarantine during which the animals were observed daily for general health. The monkeys were 3-5.6 years old and weighed 2.8-3.4 kg. Two groups of 2 cynomolgus monkeys each were injected with a single 3 mg/kg subcutaneous dose of oligonucleotide on Day 1 of the study. All animals were bled on day −6 and on days 1 (prior to dosing), 7, 14, 21, 28, 36 and 42 for serum analysis. The protocols described were approved by the Institutional Animal Care and Use Committee (IACUC). Circulating AGT levels were quantified using an ELISA specific for human angiotensinogen (and cross-reactive with cynomolgus), according to manufacturer's protocol (IBL America #27412). AGT inhibition data were expressed as percent of baseline value (Day 1 prior to dosing) and presented as a group average for each compound. There were no test article-related effects on body weight.

TABLE 42
Compound Chemistry

| | | | SEQ |
|---|---|---|---|
| Compound | | Ref ID | ID |
| Number | Modified Strand (5′-3′) | NO: | NO: |
| RD2052 | mU*fG*mA.fU.mC.fA.mU.fA.mC.fA.mC.fA.mG.fU.mA.fA.mA.fC.mA.fC.mC*mG*mG | IA0727 | 136 |
| | H2.mC*mC*mG.mG.mU.mG.mU.mU.fU.mA.fC.fU.fG.fU.mG.mU.mA.mU.mG.mA.mU.mC* | IS0833 | 297 |
| | mA*dQ | | |
| RD2053 | mU*fG*mA.fU.mC.fA.mU.fA.mC.fA.mC.fA.mG.fU.mA.fA.mA.fC.mA.fG.mC*mG*mG | IA0728 | 137 |
| | H2.mC*mC*mG.mC.mU.mG.mU.mU.fU.mA.fC.fU.fG.fU.mG.mU.mA.mU.mG.mA.mU.mC* | IS0834 | 298 |
| | mA*dQ | | |

TABLE 43
Average AGT Inhibition

| | Days | | | | |
|---|---|---|---|---|---|
| Compound | 7 | 14 | 21 | 28 | 42 |
| RD2052 | 30 | 52 | 63 | 55 | 41 |
| RD2053 | 26 | 43 | 62 | 49 | 51 |

TABLE 44
Body Weight (kg)

| Compound | Day 1 | Day 42 |
|---|---|---|
| RD2052 Cyno #1 | 3.0 | 3.4 |
| RD2052 Cyno #2 | 2.8 | 2.9 |
| RD2053 Cyno #1 | 3.1 | 3.4 |
| RD2053 Cyno #2 | 3.4 | 3.4 |

### Example 8: Effect of Modified Oligonucleotides Targeting Human AGT in Cynomolgus Monkeys

Compounds of interest were evaluated in cynomolgus monkeys. Prior to the study the monkeys were kept in quarantine during which the animals were observed daily for general health. The monkeys were 2.9-6.1 years old and weighed 2.4-3.5 kg. Three groups of 2 cynomolgus monkeys each were injected with a single 3 mg/kg subcutaneous dose of oligonucleotide on Day 1 of the study. All animals were bled on day −6 and on days 1 (prior to dosing), 4, 8, 15, 22, 29, 36, 43, 50, 57 and 64 for serum analysis. The protocols described were approved by the Institutional Animal Care and Use Committee (IACUC). Circulating AGT levels were quantified using an ELISA specific for human angiotensinogen (and cross-reactive with cynomolgus), according to manufacturer's protocol (IBL America #27412). AGT inhibition data were expressed as percent of baseline value (average of Day −6 and Day 1 prior to dosing) and presented as a group average for each compound. Clinical chemistry was performed on Day −6 and 64. There were no test article-related effects on body weight and all serum chemistry values were generally within reference ranges.

TABLE 45
Compound Chemistry

| | | | SEQ |
|---|---|---|---|
| Compound | | Ref ID | ID |
| Number | Modified Strand (5′-3′) | NO: | NO: |
| RD2263 | mU*fG*mA.fU.mC.fA.mU.fA.mC.fA.mC.fA.mG.fU.mA.fA.mA.fC.mA.fG*mG | IA0443 | 134 |
| | H4.mC*mC*mU.mG.mU.mU.fU.mA.fC.fU.fG.fU.mG.mU.mA.mU.mG.mA.mU.mC*mA*dQ | IS0970 | 295 |
| RD2264 | mU*fG*mA.fU.mC.fA.mU.fA.mC.fA.mC.fA.mG.fU.mA.fA.mA.fC.mA.fG.mG*mG*mA | IA0501 | 135 |
| | H4.mC*mC*mC.mU.mG.mU.mU.fU.mA.fC.fU.fG.fU.mG.mU.mA.mU.mG.mA.mU.mC*mA*dQ | IS0971 | 296 |
| RD2265 | mU*fG*mA.fU.mC.fA.mU.fA.mC.fA.mC.fA.mG.fU.mA.fA.mA.fC.mA.fG.mG*mA*mA | IA0500 | 133 |
| | H4.mC*mC*mU.mG.mU.mU.fU.mA.fC.fU.fG.fU.mG.mU.mA.mU.mG.mA.mU.mC*mA*dQ | IS0970 | 292 |

TABLE 46
Average AGT Inhibition

| | Days | | | | | | | | | |
|---|---|---|---|---|---|---|---|---|---|---|
| Compound | 4 | 8 | 15 | 22 | 29 | 36 | 43 | 50 | 57 | 64 |
| RD2263 | 12 | 21 | 49 | 54 | 55 | 47 | 47 | 33 | 34 | 34 |
| RD2264 | 7 | 36 | 50 | 48 | 49 | 48 | 44 | 34 | 40 | 33 |
| RD2265 | 11 | 37 | 58 | 61 | 59 | 57 | 58 | 46 | 43 | 36 |

TABLE 47
Body Weight (kg)

| Compound | Day 1 | Day 63 |
|---|---|---|
| RD2263 Cyno#1 | 3.5 | 3.4 |
| RD2263 Cyno#2 | 3.0 | 3.1 |
| RD2264 Cyno#1 | 3.0 | 2.8 |
| RD2264 Cyno#2 | 3.0 | 3.2 |
| RD2265 Cyno#1 | 2.6 | 2.7 |
| RD2265 Cyno#2 | 2.4 | 2.5 |

TABLE 48
Liver Function Markers

| | ALT | ALT | AST | AST | Bilirubin | Bilirubin | Albumin | Albumin |
|---|---|---|---|---|---|---|---|---|
| | Day 1 | Day 64 | Day 1 | Day 64 | Day 1 | Day 64 | Day 1 | Day 64 |
| Compound | [U/L] | [U/L] | [U/L] | [U/L] | [mg/dL] | [mg/dL] | [g/dL] | [g/dL] |
| RD2263 | 56 | 60 | 36 | 37 | 0.30 | 0.26 | 5.0 | 4.7 |
| Cyno#1 | | | | | | | | |
| RD2263 | 68 | 76 | 29 | 35 | 0.22 | 0.17 | 4.6 | 4.5 |
| Cyno#2 | | | | | | | | |
| RD2264 | 34 | 39 | 28 | 30 | 0.23 | 0.19 | 4.1 | 3.8 |
| Cyno#1 | | | | | | | | |
| RD2264 | 69 | 67 | 35 | 34 | 0.26 | 0.17 | 4.4 | 4.2 |
| Cyno#2 | | | | | | | | |
| RD2265 | 39 | 42 | 43 | 51 | 0.74 | 0.45 | 4.2 | 4.0 |
| Cyno#1 | | | | | | | | |
| RD2265 | 84 | 83 | 65 | 93 | 0.36 | 0.32 | 4.2 | 4.1 |
| Cyno#2 | | | | | | | | |

TABLE 49
Kidney Function Markers

| | Bun | Bun | Creatinine | Creatinine |
|---|---|---|---|---|
| | Day 1 | Day 64 | Day 1 | Day 64 |
| Compound | [mg/dL] | [mg/dL] | [mg/dL] | [mg/dL] |
| RD2263 Cyno#1 | 23 | 24 | 0.72 | 0.72 |
| RD2263 Cyno#2 | 24 | 24 | 0.66 | 0.68 |
| RD2264 Cyno#1 | 17 | 17 | 0.48 | 0.51 |
| RD2264 Cyno#2 | 17 | 15 | 0.37 | 0.34 |
| RD2265 Cyno#1 | 28 | 24 | 0.50 | 0.48 |
| RD2265 Cyno#2 | 21 | 16 | 0.40 | 0.36 |

### Example 9: Effect of Modified Oligonucleotides Targeting Human AGT in Cynomolgus Monkeys

Compounds of interest were evaluated in cynomolgus monkeys. Prior to the study the monkeys were kept in quarantine during which the animals were observed daily for general health. The monkeys were 2.9-6.1 years old and weighed 2.4-3.5 kg. Two groups of 2 cynomolgus monkeys each were injected with a single 3 mg/kg subcutaneous dose of oligonucleotide on Day 1 of the study. All animals were scheduled to be bled on day −6 and on days 1 (prior to dosing), 8, 15, 22, 29, 36, 43, 50, 57 and 64 for serum analysis. The study was ended after day 29. The protocols described were approved by the Institutional Animal Care and Use Committee (IACUC). Circulating AGT levels were quantified using an ELISA specific for human angiotensinogen (and cross-reactive with cynomolgus), according to manufacturer's protocol (IBL America #27412). AGT inhibition data is expressed as percent of baseline value (average of Day −6 and Day 1 prior to dosing) and presented as a group average for each compound. Clinical chemistry was scheduled to be performed on Day −6 and 64.

TABLE 50
Compound Chemistry

| | | | SEQ |
|---|---|---|---|
| Compound | | Ref ID | ID |
| Number | Modified Strand (5′-3′) | NO: | NO: |
| RD2497 | mU*fC*mA.fA.mG.fA.mA.fG.mU.fU.mG.fG.mC.fC.mA.fG.mC.fA.mU.fC.mC*mG*mC | IA0868 | 32 |
| | H2*mG*mG.mA.mU.mG.mC.fU.mG.fG.fC.fC.fA.mA.mC.mU.mU.mC.mU.mU.mG*mA*dQ | IS1078 | 190 |
| RD2498 | mU*fU*mU.fU.mG.fC.mU.fG.mG.fA.mA.fA.mG.fU.mG.fA.mG.fA.mC.fC.mC*mU*mC | IA0869 | 114 |
| | H2*mG*mG.mG.mU.mC.mU.fC.mA.fC.fU.fU.fU.mC.mC.mA.mG.mC.mA.mA.mA*mA*dQ | IS1079 | 273 |

TABLE 51
Average AGT Inhibition

Days

| Compound | 8 | 15 | 22 | 29 |
|---|---|---|---|---|
| RD2497 | 10 | 6 | 12 | 9 |
| RD2498 | 40 | 56 | 42 | 35 |

### Example 10: Effect of Modified Oligonucleotides Targeting Human AGT in Cynomolgus Monkeys

Compounds of interest were evaluated in cynomolgus monkeys. Prior to the study the monkeys were kept in quarantine during which the animals were observed daily for general health. The monkeys were 2.9-6.1 years old and weighed 2.4-3.5 kg. Two groups of 2 cynomolgus monkeys each were injected with a single 3 mg/kg subcutaneous dose of oligonucleotide on Day 1 of the study. All animals were scheduled to be bled on day −6 and on days 1 (prior to dosing), 4, 8, 15, 22, 29, 36, 43, 50, 57 and 64 for serum analysis. The study was ended after day 43. The protocols described were approved by the Institutional Animal Care and Use Committee (IACUC). Circulating AGT levels were quantified using an ELISA specific for human angiotensinogen (and cross-reactive with cynomolgus), according to manufacturer's protocol (IBL America #27412). AGT inhibition data is expressed as percent of baseline value (average of Day −6 and Day 1 prior to dosing) and presented as a group average for each compound. Clinical chemistry was scheduled to be performed on Day −6 and 64.

TABLE 52
Compound Chemistry

| | | | SEQ |
|---|---|---|---|
| Compound | | Ref ID | ID |
| Number | Modified Strand (5′-3′) | NO: | NO: |
| RD2499 | mA*fG*mU.fU.mU.fC.mU.fU.mC.fA.mU.fC.mC.fA.mG.fU.mU.fG.mA.fG.mG*mG*mA | IA0870 | 115 |
| | H9*mC*mC.mU.mC.mA.mA.fC.mU.fG.fG.fA.fU.mG.mA.mA.mG.mA.mA.mA.mC*mU*dQ | IS1080 | 274 |
| RD2500 | mU*fC*mU.fA.mA.fA.mC.fA.mC.fU.mG.fG.mU.fU.mC.fU.mU.fG.mC.fC.mU*mC*mC | IA0871 | 151 |
| | H2*mA*mG.mG.mC.mA.mA.fG.mA.fA.fC.fC.fA.mG.mU.mG.mU.mU.mU.mA.mG*mA*dQ | IS1081 | 311 |

TABLE 53
Average AGT Inhibition

| | Days | | | | | | |
|---|---|---|---|---|---|---|---|
| Compound | 4 | 8 | 15 | 22 | 29 | 36 | 43 |
| RD2499 | 23 | 34 | 38 | 41 | 52 | 55 | 33 |
| RD2500 | 26 | 34 | 29 | 39 | 59 | 51 | 36 |

SEQ ID NO: 1
GAAGAAGCTGCCGTTGTTCTGGGTACTACAGCAGAAGGGTATGCGGAAGCGAGCACCCCAGTCTGAGATGGCTCCTGCC
GGTGTGAGCCTGAGGGCCACCATCCTCTGCCTCCTGGCCTGGGCTGGCCTGGCTGCAGGTGACCGGGTGTACATACACCC
CTTCCACCTCGTCATCCACAATGAGAGTACCTGTGAGCAGCTGGCAAAGGCCAATGCCGGGAAGCCCAAAGACCCCACCT
TCATACCTGCTCCAATTCAGGCCAAGACATCCCCTGTGGATGAAAAGGCCCTACAGGACCAGCTGGTGCTAGTCGCTGCA
AAACTTGACACCGAAGACAAGTTGAGGGCCGCAATGGTCGGGATGCTGGCCAACTTCTTGGGCTTCCGTATATATGGCAT
GCACAGTGAGCTATGGGGCGTGGTCCATGGGGCCACCGTCCTCTCCCCAACGGCTGTCTTTGGCACCCTGGCCTCTCTCTA
TCTGGGAGCCTTGGACCACACAGCTGACAGGCTACAGGCAATCCTGGGTGTTCCTTGGAAGGACAAGAACTGCACCTCCC
GGCTGGATGCGCACAAGGTCCTGTCTGCCCTGCAGGCTGTACAGGGCCTGCTAGTGGCCCAGGGCAGGGCTGATAGCCA
GGCCCAGCTGCTGCTGTCCACGGTGGTGGGCGTGTTCACAGCCCCAGGCCTGCACCTGAAGCAGCCGTTTGTGCAGGGC
CTGGCTCTCTATACCCCTGTGGTCCTCCCACGCTCTCTGGACTTCACAGAACTGGATGTTGCTGCTGAGAAGATTGACAGG
TTCATGCAGGCTGTGACAGGATGGAAGACTGGCTGCTCCCTGATGGGAGCCAGTGTGGACAGCACCCTGGCTTTCAACAC
CTACGTCCACTTCCAAGGGAAGATGAAGGGCTTCTCCCTGCTGGCCGAGCCCCAGGAGTTCTGGGTGGACAACAGCACCT
CAGTGTCTGTTCCCATGCTCTCTGGCATGGGCACCTTCCAGCACTGGAGTGACATCCAGGACAACTTCTCGGTGACTCAAG
TGCCCTTCACTGAGAGCGCCTGCCTGCTGCTGATCCAGCCTCACTATGCCTCTGACCTGGACAAGGTGGAGGGTCTCACTT
TCCAGCAAAACTCCCTCAACTGGATGAAGAAACTATCTCCCCGGACCATCCACCTGACCATGCCCCAACTGGTGCTGCAAG
GATCTTATGACCTGCAGGACCTGCTCGCCCAGGCTGAGCTGCCCGCCATTCTGCACACCGAGCTGAACCTGCAAAAATTG
AGCAATGACCGCATCAGGGTGGGGGAGGTGCTGAACAGCATTTTTTTTGAGCTTGAAGCGGATGAGAGAGAGCCCACAG
AGTCTACCCAACAGCTTAACAAGCCTGAGGTCTTGGAGGTGACCCTGAACCGCCCATTCCTGTTTGCTGTGTATGATCAAA
GCGCCACTGCCCTGCACTTCCTGGGCCGCGTGGCCAACCCGCTGAGCACAGCATGAGGCCAGGGCCCCAGAACACAGTG
CCTGGCAAGGCCTCTGCCCCTGGCCTTTGAGGCAAAGGCCAGCAGCAGATAACAACCCCGGACAAATCAGCGATGTGTCA
CCCCCAGTCTCCCACCTTTTCTTCTAATGAGTCGACTTTGAGCTGGAAAGCAGCCGTTTCTCCTTGGTCTAAGTGTGCTGCA
TGGAGTGAGCAGTAGAAGCCTGCAGCGGCACAAATGCACCTCCCAGTTTGCTGGGTTTATTTTAGAGAATGGGGGTGGG
GAGGCAAGAACCAGTGTTTAGCGCGGGACTACTGTTCCAAAAAGAATTCCAACCGACCAGCTTGTTTGTGAAACAAAAAA
GTGTTCCCTTTTCAAGTTGAGAACAAAAATTGGGTTTTAAAATTAAAGTATACATTTTTGCATTGCCTTCGGTTTGTATTTA
GTGTCTTGAATGTAAGAACATGACCTCCGTGTAGTGTCTGTAATACCTTAGTTTTTTCCACAGATGCTTGTGATTTTTGAAC
AATACGTGAAAGATGCAAGCACCTGAATTTCTGTTTGAATGCGGAACCATAGCTGGTTATTTCTCCCTTGTGTTAGTAATA
AACGTCTTGCCACAATAAGCCTCCAAAAA

What is claimed is:

1. A compound comprising a modified oligonucleotide 14 to 23 linked nucleosides in length having a nucleobase sequence comprising at least 14, at least 15, at least 16, at least 17, at least 18, at least 19, at least 20 contiguous nucleobases of any of the nucleobase sequences of SEQ ID NOs: 10-166.

2. A compound comprising a modified oligonucleotide 14 to 23 linked nucleosides in length having a nucleobase sequence comprising the nucleobase sequence of any one of SEQ ID NOs: 10-166.

3. A compound comprising a modified oligonucleotide having a nucleobase sequence selected from any one of the nucleobase sequences of SEQ ID NOs: 10-166.

4. The compound of any one of claims 1-3, wherein the nucleobase sequence of the modified oligonucleotide is at least 80%, at least 85%, at least 90%, or at least 95% complementary to the nucleobase sequence of SEQ ID NO: 1 or 3.

5. The compound of any one of claims 1-4, wherein the modified oligonucleotide comprises at least one modification selected from a modified internucleoside linkage, a modified sugar, and a modified nucleobase.

6. The compound of any one of claims 1-5, wherein the compound is double-stranded.

7. A compound comprising a first modified oligonucleotide 14 to 23 linked nucleosides in length having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-166 and 167-327 and a second modified oligonucleotide 14 to 23 linked nucleosides in length having a region of complementarity to the first modified oligonucleotide.

8. A compound comprising a first modified oligonucleotide 14 to 23 linked nucleosides in length having a nucleobase sequence comprising the nucleobase sequence of any one of SEQ ID NOs: 10-166 or 167-327 and a second modified oligonucleotide 14 to 23 linked nucleosides in length having a region of complementarity to the first modified oligonucleotide.

9. A compound comprising a first modified oligonucleotide having a nucleobase sequence selected from the nucleobase sequences of any one of SEQ ID NOs: 10-166 and 167-327 and a second modified oligonucleotide 19 to 23 linked nucleosides in length having a region of complementarity to the first modified oligonucleotide.

10. The compound of any one of claims 7-9, wherein the nucleobase sequence of the first modified oligonucleotide has at least 80%, at least 85%, at least 90%, or at least 95% complementarity or identity to the nucleobase sequence of SEQ ID NO: 1 or 3 over its length.

11. The compound of any one of claims 7-10, wherein the nucleobase sequence of the first modified oligonucleotide has at least 1, at least 2, at least 3 mismatches to a region of the nucleobase sequence of SEQ ID NO: 1 or 3 that is the same length as the first modified oligonucleotide.

12. The compound of any one of claims 7-11, wherein the region of complementarity between the first modified oligonucleotide and the second modified oligonucleotide is 14 to 23 linked nucleosides in length.

13. The compound of any one of claims 7-11, wherein the region of complementarity between the first modified oligonucleotide and the second modified oligonucleotide is 19 to 23 linked nucleosides in length.

14. The compound of any one of claims 7-11, wherein the region of complementarity between the first modified oligonucleotide and the second modified oligonucleotide is 21 to 23 linked nucleosides in length.

15. The compound of any one of claims 7-11, wherein the first modified oligonucleotide is fully complementary to the second modified oligonucleotide.

16. The compound of any one of claims 7-15, wherein the first modified oligonucleotide comprises at least one modification selected from a modified internucleoside linkage, a modified sugar, and a modified nucleobase.

17. The compound of any one of claims 7-16, wherein the second modified oligonucleotide comprises at least one modification selected from the group consisting of a modified internucleoside linkage, a modified sugar, and a modified nucleobase.

18. The compound of any one of claims 5, 16 and 17, wherein the modified internucleoside linkage is a phosphorothioate internucleoside linkage or a methylphosphonate internucleoside linkage.

19. The compound of claim 18, wherein the phosphorothioate internucleoside linkage or methylphosphonate internucleoside linkage is at the 3′ terminus of the first or second modified oligonucleotide or at the 5′ terminus of the first modified oligonucleotide.

20. The compound of any one of claims 5, 16 and 17, wherein the modified sugar comprises a modification selected from the group consisting of a halogen, an alkoxy group and a bicyclic sugar.

21. The compound of claim 20, wherein the modified sugar comprises a 2′-F modification.

22. The compound of claim 20, wherein the modified sugar comprises a 2′-OMe modification.

23. The compound of any one of claims 7-15, wherein each nucleoside of the first modified oligonucleotide comprises a modified sugar.

24. The compound of any one of claims 7-15, wherein each nucleoside of the second modified oligonucleotide comprises a modified sugar.

25. The compound of claim 23 or 24, wherein the modified sugar comprises a modification selected from the group consisting of a halogen, an alkoxy group and a bicyclic sugar, or a combination thereof.

26. The compound of claim 25, wherein the modified sugar comprises a modification selected from group consisting of LNA, cEt, 2′-MOE, 2′-F, 2′-OMe, and 2′-deoxy, or a combination thereof.

27. The compound of claim 23, wherein the first modified oligonucleotide comprises no more than ten 2′-F sugar modifications.

28. The compound of claim 24, wherein the second modified oligonucleotide comprises no more than five 2′-F sugar modifications.

29. The compound of any preceding claim, comprising a conjugate group.

30. The compound of claim 29, wherein the conjugate group is attached to the 5′ end of the modified oligonucleotide.

31. The compound of claim 29 or 30, wherein the conjugate group comprises a targeting moiety.

32. The compound of claim 31, wherein the targeting moiety comprises one or more GalNAc.

33. The compound of claim 32, wherein the modified oligonucleotide is the second modified oligonucleotide.

34. The compound of claim 32 or 33, wherein the one or more GalNAc are attached to the 2′ or 3′ position of the ribosyl ring of the 5′ nucleoside of the modified oligonucleotide.

35. The compound of claim 34, wherein the 5′ nucleoside is of the following formula: 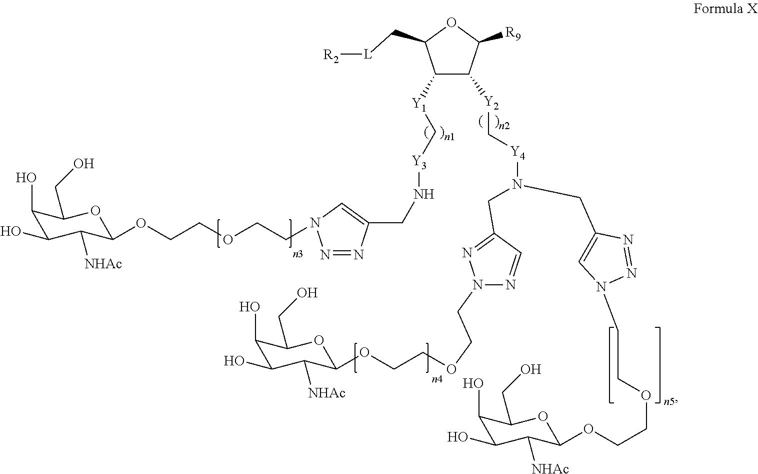
wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
L is a bond, a phosphodiester bond, a phosphorothioate bond, a triazole, a tetrazole, an amide, a reverse-amide, a carbamate, a carbonate, urea, alkyl, or heteroalkyl;
R² is the oligonucleotide sequence;
Y₁ is O, CH₂, CH₂O, or optionally substituted NH;
Y₂ is O, CH₂, CH₂O, or optionally substituted NH;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6; and
each n₁, n₃, n₄ and n₅ is independently 0, 1, 2, 3, 4, 5, 6, 7, 8, 9, or 10.

36. The compound of claim 34, wherein the 5′ nucleoside is selected from any one of Formulae I-VIII and wherein R′ is S and R is the portion of the modified oligonucleotide other than the 5′ nucleoside.

37. The compound of claim 34, wherein the 5′ nucleoside is selected from any one of Formulae I-VIII and wherein R′ is O and R is the portion of the modified oligonucleotide other than the 5′ nucleoside.

38. A compound comprising a first modified oligonucleotide selected from any one of Ref ID NOs: IA0297, IA0300, IA0301, IA0304, IA0305, IA0335-338, IA0343-359, IA0431-432, IA0435, IA440-446, IA0727-728, IA0500-501, and IA0868, and a second modified oligonucleotide 14 to 21 linked nucleosides in length fully complementary to the first modified oligonucleotide.

39. A compound comprising a first modified oligonucleotide selected from Ref ID NOs: IA0443 and IA0445 and a second modified oligonucleotide selected from Ref ID NOs: IS0505 and IS0509.

40. A compound of any one of claims 1-39, wherein the compound is in a pharmaceutically acceptable salt form.

41. The compound of claim 40, wherein the pharmaceutically acceptable salt is a sodium salt.

42. The compound of claim 40, wherein the pharmaceutically acceptable salt is a potassium salt.

43. A composition comprising the compound of any one of claims 1-42 and a pharmaceutically acceptable carrier.

44. A composition comprising a compound of any preceding claim, for use in therapy.

45. A method of treating, preventing or ameliorating a disease, disorder or condition associated with AGT in an individual comprising administering to the individual a compound targeted to AGT, thereby treating, preventing, or ameliorating the disease, disorder or condition.

46. A method of administering the compound of any one of claims 1-42 or composition of claim 43 or 44 to an individual.

47. The method of claim 45 or 46, wherein the disease, disorder or condition is RAAS related disease, disorder or condition or a symptom thereof, hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment.

48. The method of any one of claims 45-47, wherein administering the compound inhibits or reduces or improves RAAS related disease, disorder or condition or a symptom thereof, hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment.

49. A method of inhibiting expression of AGT in a cell comprising contacting the cell with a compound targeted to AGT, thereby inhibiting expression of AGT in the cell.

50. The method of claim 49, wherein the cell is in the liver of an individual.

51. The method of claim 50, wherein the individual has, or is at risk of having, RAAS related disease, disorder or condition or a symptom thereof, hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment.

52. A method of reducing or inhibiting RAAS related disease, disorder or condition or a symptom thereof, hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment in an individual, comprising administering a compound targeted to AGT to the individual, thereby reducing or inhibiting RAAS related disease, disorder or condition or a symptom thereof, hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment in the individual.

53. The method of claim 52, wherein the individual has, or is at risk of having, RAAS related disease, disorder or condition or a symptom thereof, hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment.

54. The method of any one of claims 45-53, wherein the compound is a compound targeted to AGT.

55. The method of any one of claims 45-54, wherein the compound is the compound of any one of claims 1-42 or composition of claim 43 or 44.

56. The method of claim 55, wherein the compound or composition is administered parenterally.

57. Use of a compound targeted to AGT for treating, preventing, or ameliorating a disease, disorder or condition associated with AGT.

58. The use of claim 57, wherein the disease, disorder or condition is a RAAS related disease, disorder or condition or a symptom thereof, hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment.

59. The use of claim 57 or 58, wherein the compound is a compound targeted to AGT.

60. The use of any one of claims 57-59, wherein the compound is the compound of any one of claims 1-42 or composition of claim 43 or 44.

61. Use of a compound targeted to AGT in the manufacture of a medicament for treating, preventing, or ameliorating a disease, disorder or condition associated with AGT.

62. The use of claim 61, wherein the disease is a RAAS related disease, disorder or condition or a symptom thereof, hypertension, resistant hypertension, fibrosis, kidney disease, chronic kidney disease, cardiovascular disease (e.g., coronary heart disease, heart failure, stroke, myocardial infarction, atrial fibrillation, aneurysm and peripheral artery disease), organ damage (e.g., heart, liver or kidney), inflammatory bowel disease or cognitive impairment.

63. The use of claim 61 or 62, wherein the compound is a compound targeted to AGT.

64. The use of any one of claims 61-63, wherein the compound is the compound of any one of claims 1-42 or composition of claim 43 or 44.

65. The method or use of any preceding claim, wherein the compound or composition is administered to an individual about once every three months to about once every year.

66. The method or use of any preceding claim, wherein the compound or composition is administered to an individual about once every three months, about once every six months, or about once every year.
